(19) United States
(12) Patent Application Publication
(10) Pub. No.: US 2026/0248800 A1
(43) Pub. Date: Aug. 27, 2026

(54) FERROPTOSIS INHIBITORS AND USES THEREOF

(71) Applicant: UNIVERSITY OF OTTAWA, Ottawa (CA)

(72) Inventors: Derek Andrew PRATT, Ottawa (CA); Luke Anthony FARMER, Ottawa (CA)

(21) Appl. No.: 18/874,660

(22) PCT Filed: Jun. 22, 2023
(86) PCT No.: PCT/CA2023/050872

§ 371 Date: Dec. 13, 2024

Related U.S. Application Data
(60) Provisional application No. 63/355,021, filed on Jun. 23, 2022.

(51) Int. Cl. A61K 31/538; A61P 39/06; C07D 265/38; C07D 413/04

(52) U.S. Cl. CPC A61K 31/538; A61P 39/06; C07D 265/38; C07D 413/04

(57) ABSTRACT

There are provided ferroptosis inhibitor compounds, and compositions and methods of use thereof for the prevention or treatment of a disease or disorder associated with ferroptosis, such as a neurodegenerative disease or ischemic reperfusion injury. Compounds of Formula A, and pharmaceutically acceptable salts thereof, are provided: Formula A

### CROSS REFERENCE TO RELATED APPLICATIONS

This application claims the benefit of priority from U.S. Provisional Application No. 63/355,021, filed on Jun. 23, 2022, which is hereby incorporated by reference in its entirety.

### FIELD

The present disclosure relates to the field of iron-dependent, non-apoptotic regulated cell death. There are provided inhibitors of ferroptosis, and compositions and methods of use thereof for inhibiting undesired cell death associated with diseases and disorders such as neurodegeneration and ischemic-reperfusion injury.

### BACKGROUND

Ferroptosis is an iron-dependent form of cell death resulting from a lethal accumulation of (phospho)lipid hydroperoxides (Jiang et al., 2021; Stockwell et al., 2020; Conrad and Pratt, 2019). Subsequent fragmentation of the hydroperoxides is associated with loss of plasma membrane integrity, cell swelling and eventual osmolysis of cells (Riegman et al., 2020). Ferroptosis has been linked to a variety of pathologies, including neurodegeneration and ischemic reperfusion injury (Stockwell et al., 2017; Masaldan et al., 2019). It is triggered when the cell's ability to suppress the formation of (phospho)lipid hydroperoxides is overcome. Phospholipid peroxidation can be enzyme-catalyzed (by lipoxygenases) or can occur spontaneously via autoxidation, a radical chain reaction (FIG. 1(A)) (Haeggstrom et al., 2011; Yin et al., 2011; Zielinski et al., 2017; Helberg et al., 2021). The latter process is auto-initiated since product hydroperoxides can yield initiating radicals in the presence of low-valent iron (or other good one-electron reductants). The activity of glutathione peroxidase-4 (GPX4), a selenoprotein that catalyzes the reduction of (phospho)lipid hydroperoxides to their corresponding alcohols by glutathione (GSH) precludes this step. Small molecules which chelate free iron or possess GPX4-like peroxidase activity can also be effective (Dixon et al., 2012).

In the initial characterization of ferroptosis, phenolic radical-trapping antioxidants (RTAs), such as BHT and Trolox, a water-soluble analog of α-tocopherol (α-TOH, the most biologically active form of Vitamin E), were identified as being effective suppressors (Dixon et al., 2012). RTAs trap (phospho)lipid-derived peroxyl radicals, precluding the propagation of the autoxidation chain reaction leading to the accumulation of (phospho)lipid hydroperoxides (FIGS. 1(B), 1(C)). This key observation linked lipid autoxidation to ferroptosis, a result which has since been underscored by the fact that lipoxygenase activity is not required for ferroptosis and that the most potent ferroptosis inhibitors identified to date are RTAs. For example, ferrostatin-1 (Fer-1) and liproxstatin-1 (Lip-1) (Friedmann et al., 2014) arylamines uncovered by high-through-put screening to be potent ferroptosis inhibitors, were subsequently found to be very good RTAs (FIG. 1(C)) (Zilka et al., 2017). Moreover, the recent discoveries of biochemical pathways which regenerate and/or synthesize RTAs de novo to suppress ferroptosis (e.g. FSP1/reduced coenzyme Q10 and GCH1/tetrahydro-biopterin, respectively) (Doll et al., 2019; Soula et al., 2020) further highlight the central role of lipid peroxidation in ferroptosis and of RTAs to keep it in check.

In the decades preceding the characterization of ferroptosis, the RTA activity of α-TOH (and other forms of Vitamin E) was meticulously studied in organic solutions, micelles, phospholipid bilayers and lipoproteins. On the basis of that work, it was evident that α-TOH acts by trapping (phospho)lipidperoxyl radicals that are inaccessible to ubiquitous water-soluble RTAs such as uric acid and ascorbic acid. The principal reasons it functions well in this capacity are: (1) it is highly lipophilic and spontaneously partitions into biological membranes, (2) it is significantly more reactive than (phospho)lipids to propagating peroxyl radicals, and (3) it is highly bioavailable, with its own transport protein to provide protection from primary metabolism and ensure systemic distribution. Despite the concentration of autoxidizable (phospho)lipids being anywhere from 2 to 3 orders of magnitude greater than α-TOH in lipid bilayers, it remains quite effective at supressing chain propagation because its inhibition rate constant (kᵢₙₕ) is orders of magnitude greater than the propagation rate constants (kₚ) of the (phospho)lipids (ranging from 1-320 M⁻¹s⁻¹ depending on the level of unsaturation). However, the margin by which α-TOH is more reactive than the substrates it serves to protect is strongly dependent on the environment. For example, in the inhibited autoxidation of styrene in chlorobenzene, kᵢₙₕ=3×10⁶ M⁻¹s⁻¹ (Burton et al., 1985), but in dilinoleoyl phosphatidylcholine (PC) liposomes, it is supressed ~500-fold to 6×10³ M⁻¹s⁻¹ (Barclay et al., 1990). We recently provided evidence that this suppression in reactivity is due primarily to H-bond formation between the phenolic O—H and phosphodiester moieties of the phospholipids (Shah et al., 2019).

Phenoxazine (PNX) has been shown to inhibit ferroptosis or neuronal cell death (Shah et al., 2017; Mocko et al., 2010; Hajieva et al., 2009). However, this compound is metabolically labile, undergoing P450 mediated hydroxylation at the 3- and/or 7-positions to yield phenoxazone and resorufin derivatives (FIG. 5(D)) (Sutherland et al., 2001; Burke and Mayer, 1983). Previous attemps to derivatize PNX have resulted in substantial loss of ferroptosis inhibitory activity. For example, a 2,4-Aza-PNX derivative was shown to have significantly lower activity compared to PNX (Shah et al., 2017). Others have modified the PNX structure, however the modified PNX compounds all displayed less activity than the unsubstituted PNX compound for the activities tested (see e.g., Yu et al., 1991, studying lipoxygenase-5 (LO5) inhibition).

There is a need for improved ferroptosis inhibitor compounds for therapeutic use.

### SUMMARY

It is an object of the present invention to ameliorate at least some of the deficiencies present in the prior art. Embodiments of the present technology have been developed based on the inventors' appreciation that there is a need for ferroptosis inhibitor compounds.

In a first broad aspect, there are provided compounds of Formula A, or pharmaceutically acceptable salts thereof:

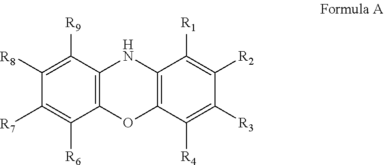

wherein: R₁, R₂, R₃, and R₄ are independently hydrogen (H), substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid, an ester, an amide, nitro (NO₂), fluoro (F), chloro (Cl), bromo (Br), iodo (I), formyl (CH(O)), acetyl (C(O)CH₃), a phosphonyl group, or a sulfonyl group; or R₁ and R₂, taken together with the atoms to which they are attached, form a substituted or unsubstituted carbocyclic ring; or R₂ and R₃, taken together with the atoms to which they are attached, form a substituted or unsubstituted carbocyclic ring; or R₃ and R₄, taken together with the atoms to which they are attached, form a substituted or unsubstituted carbocyclic ring; or R₆ and R₇, taken together with the atoms to which they are attached, form a substituted or unsubstituted carbocyclic ring; or R₇ and R₈, taken together with the atoms to which they are attached, form a substituted or unsubstituted carbocyclic ring; or R₈ and R₉, taken together with the atoms to which they are attached, form a substituted or unsubstituted carbocyclic ring; provided that R₁, R₂, R₃, R₄, R₆, R₇, R₈, and R₉ are not all H at the same time.

In some embodiments of compounds of Formula A, R₁ and R₄ are hydrogen.

In some embodiments of compounds of Formula A, R₁, R₃ and R₄ are hydrogen.

In some embodiments of compounds of Formula A, R₁, R₂ and R₄ are hydrogen.

In some embodiments of compounds of Formula A, at least one of R₇ and R₈ is trifluoromethyl (CF₃), a perfluoroalkyl (CₙF₂ₙ₊₁ where n is an integer from 1 to 6), nitro (NO₂), cyano (CN), sulfonamide (SO₂N(R)₂), trifluoromethyl ether (OCF₃), a perfluoroalkyl ether (—OCₘF₂ₘ₊₁ where m is an integer from 1 to 6), fluoro (F), chloro (Cl), bromo (Br), iodo (I), formyl (CH(O)), acetyl (C(O)CH₃), an ester, an amide, or a ketone;

In some embodiments of compounds of Formula A, at least one of R₇ and R₈ is trifluoromethyl (CF₃). In an embodiment, R₇ is trifluoromethyl (CF₃). In another embodiment, R₈ trifluoromethyl (CF₃).

In some embodiments of compounds of Formula A, at least one of R₇ and R₈ is trifluoromethyl (CF₃), and R₁ and R₄ are hydrogen.

In some embodiments of compounds of Formula A, at least one of R₇ and R₈ is trifluoromethyl (CF₃), and R₁, R₃, and R₄ are hydrogen.

In some embodiments of compounds of Formula A, at least one of R₇ and R₈ is trifluoromethyl (CF₃), and R₁, R₂, and R₄ are hydrogen.

In some embodiments of compounds of Formula A, at least one of R₂ and R₃ is not hydrogen (H). In some such embodiments, at least one of R₂ and R₃ is substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid. In some such embodiments, at least one of R₇ and R₈ is not hydrogen (H).

In some embodiments of compounds of Formula A, at least one of R₇ and R₈ is trifluoromethyl (CF₃), and at least one of R₂ and R₃ is not hydrogen (H). In some such embodiments, when at least one of R₇ and R₈ is trifluoromethyl (CF₃), at least one of R₂ and R₃ is substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid.

In an embodiment of compounds of Formula A, R₂ is substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid, and R₇ is not H.

In an embodiment of compounds of Formula A, R₂ is substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid, and R₈ is not H.

In an embodiment of compounds of Formula A, R₃ is substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid, and R₇ is not H.

In an embodiment of compounds of Formula A, R₃ is substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid, and R₈ is not H.

In some embodiments of compounds of Formula A, when at least one of R₇ and R₈ is an ester, R₂ is not an ester. In some embodiments of compounds of Formula A, when at least one of R₇ and R₈ is an amide, R₂ and/or R₃ is not an amide. In some embodiments of compounds of Formula A, when at least one of R₇ and R₈ is an amide, R₂ and/or R₃ is not an amide, and R₃ is not an acid. In some embodiments of compounds of Formula A, when at least one of R₇ and R₈ is an amide, R₂ and R₃ is not an amide, and R₃ is not an acid.

In some embodiments of compounds of Formula A, R₁ and R₂ are not COOH, COO—, or (CH₂)₂COO—.

In some embodiments of compounds of Formula A, when R₁, R₂ and R₄ are hydrogen, R₃ is not N(CH₂CH₃)₂.

In some embodiments of compounds of Formula A, when at least one of R₇ and R₈ is CF₃, R₃ is not also CF₃ and R₂ is not NO₂.

In some embodiments of compounds of Formula A, when at least one of R₇ and R₈ is CN, R₂ and R₃ are not also CN, and R₃ is not F.

In some embodiments of compounds of Formula A, when at least one of R₇ and R₈ is NO₂, R₂ and R₃ are not OCH₃.

In some embodiments of Formula A, at least one of R₇ and R₈ is trifluoromethyl (CF₃), and at least one of R₂ and R₃ is not hydrogen (H). In some such embodiments, at least one of R₂ and R₃ is substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid.

In some embodiments of compounds of Formula A, the compound demonstrates enhanced metabolic stability and/or ferroptosis inhibition activity, compared to unsubstituted phenoxazine compounds.

In some embodiments of compounds of Formula A, the compound of Formula A is a compound shown in Table 1, or a pharmaceutically acceptable salt thereof.

In some embodiments of compounds of Formula A, the compound of Formula A is a compound shown in Table 2, or a pharmaceutically acceptable salt thereof.

In some embodiments of compounds of Formula A, the compound of Formula A is compound 7, 8, 10, 11, 44, 46, 48, 49, 60, 66, 69, 72, 75, 78, 81, 84 or a pharmaceutically acceptable salt thereof.

In some embodiments of compounds of Formula A, the compound of Formula A is compound 11 or a pharmaceutically acceptable salt thereof.

In some embodiments of compounds of Formula A, the compound of Formula A is compound 44 or a pharmaceutically acceptable salt thereof.

In some embodiments of compounds of Formula A, the compound of Formula A is compound 46 or a pharmaceutically acceptable salt thereof.

In some embodiments of compounds of Formula A, the compound of Formula A is compound 48 or a pharmaceutically acceptable salt thereof.

In some embodiments of compounds of Formula A, the compound of Formula A is compound 60 or a pharmaceutically acceptable salt thereof.

In some embodiments of compounds of Formula A, the compound of Formula A is compound 66 or a pharmaceutically acceptable salt thereof.

In some embodiments of compounds of Formula A, the compound of Formula A is compound 69 or a pharmaceutically acceptable salt thereof.

In some embodiments of compounds of Formula A, the compound of Formula A is compound 72 or a pharmaceutically acceptable salt thereof.

In some embodiments of compounds of Formula A, the compound of Formula A is compound 75 or a pharmaceutically acceptable salt thereof.

In some embodiments of compounds of Formula A, the compound of Formula A is compound 78 or a pharmaceutically acceptable salt thereof.

In a second broad aspect there are provided compounds of Formula I or Formula II, or pharmaceutically acceptable salts thereof:

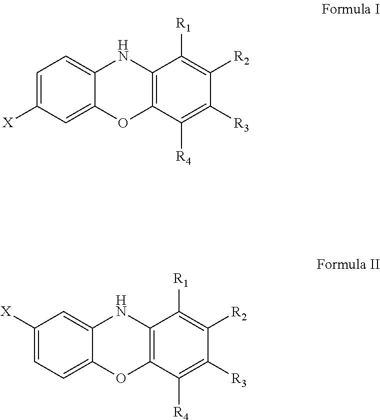

where: X is trifluoromethyl (CF₃), a perfluoroalkyl (CₙF₂ₙ₊₁ where n is an integer from 1 to 6), nitro (NO₂), cyano (CN), sulfonamide (SO₂N(R)₂), trifluoromethyl ether (OCF₃), a perfluoroalkyl ether (—OCₘF₂ₘ₊₁ where m is an integer from 1 to 6), fluoro (F), chloro (Cl), bromo (Br), iodo (I), formyl (CH(O)), acetyl (C(O)CH₃), an ester, an amide, or a ketone; and R₁, R₂, R₃, and R₄ are independently hydrogen (H), substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid, an ester, an amide, nitro (NO₂), fluoro (F), chloro (Cl), bromo (Br), iodo (I), formyl (CH(O)), acetyl (C(O)CH₃), a phosphonyl group, or a sulfonyl group; or, R₁ and R₂, taken together with the atoms to which they are attached, form a substituted or unsubstituted carbocyclic ring; or R₂ and R₃, taken together with the atoms to which they are attached, form a substituted or unsubstituted carbocyclic ring; or R₃ and R₄, taken together with the atoms to which they are attached, form a substituted or unsubstituted carbocyclic ring; provided that: R₁ is not COOH; and R₁, R₂, R₃, and R₄ are not all H at the same time.

In some embodiments of compounds of Formula I or II, when R₁, R₂ and R₄ are hydrogen, R₃ is not N(CH₂CH₃)₂.

In some embodiments of compounds of Formula I or II, when X is CF₃, R₃ is not also CF₃ and R₂ is not NO₂.

In some embodiments of compounds of Formula I or II, when X is CN, R₂ and R₃ are not also CN, and R₃ is not F.

In some embodiments of compounds of Formula I or II, when X is NO₂, R₂ and R₃ are not OCH₃.

In some embodiments of compounds of Formula I or II, when R₁, R₂ and R₄ are hydrogen, R₃ is not N(CH₂CH₃)₂; when X is CF₃, R₃ is not also CF₃ and R₂ is not NO₂; when X is CN, R₂ and R₃ are not also CN, and R₃ is not F; and/or, when X is NO₂, R₂ and R₃ are not OCH₃.

In some embodiments of compounds of Formula I or II, when R₁, R₂ and R₄ are hydrogen, R₃ is not N(CH₂CH₃)₂, and when X is NO₂, R₂ and R₃ are not OCH₃.

In some embodiments of compounds of Formula I or II, R₁ and R₄ are hydrogen.

In some embodiments of compounds of Formula I or II, R₁, R₃ and R₄ are hydrogen.

In some embodiments of compounds of Formula I or II, R₁, R₂ and R₄ are hydrogen.

In some embodiments of compounds of Formula I or II, X is trifluoromethyl (CF₃).

In some embodiments of compounds of Formula I or II, X is trifluoromethyl (CF₃), and R₁ and R₄ are hydrogen. In some embodiments of compounds of Formula I or II, X is trifluoromethyl (CF₃), and R₁, R₃ and R₄ are hydrogen. In some embodiments of compounds of Formula I or II, X is trifluoromethyl (CF₃), and R₁, R₂ and R₄ are hydrogen.

In some embodiments of Formula I or II, X is trifluoromethyl (CF₃), and at least one of R₂ and R₃ is not hydrogen (H). In some such embodiments, at least one of R₂ and R₃ is substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid.

In some embodiments of compounds of Formula I or II, when X is an ester, R₂ is not an ester. In some embodiments of compounds of Formula I or II, when X is an amide, R₂ and/or R₃ is not an amide. In some embodiments of compounds of Formula I or II, when X is an amide, R₂ and/or R₃ is not an amide, and R₃ is not an acid. In some embodiments of compounds of Formula I or II, when X is an amide, R₂ and R₃ is not an amide, and R₃ is not an acid.

In some embodiments, compounds of Formula I or II demonstrate enhanced metabolic stability and/or ferroptosis inhibition activity, compared to unsubstituted phenoxazine compounds.

In some embodiments, the compound of Formula I or II is a compound shown in Table 1, or a pharmaceutically acceptable salt thereof.

In some embodiments, the compound of Formula I or II is a compound shown in Table 2, or a pharmaceutically acceptable salt thereof.

In some embodiments, the compound of Formula I or II is compound 7, 8, 10, 11, 44, 46, 48, 49, 60, 66, 69, 72, 75, 78 or a pharmaceutically acceptable salt thereof.

In some embodiments, the compound of Formula I or II is compound 11 or a pharmaceutically acceptable salt thereof.

In some embodiments, the compound of Formula I or II is compound 44 or a pharmaceutically acceptable salt thereof.

In some embodiments, the compound of Formula I or II is compound 46 or a pharmaceutically acceptable salt thereof.

In some embodiments, the compound of Formula I or II is compound 48 or a pharmaceutically acceptable salt thereof.

In some embodiments, the compound of Formula I or II is compound 60 or a pharmaceutically acceptable salt thereof.

In some embodiments, the compound of Formula I or II is compound 66 or a pharmaceutically acceptable salt thereof.

In some embodiments, the compound of Formula I or II is compound 69 or a pharmaceutically acceptable salt thereof.

In some embodiments, the compound of Formula I or II is compound 72 or a pharmaceutically acceptable salt thereof.

In some embodiments, the compound of Formula I or II is compound 75 or a pharmaceutically acceptable salt thereof.

In some embodiments, the compound of Formula I or II is compound 78 or a pharmaceutically acceptable salt thereof.

In a third broad aspect, there is provided a ferroptosis inhibitor comprising a compound shown in Table 1 or Table 2, or a pharmaceutically acceptable salt thereof.

In a fourth broad aspect, there are provided compounds of Formula III, or pharmaceutically acceptable salts thereof:

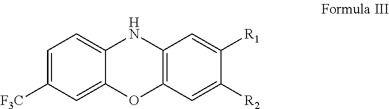

where R₁ and R₂ are independently selected from hydrogen (H), substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid, nitro (NO₂), fluoro (F), chloro (Cl), bromo (Br), iodo (I), formyl (CH(O)), acetyl (C(O)CH₃), a phosphonyl group, and a sulfonyl group; wherein at least one of R₁ and R₂ is not H.

In some embodiments, the CF₃, R₁ and/or R₂ can enhance metabolic stability and/or ferroptosis inhibition activity of the compound, compared to phenoxazine compounds without the CF₃, R₁ and/or R₂.

In some embodiments of Formula III, R₁ is hydrogen (H).

In some embodiments of Formula III, R₂ is hydrogen (H).

In some embodiments of Formula III, R₁ and R₂ are both not hydrogen (H).

In some embodiments of Formula III, at least one of R₂ and R₃ is substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid.

In some embodiments, the compound of Formula III is a compound shown in Table 1, Table 2, or a pharmaceutically acceptable salt thereof.

In a fifth broad aspect, there are provided compounds of Formula IV, or pharmaceutically acceptable salts thereof:

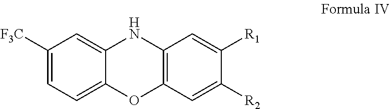

where R₁ and R₂ are independently selected from hydrogen (H), substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid, nitro (NO₂), fluoro (F), chloro (Cl), bromo (Br), iodo (I), formyl (CH(O)), acetyl (C(O)CH₃), a phosphonyl group, and a sulfonyl group; wherein at least one of R₁ and R₂ is not H.

In some embodiments, the CF₃, R₁ and/or R₂ can enhance metabolic stability and/or ferroptosis inhibition activity of the compound, compared to phenoxazine compounds without the CF₃, R₁ and/or R₂.

In some embodiments of Formula IV, R₁ is hydrogen (H).

In some embodiments of Formula IV, R₂ is hydrogen (H).

In some embodiments of Formula IV, R₁ and R₂ are both not hydrogen (H).

In some embodiments, the compound of Formula IV is a compound shown in Table 2, or a pharmaceutically acceptable salt thereof.

In some embodiments of Formula IV, at least one of R₂ and R₃ is substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid.

In some embodiments, compounds provided herein are ferroptosis inhibitor compounds. Without wishing to be limited by theory, it is believed that compounds provided herein can act as radical-trapping antioxidants (RTAs) that inhibit lipid autoxidation in membranous lipid bilayers, thereby inhibiting ferroptosis. In an embodiment, compounds provided herein inhibit have radical-trapping antioxidant activity. In some embodiments, compounds provided herein inhibit lipid autoxidation. In some embodiments, compounds provided herein inhibit non-apoptotic regulated cell death. In some embodiments, compounds provided herein reduce oxidative stress.

In some embodiments of compounds of the disclosure, the phenoxazine core structure can provide anti-ferroptotic activity; X is selected to enhance solubility of the compound in a membranous lipid bilayer and/or protect from P450-mediated hydroxylation; and/or R₁, R₂, R₃ and/or R₄ is/are selected to enhance orientation of the compound in a membranous lipid bilayer. For example, in an embodiment R₁, R₂, R₃ and/or R₄ possesses a basic amine protonated at physiological pH which can provide electrostatic interaction with negatively-charged phospholipid headgroups. In some embodiments, X is an electronegative (i.e., electron-withdrawing) moiety.

In some embodiments of the technology, the compound is a compound shown in Table 1, Table 2, or a pharmaceutically acceptable salt thereof.

In some embodiments of the technology, the compound is any one of compounds 4, 6-23, and 33-95, or a pharmaceutically acceptable salt thereof.

In some embodiments, the compound is compound 11, or a pharmaceutically acceptable salt thereof.

In some embodiments, the compound is compound 44, or a pharmaceutically acceptable salt thereof.

In some embodiments, the compound is compound 46, or a pharmaceutically acceptable salt thereof.

In some embodiments, the compound is compound 60, or a pharmaceutically acceptable salt thereof.

In some embodiments, the compound is compound 66, or a pharmaceutically acceptable salt thereof.

In some embodiments, the compound is compound 69, or a pharmaceutically acceptable salt thereof.

In some embodiments, the compound is compound 72, or a pharmaceutically acceptable salt thereof.

In some embodiments, the compound is compound 75, or a pharmaceutically acceptable salt thereof.

In some embodiments, the compound is compound 78, or a pharmaceutically acceptable salt thereof.

In another broad aspect, there are provided pharmaceutical compositions comprising a compound described herein, or a pharmaceutically acceptable salt thereof, and a pharmaceutically acceptable carrier. In some embodiments, there are provided pharmaceutical compositions comprising a compound of Formula A or a pharmaceutically acceptable salt thereof, and a pharmaceutically acceptable carrier. In some embodiments, there are provided pharmaceutical compositions comprising a compound of Formula I or a pharmaceutically acceptable salt thereof, and a pharmaceutically acceptable carrier. In some embodiments, there are provided pharmaceutical compositions comprising a compound of Formula II or a pharmaceutically acceptable salt thereof, and a pharmaceutically acceptable carrier. In some embodiments, there are provided pharmaceutical compositions comprising a compound of Formula III or a pharmaceutically acceptable salt thereof, and a pharmaceutically acceptable carrier. In some embodiments, there are provided pharmaceutical compositions comprising a compound of Formula IV or a pharmaceutically acceptable salt thereof, and a pharmaceutically acceptable carrier. In some embodiments, there are provided pharmaceutical compositions comprising a compound shown in Table 1 or Table 2, or a pharmaceutically acceptable salt thereof, and a pharmaceutically acceptable carrier. In some embodiments, there are provided pharmaceutical compositions comprising any one of compounds 4, 6-23, and 33-95, or a pharmaceutically acceptable salt thereof, and a pharmaceutically acceptable carrier.

In yet another broad aspect, there are provided methods of inhibiting ferroptosis, comprising contacting a cell with a compound described herein, or a pharmaceutically acceptable salt thereof, in an amount sufficient to inhibit ferroptosis. The compound may be, e.g., a compound of Formula A, I, II, III, or IV, or a compound shown in Table 1 or Table 2, or a pharmaceutically acceptable salt thereof. In some embodiments, the compound is any one of compounds 4, 6-23, and 33-95, or a pharmaceutically acceptable salt thereof. In some embodiments, the cell is contacted in vitro. In some embodiments, the cell is contacted ex vivo, e.g., in culture, or in vivo, e.g., in a subject.

In another broad aspect, there are provided methods of inhibiting ferroptosis in a subject, comprising administering to the subject an effective amount of a compound described herein, or a pharmaceutically acceptable salt thereof, so as to inhibit ferroptosis in the subject. In some embodiments, lipid autoxidation is inhibited or reduced in the subject. In some embodiments, oxidative stress is reduced in the subject. In some embodiments, non-apoptotic regulated cell death is reduced in the subject. Consequently, there are provided methods of treating or preventing a disease or disorder associated with ferroptosis in a subject in need of such treatment, comprising administering to the subject an effective amount of at least one compound or pharmaceutically acceptable salt thereof, or composition thereof, as described herein. In some embodiments, there are provided methods of reducing oxidative stress in a subject, comprising administering to the subject an effective amount of a compound described herein, or a pharmaceutically acceptable salt thereof, such that oxidative stress is reduced in the subject.

In some embodiments of methods provided herein, the compound is a compound of Formula A, I, II, III, or IV, as described above, or a pharmaceutically acceptable salt thereof. In some embodiments of methods provided herein, the compound is a compound shown in Table 1, Table 2, or a pharmaceutically acceptable salt thereof. In some embodiments, of methods provided herein, the compound is any one of compounds 4, 6-23, and 33-95, or a pharmaceutically acceptable salt thereof. In some embodiments of methods of the disclosure, the compound is compound 18, or a pharmaceutically acceptable salt thereof.

In another broad aspect, therapeutic methods of use of the compounds and compositions described herein for the prevention and treatment of ferroptosis-associated diseases or disorders are provided. Examples of ferroptosis-associated diseases or disorders include, without limitation, stroke, myocardial infarction, diabetes, sepsis, transplant rejection, neurodegenerative disease, a central nervous system (CNS) injury, ischemic events, and cancers. Non-limiting examples of neurodegenerative diseases include Alzheimer's disease, Huntington's disease, Parkinson's disease, amyotrophic lateral sclerosis (ALS), HIV-associated dementia, cerebral ischemia, multiple sclerosis, Lewy body disease, Menke's disease, Wilson's disease, Creutzfeldt-Jakob disease, Fahr disease, Friedreich's ataxia, and frontotemporal dementia, amyloidosis, Tay-Sachs disease and periventricular leukomalacia. Non-limiting examples of CNS injury include traumatic brain injury, spinal cord injury, and stroke. Non-limiting examples of ischemic events include cerebral ischemia, ischemic stroke, organ ischemia, myocardial infarction, mesenteric, retinal, hepatic or brain ischemic injury, and ischemic-reperfusion injury.

In an embodiment, there are provided methods of preventing or treating a neurodegenerative disease in a subject in need thereof, comprising administering to the subject an effective amount of a compound described herein, or a pharmaceutically acceptable salt thereof, so as to treat the neurodegenerative disease in the subject. In some embodiments, the compound is a compound of Formula A, I, II, III, or IV as described above, or a compound of Table 1 or Table 2, or a pharmaceutically acceptable salt thereof. In some embodiments, ferroptosis and/or oxytosis is inhibited in the subject. In some embodiments, lipid autoxidation is inhibited in the subject. In some embodiments, oxidative stress is reduced in the subject.

In an embodiment, there are provided methods of preventing or treating ischemic reperfusion injury (IRI) in a subject in need thereof, comprising administering to the subject an effective amount of a compound described herein, or a pharmaceutically acceptable salt thereof, so as to prevent or treat the IRI in the subject. In some embodiments, the compound is a compound of Formula A, I, II, III, or IV as described above, or a compound of Table 1, Table 2, or a pharmaceutically acceptable salt thereof. In some embodiments, ferroptosis and/or oxytosis is inhibited in the subject. In some embodiments, lipid autoxidation is inhibited in the subject. In some embodiments, oxidative stress is reduced in the subject.

In a further aspect, there are provided kits for treating a disease or disorder associated with ferroptosis in a subject in need thereof, comprising a compound (or a pharmaceutically acceptable salt thereof) or a pharmaceutical composition, as described herein; optionally one or more additional component such as acids, bases, buffering agents, inorganic salts, solvents, antioxidants, preservatives, or metal chelators; and instructions for use thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a better understanding of the invention and to show more clearly how it may be carried into effect, reference will now be made by way of example to the accompanying drawings, which illustrate aspects and features according to embodiments of the present invention, and in which:

FIG. 1 is a schematic drawing showing: (A) autoxidation of polyunsaturated phospholipids leads to a loss in membrane integrity and drives ferroptosis; (B) inhibition of lipid autoxidation (peroxidation) by radical-trapping antioxidants (RTAs), illustrated with BHT; and (C) the range of reactivity of representative RTAs (both phenolic and aminic) expressed in terms of their inhibition rate constants (kᵢₙₕ) as measured in solution (chlorobenzene) at 37° C.

FIG. 2 shows, in (A): a schematic diagram that shows co-autoxidation of STY-BODIPY and PBD-BODIPY can be monitored by either absorbance or fluorescence. Inhibition rate constants (kᵢₙₕ) and radical-trapping stoichiometries (n) are determined from the initial rates and duration of the inhibited periods using Eqs. 1 and 2, respectively; in (B): representative data for azine-inhibited co-autoxidation of PBD-BODIPY (10 M) and 1,4-dioxane (2.9 M) in chlorobenzene initiated with AIBN (6 mM) at 37° C. and monitored at 587 nm (123 000 M⁻¹ cm⁻¹); in (C): a kinetic scheme for the suppression in azine RTA activity due to H-bonding interactions between the RTA and solvent, where kᵢₙₕ⁵ is the inhibition rate constant in a given solvent S, K^{S} is the equilibrium constant for formation of a 1:1 H-bonded complex between the RTA and solvent, and kᵢₙₕ⁰ is the inhibition rate constant in the absence of any H-bonding interactions; in (D): representative data for azine-inhibited co-autoxidation of PBD-BODIPY (10 μM) and 1,4-dioxane (2.9 M) in 2:1 chlorobenzene:DMSO) initiated with AIBN (6 mM) at 37° C. and monitored at 587 nm (118200 M⁻¹ cm⁻¹); in (E): representative data for azine-inhibited co-autoxidation of STY-BODIPY (1 μM) and liposomal egg phosphatidylcholine (1 mM) suspended in phosphate-buffered saline (PBS, pH 7.4) initiated with MeOAMVN (200 μM) at 37° C. and monitored by fluorescence (λex/λem=488/518 nm); in (F): Top: Correlation of log kᵢₙₕ⁰ and log kᵢₙₕ^{lip} (N=29; R²=0.417); Bottom: Correlation of log (kᵢₙₕ⁰/8.3α₂^{H}) and log (kᵢₙₕ^{lip}/8.3α₂^{H}) (N=29; R²=0.991); and in (G): predicted kᵢₙₕ^{lip} for RTAs which were too reactive for it to be determined directly. Values were obtained using Eq. 5 with experimental kᵢₙₕ⁰ and α₂^{H} and β₂^{H}=0.69.

FIG. 3 shows, in (A): a schematic diagram showing a proposed mechanism of catalytic trapping of phospholipid-derived peroxyl radicals by azine RTAs and ascorbate; in (B): representative data from co-autoxidations of STY-BODIPY (1 μM) and liposomal egg phosphatidylcholine (1 mM) suspended in PBS (pH 7.4) initiated with DTUN (200 μM) at 37° C. and monitored by fluorescence (λex/λem=488/518 nm), uninhibited or in the presence of PMC, as indicated; in (C): representative data from co-autoxidations of STY-BODIPY (1 μM) and liposomal egg phosphatidylcholine (1 mM) suspended in PBS (pH 7.4) initiated with DTUN (200 μM) at 37° C. and monitored by fluorescence (λex/λem=488/518 nm), uninhibited or in the presence of PNX, as indicated; in (D): representative data from inhibited autoxidations carried out with either 4 μM RTA (solid line) or 400 nM RTA (dashed/dotted lines) with varying concentrations of ascorbate, as indicated; in (E): representative data from inhibited autoxidations carried out with either 4 μM RTA (solid line) or 400 nM RTA (dashed/dotted lines) with varying concentrations of ascorbate, as indicated; in (F): representative data from inhibited autoxidations carried out with either 4 μM RTA (solid line) or 400 nM RTA (dashed/dotted lines) with varying concentrations of ascorbate, as indicated; and in (G): a histogram showing the radical-trapping stoichiometries and regeneration coefficients for test compounds, as indicated, with No Ascorbate; 10 μM Ascorbate; or 100 μm Ascorbate.

FIG. 4 shows, in (A): a graph showing a correlation of kᵢₙₕ^{lip} values determined from DTUN-initiated autoxidations of egg PC liposomes versus those determined (or predicted) from MeOAMVN-initiated autoxidations. MeOAMVN-initiated vs DTUN-initiated+100 μM ascorbate (black dot ●); predicted from Eq. 4 (see FIG. 2C) vs DTUN-initiated+100 μM ascorbate (red dot ●); MeOAMVN-initiated vs DTUN-initiated without ascorbate (Q); and in (B): representative data from co-autoxidations of STY-BODIPY (1 μM) and liposomal egg phosphatidylcholine (1 mM) suspended in PBS (pH 7.4) initiated with DTUN (200 μM) at 37° C. and monitored by fluorescence (λex/λem=488/518 nm). Top: 3,7-Me-PNX (35), 3,7-Et-PNX (41), 3,7-iPr-PNX (42) and 3,7-tBu-PNX (34) at 4 μM in the absence of ascorbate; Bottom: aforementioned at 400 nM in the presence of 100 μM of ascorbate.

FIG. 5 shows, in (A): representative data showing RSL3 induced ferroptosis of Pfa-1 mouse-embryonic fibroblasts (MEFS) inhibited by exemplary lipid soluble RTAs, as indicated; in (B): representative data showing RSL3 induced ferroptosis of Pfa-1 MEFS inhibited by exemplary lipid soluble RTAs, as indicated; in (C): representative data showing RSL3 induced ferroptosis of Pfa-1 MEFS inhibited by exemplary lipid soluble RTAs, as indicated; in (D): results from liver microsomal stability experiments (BALB/c mouse). a: 10 μM of compound (separated by RP-UPLC and monitored by PDA); b: Compound at 1 μM (monitored by MS-ESI+); c: [Fer-1]=30 μM; d: Standard conditions: 0.5 mg/mL liver microsomes; 1 mM NADPH; 100 mM phosphate buffer (pH 7.4); 37° C.; e: Liver microsomes increased (1.0 mg/mL); f: At 0.5 mg/mL, Fer-1 was undetectable at 5 min., decreased to 0.1 mg/mL microsomes where t_{1/2}=5.3±0.1 min was observed. Results from liver microsomal stability experiments are also shown in Table 4; and in (E): a schematic diagram showing primary oxidative metabolism of PNX.

FIG. 6 is a graph showing multi-regression correlation for log EC₅₀ vs log kᵢₙₕ^{lip} and log P for Compounds 1-31 (R_{adj}²=0.816; [log EC₅₀]=0.567(log kᵢₙₕ^{lip})+0.298 (log P)+3.553).

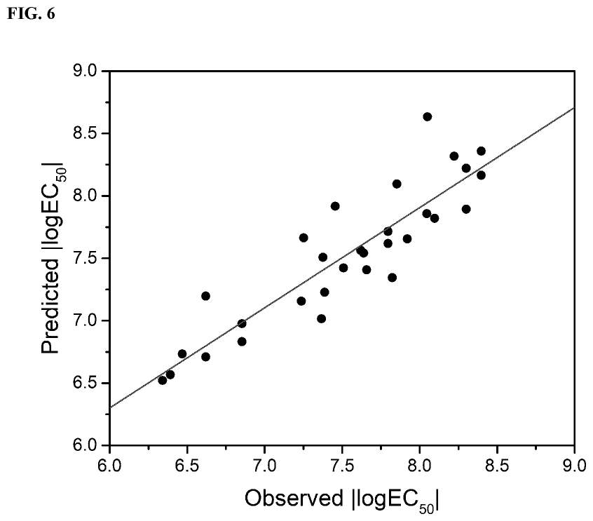

### DETAILED DESCRIPTION

### Definitions

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “about” is used to indicate that a value includes an inherent variation of error for the device or the method being employed to determine the value.

The term “derivative” as used herein, is understood as being a substance similar in structure to another compound but differing in some slight structural detail.

The present description refers to a number of chemical terms and abbreviations used by those skilled in the art. Nevertheless, definitions of selected terms are provided for clarity and consistency.

As used herein, the term “lipophilic” refers to the ability of a substance to combine with or dissolve in lipids or fats. Generally, a substance is lipophilic if it dissolves much more easily in lipid or fats than in water. In compounds of the present disclosure, lipophilic substituents can generally enhance solubility of a compound in a lipid bilayer (e.g., a cell membrane).

As used herein, the term “amphiphilic” refers to a molecule having both hydrophobic (nonpolar) and hydrophilic (polar) regions. Such molecules have both hydrophilic (water-loving) and lipophilic (fat-loving) properties. Amphiphilic molecules are also known as amphipathic molecules. Common examples of amphipathic molecules include detergents and phospholipids. In compounds of the present disclosure, amphiphilic substituents can generally enhance orientation of a compound in a membranous lipid bilayer. For example, a basic amine protonated at physiological pH can encourage electrostatic interactions with negatively charged phospholipid headgroups in a lipid bilayer.

As used herein, the term term “substituted” or “with substitution” refers to a parent compound or a moiety that has at least one (1) substituent group. The term “unsubstituted” or “without substitution” refers to a parent compound or a moiety that has no other substituent group except that the unidentified valence is chemically saturated with hydrogen atoms.

As used herein, a “substituent” or a “substituent group” refers to a group selected from halogen (F, Cl, Br, or I), hydroxy, sulfhydryl, amino, nitro, carbonyl, carboxyl, alkyl, alkoxyl, alkylamino, aryl, aryloxyl, arylamino, acyl, thionyl, sulfonyl, phosphonyl, or other organic moiety as used and accepted in general organic chemistry.

As used herein, the terms “alkyl” and “C₁₋₆ alkyl” can be straight-chain or branched. Examples of alkyl residues containing from 1 to 6 carbon atoms are methyl, ethyl, propyl, butyl, pentyl, hexyl, the n-isomers of all these residues, isopropyl, isobutyl, isopentyl, neopentyl, isohexyl, 3-methylpentyl, sec-butyl, tert-butyl, or tert-pentyl. Alkyl residues may be substituted or unsubstituted. In some embodiments, for example, alkyl may be substituted by hydroxyl, amino, carboxyl, carboxylic ester, amide, carbamate, or aminoalkyl.

Unless the number of carbons is otherwise specified, “lower” as in “lower aliphatic,” “lower alkyl,” “lower alkenyl,” and “lower alkylnyl”, as used herein means that the moiety has at least one (two for alkenyl and alkynyl) and equal to or less than 6 carbon atoms.

As used herein, the term “cycloalkyl” can be monocyclic or polycyclic, for example monocyclic, bicyclic or tricyclic, i.e., they can for example be monocycloalkyl residues, bicycloalkyl residues and tricycloalkyl residues, provided they have a suitable number of carbon atoms and the parent hydrocarbon systems are stable. A bicyclic or tricyclic cycloalkyl residue has to contain at least 4 carbon atoms. In an embodiment, a bicyclic or tricyclic cycloalkyl residue contains at least 5 carbon atoms. In a further embodiment, a bicyclic or tricyclic cycloalkyl residue contains at least 6 carbon atoms and up to the number of carbon atoms specified in the respective definition. Cycloalkyl residues can be saturated or contain one or more double bonds within the ring system. In particular they can be saturated or contain one double bond within the ring system. In unsaturated cycloalkyl residues the double bonds can be present in any suitable positions. Monocycloalkyl residues are, for example, cyclopropyl, cyclobutyl, cyclopentyl, cyclopentenyl, cyclohexyl, cyclohexenyl, cycloheptyl, cycloheptenyl, cyclooctyl, cyclononyl, cyclodecyl, cycloundecyl, cyclododecyl or cyclotetradecyl, which can also be substituted, for example by C₁₋₄ alkyl. Examples of substituted cycloalkyl residues are 4-methylcyclohexyl and 2,3-dimethylcyclopentyl. Examples of parent structures of bicyclic ring systems are norbornane, bicyclo[2.2.1]heptane, bicyclo[2.2.2]octane and bicyclo[3.2.1]octane.

As used herein, the term “aryl” means an aromatic substituent that is a single ring or multiple rings fused together. When formed of multiple rings, at least one of the constituent rings is aromatic. In an embodiment, aryl substituents include phenyl, naphthyl and anthracyl groups.

The term “heteroaryl”, as used herein, is understood as being aromatic rings of five or six atoms containing one or two O- and/or S-atoms and/or one to four N-atoms, provided that the total number of hetero-atoms in the ring is 4 or less. The heteroaryl ring is attached by way of an available carbon or nitrogen atom. Non-limiting examples of heteroaryl groups include 2-, 3-, or 4-pyridyl, 4-imidazolyl, 4-thiazolyl, 2- and 3-thienyl, and 2- and 3-furyl. The term “heteroaryl”, as used herein, is understood as also including bicyclic rings wherein the five or six membered ring containing O, S and N-atoms as defined above is fused to a benzene or pyridyl ring. Non-limiting examples of bicyclic rings include but are not limited to 2- and 3-indolyl as well as 4- and 5-quinolinyl.

As used herein, the term “arylalkyl” means an aryl group that is attached through an alkylene group to the parent moiety, wherein aryl and alkyl are as defined herein. Non-limiting examples of arylalkyl include benzyl, naphthalene-1-ylmethyl, and naphthalene-2-ylmethyl.

As used herein, the term “halogen” includes fluorine (F), chlorine (Cl), bromine (Br), and iodine (I). Similarly, the term “halo” includes fluoro, chloro, bromo, and iodo.

### Ferroptosis Inhibitor Compounds

In a broad aspect, the present disclosure relates to ferroptosis inhibitor compounds, and their use as therapeutics.

Without wishing to be limited by theory, it is believed that compounds described herein can act to inhibit or reduce undesired non-apoptotic regulated cell death in a subject suffering from a ferroptosis-associated disease or disorder, as described further hereinbelow, through one or more of the following activities: reducing oxidative stress, trapping or blocking reactive oxygen species, inhibiting lipid autoxidation, inhibiting production of reactive oxygen species, and/or inhibiting lipoxygenase activation. In some embodiments, compounds described herein can act as radical-trapping antioxidants (RTAs). Consequently, in some embodiments, the compounds described herein can inhibit ferroptosis and/or oxytosis.

In an embodiment, there is provided a compound of Formula A, or a pharmaceutically acceptable salt thereof, as described herein.

In an embodiment, there is provided a compound of Formula I, or a pharmaceutically acceptable salt thereof, as described herein.

In an embodiment, there is provided a compound of Formula II, or a pharmaceutically acceptable salt thereof, as described herein.

In an embodiment, there is provided a compound of Formula III, or a pharmaceutically acceptable salt thereof, as described herein.

In an embodiment, there is provided a compound of Formula IV, or a pharmaceutically acceptable salt thereof, as described herein.

In an embodiment, there is provided a compound of Table 1 or Table 2, e.g., compounds 4, 6-23, and 33-95, as described herein, or a pharmaceutically acceptable salt thereof.

Pharmaceutical compositions and therapeutic methods comprising the compounds described herein or pharmaceutically acceptable salts thereof, are also encompassed.

As would be understood by a person of ordinary skill in the art, the recitation of “a compound” is intended to include salts, solvates, oxides, and inclusion complexes of that compound as well as any stereoisomeric form, or a mixture of any such forms of that compound in any ratio. Thus, in accordance with some embodiments of the invention, a compound as described herein, including in the contexts of pharmaceutical compositions and methods of treatment is provided as the salt form.

Compounds described herein include, but are not limited to, their optical isomers, racemates, and other mixtures thereof. In those situations, the single enantiomers or diastereomer, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, such compounds include Z- and E-forms (or cis- and trans-forms) of compounds with carbon-carbon double bonds. Where compounds described herein exist in various tautomeric forms, the term “compound” is intended to include all tautomeric forms of the compound. Such compounds also include crystal forms including polymorphs and clathrates. Similarly, the term “salt” is intended to include all tautomeric forms and crystal forms of the compound.

The configuration of any carbon-carbon double bond appearing herein is selected for convenience only and is not intended to designate a particular configuration; thus a carbon-carbon double bond depicted arbitrarily herein as E may be Z, E, or a mixture of the two in any proportion.

The term “solvate” refers to a compound in the solid state, where molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

For compounds provided herein, it is intended that, in some embodiments, salts thereof are also encompassed, including pharmaceutically acceptable salts. Those skilled in the art will appreciate that many salt forms (e.g., TFA salt, tetrazolium salt, sodium salt, potassium salt, etc,) are possible; appropriate salts are selected based on considerations known in the art. The term “pharmaceutically acceptable salt” refers to salts prepared from pharmaceutically acceptable non-toxic acids or bases including inorganic acids and bases and organic acids and bases. For example, for compounds that contain a basic nitrogen, salts may be prepared from pharmaceutically acceptable non-toxic acids including inorganic and organic acids. Suitable pharmaceutically acceptable acid addition salts for the compounds of the present invention include without limitation acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds of the present invention include without limitation metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine), and procaine.

### Compositions

There are provided pharmaceutical compositions comprising a compound described herein, or a pharmaceutically acceptable salt thereof, and a pharmaceutically acceptable carrier.

In an embodiment, there is provided a pharmaceutical composition comprising a compound of Formula A, Formula I, Formula II, Formula III, or Formula IV, or a pharmaceutically acceptable salt thereof, and a pharmaceutically acceptable carrier. In an embodiment, there is provided a pharmaceutical composition comprising a compound shown in Table 1, Table 2, or a pharmaceutically acceptable salt thereof, and a pharmaceutically acceptable carrier. In an embodiment, there is provided a pharmaceutical composition comprising any one of compounds 4, 6-23, and 33-95, or a pharmaceutically acceptable salt thereof, and a pharmaceutically acceptable carrier. In another embodiment, there is provided a pharmaceutical composition comprising compound 11, or a pharmaceutically acceptable salt thereof, and a pharmaceutically acceptable carrier.

The preparation of pharmaceutical compositions can be carried out as known in the art (see, for example, Remington: The Science and Practice of Pharmacy, 20^{th} Edition, 2000). For example, a therapeutic compound and/or composition, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human or veterinary medicine. Pharmaceutical preparations can also contain additives, of which many are known in the art, for example fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

The term “pharmaceutical composition” means a composition comprising a compound as described herein and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms.

The term “pharmaceutically acceptable carrier” is used to mean any carrier, diluent, adjuvant, excipient, or vehicle, as described herein. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates. Examples of lubricants include magnesium stearate, sodium lauryl sulphate, talc, as well as high molecular weight polyethylene glycols.

The term “pharmaceutically acceptable” means it is, within the scope of sound medical judgment, suitable for use in contact with the cells of a subject, e.g., humans and animals, without undue toxicity, irritation, allergic response, and the like, and are commensurate with a reasonable benefit/risk ratio.

A pharmaceutically acceptable carrier may include any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier may be suitable for intravenous, intraperitoneal, intramuscular, sublingual or oral administration. In other embodiments, the carrier is suitable for topical administration or for administration via inhalation. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions provided herein is contemplated. Supplementary active compounds can also be incorporated into the compositions. For example, a pharmaceutical composition provided herein may further comprise at least one additional therapeutic, e.g., an additional therapeutic for treating neurodegenerative disease, as discussed below.

A pharmaceutical composition provided herein can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories. Administration can also be carried out parenterally, for example subcutaneously, intramuscularly or intravenously in the form of solutions for injection or infusion. Other suitable administration forms are, for example, percutaneous or topical administration, for example in the form of ointments, creams, tinctures, sprays or transdermal therapeutic systems, or the inhalative administration in the form of nasal sprays or aerosol mixtures, or, for example, microcapsules, implants or wafers.

Pharmaceutical compositions typically must be sterile and stable under the conditions of manufacture and storage. A composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of injectable compositions can be brought about by including in the composition an agent which delays absorption, for example, monostearate salts and gelatin. Moreover, a compound can be administered in a time release formulation, for example in a composition which includes a slow-release polymer. The compound can be prepared with carriers that will protect against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG).

Many methods for the preparation of such formulations are generally known to those skilled in the art. Sterile injectable solutions can be prepared by incorporating an active compound, such as a ferroptosis inhibitor compound provided herein, in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, common methods of preparation include vacuum drying and freeze-drying which yields a powder of the active ingredient plus any additional desired ingredient from a previously sterile-filtered solution thereof. Compounds may also be formulated with one or more additional compounds that enhance their solubility.

It is often advantageous to formulate compositions (such as parenteral compositions) in dosage unit form for ease of administration and uniformity of dosage. The term “unit dosage form” refers to a physically discrete unit suitable as unitary dosages for human subjects and other animals, each unit containing a predetermined quantity of active material calculated to produce the desired therapeutic effect, in association with a suitable pharmaceutical carrier. The specification for the dosage unit forms of the invention may vary and are dictated by and directly dependent on (a) the unique characteristics of the therapeutic compound and the particular therapeutic effect to be achieved, and (b) the limitations inherent in the art of compounding such a therapeutic compound for the prevention or treatment of a ferroptosis-associated disease or disorder. Dosages are discussed further below.

In some embodiments, there are provided pharmaceutical compositions that comprise an effective amount of a compound and/or composition described herein, and a pharmaceutically acceptable carrier. In an embodiment, there are provided pharmaceutical compositions for the treatment or prevention of a ferroptosis-associated disease or disorder, such as for example a neurodegenerative disease, comprising a compound described herein, or a pharmaceutically acceptable salt thereof, and a pharmaceutically acceptable carrier.

### Methods of Use of Compounds and Compositions

There are provided methods of treating or preventing a disease or disorder associated with ferroptosis in a subject in need of such treatment, comprising administering to the subject an effective amount of at least one compound or pharmaceutically acceptable salt thereof, or composition thereof, as described herein.

Ferroptosis is an oxidative stress dependent, non-apoptotic cell death pathway. It typically involves glutathione depletion, reactive oxygen species production, lipoxygenase activation, and calcium influx, and is iron-dependent. Ferroptosis is highly similar (and perhaps identical) to oxytosis, a form of regulated cell death induced by glutathione depletion which has been implicated in nerve cell death in a variety of neurological diseases (Lewerenz et al., 2018). Ferroptosis and oxytosis are associated with pathological or undesired cell death in a large number of diseases and disorders. Compounds and compositions of the disclosure may be used to inhibit ferroptosis and/or oxytosis and are useful therapeutically for a large number of associated diseases and disorders.

As used herein, the terms “disease or disorder associated with ferroptosis” and “ferroptosis-associated disease or disorder” are used interchangeably to refer to any pathological medical condition that would benefit from treatment with a ferroptosis inhibitor compound of the disclosure or pharmaceutical composition thereof as described herein. This includes chronic and acute disorders or diseases including those pathological conditions that predispose the subject to the disease or disorder in question. A ferroptosis-associated disease or disorder is thus a disease or disorder that may be ameliorated through inhibition of ferroptosis, such as, without limitation, through inhibition of lipid autoxidation, inhibition of reactive oxygen species production, inhibition of lipoxygenase activation, inhibition of lipid autoxidation, trapping or blocking of reactive oxygen species, e.g., by an antioxidant, e.g., an RTA, and reduction of oxidative stress. As such, there are provided methods for prevention or treatment of a ferroptosis-associated disease or disorder in a subject, the methods comprising administering a therapeutically effective amount of the inhibitor compound or pharmaceutical composition described herein. Inhibitor compounds are generally administered in the form of a pharmaceutical composition. A subject may be in need of such treatment, i.e., having, suspected of having, or at risk of having a disease or disorder associated with ferroptosis.

Ferroptosis-associated diseases or disorders may include a wide range of diseases and disorders associated with cell death and oxidative stress. For example and without limitation, in some embodiments ferroptosis-associated diseases or disorders include: neurodegenerative or neurological diseases (e.g., disorders of the central nervous system (CNS), e.g., Alzheimer's disease, Parkinson's disease, Huntington's disease, Amyotrophic lateral sclerosis (ALS, also known as Lou Gehrig's disease), multiple sclerosis, Prion disease, spinocerebellar ataxias); stroke (e.g., cerebral ischemia, ischemic stroke, hemorrhagic stroke); organ ischemia (e.g., stroke, myocardial infarction, mesenteric, retinal, hepatic or brain ischemic injury); ischemia-reperfusion injury (e.g., associated with surgery or other ischemic injury); central nervous system (CNS) injury such as traumatic brain injury (TBI), spinal cord injury (SCI), and stroke; neuropathy; cancer (e.g., cancer metastasis, leukemia, melanoma, tumors); autoimmune diseases; inflammatory diseases; fibrosis; degenerative joint disease such as osteoarthritis; muscular dystrophy; muscle wasting; retinal necrosis; cardiovascular diseases; heptic injury; renal injury; and kidney disease.

In some embodiments, ferroptosis-associated diseases or disorders include neurodegenerative diseases such as, for example and without limitation, Alzheimer's disease, amyotrophic lateral sclerosis (ALS), Huntington's disease, Parkinson's′ disease, Prion disease, multiple sclerosis and spinocerebellar ataxias. Consequently, in some embodiments there are provided methods for preventing or treating a neurodegenerative disease.

In some embodiments, ferroptosis-associated diseases or disorders include CNS injury such as, for example and without limitation, traumatic brain injury, spinal cord injury, and stroke. Consequently, in some embodiments there are provided methods for treating a CNS injury.

In some embodiments, ferroptosis-associated diseases or disorders include ischemic events such as, for example and without limitation, cerebral ischemia, ischemic stroke, organ ischemia, myocardial infarction, mesenteric, retinal, hepatic or brain ischemic injury, or ischemic-reperfusion injury. Consequently, in some embodiments there are provided methods for treating an ischemic event, e.g., methods for treating or preventing ischemic-reperfusion injury.

In some embodiments, ferroptosis-associated diseases or disorders include, for example and without limitation, a cancer. A cancer may be a blood-cell derived cancer such as, without limitation, a lymphoma, a leukemia, or a myeloma, or a solid organ tumor such as, without limitation, a tumor of the colon, breast, lung, prostate, brain, pancreas, ovary, or skin. In an embodiment, the cancer is a glioma, such as a malignant glioma or a glioblastoma, e.g., glioblastoma multiforme (GBM). Consequently, in some embodiments there are provided methods for preventing or treating a cancer, e.g., recurrence of a cancer after treatment, tumor growth, cancer metastasis, etc.

In some embodiments, there are provided methods for treating a neurodegenerative disease in a subject in need of such treatment, comprising administering a compound or a composition as described herein to the subject. For example, there are provided methods for treating Huntington's disease, ALS, Parkinson's disease and/or Alzheimer's disease. Such methods may include treating memory and/or cognitive impairment associated with the neurodegenerative disease in the subject. Exemplary neurodegenerative diseases or disorders include, but are not limited to, Alzheimer's disease, Huntington's disease, Parkinson's disease, amyotrophic lateral sclerosis (ALS), HIV-associated dementia, cerebral ischemia, multiple sclerosis, Lewy body disease, Menke's disease, Wilson's disease, Creutzfeldt-Jakob disease, Fahr disease, Friedreich's ataxia, and frontotemporal dementia, amyloidosis, Tay-Sachs disease and periventricular leukomalacia.

In some embodiments, there are provided methods for treating ischemic-reperfusion injury in a subject in need of such treatment, comprising administering a compound or a composition as described herein to the subject. Ischemic-reperfusion injury (IRI) is a primary complication of transplant surgery, accounting for the majority of liver and kidney transplant failures, acute kidney tubular necrosis and delayed graft function. Indeed, non-apoptotic regulated cell death (e.g., ferroptosis) is believed to be a main contributor to IRI. In contrast to apoptosis where cell death is limited to the affected cell, for non-apoptotic regulated cell death, cell death may cause inflammation which causes damage to surrounding tissue. Compounds and compositions of the disclosure can thus be useful to reduce IRI and to, e.g., reduce organ trauma upon transplantation and treat or prevent other diseases or conditions caused by ischemic-reperfusion.

In some embodiments, compounds or compositions of the disclosure may be used in the treatment (including prophylactic treatment) of a condition, disorder or disease that is selected from the group consisting of: a neurodegenerative disease of the central or peripheral nervous system, a condition or disorder caused by and forms of neurodegeneration; muscle wasting or muscular dystrophy; organ ischemia (e.g., stroke, myocardial infarction and heart, mesenteric, retinal, hepatic or brain ischemic injury), ischemic-reperfusion injury (such as associated with surgery, especially solid organ transplantation), ischemic injury during organ storage, limb or organ ischemic injury (such as associated with surgery, tourniquet use or trauma); compartment syndrome, gangrene, pressure sores, sepsis (e.g., aseptic necrosis), degenerative arthritis; retinal necrosis (e.g., acute retinal necrosis (ARN) cased by or associated with optic nerve detachment); cardiovascular (heart) disease, including stroke, coronary heart disease, cardiomyopathy; liver, gastrointestinal or pancreatic disease (e.g., acute necrotizing pancreatitis); avascular necrosis (e.g., bone avascular necrosis), diabetes, sickle cell disease, alteration of blood vessels (e.g., vascular dystrophy or cerebrovascular disease); cancer-chemo/radiation therapy-induced cell-death (e.g., mucositis or chemotherapy induced alopecia (CIA)); and cell, tissue, organ or organism intoxication (e.g., nephrotoxicity), such as that the result of, arising from or associated with drug treatment (e.g., complications from steroid treatment, kidney toxicity from cisplatin, cardiotoxicity from doxorubicin or ototoxicity from gentamicin), drug overdose (e.g., liver toxicity from paracetamol) or acute poisoning (e.g., from alcohol, paraquat or environmental toxins), or contrast-agent-induced toxicity; and priapism; or is the result of, arises from or is associated with any of the foregoing.

In some embodiments, compounds or compositions of the disclosure may be used in the treatment (including prophylactic treatment) of a condition, disorder or disease that is the result of, arises from or is associated with a circumstance selected from the group consisting of: forms of infection of viruses, bacteria, fungi or other microorganisms (e.g., septic shock, tuberculosis); a reduction in cell-proliferation, or an alteration in cell-differentiation or intracellular signalling; an undesirable inflammation, such as an immune disorder; retinal neuronal cell death, cell death of cardiac muscle, cell death of cells of the immune system, cell death associated with renal failure; neonatal respiratory distress, asphyxia, incarcerated hernia, placental infarct, iron-load complications, endometriosis, congenital disease, including congenital mitochondrial disease (e.g., tyrosinemia, phenylketonuria, Anderson disease); head trauma/traumatic brain injury, liver injury; injuries from environmental radiation (e.g., UV exposure and sunburn); burns; cold injuries (e.g., hyperthermia), mechanical injuries (e.g., brain and spinal cord injuries); and decompression sickness; snake, scorpion or spider bites; and side effects of medications.

Without wishing to be bound by theory, in some cases apoptosis is believed to occur under or as a result of normal physiological conditions or events in a highly programmed manner as part of normal tissue homeostasis and cell turnover, whereas, in contrast, non-apoptotic regulated cell death (e.g., ferroptosis/oxytosis) is triggered by abnormal physiological conditions or events such as external damaging stimuli and/or oxidative stress. In certain embodiments, compounds that inhibit non-apoptotic regulated cell-death but do not inhibit apoptotic cell-death are used in the methods and applications of the present disclosure, so as to avoid interfering with a subject's innate cell-death mechanism and regulation, and to inhibit preferentially only cell death caused by abnormal physiological conditions or events such as external damaging stimuli and/or oxidative stress and/or triggering events of the immune system.

In some embodiments, compounds or compositions of the disclosure may be used in the treatment (including prophylactic treatment) of a condition, disorder or disease selected from: muscle wasting (e.g., that associated with cancer, AIDS, congestive heart failure, chronic obstructive disease, and necrotizing myopathy of intensive care). In particular embodiments the condition, disorder or disease is muscular dystrophies or related diseases (e.g., Becker's muscular dystrophy, Duchenne muscular dystrophy, myotonic dystrophy, limb-girdle muscular dystrophy, Landouzy-Dejerine muscular dystrophy, facioscapulohumeral muscular dystrophy (Steinert's disease), myotonia congenita, Thomsen's disease, and Pompe's disease), or is a condition or symptom the result of, arising from or associated with such condition, disorder or disease. In other embodiments, the condition, disorder or disease is cell, tissue, organ or organism intoxication, such as that the result of, arising from or associated with drug treatment, drug overdose or acute poisoning. Exemplary circumstances of such intoxication include alcoholism and administration and/or self-administration with, and/or exposure to, illicit drugs (e.g., cocaine, heroin, crack), medical drugs (e.g., anti-cancer agents, paracetamol, antibiotics, adriamycin, NSAID, cyclosporine), chemical toxins (e.g., carbon tetrachloride, cyanide, methanol, ethylene glycol and mustard gas, agrochemicals such as organophosphates and paraquat, and warfare organophosphates), or heavy metals (e.g., lead, mercury).

In other particular embodiments, the condition, disorder or disease is the result of, arising from or associated with one or more forms of infection of viruses (e.g., acute, latent and/or persistent), bacteria, fungi, or other microorganisms, or is one in which a reduction in cell-proliferation, or an alteration in cell-differentiation or intracellular signalling, is a causative factor, and include infection e.g., by viruses (e.g., acute, latent and/or persistent), bacteria, fungi, or other microorganisms, and mycoplasma disease. Exemplary viruses include, but are not limited to, human immunodeficiency virus (HIV), Epstein-Barr virus (EBV), cytomegalovirus (CMV)(e.g., CMV5), human herpesviruses (HHV)(e.g., HHV6, 7 or 8), herpes simplex viruses (HSV), bovine herpes virus (BHV)(e.g., BHV4), equine herpes virus (EHV)(e.g., EHV2), human T-Cell leukemia viruses (HTLV)5, Varicella-Zoster virus (VZV), measles virus, papovaviruses (JC and BK), hepatitis viruses (E.g., HBV or HCV), myxoma virus, adenovirus, parvoviruses, polyoma virus, influenza viruses, papillomaviruses and poxviruses such as vaccinia virus, and molluscum contagiosum virus (MCV), and lyssaviruses. Such virus may or may not express an apoptosis inhibitor. Exemplary diseases caused by viral infection include, but are not limited to, chicken pox, Cytomegalovirus infections, genital herpes, Hepatitis B and C, influenza, and shingles, and rabies.

Exemplary bacteria include, but are not limited to, Campylobacter jejuni, Enterobacter species, Enterococcus faecium, Enterococcus faecalis, Escherichia coli (e.g., E. coli O157:H7), Group A streptococci, Haemophilus influenzae, Helicobacter pylori, listeria, Mycobacterium tuberculosis, Pseudomonas aeruginosa, S. pneumoniae, Salmonella, Shigella, Staphylococcus aureus, and Staphylococcus epidermidis, and Borrelia and Rickettsia. Exemplary diseases caused by bacterial infection include, but are not limited to, anthrax, cholera, diphtheria, foodborne illnesses, leprosy, meningitis, peptic ulcer disease, pneumonia, sepsis, septic shock, syphilis, tetanus, tuberculosis, typhoid fever, and urinary tract infection, and Lyme disease and Rocky Mountain spotted fever.

In some embodiments, the condition, disorder or disease is the result of, arising from or associated with undesirable inflammation, such as an immune disorder. Exemplary immune disorders include, but are not limited to, autoimmune diseases (for example, diabetes mellitus, arthritis—including rheumatoid arthritis, juvenile rheumatoid arthritis, osteoarthritis and psoriatic arthritis-, multiple sclerosis, encephalomyelitis, myasthenia gravis, systemic lupus erythematosis, autoimmune thyroiditis, dermaitis (including atopic dermatitis and eczematous dermatitis), psoriasis, Sjogren's Syndrome, Crohn's disease, aphthous ulcer, iritis, conjunctivitis, keratoconjunctivitis, ulcerative colitis, asthma, allergic asthma, sepsis and septic shock, inflammatory bowel disorder, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorimeural hearing loss, aplastic anemia, pure red cell anemia, idiopathic thrombocytopenia, polychondritis, Wegener's granulomatosis, chronic active hepatitis, Stevens-Johnson syndrome, glomerulonephritis, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy. Compounds and compositions of the disclosure can additionally be used to boost the immune system.

Oxidative stress plays a major role in the pathogenesis of multiple sclerosis (MS). Reactive oxygen species (ROS) have been implicated as mediators of demyelination and axonal damage in both MS and its animal model, experimental autoimmune encephalomyelitis (EAE). Experimental autoimmune encephalomyelitis (EAE) is the most commonly used experimental model for the human inflammatory demyelinating disease, multiple sclerosis (MS). Accordingly, in some embodiments, there are provided methods for treatment of MS in a subject need thereof, comprising administration of an effective amount of a compound or composition disclosed herein to the subject.

In some embodiments, compounds or compositions provided herein may be used alone or in combination with other therapeutic agents, e.g., other therapies for ferroptosis-associated diseases or disorders. Thus, compounds and/or compositions described herein may be administered alone or in combination with one or more additional therapy. The latter can be administered before, after or simultaneously with the administration of the compounds and/or compositions described herein.

In some embodiments, the compound or composition of the disclosure and the one or more additional agents are present in a combined composition. In some embodiments, the compound or composition of the disclosure and the one or more additional agents are administered separately. Also provided are pharmaceutical compositions comprising at least one compound or pharmaceutically acceptable salt thereof described herein and one or more additional pharmaceutical agent used in the treatment of a disease or disorder associated with ferroptosis. Similarly, also provided are packaged pharmaceutical compositions containing a first pharmaceutical composition comprising at least one compound or composition described herein, and another composition comprising one or more additional pharmaceutical agents used in the treatment of a disease or disorder associated with ferroptosis.

Consequently, there are provided methods for inhibition of ferroptosis in a subject by administering an effective amount of a compound or composition described herein. The term “subject” includes living organisms with a ferroptosis-associated disease or disorder (e.g., a neurodegenerative disease), or who are susceptible to or at risk of a ferroptosis-associated disease or disorder, e.g., due to a genetic predisposition, environmental exposure to carcinogens, and the like. Examples of subjects include humans, monkeys, cows, rabbits, sheep, goats, pigs, dogs, cats, rats, mice, and transgenic species thereof. The term “subject” generally includes animals susceptible to states associated with ferroptosis, such as without limitation, mammals, e.g. primates, e.g. humans. The animal can also be an animal model for a disorder, e.g., a mouse model for Alzheimer's disease, and the like.

In some embodiments, a subject is in need of treatment by the methods provided herein and is selected for treatment based on this need. A subject in need of treatment is art-recognized, and includes subjects that have been identified as having a disease or condition (e.g., a neurodegenerative disease), or having a symptom of such a disease or condition, or being at risk of such a disease or condition, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder).

As used herein, “treating” or “treatment” of a disease or condition refers, in some embodiments, to ameliorating at least one disease or condition (i.e., arresting or reducing the development of a disease or condition or at least one of the clinical symptoms thereof). In certain embodiments “treating” or “treatment” refers to ameliorating at least one physical parameter, such as e.g. tumor size, growth, or migration. In certain embodiments, “treating” or “treatment” refers to inhibiting or improving a disease or condition, either physically (e.g., stabilization of a discernible symptom), physiologically (e.g., stabilization of a physical parameter), or both. In certain embodiments, “treating” or “treatment” refers to delaying the onset (or recurrence) of a disease or condition. The term “treating” or “treatment” may refer to any indicia of success in the treatment or amelioration of a disease or condition, including any objective or subjective parameter such as abatement; remission; diminishing of symptoms or making the disease or condition more tolerable to the subject; improving a subject's physical or mental well-being, such as reducing pain experienced by the patient; and, in some situations additionally improving at least one parameter of a disease or condition.

As used herein, “preventing” or “prevention” is intended to refer at least to the reduction of the likelihood of, or the risk of, or susceptibility to acquiring a disease or disorder (i.e., causing at least one of the clinical symptoms of the disease not to develop in a patient that may be exposed to or predisposed to or at risk of the disease but does not yet experience or display symptoms of the disease). The term “prevention” or “preventing” is also used to describe the administration of a compound or composition described herein to a subject who is at risk of (or susceptible to) such a disease or condition. Subjects amenable to treatment for prevention of a disease or condition include individuals at risk of the disease or condition but not showing symptoms, as well as patients presently showing symptoms. In some embodiments, “prevention” or “preventing” is used to describe the administration of a compound or composition described herein to a subject who has been diagnosed with or treated for a disease or condition and is at risk of recurrence of the disease or condition.

In some embodiments, treatment or prevention are within the context of the present invention if there is a measurable difference between the performances of subjects treated using the compounds and methods provided herein as compared to members of a placebo group, historical control, or between subsequent tests given to the same subject.

The term “inhibition” or “inhibiting” is used herein to refer generally to reducing, slowing, restricting, delaying, suppressing, blocking, hindering, or preventing a process, such as without limitation reducing or slowing neuronal degeneration, neuronal cell loss, cognitive decline, and the like.

The term “effective amount” as used herein means that amount or dose of a compound or composition, upon single or multiple dose administration to a subject, which provides the desired effect (e.g., the desired biological or medicinal response, e.g., to ameliorate, lessen or prevent a disease, disorder or condition) in the subject being treated. In some embodiments, an effective amount is an amount or dose of a compound or composition that prevents or treats a ferroptosis-associated disease or disorder in a subject, as described herein. The terms “effective amount” and “therapeutically effective amount” are used interchangeably herein.

It should be understood that the dosage or amount of a compound and/or composition used, alone or in combination with one or more active compounds to be administered, depends on the individual case and is, as is customary, to be adapted to the individual circumstances to achieve an optimum effect. Dosing and administration regimens are within the purview of the skilled artisan, and appropriate doses depend upon a number of factors within the knowledge of the ordinarily skilled physician, veterinarian, or researcher (e.g., see Wells et al. eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif (2000)). For example, dosing and administration regimens depend on the nature and the severity of the disorder to be treated, and also on the sex, age, weight and individual responsiveness of the human or animal to be treated, on the efficacy and duration of action of the compounds used, on whether the therapy is acute or chronic or prophylactic, and/or on whether other active compounds are administered in addition to the therapeutic molecule(s).

Thus the dose(s) of a compound or composition will vary depending upon a variety of factors including, but not limited to: the activity, biological and pharmacokinetic properties and/or side effects of the compound being used; the age, body weight, general health, gender, and diet of the subject; the time of administration, the route of administration, the rate of excretion, and any drug combination, if applicable; the effect which the practitioner desires the compound to have upon the subject; and the properties of the compound being administered (e.g. bioavailability, stability, potency, toxicity, etc). Such appropriate doses may be determined as known in the art. When one or more of the compounds of the invention is to be administered to humans, a physician may for example, prescribe a relatively low dose at first, subsequently increasing the dose until an appropriate response is obtained.

There are no particular limitations on the dose of each of the compounds for use in compositions provided herein. Exemplary doses include milligram or microgram amounts of the compound per kilogram of subject or sample weight (e.g., about 50 micrograms per kilogram to about 500 milligrams per kilogram, about 1 milligram per kilogram to about 100 milligrams per kilogram, about 1 milligram per kilogram to about 50 milligrams per kilogram, about 1 milligram per kilogram to about 10 milligrams per kilogram, or about 3 milligrams per kilogram to about 5 milligrams per kilogram). Additional exemplary doses include doses of about 5 to about 500 mg, about 25 to about 300 mg, about 25 to about 200 mg, about 50 to about 150 mg, or about 50, about 100, about 150 mg, about 200 mg or about 250 mg, and, for example, daily or twice daily, or lower or higher amounts.

In some embodiments, the dose range for adult humans is generally from 0.005 mg to 10 g/day orally. Tablets or other forms of presentation provided in discrete units may conveniently contain an amount of a compound (e.g., of Formula A, of Formula I, of Formula II, of Formula III, of Formula IV, or in Table 1 or 2) which is effective at such dosage or as a multiple of the same, for instance, units containing 5 mg to 500 mg, usually around 10 mg to 200 mg. A dosage unit (e.g., an oral dosage unit) can include from, for example, 1 to 30 mg, 1 to 40 mg, 1 to 100 mg, 1 to 300 mg, 1 to 500 mg, 2 to 500 mg, 3 to 100 mg, 5 to 20 mg, 5 to 100 mg (e.g. 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, or 500 mg) of a compound described herein.

Administration of compounds and compositions provided herein can be carried out using known procedures, at dosages and for periods of time effective to achieve a desired purpose. Dosage regimens can be adjusted to provide the optimum therapeutic response. For example, several divided doses may be administered daily, or the dose may be proportionally reduced as indicated by the exigencies of the therapeutic situation. In some embodiments, a compound or composition is administered at an effective dosage sufficient to prevent or treat a ferroptosis-associated disease or disorder, e.g., a neurodegenerative disease, in a subject. Further, a compound or composition may be administered using any suitable route or means, such as without limitation via oral, parenteral, intravenous, intraperitoneal, intramuscular, sublingual, topical, or nasal administration, via inhalation, or via such other routes as are known in the art.

### Kits

Compound and compositions provided herein may be packaged as part of a kit, optionally including a container (e.g. packaging, a box, a vial, etc). The kit may be commercially used according to the methods described herein and may include instructions for use in such methods. Additional kit components may include acids, bases, buffering agents, inorganic salts, solvents, antioxidants, preservatives, or metal chelators. The additional kit components may be present as pure compositions, or as aqueous or organic solutions that incorporate one or more additional kit components. Any or all of the kit components optionally further comprise buffers.

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention.

### Example 1. Design of Ferroptosis Inhibitor Compounds

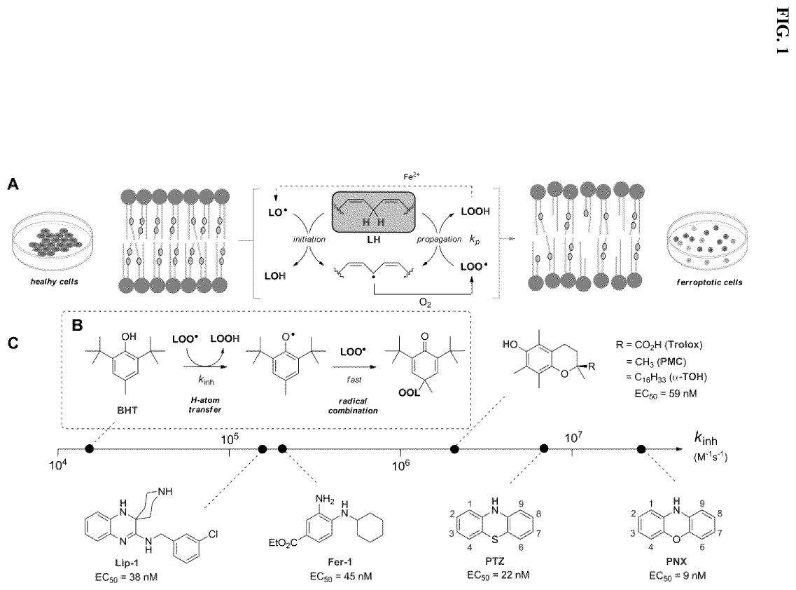

We recently developed a high-throughput assay of RTA activity in phospholipid bilayers dubbed FENIX (Fluorescence ENabled Inhibited autoXidation) which is predictive of potency to inhibit ferroptosis (illustrated with mouse embryonic fibroblasts, or MEFs) for a wide variety of compounds (Shah et al., 2019). Herein, we used the FENIX approach to carry out a structure-reactivity-potency study of an inhibitor scaffold for the rational design of a ferroptosis inhibitor, from a reactivity standpoint. We chose phenoxazine (PNX, FIG. 1(C)) as the starting scaffold for improvement since it is a highly reactive RTA (compare kᵢₙₕ ~4×10⁷ M⁻¹s⁻¹ vs. 2×10⁵ and 3×10⁵ M⁻¹s⁻¹ for Lip-1 and Fer-1, respectively, at 37° C. in PhCl) (Farmer et al., 2017; Lucarini et al., 1999) and a potent inhibitor of ferroptosis (EC₅₀=9 nM vs. 35 and 45 nM for inhibition of RSL3-induced ferroptosis in MEFs, respectively) (Shah et al., 2017). We also explored a set of analogous phenothiazine (PTZ, FIG. 1(C)) derivatives. PTZ is structurally similar to PNX but has lower inherent RTA activity (kᵢₙₕ ~8×10⁶ M⁻¹s⁻¹ at 37° C. in PhCl) (Farmer et al., 2017). Design and testing of these RTA-based ferroptosis inhibitors are described herein below.

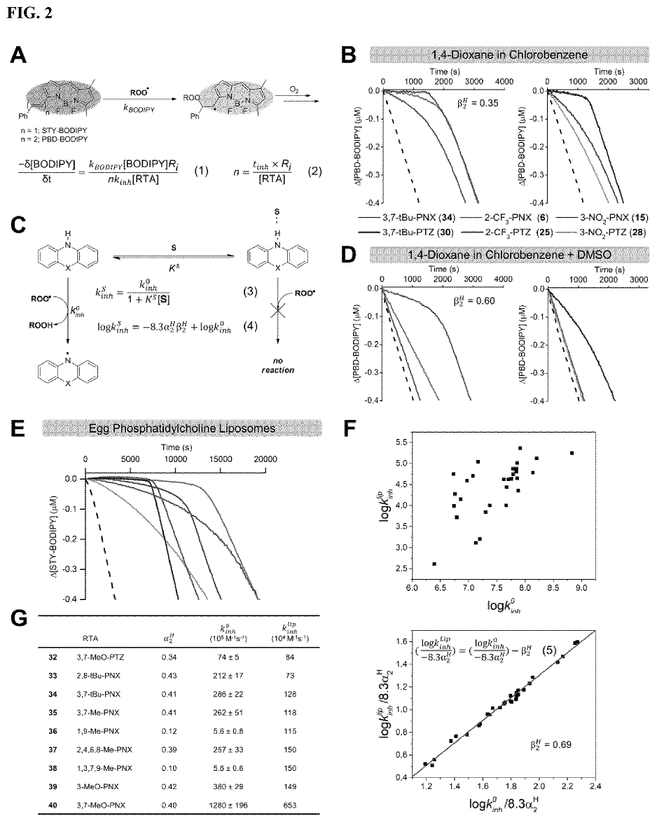

Compounds 1-95 were either purchased or designed and synthesized as described below. Derivatives of PNX and PTZ were designed to add electron-donating (ED) or electron-withdrawing (EW) groups, or combinations thereof, at various positions on the two aromatic rings, in order to systematically probe structure-function relationships for RTA activity and ferroptosis inhibition. Structures of compounds 1-40 are shown in Table 1 and FIG. 2, abbreviated according to the substituent(s) and its (their) position(s) according to the convention in FIG. 1. Structures of compounds 4, 6-23, 33-95 are shown in Table 2.

TABLE 1
Inhibition Rate Constants (kᵢₙₕ^{lip}) and Radical-Trapping
Stoichiometries (n) of PNX and PTZ Derivative Compounds
Determined from Co-Autoxidations of STY-BODIPY
and Polyunsaturated Phospholipids of Egg PC Liposomes.

| | | | kᵢₙₕ⁰ | kᵢₙₕ^{lip} | |
|---|---|---|---|---|---|
| | | α₂^{H a} | (10⁵ M⁻¹s⁻¹) ^{b} | (10⁵ M⁻¹s⁻¹)^{d} | n |
| 1 | PNX | 0.44 | (820 ± 77)^{c} | (232 ± 23)^{e} | 2.0 ± 0.1 |
| 2 | PTZ | 0.38 | (147 ± 6) | (110 ± 20)^{e} | 1.9 ± 0.1 |
| 3 | PSZ | 0.36 | (54 ± 2) | (56 ± 3)^{e} | 2.3 ± 0.1 |
| 4 | Benzo(b)-PNX | 0.49 | (723 ± 42)^{c} | (101 ± 12) | 2.4 ± 0.1 |
| 5 | Benzo(b)-PTZ | 0.40 | (120 ± 3) | (51 ± 3) | 1.9 ± 0.1 |
| 6 | 2-CF₃-PNX | 0.51 | (610 ± 30) | (75 ± 11) | 3.0 ± 0.1 |
| 7 | 2-CF₃-7-tBu-PNX | 0.50 | (703 ± 8)^{c} | (75 ± 13) | 2.8 ± 0.1 |
| 8 | 2-CF₃-8-tBu-PNX | 0.51 | (699 ± 15)^{c} | (64 ± 9) | 2.3 ± 0.1 |
| 9 | 3-CF₃-PNX | 0.53 | (508 ± 56) | (42 ± 2) | 3.0 ± 0.1 |
| 10 | 3-CF₃-7-tBu-PNX | 0.51 | (613 ± 14)^{c} | (55 ± 10) | 2.8 ± 0.1 |
| 11 | 3-CF₃-8-tBu-PNX | 0.52 | (574 ± 4)^{c} | (44 ± 3) | 2.6 ± 0.1 |
| 12 | 3-CHO-PNX | 0.55 | (421 ± 57) | (42 ± 2) | 2.9 ± 0.1 |
| 13 | 3,7-CHO-PNX | 0.63 | (238 ± 37) | (10 ± 1) | 3.2 ± 0.1 |
| 14 | 3-CHO-7-CN-PNX | 0.64 | (200 ± 12) | (7.1 ± 0.1) | nd^{f} |
| 15 | 3-NO₂-PNX | 0.62 | (463 ± 14) | (10 ± 1)^{e} | 2.2 ± 0.1 |
| 16 | 3-NO₂-7-Me-PNX | 0.60 | (757 ± 92) | (23 ± 2)^{e} | 3.8 ± 0.3 |
| 17 | 3-NO₂-7-MeO-PNX | 0.60 | (1370 ± 186) | (61 ± 4)e | 2.9 ± 0.2 |
| 18 | 3-NO₂-7-NEt₂-PNX | 0.58 | (6784 ± 342) | (177 ± 37)^{e} | 2.4 ± 0.4 |
| 19 | 3,7-NO₂-PNX | 0.72 | (134 ± 8) | (1.3 ± 0.1)^{e} | nd^{f} |
| 20 | 3-CN-PNX | 0.56 | (472 ± 33) | (27 ± 2) | 3.6 ± 0.1 |
| 21 | 3-CN-7-Me-PNX | 0.55 | (726 ± 43) | (44 ± 6) | 3.5 ± 0.1 |
| 22 | 3-CN-7-MeO-PNX | 0.55 | (1605 ± 154) | (133 ± 15)^{e} | 3.1 ± 0.3 |
| 23 | 3-CN-7-NO₂-PNX | 0.69 | (158 ± 2) | (1.6 ± 0.1)^{e} | nd^{f} |
| 24 | 2-Cl-PTZ | 0.43 | (94 ± 8) | (39 ± 4) | 2.1 ± 0.1 |
| 25 | 2-CF₃-PTZ | 0.44 | (57 ± 3) | (19 ± 1) | 2.5 ± 0.1 |
| 26 | 3-CF₃-PTZ | 0.45 | (55 ± 3) | (9.8 ± 0.5) | 2.6 ± 0.1 |
| 27 | 3-CHO-PTZ | 0.47 | (72 ± 4) | (14 ± 1) | 2.2 ± 0.1 |
| 28 | 3-NO₂-PTZ | 0.52 | (62 ± 2) | (5.3 ± 0.3) | 2.0 ± 0.1 |
| 29 | 3,7-NO₂-PTZ | 0.62 | (25 ± 2) | (0.41 ± 0.04) | nd^{f} |
| 30 | 3,7-tBu-PTZ | 0.35 | (199 ± 11)^{c} | (106 ± 14) | 1.6 ± 0.1 |
| 31 | 1,9-Me-PTZ | 0.06 | (3.7 ± 0.2) | (98 ± 5) | 1.7 ± 0.1 |
| | PMC | 0.38 | (52 ± 3) | (29 ± 2) | (2) |
| 32 | 3,7-MeO-PTZ | 0.34 | (740 ± 50) | 840 ^{f} | |
| 33 | 2,8-tBu-PNX | 0.43 | (2120 ± 170) | 730 ^{f} | |
| 34 | 3,7-tBu-PNX | 0.41 | (2860 ± 220) | 1280 ^{f} | |
| 35 | 3,7-Me-PNX | 0.41 | (2620 ± 510) | 1180 ^{f} | |
| 36 | 1,9-Me-PNX | 0.12 | (56 ± 8) | 1150 ^{f} | |
| 37 | 2,4,6,8-Me-PNX | 0.39 | (2570 ± 330) | 1500 ^{f} | |
| 38 | 1,3,7,9-Me-PNX | 0.10 | (56 ± 6) | 1500 ^{f} | |
| 39 | 3-MeO-PNX | 0.42 | (3800 ± 290) | 1490 ^{f} | |
| 40 | 3,7-MeO-PNX | 0.40 | (12800 ± 1960) | 6530 ^{f} | |

In Table 1, (a): Estimated from the ¹H NMR chemical shift difference of the amine proton in DMSO-d₆ and benzene-d₆; (b): Determined from inhibition rate constants measured in 1:3 dioxane:PhCl (kᵢₙₕ^{dioxane}), which has β₂^{H}=0.35 and α₂^{H} from the preceding column using Eq. 4; (c): Determined from inhibition rate constants measured in 1:2:1 dioxane:PhCl:DMSO (kᵢₙₕ^{DMSO}), which has β₂^{H}=0.60 and α₂^{H} from the preceding column using Eq. 4; (d): Apparent inhibition rate constants from MeOAMVN-initiated autoxidations of egg PC liposomes determined by fluorescence; (e): Apparent inhibition rate constants from MeOAMVN-initiated autoxidations of egg PC liposomes determined by absorbance (STY-BODIPY, 10-20 μM, λmax=565 nm, ε=123 676 M⁻¹ cm⁻¹); (f):Inhibition rate constants predicted from log kᵢₙₕ⁰, α₂^{H} and β₂^{H}=0.69 using Eq. 4; (g): Not determined (nd) due to the lack of a well-defined inhibited period. An n=2 was used in the calculation of kᵢₙₕ^{lip}.

TABLE 2
Exemplary compounds of the invention.

| | |
|---|---|
| 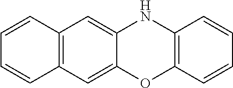 [SMILES: c1ccc2c(c1)Nc1cc3ccccc3cc1O2] | (4) |
| 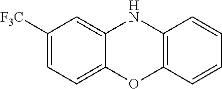 [SMILES: FC(F)(F)c1ccc2c(c1)Nc1ccccc1O2] | (6) |
| 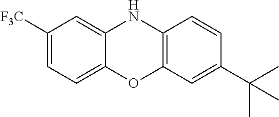 [SMILES: CC(C)(C)c1ccc2c(c1)Oc1ccc(C(F)(F)F)cc1N2] | (7) |
| 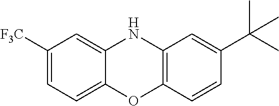 [SMILES: CC(C)(C)c1ccc2c(c1)Nc1cc(C(F)(F)F)ccc1O2] | (8) |
| 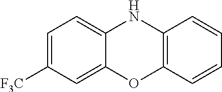 [SMILES: FC(F)(F)c1ccc2c(c1)Oc1ccccc1N2] | (9) |
| 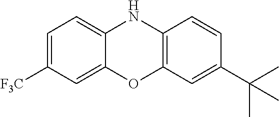 [SMILES: CC(C)(C)c1ccc2c(c1)Oc1cc(C(F)(F)F)ccc1N2] | (10) |
| 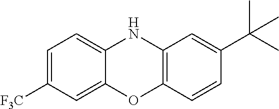 [SMILES: CC(C)(C)c1ccc2c(c1)Nc1ccc(C(F)(F)F)cc1O2] | (11) |
| 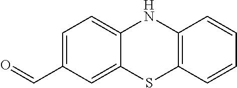 [SMILES: O=Cc1ccc2c(c1)Sc1ccccc1N2] | (12) |
| 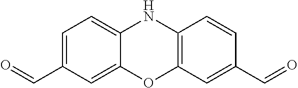 [SMILES: O=Cc1ccc2c(c1)Oc1cc(C=O)ccc1N2] | (13) |
| 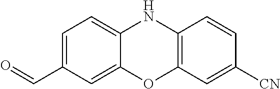 [SMILES: N#Cc1ccc2c(c1)Oc1cc(C=O)ccc1N2] | (14) |
| 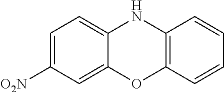 [SMILES: O=[N+]([O-])c1ccc2c(c1)Oc1ccccc1N2] | (15) |
| 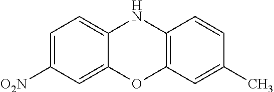 [SMILES: Cc1ccc2c(c1)Oc1cc([N+](=O)[O-])ccc1N2] | (16) |
| 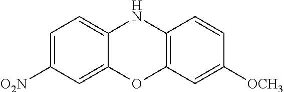 [SMILES: COc1ccc2c(c1)Oc1cc([N+](=O)[O-])ccc1N2] | (17) |
| 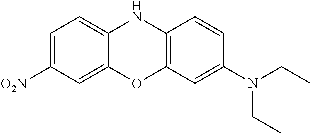 [SMILES: CCN(CC)c1ccc2c(c1)Oc1cc([N+](=O)[O-])ccc1N2] | (18) |
| 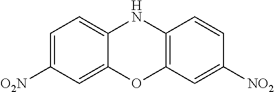 [SMILES: O=[N+]([O-])c1ccc2c(c1)Oc1cc([N+](=O)[O-])ccc1N2] | (19) |
| 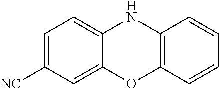 [SMILES: N#Cc1ccc2c(c1)Oc1ccccc1N2] | (20) |
| 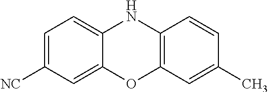 [SMILES: Cc1ccc2c(c1)Oc1cc(C#N)ccc1N2] | (21) |
| 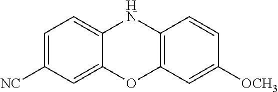 [SMILES: COc1ccc2c(c1)Oc1cc(C#N)ccc1N2] | (22) |
| 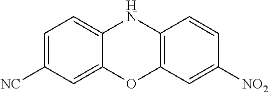 [SMILES: N#Cc1ccc2c(c1)Oc1cc([N+](=O)[O-])ccc1N2] | (23) |
| 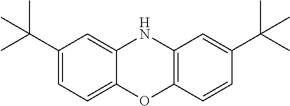 [SMILES: CC(C)(C)c1ccc2c(c1)Nc1cc(C(C)(C)C)ccc1O2] | (33) |
| 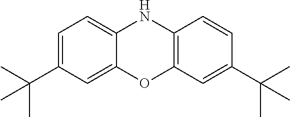 [SMILES: CC(C)(C)c1ccc2c(c1)Oc1cc(C(C)(C)C)ccc1N2] | (34) |
| 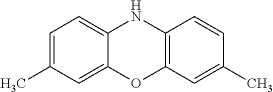 | (35) |
| 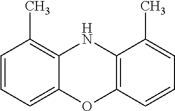 [SMILES: Cc1cccc2c1Nc1c(C)cccc1O2] | (36) |
| 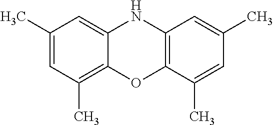 | (37) |
| 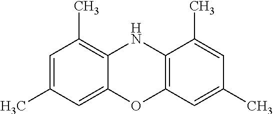 | (38) |
| 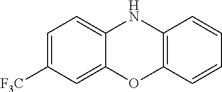 [SMILES: FC(F)(F)c1ccc2c(c1)Oc1ccccc1N2] | (39) |
| 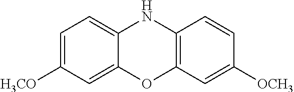 [SMILES: COc1ccc2c(c1)Oc1cc(OC)ccc1N2] | (40) |
| 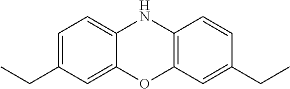 [SMILES: CCc1ccc2c(c1)Oc1cc(CC)ccc1N2] | (41) |
| 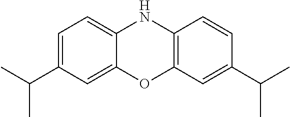 [SMILES: CC(C)c1ccc2c(c1)Oc1cc(C(C)C)ccc1N2] | (42) |
| 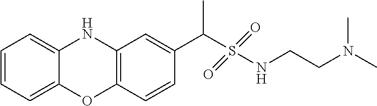 [SMILES: CC(c1ccc2c(c1)Nc1ccccc1O2)S(=O)(=O)NCCN(C)C] | (43) |
| 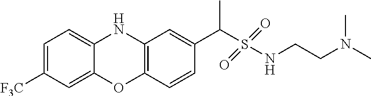 [SMILES: CC(c1ccc2c(c1)Nc1ccc(C(F)(F)F)cc1O2)S(=O)(=O)NCCN(C)C] | (44) |
| 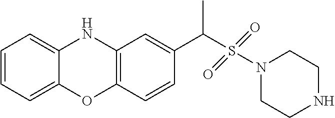 [SMILES: CC(c1ccc2c(c1)Nc1ccccc1O2)S(=O)(=O)N1CCNCC1] | (45) |
| 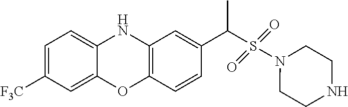 [SMILES: CC(c1ccc2c(c1)Nc1ccc(C(F)(F)F)cc1O2)S(=O)(=O)N1CCNCC1] | (46) |
| 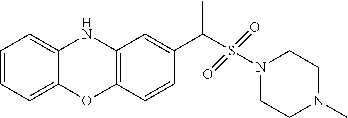 [SMILES: CC(c1ccc2c(c1)Nc1ccccc1O2)S(=O)(=O)N1CCN(C)CC1] | (47) |
| 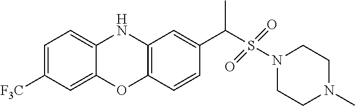 [SMILES: CC(c1ccc2c(c1)Nc1ccc(C(F)(F)F)cc1O2)S(=O)(=O)N1CCN(C)CC1] | (48) |
| 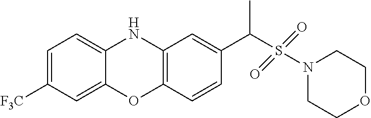 [SMILES: CC(c1ccc2c(c1)Nc1ccc(C(F)(F)F)cc1O2)S(=O)(=O)N1CCOCC1] | (49) |
| 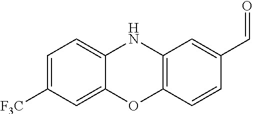 [SMILES: O=Cc1ccc2c(c1)Nc1ccc(C(F)(F)F)cc1O2] | (50) |
| 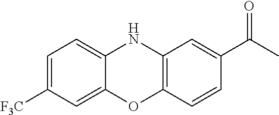 [SMILES: CC(=O)c1ccc2c(c1)Nc1ccc(C(F)(F)F)cc1O2] | (51) |
| 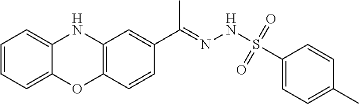 [SMILES: C/C(=N\NS(=O)(=O)c1ccc(C)cc1)c1ccc2c(c1)Nc1ccccc1O2] | (52) |
| 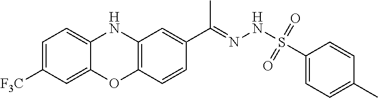 [SMILES: C/C(=N\NS(=O)(=O)c1ccc(C)cc1)c1ccc2c(c1)Nc1ccc(C(F)(F)F)cc1O2] | (53) |
| 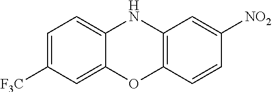 [SMILES: O=[N+]([O-])c1ccc2c(c1)Nc1ccc(C(F)(F)F)cc1O2] | (54) |
| 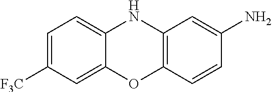 [SMILES: Nc1ccc2c(c1)Nc1ccc(C(F)(F)F)cc1O2] | (55) |
| 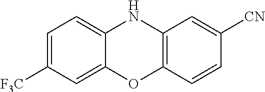 [SMILES: N#Cc1ccc2c(c1)Nc1ccc(C(F)(F)F)cc1O2] | (56) |
| 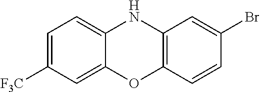 [SMILES: FC(F)(F)c1ccc2c(c1)Oc1ccc(Br)cc1N2] | (57) |
| 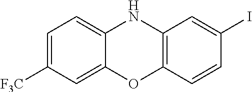 [SMILES: FC(F)(F)c1ccc2c(c1)Oc1ccc(I)cc1N2] | (58) |
| 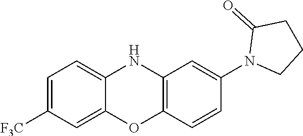 [SMILES: O=C1CCCN1c1ccc2c(c1)Nc1ccc(C(F)(F)F)cc1O2] | (59) |
| 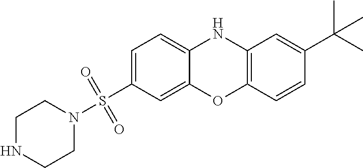 [SMILES: CC(C)(C)c1ccc2c(c1)Nc1ccc(S(=O)(=O)N3CCNCC3)cc1O2] | (60) |
| 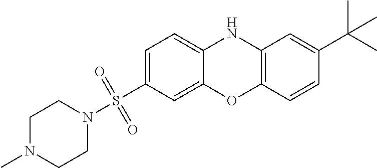 [SMILES: CN1CCN(S(=O)(=O)c2ccc3c(c2)Oc2ccc(C(C)(C)C)cc2N3)CC1] | (61) |
| 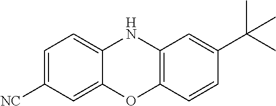 [SMILES: CC(C)(C)c1ccc2c(c1)Nc1ccc(C#N)cc1O2] | (62) |
| 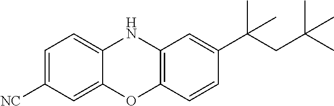 [SMILES: CC(C)(C)CC(C)(C)c1ccc2c(c1)Nc1ccc(C#N)cc1O2] | (63) |
| 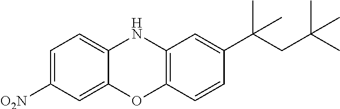 [SMILES: CC(C)(C)CC(C)(C)c1ccc2c(c1)Nc1ccc([N+](=O)[O-])cc1O2] | (64) |
| 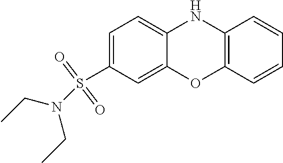 [SMILES: CCN(CC)S(=O)(=O)c1ccc2c(c1)Oc1ccccc1N2] | (65) |
| 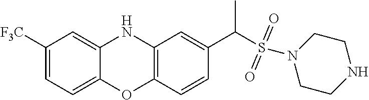 [SMILES: CC(c1ccc2c(c1)Nc1cc(C(F)(F)F)ccc1O2)S(=O)(=O)N1CCNCC1] | (66) |
| 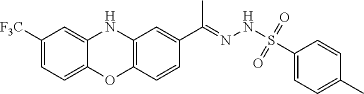 [SMILES: C/C(=N\NS(=O)(=O)c1ccc(C)cc1)c1ccc2c(c1)Nc1cc(C(F)(F)F)ccc1O2] | (67) |
| 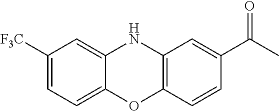 [SMILES: CC(=O)c1ccc2c(c1)Nc1cc(C(F)(F)F)ccc1O2] | (68) |
| 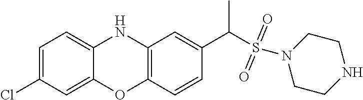 [SMILES: CC(c1ccc2c(c1)Nc1ccc(Cl)cc1O2)S(=O)(=O)N1CCNCC1] | (69) |
| 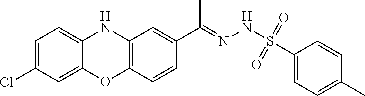 [SMILES: C/C(=N\NS(=O)(=O)c1ccc(C)cc1)c1ccc2c(c1)Nc1ccc(Cl)cc1O2] | (70) |
| 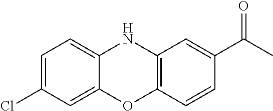 [SMILES: CC(=O)c1ccc2c(c1)Nc1ccc(Cl)cc1O2] | (71) |
| 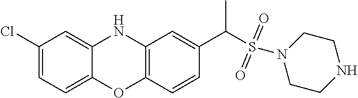 [SMILES: CC(c1ccc2c(c1)Nc1cc(Cl)ccc1O2)S(=O)(=O)N1CCNCC1] | (72) |
| 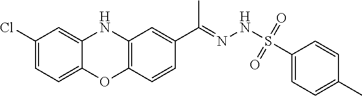 [SMILES: C/C(=N\NS(=O)(=O)c1ccc(C)cc1)c1ccc2c(c1)Nc1cc(Cl)ccc1O2] | (73) |
| 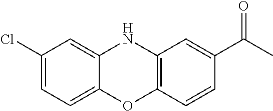 [SMILES: CC(=O)c1ccc2c(c1)Nc1cc(Cl)ccc1O2] | (74) |
| 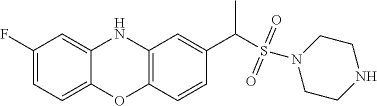 [SMILES: CC(c1ccc2c(c1)Nc1cc(F)ccc1O2)S(=O)(=O)N1CCNCC1] | (75) |
| 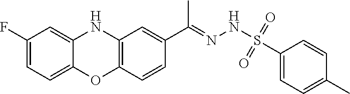 [SMILES: C/C(=N\NS(=O)(=O)c1ccc(C)cc1)c1ccc2c(c1)Nc1cc(F)ccc1O2] | (76) |
| 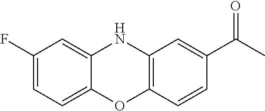 [SMILES: CC(=O)c1ccc2c(c1)Nc1cc(F)ccc1O2] | (77) |
| 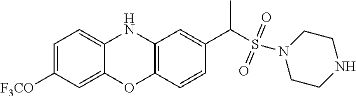 [SMILES: CC(c1ccc2c(c1)Nc1ccc(OC(F)(F)F)cc1O2)S(=O)(=O)N1CCNCC1] | (78) |
| 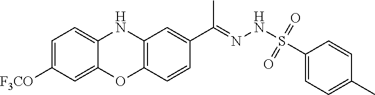 [SMILES: C/C(=N\NS(=O)(=O)c1ccc(C)cc1)c1ccc2c(c1)Nc1ccc(OC(F)(F)F)cc1O2] | (79) |
| 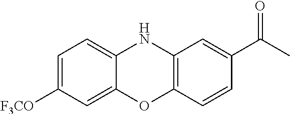 [SMILES: CC(=O)c1ccc2c(c1)Nc1ccc(OC(F)(F)F)cc1O2] | (80) |
| 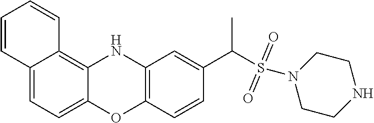 [SMILES: CC(c1ccc2c(c1)Nc1c(ccc3ccccc13)O2)S(=O)(=O)N1CCNCC1] | (81) |
| 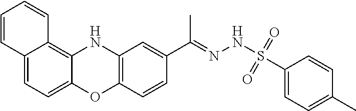 [SMILES: C/C(=N\NS(=O)(=O)c1ccc(C)cc1)c1ccc2c(c1)Nc1c(ccc3ccccc13)O2] | (82) |
| 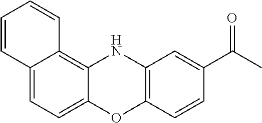 [SMILES: CC(=O)c1ccc2c(c1)Nc1c(ccc3ccccc13)O2] | (83) |
| 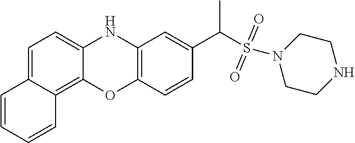 [SMILES: CC(c1ccc2c(c1)Nc1ccc3ccccc3c1O2)S(=O)(=O)N1CCNCC1] | (84) |
| 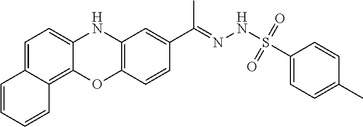 [SMILES: C/C(=N\NS(=O)(=O)c1ccc(C)cc1)c1ccc2c(c1)Nc1ccc3ccccc3c1O2] | (85) |
| 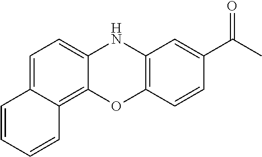 [SMILES: CC(=O)c1ccc2c(c1)Nc1ccc3ccccc3c1O2] | (86) |
| 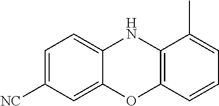 [SMILES: Cc1cccc2c1Nc1ccc(C#N)cc1O2] | (87) |
| 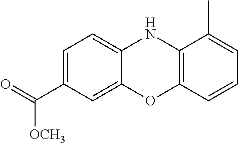 [SMILES: COC(=O)c1ccc2c(c1)Oc1cccc(C)c1N2] | (88) |
| 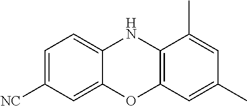 [SMILES: Cc1cc(C)c2c(c1)Oc1cc(C#N)ccc1N2] | (89) |
| 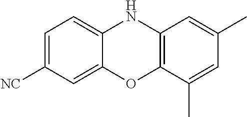 [SMILES: Cc1cc(C)c2c(c1)Nc1ccc(C#N)cc1O2] | (90) |
| 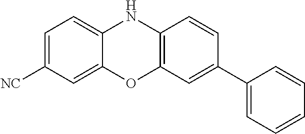 [SMILES: N#Cc1ccc2c(c1)Oc1cc(-c3ccccc3)ccc1N2] | (91) |
| 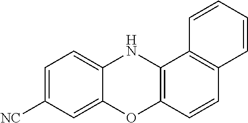 [SMILES: N#Cc1ccc2c(c1)Oc1ccc3ccccc3c1N2] | (92) |
| 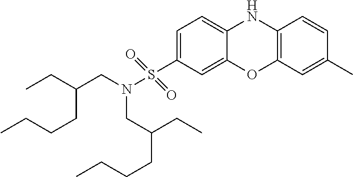 [SMILES: CCCCC(CC)CN(CC(CC)CCCC)S(=O)(=O)c1ccc2c(c1)Oc1cc(C)ccc1N2] | (93) |
| 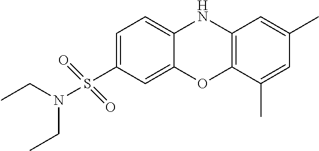 [SMILES: CCN(CC)S(=O)(=O)c1ccc2c(c1)Oc1c(C)cc(C)cc1N2] | (94) |
| 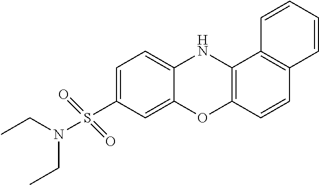 [SMILES: CCN(CC)S(=O)(=O)c1ccc2c(c1)Oc1ccc3ccccc3c1N2] | (95) |

### Example 2. Synthesis and Characterization of Ferroptosis Inhibitor Compounds

General. Reagents and solvents were procured from commercial sources and were used without further purification unless otherwise indicated. The preparation of phenoselenazine (3) (Tin, G.; Mohamed, T.; Gondora, N.; Beazely, M. A.; Rao, P. P. N. Tricyclic Phenothiazine and Phenoselenazine Derivatives as Potential Multi-Targeting Agents to Treat Alzheimer's Disease. Medchemcomm 2015, 6 (11), 1930-1941), 12H-Benzo[b]phenoxazine (4), 12H-Benzo[b]phenothiazine (5) (Crivello, J. V. Benzophenothiazine and Benzophenoxazine Photosensitizers for Triarylsulfonium Salt Cationic Photoinitiators. J. Polym. Sci. Part A Polym. Chem. 2008, 46 (11), 3820-3829), 2-trifluoromethylphenoxazine (6), 3-trifluoromethylphenoxazine (9), 3-cyanophenoxazine (20), 3-cyano-7-nitrophenoxazine (23), 3-nitrophenoxazine (15), 3,7-di-tert-butylphenoxazine (34), 3-nitro-7-methoxyphenoxazine (17), 3-methoxyphenoxazine (39), 2,8-di-tert-butylphenoxazine (33), 3,7-dimethoxyphenoxazine (40), N,N-diethyl-3-sulfonamidephenoxazine (65) (Farmer, L. A.; Haidasz, E. A.; Griesser, M.; Pratt, D. A. Phenoxazine: A Privileged Scaffold for Radical-Trapping Antioxidants. J. Org. Chem. 2017, 82 (19), 10523-10536), 3-trifluoromethylphenothiazine (26), (Roe, A.; Little, W. F. The Preparation of Some Fluoro- and Trifluoromethyl-Phenothiazines, and Some Observations Regarding Determination of Their Structure by Infrared Spectroscopy. J. Org. Chem. 1955, 20 (11), 1577-1590), 3-nitrophenothiazine (28) (Mital, R. L.; Jain, S. K. Synthesis of Some 5-Substituted 2-Aminobenzenethiols and Their Conversion into Phenothiazines via Smiles Rearrangement. J. Chem. Soc. C Org. 1969, No. 16, 2148-2150), 3-formylphenothiazine (27) (Gaina, L.; Porumb, D.; Silaghi-Dumitrescu, I.; Cristea, C.; Silaghi-Dumitrescu, L. On the Microwave-Assisted Synthesis of Acylphenothiazine Derivatives Experiment versus Theory Synergism. Can. J. Chem. 2009, 88 (1), 42-49), 1,9-dimethylphenothiazine (31), 3,7-dimethoxyphenothiazine (32) (Lucarini, M.; Pedrielli, P.; Pedulli, G. F.; Valgimigli, L.; Gigmes, D.; Tordo, P. Bond Dissociation Energies of the N—H Bond and Rate Constants for the Reaction with Alkyl, Alkoxyl, and Peroxyl Radicals of Phenothiazines and Related Compounds. J. Am. Chem. Soc. 1999, 121, 11546-11553), and 2-acetylphenoxazine (VANDERHAEGHE, H. Phenoxazines. I. Ring-Substituted Derivatives. J. Org. Chem. 1960, 25 (5), 747-753) were carried out in a manner previously described.

2-((2,4-Dinitrophenyl)amino)-5-methylphenol (16a). In a round bottom flask affixed with a reflux condenser were combined ethanol (60 ml), 2-amino-5-methylphenol (1.85 g, 15 mmol), 1-chloro-2,4-dinitrobenzene (3.04 g, 15 mmol) and sodium acetate (1.23 g, 15 mmol). With stirring the mixture was refluxed (80° C.) for 4 hours after which the solution was allowed to cool to room temperature. The solution was poured into water and the red solid was filtered and rinsed with excess water. The filtered solid was recrystallized out of ethanol to afford the title product as red/orange needles (3.33 g, 77%). ¹H NMR (400 MHz; DMSO-d6): 9.87 (br, 2H), 8.89 (d, J=2.7 Hz, 1H), 8.21 (dd, J=2.7, 9.6 Hz, 1H), (d, J=7.9 Hz, 1H), 6.85-6.83 (m, 2H), 6.74 (dd, J=1.0, 8.0 Hz, 1H), 2.28 (s, 3H). 13C NMR (101 MHz; DMSO-d6): δ 152.0, 147.2, 138.3, 135.9, 130.6, 129.7, 127.1, 123.3, 121.6, 120.4, 117.2, 117.1, 20.9. (EI, magnetic sector): calcd for C13H11N3O5 289.06987, found 289.06685. mp=166-168° C. (lit. 166-167° C.) (Roberts, K. C.; Rhys, J. A. 8. A Rearrangement of o-Aminodiphenyl Ethers. Part V. J. Chem. Soc. 1937, No. 0, 39-41).

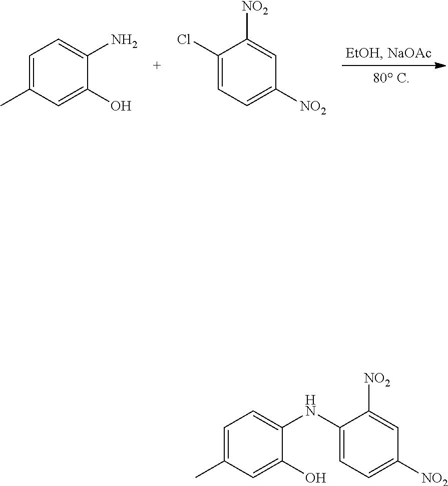

2-((2,4-dinitrophenyl)amino)-4-(2,4,4-trimethylpentan-2-yl)phenol (64a). The synthesis of the title product was carried out in the same manner as (16a) on a 20 mmol scale, using 2-amino-5-tert-octylphenol as the nucleophile. The crude solid was recrystallized out of ethanol, affording the title product as red/orange needles in 74% yield. ¹H NMR (400 MHz; DMSO-d6): 9.93 (br, 1H), 9.73 (br, 1H), 8.90 (d, J=2.7 Hz, 1H), 8.24 (dd, J=9.6, 2.7 Hz, 1H), 7.25-7.22 (m, 2H), 6.93 (d, J=8.8 Hz, 1H), 6.76 (d, J=9.6 Hz, 1H), 1.67 (s, 2H), 1.30 (s, 6H), 0.72 (s, 9H). 13C NMR (101 MHz; DMSO-d6): δ 149.7, 147.1, 141.1, 135.9, 130.6, 129.6, 126.2, 125.0, 123.3, 123.2, 117.0, 116.0, 56.3, 37.6, 32.1, 31.6, 31.4. (EI, magnetic sector): calcd for C20H25N3O5 387.17942, found 387.17865.

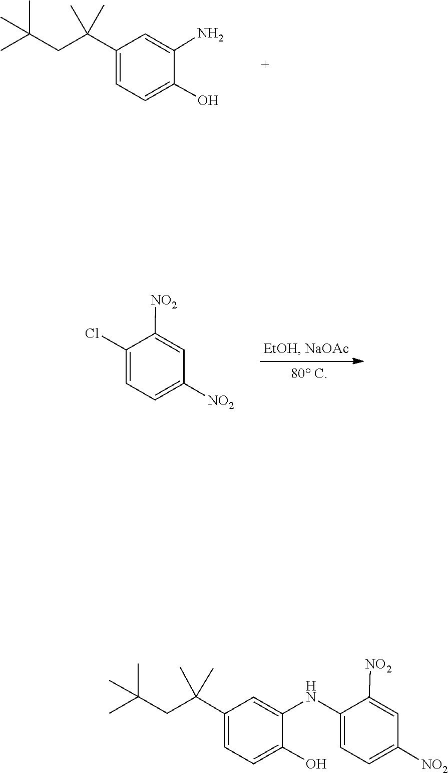 [SMILES: CC(C)(C)CC(C)(C)c1ccc(O)c(N)c1 ; O=[N+]([O-])c1ccc(Cl)c([N+](=O)[O-])c1 ; CC(C)(C)CC(C)(C)c1ccc(O)c(Nc2ccc([N+](=O)[O-])cc2[N+](=O)[O-])c1]

3-Methyl-7-nitrophenoxazine (16). 2-((2,4-Dinitrophenyl)amino)-5-methylphenol (16a) (3.30 g, 11.4 mmol) was dissolved in dry DMF sparged with N2 (57 ml) and heated to 100° C. with stirring under a N2 atmosphere. At 100° C., crushed NaOH was added (1.14 g, 28.5 mmol) and the mixture was heated to 120° C. for 4 hr. The reaction mixture was subsequently poured into ~250 ml of water and extracted with ethyl acetate three times. The organic phase was washed several times with 1M NaOH solution (to remove unreacted phenolic starting material which is inseparable by chromatography otherwise). The organic layer was dried with MgSO4, filtered, and the solvent removed under reduced pressure. The title compound was isolated by column chromatography (40% EtOAc in hexanes) appearing as a black solid which makes a deep red solution when dissolved (1.72 g, 62%). ¹H NMR (400 MHz; DMSO-d6): 9.28 (br, 1H), 7.68 (dd, J=2.5, 8.7 Hz, 1H), 7.30 (d, J=2.6 Hz, 1H), 6.60 (dd, J=1.6, 7.9 Hz, 1H), 6.51 (d, J=1.3 Hz, 1H), 6.48 (d, J=8.8 Hz, 1H), 6.42 (d, J=7.8 Hz, 1H), 2.11 (s, 3H). 13C NMR (101 MHz; DMSO-d6): δ 142.0(3), 141.9(7), 139.8, 139.1, 132.0, 126.7, 124.6, 121.9, 115.9, 114.0, 111.7, 110.0, 20.2. (EI, magnetic sector): calcd for C13H10N2O3 242.06914, found 242.07162.

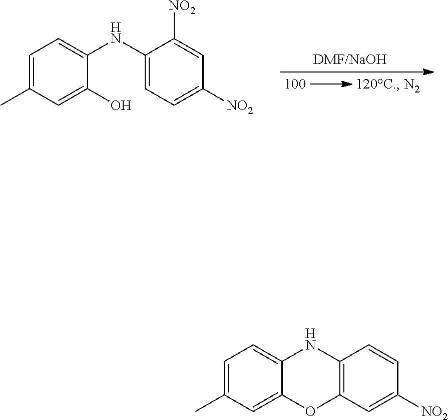 [SMILES: Cc1ccc(Nc2ccc([N+](=O)[O-])cc2[N+](=O)[O-])c(O)c1 ; Cc1ccc2c(c1)Oc1cc([N+](=O)[O-])ccc1N2]

2-tert-Octyl-7-nitrophenoxazine (64). The synthesis of the title product was carried out in the same manner as (16) on a 11 mmol scale, using 2-((2,4-dinitrophenyl)amino)-4-(2,4,4-trimethylpentan-2-yl)phenol (64a). The title compound was isolated by column chromatography (30% EtOAc in hexanes) appearing as a dark red solid at yield of 64%. ¹H NMR (400 MHz; DMSO-d6): 9.29 (br, 1H), 7.68 (dd, J=8.8, 2.6 Hz, 1H), 7.31 (d, J=2.5 Hz, 1H), 6.70 (dd, J=8.3, 2.2 Hz, 1H), 6.58 (d, J=8.4 Hz, 1H), 6.53 (d, J=2.2 Hz, 1H), 6.48 (d, J=8.8 Hz, 1H), 1.61 (s, 2H), 1.23 (s, 6H), 0.73 (s, 9H). 13C NMR (101 MHz; DMSO-d6): δ 146.0, 142.3, 139.9, 139.7, 139.4, 128.5, 121.9, 120.1, 114.5, 112.2, 111.9, 110.0, 56.0, 37.7, 32.0, 31.5, 31.2. (EI, magnetic sector): calcd for C20H24N2O3 340.17869, found 340.18078.

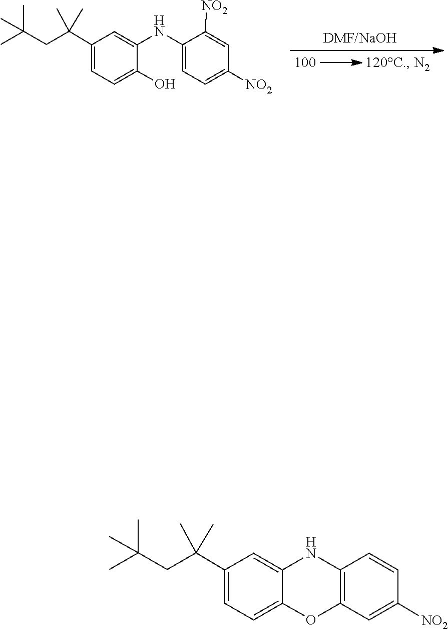 [SMILES: CC(C)(C)CC(C)(C)c1ccc(O)c(Nc2ccc([N+](=O)[O-])cc2[N+](=O)[O-])c1 ; CC(C)(C)CC(C)(C)c1ccc2c(c1)Nc1ccc([N+](=O)[O-])cc1O2]

5-(Diethylamino)-2-nitrophenol (18a). 5-Fluoro-2-nitrophenol (6.28 g, 40 mmol), acetonitrile (80 ml) and diethylamine were combined in a round bottom flask and stirred for 12 hr at 60° C. The solvent was removed under reduced pressure and the yellow residue was purified by column chromatography (15% EtOAc in hexanes) and the product was subsequently further purified by recrystallization out of hexanes to afford yellow needles (2.93 g, 35%). ¹H NMR (400 MHz; DMSO-d6): δ 11.11 (br, 1H), 7.85 (d, J=9.7 Hz, 1H), 6.45 (dd, J=2.7, 9.7 Hz, 1H), 6.16 (d, J=2.7 Hz, 1H), 3.45 (q, J=7.1 Hz, 4H), 1.12 (t, J=7.1 Hz, 6H). 13C NMR (101 MHz; DMSO-d6): δ 157.2, 154.3, 127.4, 122.9, 106.1, 96.6, 44.5, 12.4. HRMS (EI, magnetic sector): calcd for C10H14N2O3 210.10044, found 210.09749. mp=110-113° C.

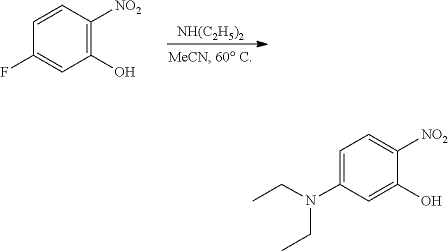

5-(Diethylamino)-2-((2,4-dinitrophenyl)amino)phenol (18b). In a Schlenk flask flushed with N2 were combined 5-(Diethylamino)-2-nitrophenol (18a) (2.91 g, 13.8 mmol) and degassed absolute ethanol (55 mL) with a magnetic stir bar. Under a stream of N2 10 wt. % Pd/C (0.29 g) was added and the flask was sealed. The N2 was evacuated and replaced with H2 with a balloon of excess H2 appended through the rubber septum. The reaction was heated to 50° C. and was vigorously stirred until the starting material had been consumed (monitored by TLC). In a separate flask equipped with a stir bar was measured 1-chloro-2,4-dinitrobenzene (2.62 g, 13 mmol). After completion of the hydrogenation, the first solution was rapidly filtered through celite (to remove the Pd/C) directly into the flask containing 1-chloro-2,4-dinitrobenzene. This was followed by 40 ml of clean ethanol through the celite (the transfer step should be performed quickly as the colourless aminophenol solution from the hydrogenation rapidly oxidizes when exposed to oxygen giving the solution a deep blue colour.) While sparging the solution with N2, triethylamine (2.1 ml) was added and the flask was evacuated then backfilled with N2. The reaction was stirred overnight at 40° C. after which the solvent was removed under reduced pressure. The title product was isolated by column chromatography (30-50% EtOAc gradient in hexanes) appearing as a black solid which makes a deep red solution when dissolved (2.75 g, 61%). ¹H NMR (400 MHz; DMSO-d6): δ 9.76 (br, 1H), 9.50 (br, 1H), 8.88 (d, J=2.7 Hz, 1H), 8.20 (dd, J=2.7, 9.6 Hz, 1H), 7.01 (d, J=8.7 Hz, 1H), 6.86 (d, J=9.7 Hz, 1H), 6.28 (d, J=2.6 Hz, 1H), 6.23 (dd, J=2.7, 8.7 Hz, 1H), 3.31 (q, J=7.0 Hz, 4H), 1.11 (t, J=7.0 Hz, 6H). 13C NMR (101 MHz; DMSO-d6): δ 153.2, 148.0(6), 148.0(3), 135.4, 130.1, 129.6, 128.2, 123.4, 117.2, 111.9, 103.2, 98.9, 43.9, 12.5. HRMS (EI, magnetic sector): calcd for C16H18N4O5 346.12772, found 346.12509. mp=193-197° C. (decomp.).

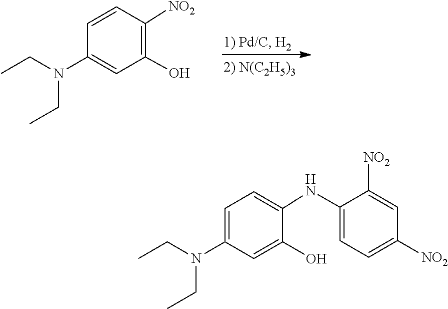 [SMILES: CCN(CC)c1ccc([N+](=O)[O-])c(O)c1 ; CCN(CC)c1ccc(Nc2ccc([N+](=O)[O-])cc2[N+](=O)[O-])c(O)c1]

N,N-diethyl-3-amine-7-nitrophenoxazine (18). 5-(Diethylamino)-2-((2,4-dinitrophenyl)amino)phenol (#b) (2.75 g, 8 mmol) was dissolved in dry DMF sparged with N2 (40 ml) and heated to 100° C. with stirring under a N2 atmosphere. At 100° C., crushed NaOH was added (0.80 g, 20 mmol) and the mixture was heated to 120° C. for 4 hr. The reaction mixture was subsequently poured into ~250 ml of water and extracted with ethyl acetate three times. The organic phase was washed several times with 1M NaOH solution (to remove unreacted phenolic starting material which is inseparable by chromatography otherwise). The organic layer was dried with MgSO4, filtered, and the solvent removed under reduced pressure. The title compound was isolated by column chromatography (40% EtOAc in hexanes) appearing as a dark purple solid (0.64 g, 27%). ¹H NMR (400 MHz; DMSO-d6): δ 9.18 (br, 1H), 7.65 (dd, J=2.4, 8.8 Hz, 1H), 7.27 (d, J=2.2 Hz, 1H), 6.42-6.40 (m, 2H), 6.13-6.06 (m, 2H), 3.21 (q, J=7.0 Hz, 4H), 1.02 (t, J=7.0 Hz, 6H). 13C NMR (101 MHz; DMSO-d6): δ 144.3, 143.1, 141.4, 140.2, 138.0, 122.2, 117.6, 117.6, 115.2, 111.1, 109.9, 107.0, 100.0, 43.8, 12.4. HRMS (EI, magnetic sector): calcd for C16H17N3O3 299.12699, found 299.12670. mp=185-188° C. (decomp.).

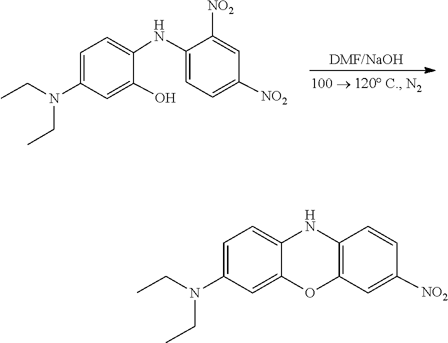 [SMILES: CCN(CC)c1ccc(Nc2ccc([N+](=O)[O-])cc2[N+](=O)[O-])c(O)c1 ; CCN(CC)c1ccc2c(c1)Oc1cc([N+](=O)[O-])ccc1N2]

3,7-Dinitro-10-acetylphenoxazine (19a). Phenoxazine (2.74 g, 15.0 mmol) and acetic anhydride (10 ml) were placed into a round bottom flask with a reflux condenser affixed to it. The mixture was refluxed for 3 hours after which the flask was cooled to room temperature then further cooled in a freezer. The 10-acetylphenoxazine intermediate crystallized and the acetic anhydride was decanted. The crystalline solid was suspended in glacial acetic acid (24 ml) and 1:1 mixture of 70% nitric acid (4 ml) in acetic acid (4 ml) was added. The fuming red solution was swirled for about 5 minutes, and the flask was placed in an ice bath and stood overnight. The next morning scratching promoted spontaneous crystallization. The solid recrystallized out of ethanol to afford the product (2.34 g, 49%) as orange needles. ¹H NMR (300 MHz; DMSO-d6): δ 8.12 (dd, J=2.5, 8.8 Hz, 2H), 8.03 (d, J=2.5 Hz, 2H), 7.91 (d, J=8.8 Hz, 2H), 2.37 (s, 3H). 13C NMR (75 MHz; DMSO-d6): δ 149.3, 145.6, 134.3, 125.9, 119.7, 112.1, 23.2. HRMS (EI, magnetic sector): calcd for C14H9N3O6 315.04914, found 315.05416. mp 187-190° C.

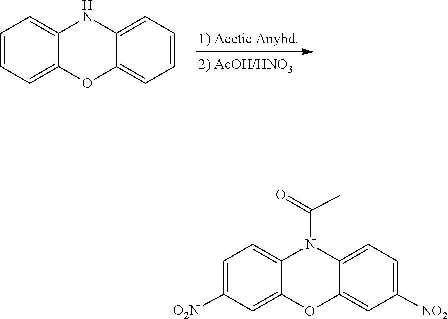 [SMILES: c1ccc2c(c1)Nc1ccccc1O2 ; CC(=O)N1c2ccc([N+](=O)[O-])cc2Oc2cc([N+](=O)[O-])ccc21]

3,7-Dinitrophenoxazine (19). In a round bottom flask equipped with a reflux condenser and magnetic stir bar were added 3,7-dinitro-10-acetylphenoxazine (19a) (2.15 g, 6.8 mmol), ethanol (50 ml) and 33% aqueous HCl (15 ml). The mixture was refluxed for several hours until the starting material had been completely converted to the title product. The product was recrystallized out of glacial acetic acid to afford red needles (1.55 g, 81%). ¹H NMR (400 MHz; DMSO-d6): δ 10.11 (s, 1H), 7.73 (dd, J=2.5, 8.7 Hz, 2H), 7.39 (d, J=2.5 Hz, 2H), 6.62 (d, J=8.7 Hz, 2H). 13C NMR (101 MHz; DMSO-d6): δ 142.0, 141.2, 137.1, 121.9, 113.4, 110.4. HRMS (EI, magnetic sector): calcd for C12H7N305 273.03857, found 273.04144. mp=220° C. (decomp.).

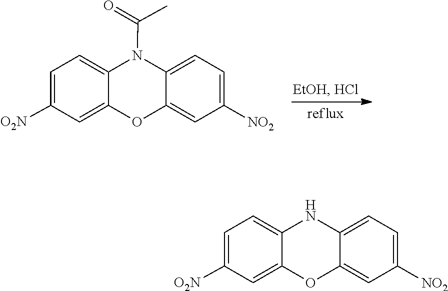 [SMILES: CC(=O)N1c2ccc([N+](=O)[O-])cc2Oc2cc([N+](=O)[O-])ccc21 ; O=[N+]([O-])c1ccc2c(c1)Oc1cc([N+](=O)[O-])ccc1N2]

3,7-Dinitrophenothiazine (29). Preparation of 3,7-dinitrophenothiazine was conducted in accordance to a previously described method.15 Into a round bottom flask equipped with a magnetic stir bar was dissolved phenothiazine (5 g, 25 mmol) in a chloroform (50 ml) acetic acid (20 ml) mixture. With stirring, NaNO2 was added (5 g, 73 mmol) and the reaction was allowed to stir for several hours. The brown precipitate was filtered off and rinsed with acetic acid, H2O then ethanol. The crude product was recrystallized twice out of DMF and rinsed with acetone to afford the title product as dark red needles (2.53 g, 35%). ¹H NMR (400 MHz; DMSO-d6): δ 10.13 (s, 1H), 7.88 (dd, J=2.4, 8.8 Hz, 2H), 7.79 (dd, J=2.3 Hz, 2H), 6.74 (dd, J=8.9 Hz, 2H). 13C NMR (101 MHz; DMSO-d6): δ 145.2, 142.6, 124.8, 121.8, 116.8, 114.8. HRMS (EI, magnetic sector): calcd for C12H7N3O4S 289.01573, found 289.01762.

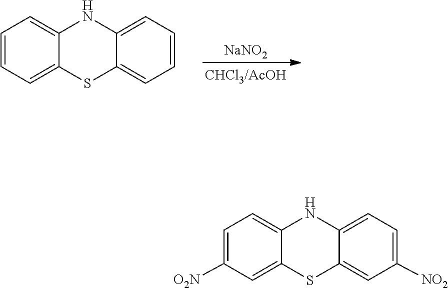

3-Methyl-7-cyanophenoxazine (21). 3,4-Difluorobenzonitrile (0.84 g, 6.0 mmol), 2-amino-5-methylphenol (0.74 g, 6.0 mmol), K2CO3 (1.71 g, 12.4 mmol) and DMSO (30 mL) were placed in a round bottom flask. Under N2 the reaction mixture was heated to 135° C. for 14 hours with vigorous stirring after which the reaction was cooled, quenched with water and the aqueous mixture extracted three times with EtOAc. The organic phase was washed twice with water, dried over MgSO4, filtered and the solvent removed under reduced pressure. The crude solid was purified by column chromatography (50% EtOAc in hexanes) to render the product as a yellow solid (0.67 g, 50%). ¹H NMR (400 MHz; DMSO-d6): δ 8.81 (s, 1H), 7.14 (dd, J=1.9, 8.2 Hz, 1H), 6.95 (d, J=1.7 Hz, 1H), 6.58-6.56 (m, 1H), 6.46-6.44 (m, 2H), 6.37 (d, J=7.8 Hz, 1H), 2.09 (s, 3H). 13C NMR (101 MHz; DMSO-d6): δ 142.6, 142.2, 137.6, 131.1, 129.6, 127.6, 124.4, 119.2, 117.6, 115.8, 113.7, 113.1, 100.6, 20.11. HRMS (EI, magnetic sector): calcd for C14H10N2O 222.07931, found 222.08020. mp=215-217° C. (decomp.).

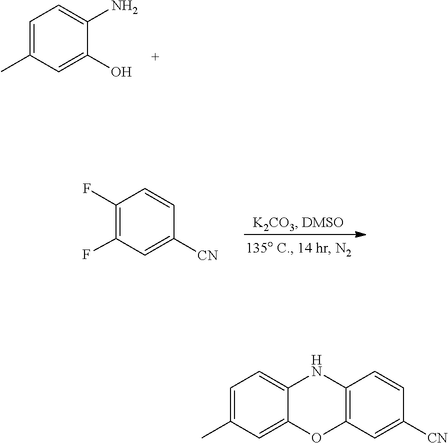

3-Methoxy-7-cyanophenoxazine (22). With 3,4-Difluorobenzonitrile (1.39 g, 10.0 mmol) and 2-amino-5-methoxyphenol (1.39 g, 10.0 mmol), the synthesis was carried out in a fashion analogous to (21). The crude solid was purified by column chromatography (40% EtOAc in hexanes) to render the product as a light-yellow solid (1.55 g, 65%). ¹H NMR (400 MHz; DMSO-d6): δ 8.75 (s, 1H), 7.15 (dd, J=1.8, 8.1 Hz, 1H), 6.95 (d, J=1.9 Hz, 1H), 6.46-6.41 (m, 2H), 6.37 (dd, J=2.7, 8.5 Hz, 1H), 6.32 (d, J=2.6 Hz, 1H), 3.64 (s, 3H). 13C NMR (101 MHz; DMSO-d6): δ 154.6, 143.0, 141.9, 137.7, 129.8, 123.4, 119.3, 117.6, 114.1, 113.0, 108.5, 102.5, 100.2, 55.3. HIRMS (EI, magnetic sector): calcd for C14H10N2O2 238.07423, found 238.07274. mp=194-200 (decomp).

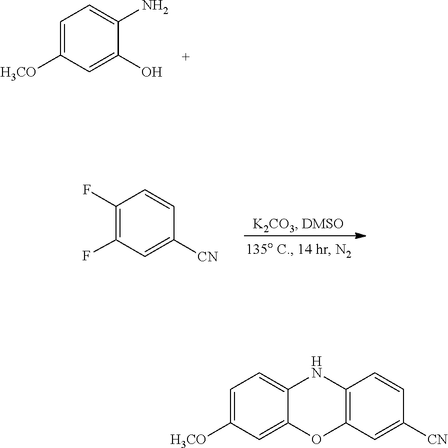 [SMILES: COc1ccc(N)c(O)c1 ; N#Cc1ccc(F)c(F)c1 ; COc1ccc2c(c1)Oc1cc(C#N)ccc1N2]

2-tert-Butyl-7-cyanophenoxazine (62). The synthesis of the title product was carried out in the same manner as (21) on an 8 mmol scale, using 2-amino-4-tert-butylphenol as the nucleophile. The title product was isolated by silica gel chromatography (35% EtOAc in hexanes) at yield of 35%, appearing as a light-yellow solid. ¹H NMR (400 MHz; Benzene-d6): δ 6.57 (dd, J=8.0, 1.8 Hz, 1H), 6.50-6.44 (m, 3H), 5.88 (d, J=1.9 Hz, 1H), 5.51 (d, J=8.0 Hz, 1H), 4.17 (br, 1H), 1.17 (s, 9H). 13C NMR (101 MHz; Benzene-d6): δ 147.5, 144.2, 141.5, 136.5, 129.5, 128.9, 119.7, 119.4, 118.9, 115.9, 113.3, 111.8, 104.4, 34.6, 31.7. HRMS (EI, magnetic sector): calcd for C17H16N2O 264.12626, found 264.12469.

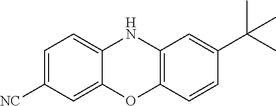 [SMILES: CC(C)(C)c1ccc2c(c1)Nc1ccc(C#N)cc1O2]

2-tert-Octyl-7-cyanophenoxazine (63). The synthesis of the title product was carried out in the same manner as (21) on a 10 mmol scale, using 2-amino-4-tert-butylphenol as the nucleophile. The title product was isolated by silica gel chromatography (35% EtOAc in hexanes) at yield of 45%, appearing as a light-yellow solid. ¹H NMR (400 MHz; DMSO-d6): δ 8.83 (s, 1H), 7.15 (dd, J=8.2, 1.9 Hz, 1H), 6.96 (d, J=1.8 Hz, 1H), 6.65 (dd, J=8.4, 2.1 Hz, 1H), 6.54 (d, J=8.4 Hz, 1H), 6.49 (d, J=2.2 Hz, 1H), 6.46 (d, J=8.1 Hz, 1H), 1.61 (s, 2H), 1.23 (s, 6H), 0.73 (s, 9H). 13C NMR (101 MHz; DMSO-d6): δ 145.8, 142.8, 140.1, 137.5, 129.5, 129.4, 119.3, 119.1, 117.6, 114.4, 113.2, 111.9, 100.8, 56.0, 37.7, 32.0, 31.5, 31.2. HRMS (EI, magnetic sector): calcd for C21H24N2O 320.18886, found 320.18696.

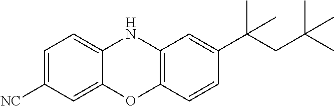 [SMILES: CC(C)(C)CC(C)(C)c1ccc2c(c1)Nc1ccc(C#N)cc1O2]

1-Methyl-7-cyanophenoxazine (87). The synthesis of the title product was carried out in the same manner as (21) on a 5 mmol scale, using 2-amino-3-methylphenol as the nucleophile. The title product was isolated by silica gel chromatography (35% EtOAc in hexanes) at yield of 41%, appearing as a beige solid. ¹H NMR (400 MHz; DMSO-d6): δ 8.11 (s, 1H), 7.16 (dd, J=8.1, 1.8 Hz, 1H), 6.96 (d, J=1.7 Hz, 1H), 6.75 (d, J=8.2 Hz, 1H), 6.64 (d, J=7.5 Hz, 1H), 6.56 (dd, J=7.8, 7.6 Hz, 1H), 6.47 (d, J=7.6 Hz, 1H), 2.07 (s, 3H). 13C NMR (101 MHz; DMSO-d6): δ 142.8, 142.3, 137.4, 129.4, 128.3, 125.9, 122.5, 121.2, 119.1, 117.4, 114.1, 113.0, 101.3, 16.6. HRMS (EI, magnetic sector): calcd for C14H10N2O 222.07931, found 222.07767.

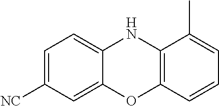 [SMILES: Cc1cccc2c1Nc1ccc(C#N)cc1O2]

1,3-Dimethyl-7-cyanophenoxazine (89). The synthesis of the title product was carried out in the same manner as (21) on a 5 mmol scale, using 2-amino-3,5-dimethylphenol hydrochloride as the nucleophile (the K₂CO₃ was also increased from 2.2 to 3.5 molar equivalents). The title product was isolated by silica gel chromatography (35% EtOAc in hexanes) at yield of 32%, appearing as a light-yellow solid. ¹H NMR (400 MHz; DMSO-d6): δ 8.05 (s, 1H), 7.15 (dd, J=8.2, 1.9 Hz, 1H), 6.93 (d, J=1.8 Hz, 1H), 6.71 (d, J=8.2 Hz, 1H), 6.45 (br, 1H), 6.31 (br, 1H), 2.06 (s, 3H), 2.03 (s, 3H). 13C NMR (101 MHz; DMSO-d6): δ 142.7, 142.1, 137.6, 130.4, 129.3, 126.2, 125.6, 122.2, 119.2, 117.4, 113.9, 113.6, 100.8, 20.0, 16.6. HRMS (EI, magnetic sector): calcd for C15H12N2O 236.09496, found 236.09644.

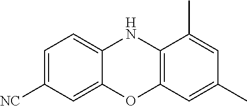 [SMILES: Cc1cc(C)c2c(c1)Oc1cc(C#N)ccc1N2]

2,4-Dimethyl-7-cyanophenoxazine (90). The synthesis of the title product was carried out in the same manner as (21) on a 5 mmol scale, using 2-amino-4,6-dimethylphenol as the nucleophile (the K₂CO₃ was also increased from 2.2 to 3 molar equivalents). The title product was isolated by reducing volume of ethyl acetate (from the extraction) to ~6 ml, where the precipitated product was filtered and rinsed with diethyl ether. The title product appeared as light-yellow crystals with an isolated yield of 57%. ¹H NMR (400 MHz; DMSO-d6): δ 8.77 (s, 1H), 7.13 (dd, J=8.1, 1.7 Hz, 1H), 6.94 (d, J=1.5 Hz, 1H), 6.46 (d, J=8.1 Hz, 1H), 6.31 (br, 1H), 6.12 (br, 1H), 2.05 (s, 3H), 2.00 (s, 3H). 13C NMR (101 MHz; DMSO-d6): δ 142.9, 138.3, 137.5, 132.5, 129.7, 129.5, 123.9, 123.8, 119.2, 117.6, 113.0, 112.2, 100.7, 20.2, 14.6. HRMS (EI, magnetic sector): calcd for C15H12N2O 236.09496, found 236.09343.

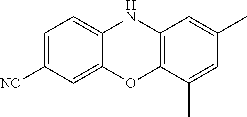 [SMILES: Cc1cc(C)c2c(c1)Nc1ccc(C#N)cc1O2]

3-Cyano-7-phenylphenoxazine (91). The synthesis of the title product was carried out in the same manner as (21) on a 3.5 mmol scale, using 2-amino-5-phenylphenol as the nucleophile (the K₂CO₃ was also increased from 2.2 to 3.5 molar equivalents). The title product was isolated by reducing volume of ethyl acetate (from the extraction) to ~6 ml, where the precipitated product was filtered and rinsed with diethyl ether. The title product appeared as light-green solid with an isolated yield of 53%. ¹H NMR (400 MHz; DMSO-d6): δ 9.08 (s, 1H), 7.56 (dd, J=8.4, 1.3 Hz, 2H), 7.39 (dd, J=7.9, 7.3 Hz, 2H), 7.29 (dd, J=7.3, 7.3 Hz, 1H), 7.18 (dd, J=8.1, 1.8 Hz, 1H), 7.11 (dd, J=8.0, 2.0 Hz, 1H), 7.01 (d, J=1.8 Hz, 1H), 6.95 (d, J=2.0 Hz, 1H), 6.57 (d, J=8.1 Hz, 1H), 6.51 (d, J=8.1 Hz, 1H). 13C NMR (101 MHz; DMSO-d6): δ 142.9, 142.6, 139.0, 133.8, 129.8, 128.9, 127.0, 125.7, 122.5, 119.1, 117.7, 114.3, 113.4, 113.3, 101.2. HRMS (EI, magnetic sector): calcd for C19H12N2O 284.09496, found 284.09257.

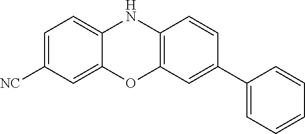 [SMILES: N#Cc1ccc2c(c1)Oc1cc(-c3ccccc3)ccc1N2]

12H-benzo[a]phenoxazine-9-carbonitrile (92). The synthesis of the title product was carried out in the same manner as (21) on a 5 mmol scale, using 1-amino-2-naphthol hydrochloride as the nucleophile (the K₂CO₃ was also increased from 2.2 to 3.5 molar equivalents). The title product was isolated by reducing volume of ethyl acetate (from the extraction) to ~6 ml, where the precipitated product was filtered and rinsed with diethyl ether. The title product appeared as green solid with an isolated yield of 62%. ¹H NMR (400 MHz; DMSO-d6): δ 8.82 (s, 1H), 7.95 (d, J=8.6 Hz, 1H), 7.76 (d, J=8.1 Hz, 1H), 7.49 (dd, J=8.3, 8.2 Hz, 1H), 7.39-7.31 (m, 2H), 7.22 (dd, J=8.2, 1.8 Hz, 1H), 7.00 (d, J=1.6 Hz, 1H), 6.93 (d, J=8.7 Hz, 1H), 6.77 (d, J=8.1 Hz, 1H). 13C NMR (101 MHz; DMSO-d6): δ 143.4, 138.3, 137.9, 130.7, 129.9, 128.3, 126.0, 124.8, 123.5, 121.6, 121.4, 120.4, 119.1, 117.6, 116.3, 114.2, 101.9. HRMS (EI, magnetic sector): calcd for C17H10N2O 258.07931, found 258.07954.

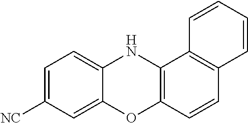 [SMILES: N#Cc1ccc2c(c1)Oc1ccc3ccccc3c1N2]

2-tert-Butyl-7-(piperazin-1-ylsulfonyl)-phenoxazine (60). 2-Amino-4-tert-butylphenol (0.50 g, 3 mmol), 1-((3,4-difluorophenyl)sulfonyl)piperazine (0.79 g, 3 mmol), potassium carbonate (0.42 g, 9 mmol), DMF (0.2M, 15 ml) and magnetic stir bar were placed in an Anton Parr G30 borosilicate microwave vessel. The solution was sparged with N2 for 10 min. after which the vessel was sealed and the reaction vessel was placed in the microwave reactor (Monowave 400). With stirring (600 RPM) the tube was heated to 200° C. (held for 60 min.) then cooled to room temperature. After cooling, the reaction mixture was immediately poured into aqeous 1M NaOH solution and the product was extracted with ethyl acetate. The organic phase was washed with water 6 times, after which the organic solution was dried with MgSO4, filtered and solvent removed under reduced pressure. Trituration with MeOH removed impurities, affording the title product in a 58% yield (0.68 g) appearing as an off-white solid. ¹H NMR (400 MHz; DMSO-d6): δ 8.84 (s, 1H), 7.06 (dd, J=8.2, 2.0 Hz, 1H), 6.76 (d, J=1.8 Hz, 1H), 6.66 (dd, J=8.3, 2.2 Hz, 1H), 6.56 (d, J=8.3 Hz, 1H), 6.55 (d, J=8.2 Hz, 1H), 6.51 (d, J=2.2 Hz, 1H), 2.74-2.70 (m, 8H), 1.19 (s, 9H). HRMS (EI, magnetic sector): calcd for C20H25N3O3S 387.16166, found 387.16248.

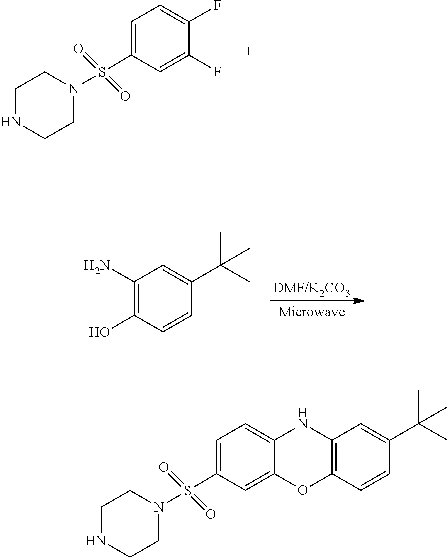 [SMILES: O=S(=O)(c1ccc(F)c(F)c1)N1CCNCC1 ; CC(C)(C)c1ccc(O)c(N)c1 ; CC(C)(C)c1ccc2c(c1)Nc1ccc(S(=O)(=O)N3CCNCC3)cc1O2]

2-tert-Butyl-7-(4-methylpiperazin-1-ylsulfonyl)-phenoxazine (61). The synthesis and isolation of the title product was carried out in the same manner as (60) on a 2 mmol scale, using 1-((3,4-difluorophenyl)sulfonyl)-4-methylpiperazine as the electrophile. The product was isolated in a yield of 47%, appearing was greyish solid. ¹H NMR (400 MHz; DMSO-d6): δ 8.84 (s, 1H), 7.06 (dd, J=8.2, 2.0 Hz, 1H), 6.77 (d, J=2.0 Hz, 1H), 6.65 (dd, J=8.4, 2.3 Hz, 1H), 6.56 (d, J=8.3 Hz, 1H), 6.55 (d, J=8.2 Hz, 1H), 6.50 (d, J=2.3 Hz, 1H), 2.84 (br, 4H), 2.34 (br, 4H), 2.14 (s, 3H), 1.19 (s, 9H). 13C NMR (101 MHz; DMSO-d6): δ 146.9, 142.6, 140.2, 137.3, 129.9, 124.9, 124.7, 118.2, 114.7, 113.8, 112.7, 111.0, 53.5, 45.7, 45.3, 33.9, 31.0. HRMS (EI, magnetic sector): calcd for C21H27N3O3S 401.17731, found 401.17586.

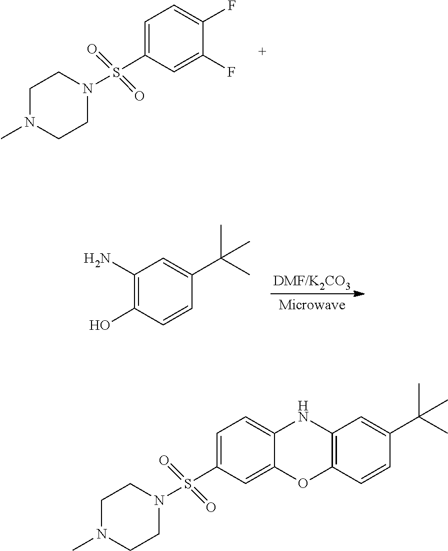 [SMILES: CN1CCN(S(=O)(=O)c2ccc(F)c(F)c2)CC1 ; CC(C)(C)c1ccc(O)c(N)c1 ; CN1CCN(S(=O)(=O)c2ccc3c(c2)Oc2ccc(C(C)(C)C)cc2N3)CC1]

N,N-diethyl-6,8-dimethyl-10H-phenoxazine-3-sulfonamide (94). N,N-Diethyl-3,4-difluorobenzenesulfonamide (1.25 g, 5.0 mmol), 2-amino-4,6-dimethylphenol (0.69 g, 5.0 mmol), K2CO3 (2.07 g, 15 mmol) and DMSO (10 mL) were placed in a Schlenk flask and the solution was sparged with a constant stream of argon for 10 minutes. After sealing the Schlenk, the reaction mixture was heated to 140° C. for 14 hours with vigorous stirring after which the reaction was cooled, quenched with water and the aqueous mixture extracted twice with EtOAc. The organic phase was washed twice with water, dried over Na2SO4, filtered and the solvent removed under reduced pressure. The crude solid was purified by column chromatography (30% EtOAc in hexanes) to render the product as an off-white solid (0.81 g, 47%). ¹H NMR (400 MHz; DMSO-d6): δ 8.67 (s, 1H), 7.11 (dd, J=8.2, 2.1 Hz, 1H), 6.85 (d, J=2.0 Hz, 1H), 6.50 (d, J=8.2 Hz, 1H), 6.31 (br, 1H), 6.12 (d, J=2.0 Hz, 1H), 3.10 (q, J=7.1 Hz, 4H), 2.05 (s, 3H), 2.02 (s, 3H), 1.04 (t, J=7.1 Hz, 6H). 13C NMR (101 MHz; DMSO-d6): δ 142.8, 138.3, 136.8, 132.5, 130.0, 129.9, 123.8(3), 123.7(1), 123.6(9), 113.2, 112.5, 112.0, 41.8, 20.2, 14.6, 14.1. HRMS (EI, magnetic sector): calcd for C18H22N2O3S 346.13511, found 346.13393.

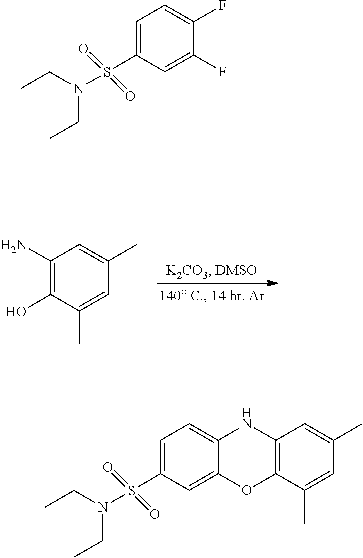

N,N-diethyl-12H-benzo[a]phenoxazine-9-sulfonamide (95). The synthesis of the title product was carried out in the same manner as (94) on a 5 mmol scale, using 1-amino-2-naphthol hydrochloride as the nucleophile (the K₂CO₃ was also increased from 3 to 3.5 molar equivalents). The title product was isolated by reducing volume of ethyl acetate (from the extraction) to −15 ml, where the precipitated product was filtered and rinsed with diethyl ether. The title product appeared as a yellowish-brown crystalline solid with an isolated yield of 45%. ¹H NMR (400 MHz; DMSO-d6): δ 8.75 (s, 1H), 7.97 (d, J=8.5 Hz, 1H), 7.75 (d, J=8.0 Hz, 1H), 7.48 (ddd, J=8.3, 8.1, 1.2 Hz, 1H), 7.37 (dd, J=7.8, 7.8 Hz, 1H), 7.32 (d, J=8.7 Hz, 1H), 7.20 (dd, J=8.2, 2.0 Hz, 1H), 6.94 (d, J=8.7 Hz, 1H), 6.87 (d, J=2.1 Hz, 1H), 6.83 (d, J=8.2 Hz, 1H), 3.13 (q, J=7.1 Hz, 4H), 1.05 (t, J=7.1 Hz, 6H). 13C NMR (101 MHz; DMSO-d6): δ 143.3, 138.1, 137.1, 131.1, 130.7, 128.3, 125.9, 124.8, 124.1, 123.9, 121.4(3), 121.3(6), 120.4, 116.3, 113.7, 113.0, 41.8, 14.1. HRMS (EI, magnetic sector): calcd for C18H22N2O3S 368.11946, found 368.11662.

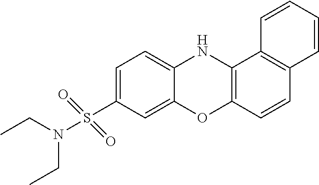 [SMILES: CCN(CC)S(=O)(=O)c1ccc2c(c1)Oc1ccc3ccccc3c1N2]

N,N-bis(2-ethylhexyl)-7-methyl-10H-phenoxazine-3-sulfonamide (93). The synthesis of the title product was carried out in the same manner as (94) on a 5 mmol scale, using 2-amino-5-methylphenol as the nucleophile and N,N-bis(2-ethylhexyl)-3,4-difluorobenzenesulfonamide as the electrophile. The title product was isolated by silica gel chromatography (25% EtOAc in hexanes) at yield of 32%, appearing as an off-white solid. ¹H NMR (400 MHz; DMSO-d6): δ 8.73 (br, 1H), 7.10 (dd, J=8.2, 2.0 Hz, 1H), 6.80 (d, J=2.0 Hz, 1H), 6.57 (dd, J=7.8, 1.0 Hz, 1H), 6.50 (d, J=8.2 Hz, 1H), 6.46 (d, J=1.3 Hz, 1H), 6.37 (d, J=7.8 Hz, 1H), 2.89-2.73 (m, 4H), 2.10 (s, 3H), 1.50 (br, 2H), 1.36-1.14 (m, 16H), 0.86-0.78 (m, 12H). 13C NMR (101 MHz; DMSO-d6): δ 142.4, 142.2, 137.0, 130.8, 128.8, 128.0, 124.4, 124.3, 115.8, 113.5(4), 113.3(5), 112.5, 53.3, 37.4, 29.8, 28.1, 23.1, 22.5, 20.1, 13.9, 10.4. HRMS (EI, magnetic sector): calcd for C29H44N2O3S 500.30726, found 500.30651.

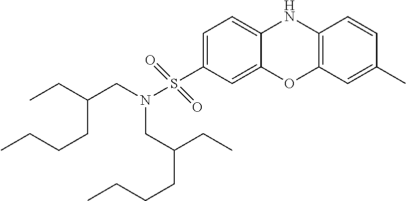 [SMILES: CCCCC(CC)CN(CC(CC)CCCC)S(=O)(=O)c1ccc2c(c1)Oc1cc(C)ccc1N2]

methyl 9-methyl-10H-phenoxazine-3-carboxylate (88). In a flask affixed with a reflux condenser were added 1-methyl-7-cyanophenoxazine (87) (375 mg, 1.59 mmol), ethylene glycol (4 ml), crushed NaOH (2 g), and magnetic stir bar. With stirring, this was heated to 200° C. for 1 hr, and after cooling it was poured into ~60 ml of water, and the aqeous phase was washed with diethyl ether once. The water was then acidified, and the crude carboxylic acid precipitate was filtered and dried. Once dry, the crude intermediate was placed in a Schlenk tube and dissolved in acetone (5 ml) which was sparged with N₂ for 10 min. After adding K₂CO₃ (0.13 g) and iodomethane (70 L), the tube was sealed and allowed to stir for ~40 hr. The reaction was quenched with water and the aqueous mixture extracted three times with EtOAc after which the organic was dried over MgSO4, filtered and the solvent removed under reduced pressure. This crude solid was purified by column chromatography (30% EtOAc in hexanes) to render the title product (152 mg, 38% yield). ¹H NMR (300 MHz; DMSO-d6): δ 8.03 (br, 1H), 7.37 (dd, J=8.1, 1.9 Hz, 1H), 7.03 (d, J=1.9 Hz, 1H), 6.75 (d, J=8.3 Hz, 1H), 6.63 (d, J=7.5 Hz, 1H), 6.55 (dd, J=7.7, 7.7 Hz, 1H), 6.47 (d, 7.7 Hz, 1H), 3.75 (s, 3H), 2.08 (s, 3H). 13C NMR (151 MHz; DMSO-d6): δ 165.4, 142.5, 142.4, 137.3, 128.7, 126.2, 125.7, 122.3, 121.2, 121.0, 115.0, 113.4, 113.0, 51.7, 16.7. HRMS (EI, magnetic sector): calcd for C15H13NO3 255.08954, found 255.08786.

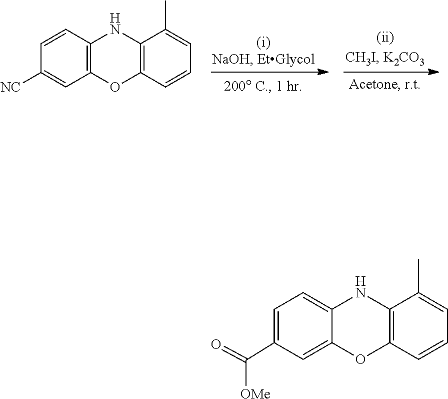 [SMILES: Cc1cccc2c1Nc1ccc(C#N)cc1O2 ; COC(=O)c1ccc2c(c1)Oc1cccc(C)c1N2]

3-Formylphenoxazine (12). Phenoxazine (0.92 g, 5 mmol), hexamine (1.40 g, 10 mmol), glacial acetic acid (20 ml) and magnetic stir bar were placed in an Anton Parr G30 borosilicate microwave vessel. The solution was sparged with N2 for 10 min. after which the vessel was sealed and the reaction vessel was placed in the microwave reactor (Monowave 400). With stirring (600 RPM) the tube was heated to 120° C. (held for 30 min.), then 150° C. (held for 80 min.), back to 120° C. (held for 30 min.) then cooled to room temperature. The reaction mixture was poured into water and Na2CO3 was added to neutralize the solution. The slurry was extracted several times with dichloromethane, after which the organic solution was dried with MgSO4, filtered and solvent removed under reduced pressure. 3-Formylphenoxazine (12) (1:1 EtOAc:hexanes Rf=0.61) was isolated by column chromatography (40% EtOAc gradient in hexanes) to afford a brownish orange solid that makes a fluorescent solution when dissolved (0.14 g, 13%). ¹H NMR (400 MHz; DMSO-d6): δ 9.59 (s, 1H), 9.04 (br, 1H), 7.32 (dd, J=1.8, 8.1 Hz, 1H), 6.98 (d, J=1.5 Hz, 1H), 6.79-6.74 (m, 1H), 6.66-6.63 (m, 2H), 6.54 (d, J=8.0 Hz, 1H), 6.51 (d, J=7.4 Hz, 1H). 13C NMR (101 MHz; DMSO-d6): δ 189.8, 143.0, 142.6, 138.7, 130.2, 129.3, 128.9, 124.2, 121.9, 115.3, 113.9, 112.6. (EI, magnetic sector): calcd for C13H9NO2 211.06333, found 211.06383. mp=169-172° C. 3,7-Diformylphenoxazine (13). 3,7-Diformylphenoxazine (13) (1:1 EtOAc:hexanes Rf=0.29) eluted after (12). The fractions containing the product were combined in a single flask and the solvent was removed under reduced pressure. The residue was rinsed with a minimum of acetone to remove coeluted impurities rendering an orange solid that makes a strongly fluorescent solution when dissolved (90 mg, 7.5%). ¹H NMR (400 MHz; DMSO-d6): δ 9.67 (br, 1H), 9.64 (s, 2H), 7.36 (dd, J=1.7, 8.0 Hz, 2H), 7.04 (d, J=1.8 Hz, 2H), 6.63 (d, J=8.0 Hz, 2H). 13C NMR (101 MHz; DMSO-d6): δ 190.1, 142.9, 136.6, 130.6, 128.6, 114.2, 113.6. (EI, magnetic sector): calcd for C14H9NO3 239.13101, found 239.129113. mp=250° C. (decomp.

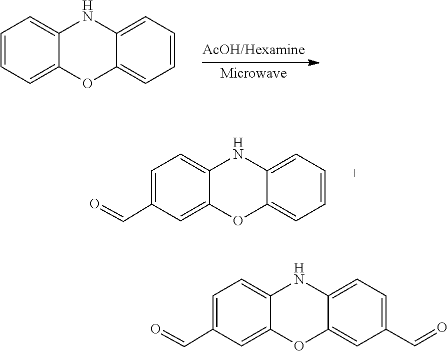 [SMILES: c1ccc2c(c1)Nc1ccccc1O2 ; O=Cc1ccc2c(c1)Oc1ccccc1N2 ; O=Cc1ccc2c(c1)Oc1cc(C=O)ccc1N2]

3-Cyano-7-formylphenoxazine (14). The reaction was carried out in an analogous fashion to the preparation of 3-formylphenoxazine (12) using 3-cyanophenoxazine (20) as a starting material (1.04 g, 5 mmol). The title product was isolated by column chromatography (40% EtOAc in hexanes) which appeared as a bright yellow solid (0.10 g, 8.5%). ¹H NMR (400 MHz; DMSO-d6): δ 9.63 (s, 1H), 9.59 (br, 1H), 7.36 (dd, J=1.7, 8.0 Hz, 1H), 7.22 (dd, J=1.8, 8.1 Hz, 1H), 7.07 (d, J=1.8 Hz, 1H), 7.01 (d, J=1.7 Hz, 1H), 6.60 (d, J=8.0 Hz, 1H), 6.56 (d, J=8.1 Hz, 1H). 13C NMR (101 MHz; DMSO-d6): δ 190.1, 142.8, 142.7, 136.7, 135.5, 130.5, 129.9, 128.8, 118.8, 118.0, 114.1(6), 114.1(3), 113.6, 102.8. (EI, magnetic sector): calcd for C14H8N2O2 236.05858, found 236.05727. mp=270° C. (decomp.).

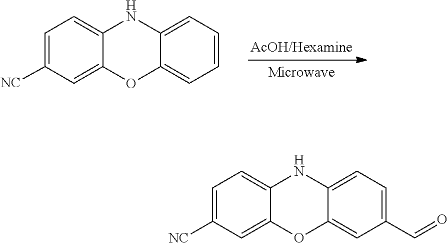 [SMILES: N#Cc1ccc2c(c1)Oc1ccccc1N2 ; N#Cc1ccc2c(c1)Oc1cc(C=O)ccc1N2]

3,7-Dimethylphenoxazine (35). In a sealable tube was charged 5-methyl-2-aminophenol (7.02 g, 57 mmol) and 1 equivalent of 5-methyl-2-aminophenol hydrochloride (9.12 g, 57 mmol). The tube was purged under a stream of N2 and sealed with the Teflon screwcap. The tube was placed in a sand bath and was heated to ~255° C. for 2.5 hours then allowed to cool to room temperature. The black residue was broken up and treated with hot acetone (Soxhlet extraction for several hours). The residue from the extraction was purified by column chromatography (20% EtOAc in hexanes). The title product were colourless crystals which precipitated out of the eluent. The eluent which contained the product were collected (~400 ml) and the volume was reduced to ~100 ml under reduced pressure and the flask was placed in a freezer overnight. The solvent was decanted and the solid was recrystallized out of ethanol to afford white/beige platelets (0.96 g, 7.9%). ¹H NMR (400 MHz; DMSO-d6): δ 7.89 (br, 1H), 6.52 (dd, J=1.7, 7.8 Hz, 2H), 6.43 (d, J=1.4 Hz, 2H), 6.32 (d, J=7.8 Hz, 2H), 2.08 (s, 6H). 13C NMR (151 MHz; C6D6): δ 144.3, 131.2, 130.0, 124.2, 117.3, 113.9, 20.9. (EI, magnetic sector): calcd for C14H13NO 211.09971, found 211.09715. mp=203-205° C.

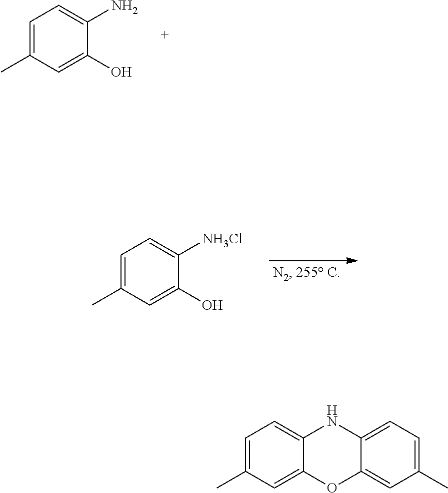

1,9-Dimethylphenoxazine (36). The synthesis was carried out in an analogous fashion to (35) using 3-methyl-2-aminophenol (6.16 g, 50 mmol) and 3-methyl-2-aminophenol hydrochloride (7.98 g, 50 mmol) as the starting materials. The product was isolated by column chromatography (10% EtOAc in hexanes) and was subsequently recrystallized out of a minimum of cold hexanes (product very soluble in hexanes at room temperature) to afford beige needles (0.73 g, 6.9%). ¹H NMR (400 MHz; DMSO-d6): δ 6.64 (d, J=7.3 Hz, 2H), 6.56 (dd, J=7.5, 7.8 Hz, 2H), 6.49 (d, J=7.6 Hz, 2H), 5.96 (br, 1H), 2.13 (s, 6H). 13C NMR (101 MHz; DMSO-d6): δ 143.2, 129.6, 125.2, 123.5, 120.7, 112.8, 16.2. (EI, magnetic sector): calcd for C14H13NO 211.09971, found 211.09768. mp=97-100° C.

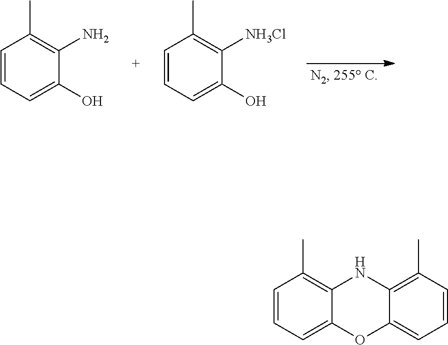

1,3,7,9-Tetramethylphenoxazine (38). The synthesis was carried out in an analogous fashion to (35) using 3,5-dimethyl-2-aminophenol (2.74 g, 20 mmol) and 3,5-dimethyl-2-aminophenol hydrochloride (3.47 g, 20 mmol) as the starting materials. The product was isolated by column chromatography (10% EtOAc in hexanes) and was subsequently recrystallized out of a minimum of cold hexanes (product very soluble in hexanes at room temperature) to afford beige needles (0.34 g, 7.2%). ¹H NMR (400 MHz; DMSO-d6): δ 6.45 (d, J=1.1 Hz, 2H), 6.32 (d, J=1.2 Hz, 2H), 5.78 (br, 1H), 2.08 (s, 12H). 13C NMR (101 MHz; DMSO-d6): δ 142.9, 129.4, 127.1, 125.4, 123.3, 113.4, 20.1, 16.2. (EI, magnetic sector): calcd for C16H17NO 239.13101, found 239.13357. mp=91-93° C.

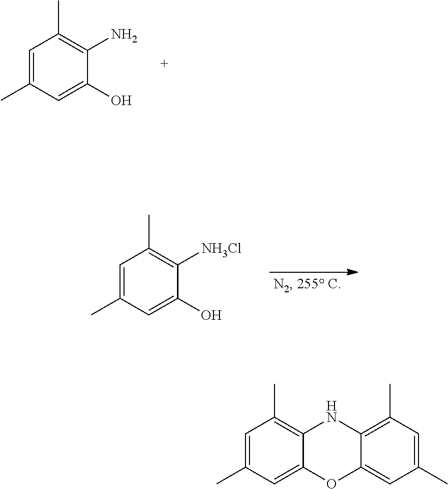

2,4,6,8-Tetramethylphenoxazine (37). The synthesis was carried out in an analogous fashion to (35) using 4,6-dimethyl-2-aminophenol (2.33 g, 17 mmol) and 4,6-dimethyl-2-aminophenol hydrochloride (2.95 g, 17 mmol) as the starting materials. The product was isolated by column chromatography (15% EtOAc in hexanes) and was subsequently recrystallized out of hexanes to afford white needles (0.31 g, 7.6%). ¹H NMR (400 MHz; DMSO-d6): δ 7.86 (br, 1H), 6.25 (d, J=1.2 Hz, 2H), 6.09 (d, J=1.6 Hz, 2H), 2.05 (s, 6H), 2.03 (s, 6H). 13C NMR (101 MHz; DMSO-d6): δ 138.8, 132.0, 131.6, 123.5, 122.3, 111.5, 20.4, 14.7. (EI, magnetic sector): calcd for C16H17NO 239.13101, found 239.12913. mp=199-201° C.

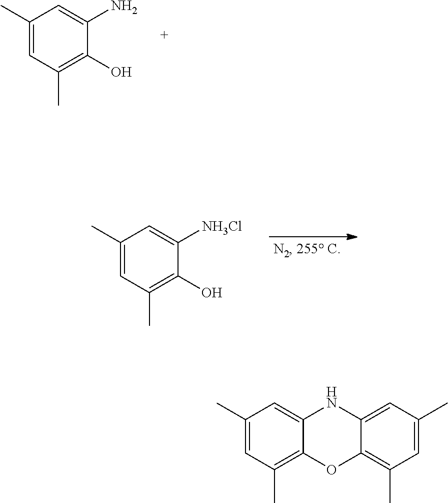

3,7-Diethylphenoxazine (41). The synthesis was carried out in an analogous fashion to (35) using 5-ethyl-2-aminophenol (2.20 g, 16 mmol) and 5-ethyl-2-aminophenol hydrochloride (2.95 g, 16 mmol) as the starting materials. The product was isolated by column chromatography (15% EtOAc in hexanes) and was subsequently recrystallized out of hexanes to afford beige needles (0.29 g, 7.6%). ¹H NMR (400 MHz; DMSO-d6): δ 7.93 (br, 1H), 6.55 (dd, J=1.9, 7.8 Hz, 2H), 6.34 (d, J=7.8 Hz, 2H), 2.38 (q, J=7.6 Hz, 4H), 1.08 (t, J=7.5 Hz, 6H). 13C NMR (101 MHz; DMSO-d6): δ 142.5, 135.8, 130.2, 122.7, 114.5, 113.0, 27.3, 15.6. (EI, magnetic sector): calcd for C16H17NO 239.13101, found 239.12834. mp=100-102° C.

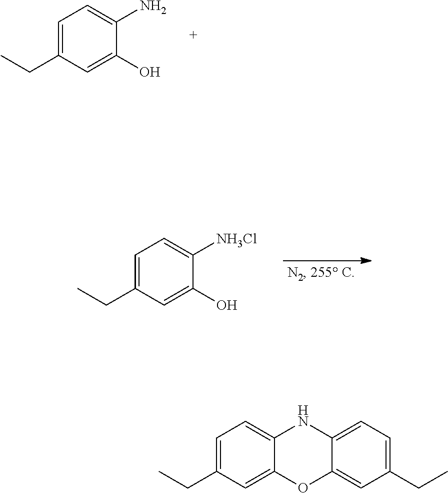

4-(tert-Butyl)-2-iodo-1-(2-nitro-4-(trifluoromethyl)phenoxy)benzene (8a). 2-Iodo-4-tert-butylphenol (2.8 g, 10 mmol) and 1 equivalent of 4-chloro-3-nitrobenzotrifluoride (2.3 g, 10 mmol) were dissolved in DMSO (15 mL, 0.67M) to which 2 equivalents of K2CO3 was added (2.8 g, 20 mmol) and the reaction mixture was placed under a N2 atmosphere. With vigorous stirring, the reaction was heated to 100° C. overnight. The reaction mixture was poured into water and extracted three times with EtOAc. The organic phase was washed twice with water, dried over MgSO4, filtered and the solvent removed under reduced pressure. The product was purified by silica gel chromatography (10% EtOAc in hexanes) and subsequently recrystallized out of hexanes to afford white needles (2.93 g, 63%). ¹H NMR (400 MHz; CDCl3): δ 8.28 (d, J=2.1 Hz, 1H), 7.90 (d, J=2.4 Hz, 1H), 7.69 (dd, J=2.3, 8.8 Hz, 1H), 7.45 (dd, J=2.1, 8.4 Hz, 1H), 7.05 (d, J=8.5 Hz, 1H), 6.88 (d, J=8.8 Hz, 1H), 1.35 (s, 9H); 13C NMR (101 MHz; CDCl3): δ 153.3, 151.5, 151.3, 139.6, 137.5, 130.8 (q, JC-F=3.3 Hz), 127.5, 124.9 (q, JC-F=34.5 Hz), 123.7 (q, JC-F=3.7 Hz), 122.9 (q, JC-F=272.2 Hz), 121.1, 118.3, 89.3, 34.6, 31.3. 19F NMR (376 MHz; CDCl3) δ −63.3. HRMS (EI, magnetic sector): calcd for C17H15F3INO3 465.00487, found 465.00109. mp=88-89° C.

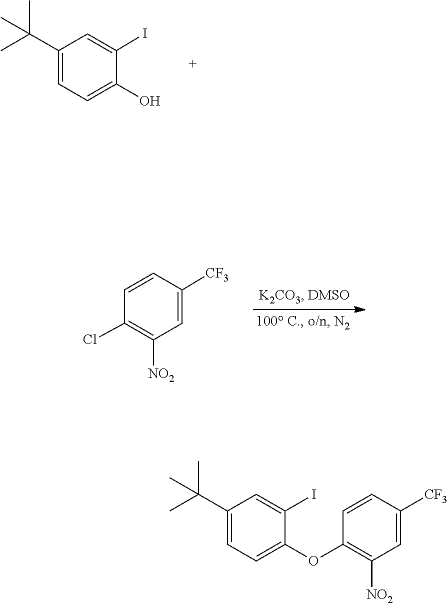 [SMILES: CC(C)(C)c1ccc(O)c(I)c1 ; O=[N+]([O-])c1cc(C(F)(F)F)ccc1Cl ; CC(C)(C)c1ccc(Oc2ccc(C(F)(F)F)cc2[N+](=O)[O-])c(I)c1]

4-(tert-butyl)-1-iodo-2-(2-nitro-4-(trifluoromethyl)phenoxy)benzene (7a). Synthesis was carried out in an analogous fashion to (8a) using 2-iodo-5-tert-butylphenol. The product was purified to the extent that was possible by column chromatography (10% EtOAc in hexanes) affording a light-coloured oil (3.3 g, 72%) which was used as-is in the preparation of (##b). ¹H NMR (400 MHz; CDCl3): δ 8.30 (d, J=2.0 Hz, 1H), 7.82 (d, J=8.3 Hz, 1H), 7.70 (dd, J=2.0, 8.8 Hz, 1H), 7.18 (d, J=2.3 Hz, 1H), 7.11 (dd, J=2.3, 8.3 Hz, 1H), 6.82 (d, J=8.7 Hz, 1H), 1.32 (s, 9H). 13C NMR (101 MHz; CDCl3): δ 155.0, 153.3, 153.2, 140.0, 130.9 (q, JC-F=3.7 Hz), 125.5, 124.7 (q, JC-F=34.5 Hz), 123.8 (q, JC-F=3.7 Hz), 122.9 (q, JC-F=272.2 Hz), 119.4, 117.8, 85.5, 34.9, 31.1. 19F NMR (376 MHz; CDCl3) δ −63.3. HRMS (EI, magnetic sector): calcd for C17H15F3INO3 465.00487, found 465.00223.

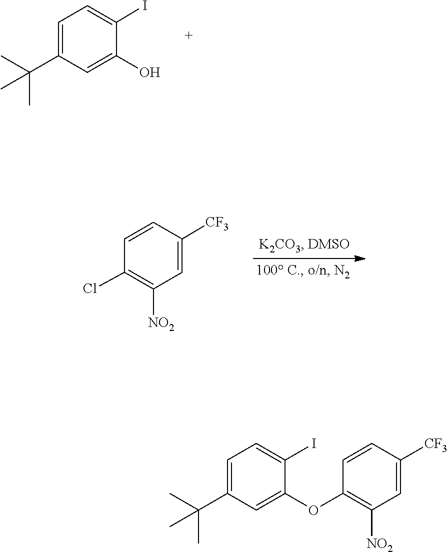 [SMILES: CC(C)(C)c1ccc(I)c(O)c1 ; O=[N+]([O-])c1cc(C(F)(F)F)ccc1Cl ; CC(C)(C)c1ccc(I)c(Oc2ccc(C(F)(F)F)cc2[N+](=O)[O-])c1]

4-(tert-butyl)-1-iodo-2-(2-nitro-5-(trifluoromethyl)phenoxy)benzene (10a). Synthesis was carried out in an analogous fashion to (8a) using 2-iodo-5-tert-butylphenol and 3-chloro-4-nitrobenzotrifluoride at a slightly reduced scale (7 mmol). The product was purified to the extent that was possible by column chromatography (10% EtOAc in hexanes) affording a light-coloured oil (2.3 g, 70%) which was used as-is in the preparation of (###b). ¹H NMR (400 MHz; CDCl3): δ 8.07 (d, J=8.4 Hz, 1H), 7.82 (d, J=8.3 Hz, 1H), 7.45 (d, J=1.8, 8.4 Hz, 1H), 7.13 (d, J=2.3 Hz, 1H), 7.10 (dd, J=2.3, 8.2 Hz, 1H), 7.00 (d, J=1.6 Hz, 1H), 1.30 (s, 9H). 13C NMR (101 MHz; CDCl3): δ 154.8, 153.3, 150.7, 142.2, 140.0, 135.5 (q, JC-F=33.7 Hz), 126.4, 125.2, 122.5 (q, JC-F=273.3 Hz), 119.3 (q, JC-F=3.7 Hz), 118.7, 115.0 (q, JC-F=3.7 Hz), 85.1, 34.9, 31.0. 19F NMR (376 MHz, CDCl3): δ −64.5. HRMS (EI, magnetic sector): calcd for C17H15F3INO3 465.00487, found 465.00231.

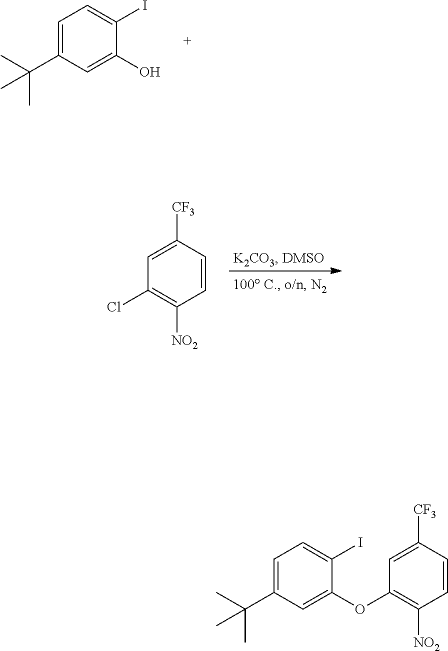 [SMILES: CC(C)(C)c1ccc(I)c(O)c1 ; O=[N+]([O-])c1ccc(C(F)(F)F)cc1Cl ; CC(C)(C)c1ccc(I)c(Oc2cc(C(F)(F)F)ccc2[N+](=O)[O-])c1]

4-(tert-butyl)-2-iodo-1-(2-nitro-5-(trifluoromethyl)phenoxy)benzene (11a). Synthesis was carried out in an analogous fashion to (8a) using 3-chloro-4-nitrobenzotrifluoride at a slightly reduced scale (7 mmol). The product was recrystallized out of hexanes to afford white needles (64% 2.1 g). ¹H NMR (400 MHz; CDCl3): δ 8.06 (d, J=8.4 Hz, 1H), 7.90 (d, J=2.3 Hz, 1H), 7.47-7.41 (m, 2H), 7.06 (d, J=1.4 Hz, 1H). 6.99 (d, J=8.5 Hz, 1H), 1.35 (s, 9H). 13C NMR (101 MHz); CDCl3): δ 151.5, 151.1, 150.6, 142.5, 137.6, 135.5 (q, JC-F=33.7 Hz), 127.5, 126.4, 122.5 (q, JC-F=273.6 Hz), 120.2, 119.6 (q, JC-F=3.7 Hz), 115.6 (q, JC-F=3.7 Hz) 88.8, 34.6, 31.3. 19F NMR (376 MHz; CDCl3): δ −64.3. HRMS (EI, magnetic sector): calcd for C17H15F3INO3 465.00487, found 465.00748. mp=92-95° C.

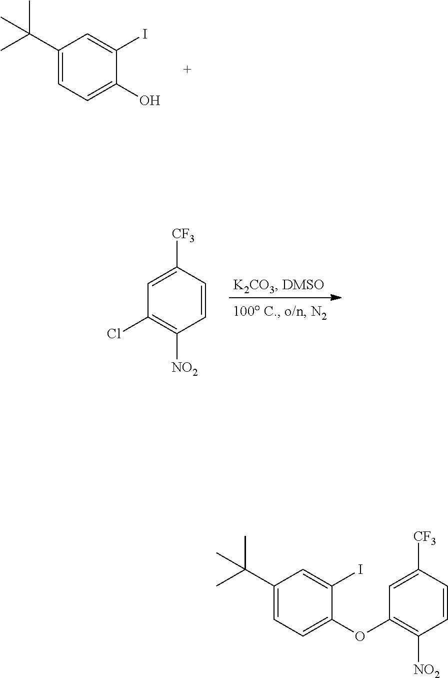 [SMILES: CC(C)(C)c1ccc(O)c(I)c1 ; O=[N+]([O-])c1ccc(C(F)(F)F)cc1Cl ; CC(C)(C)c1ccc(Oc2cc(C(F)(F)F)ccc2[N+](=O)[O-])c(I)c1]

N-(2-(4-(tert-butyl)-2-iodophenoxy)-5-(trifluoromethyl)phenyl)acetamide (8b). 4-(tert-butyl)-2-iodo-1-(2-nitro-4-(trifluoromethyl)phenoxy)benzene (8a) (2.9 g, 6.2 mmol) and 5 equivalents of SnCl2 (5.9 g, 31 mmol) were dissolved in EtOAc. The reaction stirred at room temperature overnight, then was poured into water and extracted three times with EtOAc. The organic phase was washed twice with water, dried over MgSO4, filtered and the solvent removed under reduced pressure. The residue was dissolved in neat acetic anhydride (4.4 mL) and allowed to stir overnight. The reaction was quenched with saturated Na2CO3 solution and extracted three times with EtOAc. The organic phase was washed once with water, dried over MgSO4, filtered and the solvent removed under reduced pressure. The product was isolated by silica gel chromatography (15% EtOAc in hexanes) and subsequently recrystallized out of hexanes to afford white platelets (2.22 g, 75%). ¹H NMR (400 MHz; CDCl3): δ 8.82 (d, J=1.4 Hz, 1H), 7.93 (br, 1H), 7.89 (d, J=2.3 Hz, 1H), 7.41 (dd, J=2.4, 8.5 Hz, 1H), 7.22 (dd, J=1.6, 8.5 Hz, 1H), 6.97 (d, J=8.5 Hz, 1H), 6.70 (d, J=8.4 Hz, 1H), 2.27 (s, 3H), 1.35 (s, 9H). 13C NMR (101 MHz; CDCl3): δ 168.4, 151.7, 150.8, 147.8, 137.2, 129.0, 127.3, 125.6 (q, JC-F=32.6 Hz), 123.9 (q, JC-F=272.2 Hz), 120.6 (q, JC-F=3.7 Hz), 120.4, 117.8 (q, JC-F=3.7 Hz), 114.9, 89.3, 34.5, 31.3, 25.0. 19F NMR (376 MHz; CDCl3): δ −63.1. HRMS (EI, magnetic sector): calcd for C19H19F3INO2 477.04126, found 477.03980. mp=107-108° C.

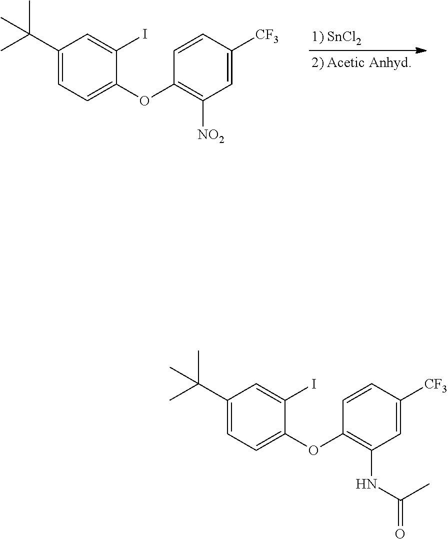 [SMILES: CC(C)(C)c1ccc(Oc2ccc(C(F)(F)F)cc2[N+](=O)[O-])c(I)c1 ; CC(=O)Nc1cc(C(F)(F)F)ccc1Oc1ccc(C(C)(C)C)cc1I]

N-(2-(5-(tert-butyl)-2-iodophenoxy)-5-(trifluoromethyl)phenyl)acetamide (7b). Synthesis was carried out in an analogous fashion to (8b) using 4-(tert-butyl)-1-iodo-2-(2-nitro-4-(trifluoromethyl)phenoxy)benzene (7a). The product was purified by column chromatography (15% EtOAc in hexanes) to afford an amorphous solid (66% 2.23 g). ¹H NMR (400 MHz; CDCl3): δ 8.83 (d, J=1.4 Hz, 1H), 7.96 (br, 1H), 7.81 (d, J=8.3 Hz, 1H), 7.22 (dd, J=1.6, 8.5 Hz, 1H), 7.11 (d, J=2.3 Hz, 1H), 7.08 (dd, J=2.3, 8.3 Hz, 1H), 2.28 (s, 3H), 1.30 (s, 9H). 13C NMR (101 MHz; CDCl3): δ 168.5, 154.7, 153.6, 147.8, 139.6, 128.7, 125.3 (q, JC-F=32.6 Hz), 124.8, 123.0 (q, JC-F=272.2), 120.7 (q, JC-F=3.7 Hz), 118.9, 117.8 (q, JC-F=3.7 Hz), 114.2, 85.7, 34.9, 31.1, 25.0. 19F NMR (376 MHz; CDCl3): δ −63.0. HRMS (EI, magnetic sector): calcd for C19H19F31NO2 477.04126, found 477.04069.

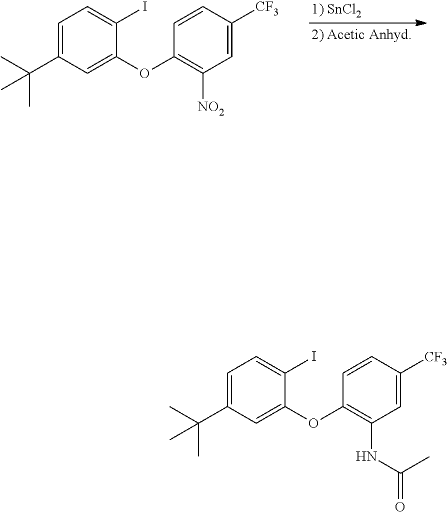 [SMILES: CC(C)(C)c1ccc(I)c(Oc2ccc(C(F)(F)F)cc2[N+](=O)[O-])c1 ; CC(=O)Nc1cc(C(F)(F)F)ccc1Oc1cc(C(C)(C)C)ccc1I]

N-(2-(5-(tert-butyl)-2-iodophenoxy)-4-(trifluoromethyl)phenyl)acetamide (10b). Synthesis was carried out in an analogous fashion to (8b) using 4-(tert-butyl)-1-iodo-2-(2-nitro-5-(trifluoromethyl)phenoxy)benzene (10a). The product was purified by column chromatography (15% EtOAc in hexanes) then subsequently recrystallized out of hexanes (74% 1.65 g). ¹H NMR (400 MHz; CDCl3): δ 8.63 (d, J=8.6 Hz, 1H), 8.00 (br, 1H), 7.81 (d, J=8.6 Hz, 1H), 7.37 (dd, J=1.3, 8.6 Hz, 1H), 7.07-7.05 (m, 2H), 6.86 (d, J=1.7 Hz, 1H), 2.28 (s, 3H), 1.29 (s, 9H). 13C NMR (101 MHz; CDCl3): δ 168.6, 154.6, 153.7, 145.1, 139.7, 131.8, 125.4 (q, JC-F=33.0 Hz), 124.5, 123.7 (q, JC-F=271.8 Hz), 120.6 (q, JC-F=3.8 Hz), 120.4, 118.0, 112.0 (q, JC-F=3.7 Hz), 85.1, 34.9, 31.0, 25.0. 19F NMR (376 MHz; CDCl3): δ −63.4. HRMS (EI, magnetic sector): calcd for C19H19F3INO2 477.04126, found 477.04121. mp=128-131° C.

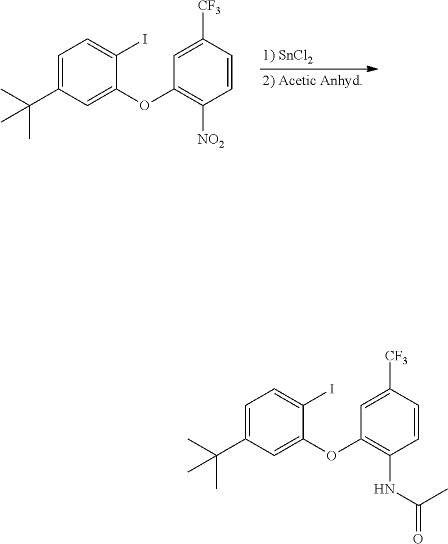 [SMILES: CC(C)(C)c1ccc(I)c(Oc2cc(C(F)(F)F)ccc2[N+](=O)[O-])c1 ; CC(=O)Nc1ccc(C(F)(F)F)cc1Oc1cc(C(C)(C)C)ccc1I]

N-(2-(4-(tert-butyl)-2-iodophenoxy)-4-(trifluoromethyl)phenyl)acetamide (11b). Synthesis was carried out in an analogous fashion to (8b) using 4-(tert-butyl)-2-iodo-1-(2-nitro-5-(trifluoromethyl)phenoxy)benzene (11a). The product was purified by column chromatography (15% EtOAc in hexanes) then subsequently recrystallized out of hexanes (70% 1.46 g). ¹H NMR (400 MHz; CDCl3): δ 8.62 (d, J=8.6 Hz, 1H), 7.97 (br, 1H), 7.89 (d, J=2.3 Hz, 1H), 7.41-7.36 (m, 2H), 6.93-6.91 (m, 2H), 2.27 (s, 3H), 1.35 (s, 9H). 13C NMR (101 MHz; CDCl3): δ 168.6, 151.9, 150.4, 145.0, 137.23, 132.1, 127.3, 125.5 (q, JC-F=33.4 Hz), 123.7 (q, JC-F=271.8 Hz), 120.9 (q, JC-F=4.0 Hz), 120.5, 119.4, 112.8 (q, JC-F=3.7 Hz), 88.7, 34.5, 31.3, 25.0. 19F NMR (376 MHz; CDCl3): δ −63.2. HRMS (EI, magnetic sector): calcd for C19H19F3INO2 477.04126, found 477.03974. mp=139-141° C.

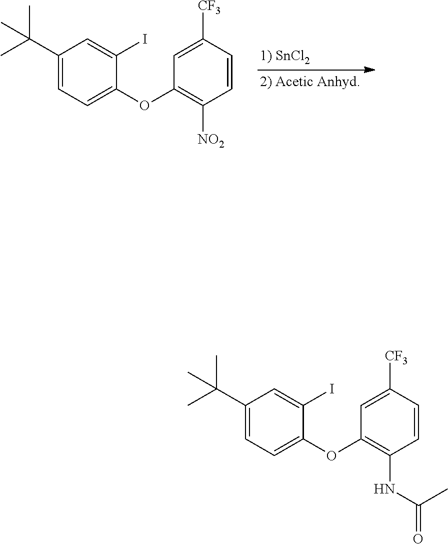 [SMILES: CC(C)(C)c1ccc(Oc2cc(C(F)(F)F)ccc2[N+](=O)[O-])c(I)c1 ; CC(=O)Nc1ccc(C(F)(F)F)cc1Oc1ccc(C(C)(C)C)cc1I]

10-Acetyl-2-tert-butyl-8-trifluoromethylphenoxazine (8c). Into a sealable tube were charged N-(2-(4-(tert-butyl)-2-iodophenoxy)-5-(trifluoromethyl)phenyl)acetamide (8b) (2.20 g, 4.6 mmol), K2CO3 (1.27 g, 9.2 mmol), copper(I) iodide (44 mg, 0.05 equiv), N,N-dimethylethylenediamine (45 μL, 0.10 equiv), and toluene (25 mL). The reaction mixture was sparged with a steady stream of N2 after which the Teflon screwcap was affixed. The reaction mixture was heated to 120° C. for 10 h with rapid stirring. After completion, the reaction mixture was diluted with dichloromethane and passed through a plug of silica, after which the solvents were removed under reduced pressure. The crude product was purified by column chromatography (20% EtOAc in hexanes) to render 10-acetyl-2-tert-butyl-8-trifluoromethylphenoxazine as an off-white solid (1.42 g, 88%). ¹H NMR (300 MHz; CDCl3): δ 7.91 (d, J=1.9 Hz, 1H), 7.45 (dd, J=1.9, 8.5 Hz, 1H), 7.39 (d, J=2.2 Hz, 1H), 7.26 (dd, J=2.2, 8.5 Hz, 1H), 7.19 (d, J=8.5 Hz, 1H), 7.08 (d, J=8.5, 1H), 2.35 (s, 3H), 1.34 (s, 9H). 13C NMR (75 MHz; CDCl3): δ 169.1, 148.1, 147.4, 129.6, 128.4, 125.5 (q, JC-F=33.0 Hz), 124.1, 123.9 (q, JC-F=4.4 Hz) 123.8 (q, JC-F=271.8 Hz), 123.0 (q, JC-F=4.4 Hz), 122.1, 117.1, 116.3, 34.6, 31.3, 22.9. 19F NMR (376 MHz; CDCl3): δ −63.0. HRMS (EI, magnetic sector): calcd for C19H18F3NO2 349.12896, found 349.12775. mp=102-105° C.

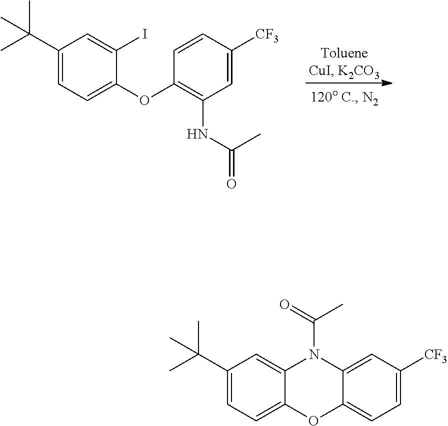 [SMILES: CC(=O)Nc1cc(C(F)(F)F)ccc1Oc1ccc(C(C)(C)C)cc1I ; CC(=O)N1c2cc(C(C)(C)C)ccc2Oc2ccc(C(F)(F)F)cc21]

10-Acetyl-3-tert-butyl-8-trifluoromethylphenoxazine (7c). Synthesis was carried out in an analogous fashion to (8c) using N-(2-(5-(tert-butyl)-2-iodophenoxy)-5-(trifluoromethyl)phenyl)acetamide (7b). The product was purified by column chromatography (20% EtOAc in hexanes) then subsequently recrystallized out of hexanes to afford white needles (1.48 g, 92%). ¹H NMR (400 MHz, CDCl3): δ 7.87 (d, J=1.7 Hz, 1H), 7.46 (dd, J=2.2, 8.5 Hz, 1H), 7.32 (d, J=8.2 Hz, 1H), 7.21-7.17 (m, 3H), 2.36 (s, 3H), 1.33 (s, 9H). 13C NMR (101 MHz; CDCl3): δ 169.1, 153.4, 151.3, 150.0, 129.6, 126.2, 125.6 (q, JC-F=33.0 Hz), 124.1, 123.9 (q, JC-F=3.7 Hz), 123.8 (q, JC-F=271.8 Hz), 122.9 (q, JC-F=3.7 Hz), 121.0, 117.1, 114.2, 34.7, 31.2, 22.9. 19F NMR (376 MHz; CDCl3): δ −63.0. HRMS (EI, magnetic sector): calcd for C19H18F3NO2 349.12896, found 349.12727. mp=69-71° C.

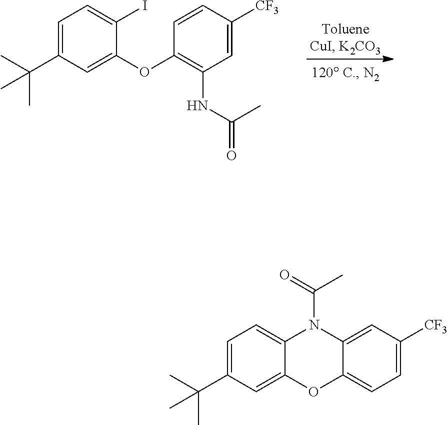 [SMILES: CC(=O)Nc1cc(C(F)(F)F)ccc1Oc1cc(C(C)(C)C)ccc1I ; CC(=O)N1c2ccc(C(C)(C)C)cc2Oc2ccc(C(F)(F)F)cc21]

10-Acetyl-3-tert-butyl-7-trifluoromethylphenoxazine (10c). Synthesis was carried out in an analogous fashion to (8c) using N-(2-(5-(tert-butyl)-2-iodophenoxy)-4-(trifluoromethyl)phenyl)acetamide (10b). The product was purified by column chromatography (20% EtOAc in hexanes) then subsequently recrystallized out of hexanes to afford white needles (0.90 g, 81%). ¹H NMR (400 MHz, CDCl3): δ 7.69 (d, J=8.0 Hz, 1H), 7.41-7.38 (m, 2H), 7.32 (dd, J=1.1, 7.7 Hz, 1H), 7.20-7.17 (m, 2H), 2.36 (s, 3H), 1.33 (s, 9H). 13C NMR (101 MHz; CDCl3): δ 169.1, 151.3, 151.0, 150.2, 132.6, 128.9 (q, JC-F=33.4 Hz), 126.2, 125.7, 124.1, 123.5 (q, JC-F=272.2 Hz), 120.9, 120.2 (q, JC-F=3.7 Hz), 114.2, 114.2 (q, JC-F=3.7 Hz), 34.7, 31.2, 23.0. 19F NMR (376 MHz; CDCl3): δ −63.7. HRMS (EI, magnetic sector): calcd for C19H18F3NO2 349.12896, found 349.13058. mp=102-104° C.

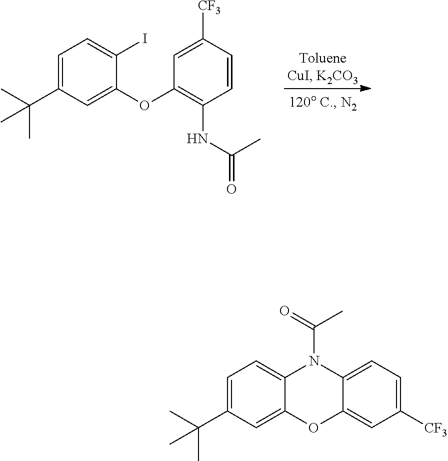

10-Acetyl-2-tert-butyl-7-trifluoromethylphenoxazine (11c). Synthesis was carried out in an analogous fashion to (8c) using N-(2-(4-(tert-butyl)-2-iodophenoxy)-4-(trifluoromethyl)phenyl)acetamide (11b). The product was purified by column chromatography (20% EtOAc in hexanes) then subsequently recrystallized out of hexanes to afford white needles (0.63 g, 64%). ¹H NMR (400 MHz, CDCl3): δ 7.73 (d, J=8.3 Hz, 1H), 7.42-7.37 (m, 3H), 7.26 (dd, J=2.2, 8.5 Hz, 1H), 7.08 (d, J=8.5 Hz, 1H), 2.36 (s, 3H), 1.34 (s, 9H). 13C NMR (101 MHz; CDCl3): δ 169.2, 160.0, 148.3, 147.3, 132.5, 128.9 (q, JC-F=33.4 Hz), 128.4, 125.9, 124.1, 123.5 (q, JC-F=271.8 Hz), 122.0, 120.1 (q, JC-F=3.7 Hz), 116.3, 114.1 (q, JC-F=3.7 Hz), 34.6, 31.3, 23.0. 19F NMR (376 MHz; CDCl3): δ −63.7. HRMS (EI, magnetic sector): calcd for C19H18F3NO2 349.12896, found 349.13046. mp=146-148° C.

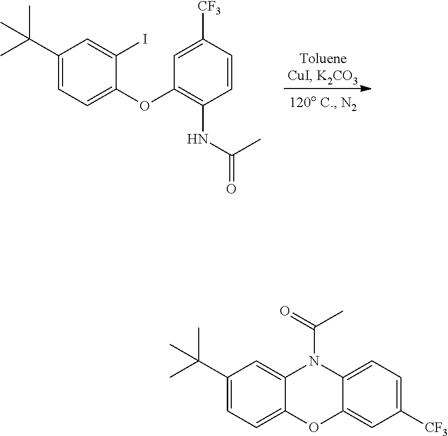

2-tert-Butyl-8-trifluoromethylphenoxazine (8). Into a double-neck flask were charged 10-Acetyl-2-tert-butyl-8-trifluoromethylphenoxazine (8c) (1.40 g, 4.0 mmol) and ethanol (25 mL). The ethanol was degassed under a continuous stream of N2 and degassed 33% aqueous HCl (4 mL) was added. (If the solutions contain significant amounts of 02 the HCl will promote the product (#) to oxidize giving the solution a strong magenta colour as the reaction proceeds). While under N2 the solution was heated to 70° C. with stirring and the reaction was monitored by TLC. When the starting material had been completely consumed (≥2 hr), the reaction was quenched with Na2CO3 solution. The aqueous mixture was extracted 3 times with EtOAc and the organic phase was washed twice with water. The organic solution was dried over MgSO4, and filtered, and the solvent was removed under reduced pressure. The product was recrystallized from hexanes to afford white needles (1.13 g, 92%). ¹H NMR (400 MHz; DMSO-d6): δ 8.45 (br, 1H), 6.87 (dd, J=2.0, 8.2 Hz, 1H), 6.73 (d, J=8.2 Hz, 1H), 6.64-6.61 (m, 2H), 6.56 (d, J=8.3 Hz, 1H), 6.46 (d, J=2.2 Hz, 1H), 1.19 (s, 9H). 13C NMR (101 MHz; DMSO-d6): δ 147.0, 145.9, 133.4, 130.5, 124.4 (q, JC-F=31.9 Hz), 124.0 (q, JC-F=271.4 Hz), 117.5, 117.3 (q, JC-F=4.0 Hz), 115.3, 114.7, 110.7, 109.2 (q, JC-F=3.7 Hz), 33.9, 31.0. 19F NMR (376 MHz; DMSO-d6): δ −61.5. HRMS (EI, magnetic sector): calcd for C17H16F3NO 307.11840, found 307.11644. mp=74-76° C.

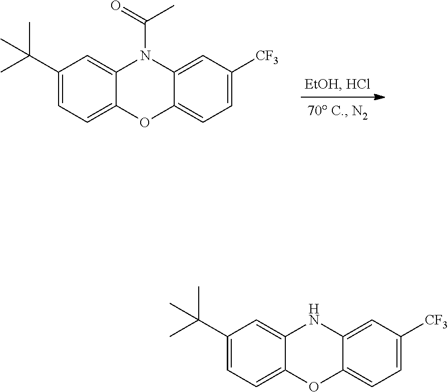 [SMILES: CC(=O)N1c2cc(C(C)(C)C)ccc2Oc2ccc(C(F)(F)F)cc21 ; CC(C)(C)c1ccc2c(c1)Nc1cc(C(F)(F)F)ccc1O2]

3-tert-Butyl-8-trifluoromethylphenoxazine (7). Synthesis was carried out in an analogous fashion to (8) using 10-Acetyl-3-tert-butyl-8-trifluoromethylphenoxazine (7c). The product was recrystallized from hexanes to afford white needles (1.09 g, 89%). ¹H NMR (400 MHz; DMSO-d6): δ 8.43 (br, 1H), 6.87 (dd, J=2.2, 8.2 Hz, 1H), 6.77 (dd, J=2.1, 8.2 Hz, 1H), 6.72 (d, J=8.2 Hz, 1H), 6.66 (d, J=2.1 Hz, 1H), 6.65 (d, J=2.1 Hz, 1H), 6.39 (d, J=8.0 Hz, 1H), 1.18 (s, 9H). 13C NMR (101 MHz; DMSO-d6): δ 145.8, 144.0, 141.8, 133.5, 128.5, 124.4 (q, JC-F=31.9 Hz), 124.0 (q, JC-F=271.4 Hz), 120.7, 117.2 (q, JC-F=4.0 Hz), 115.3, 113.0, 112.6, 109.1 (q, JC-F=3.7 Hz), 33.8, 31.0. 19F NMR (376 MHz; DMSO-d6): δ −61.5. HRMS (EI, magnetic sector): calcd for C17H16F3NO 307.11840, found 307.11403. mp=167-169° C.

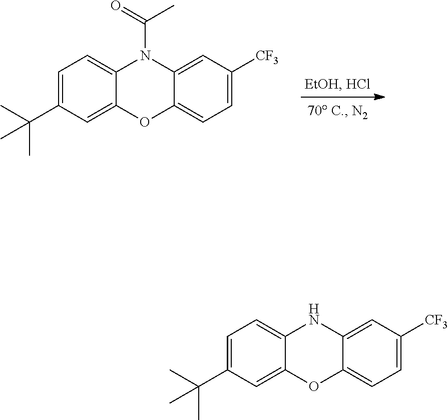 [SMILES: CC(=O)N1c2ccc(C(C)(C)C)cc2Oc2ccc(C(F)(F)F)cc21 ; CC(C)(C)c1ccc2c(c1)Oc1ccc(C(F)(F)F)cc1N2]

3-tert-Butyl-7-trifluoromethylphenoxazine (10). Synthesis was carried out in an analogous fashion to (8) using 10-Acetyl-3-tert-butyl-7-trifluoromethylphenoxazine (10c). The product was recrystallized from hexanes to afford white needles (0.70 g, 94%). ¹H NMR (400 MHz; DMSO-d6): δ 8.63 (br, 1H), 7.05 (dd, J=1.9, 8.2 Hz, 1H), 6.82 (d, J=1.9 Hz, 1H), 6.77 (dd, J=2.2, 8.2 Hz, 1H), 6.64 (d, J=2.1 Hz, 1H), 6.52 (d, J=8.2 Hz, 1H), 6.41 (d, J=8.1 Hz, 1H), 1.18 (s, 9H). 13C NMR (101 MHz; DMSO-d6): δ 144.3, 142.8, 142.0, 136.5, 128.3, 124.2 (q, JC-F=270.7 Hz), 121.4 (q, JC-F=4.0 Hz), 120.5, 119.9 (q, JC-F=32.6 Hz), 113.1, 112.8, 112.4, 111.6 (q, JC-F=3.7 Hz), 33.8, 30.9. 19F NMR (376 MHz; DMSO-d6): δ −60.6. HRMS (EI, magnetic sector): calcd for C17H16F3NO 307.11840, found 307.11665. mp=95-97° C.

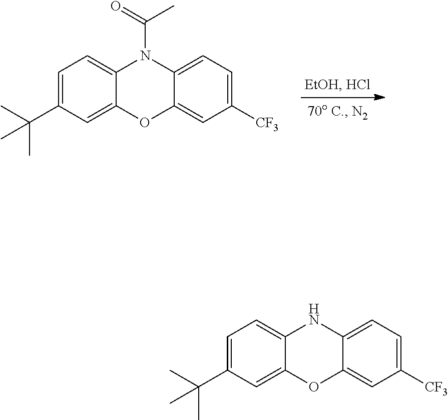 [SMILES: CC(=O)N1c2ccc(C(C)(C)C)cc2Oc2cc(C(F)(F)F)ccc21 ; CC(C)(C)c1ccc2c(c1)Oc1cc(C(F)(F)F)ccc1N2]

2-tert-Butyl-7-trifluoromethylphenoxazine (11). Synthesis was carried out in an analogous fashion to (8) using 10-Acetyl-2-tert-butyl-7-trifluoromethylphenoxazine (11c). The product was recrystallized from hexanes to afford white needles (0.47 g, 96%). ¹H NMR (400 MHz; DMSO-d6): δ 8.65 (br, 1H), 7.05 (dd, J=1.9, 8.2 Hz, 1H), 6.84 (d, J=1.9 Hz, 1H), 6.64 (dd, J=2.3, 8.3 Hz, 1H), 6.56-6.51 (m, 2H), 6.49 (d, J=2.3 Hz, 1H), 1.19 (s, 9H). 13C NMR (101 MHz; DMSO-d6): δ 146.8, 142.9, 140.2, 136.3, 130.3, 124.2 (q, J=270.7 Hz), 121.4 (q, J=4.0 Hz), 120.2 (q, J=32.3 Hz), 117.8, 114.6, 112.9, 111.6 (q, J=3.7 Hz), 110.8, 33.8, 31.0. 19F NMR (376 MHz; DMSO-d6): δ −60.7. HRMS (EI, magnetic sector): calcd for C17H16F3NO 307.11840, found 307.11864. mp=162-165° C.

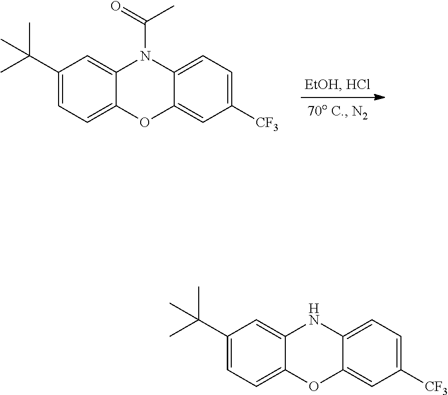 [SMILES: CC(=O)N1c2ccc(C(F)(F)F)cc2Oc2ccc(C(C)(C)C)cc21 ; CC(C)(C)c1ccc2c(c1)Nc1ccc(C(F)(F)F)cc1O2]

N-(5-isopropyl-2-nitrophenyl)acetamide (42a). Into a 50 ml round bottom flask equipped with a magnetic stir bar was charged acetic anhydride (15 ml). The flask was chilled on an ice bath, and with stirring 3-isopropylaniline (4.05 g, 30 mmol) was gradually added. After ~30 minutes copper (II) nitrate trihydrate (3.38 g) was added and the reaction mixture was allowed to heat to room temperature overnight. After 16 hours the reaction was quenched in a large flask of saturated NaHCO₃ solution. The aqueous mixture was extracted 3 times with EtOAc and the organic phase was washed twice with water. The organic solution was dried over MgSO4, and filtered, and the solvent was removed under reduced pressure. The title product was isolated by column chromatography (20% EtOAc in hexanes) appearing as yellow needle like crystals (3.20 g, 48%). ¹H NMR (400 MHz; DMSO-d6): δ 10.23 (br, 1H), 7.87 (d, J=8.4 Hz, 1H), 7.51 (d, J=1.9 Hz, 1H), 7.23 (dd, J=2.0, 8.5 Hz, 1H), 2.97 (sept, J=7.0 Hz, 1H), 2.06 (s, 3H), 1.21 (d, J=7.0 Hz, 6H). 13C NMR (101 MHz; DMSO-d6): δ 168.5, 155.1, 140.3, 131.5, 125.0, 123.1, 122.7, 33.33, 23.4, 23.2. HRMS (EI, magnetic sector): calcd for C11H14N2O3 222.10044, found 222.10045. mp=69-71° C.

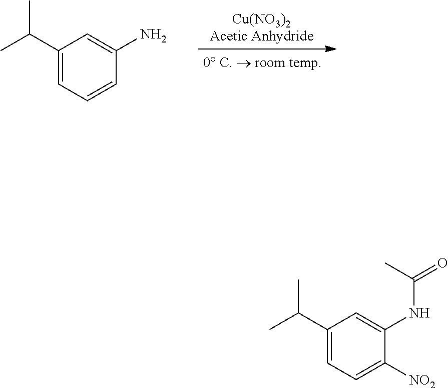

5-Isopropyl-2-nitroaniline (42b). Into a double-neck flask were charged N-(5-isopropyl-2-nitrophenyl)acetamide (42a) (3.11 g, 14.0 mmol) and ethanol (100 ml). The ethanol was degassed under a continuous stream of N2 and concentrated HCl was added (5 ml), after which the flask was sealed with a rubber septa and balloon with N2 affixed to a needle was poked into the septa. With stirring the mixture was heated to 80° C. for 16 hours. Under reduced pressure, the solvent was reduced to ~20 ml and was poured into 1M NaOH solution. The aqueous suspension was extracted with Et2O 3 times, then the organic extracts were dried over MgSO4, and filtered, and the solvent was removed under reduced pressure. The product appeared as a yellow oil and was used without further purification (2.50 g, 99%). ¹H NMR (400 MHz; DMSO-d6): δ 7.88 (d, J=8.9 Hz, 1H), 7.36 (br, 2H), 6.85 (d, J=1.7 Hz, 1H), 6.53 (dd, J=1.9, 9.0 Hz, 1H), 2.79 (sept, J=6.9 Hz, 1H), 1.16 (d, J=7.0 Hz, 6H). 13C NMR (101 MHz; DMSO-d6): δ 156.7, 146.4, 128.6, 125.5, 115.7, 114.8, 33.4, 22.9. HIRMS (EI, magnetic sector): calcd for C9H12N2O2 180.08988, found 180.08705.

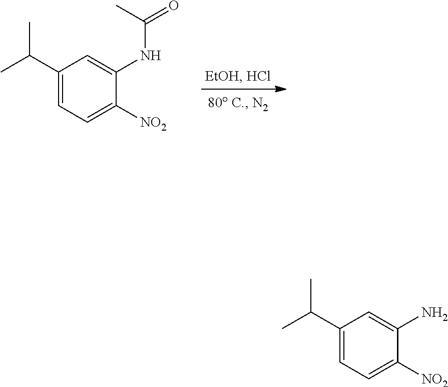 [SMILES: CC(=O)Nc1cc(C(C)C)ccc1[N+](=O)[O-] ; CC(C)c1ccc([N+](=O)[O-])c(N)c1]

2-Chloro-4-isopropyl-1-nitrobenzene (42c). The procedure is based on the method described by Doyle et al.16 In a double-necked round-bottom flask equipped with a magnetic stir bar and reflux condenser was added anhydrous MeCN (47 mL), anhydrous copper (II) chloride (2.10 g, 15.6 mmol) and 90% tert-butyl nitrite (2.23 g, 19.5 mmol). The mixture was stirred and heated to 65° C. for ~5 minutes after which 5-isopropyl-2-nitroaniline (#b) (2.34 g, 13.0 mmol) dissolved in MeCN (5 mL) was added by dropping funnel over a period of 5-10 min. When evolution of N2 had ceased (approx. 10 min.) the reaction was cooled to room temperature and the mixture was poured into 20% aqueous HCl. The aqueous suspension was extracted with Et2O, and the organic phase was washed once with 20% aqueous HCl. The organic extracts were dried over MgSO4, and filtered, and the solvent was removed under reduced pressure. The product was isolated by column chromatography (10% EtOAc in hexanes) to afford the title product which appeared as a light-yellow oil (2.21 g, 85%). %). ¹H NMR (400 MHz; CDCl3): δ 7.86 (d, J=8.3 Hz, 1H), 7.40 (d, J=1.9 Hz, 1H), 7.26 (dd, J=1.9, 8.4 Hz, 1H), 2.98 (sept, J=7.0 Hz, 1H), 1.29 (d, J=7.0 Hz, 6H). 13C NMR (101 MHz; CDCl3): δ 155.5, 129.9, 127.2, 125.9, 125.7, 34.0, 23.4. HRMS (EI, magnetic sector): calcd for C9H10ClNO2 199.04001, found 199.03838.

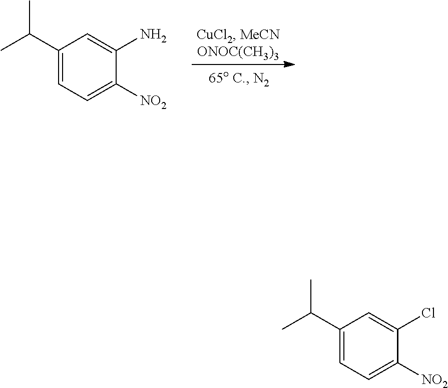

2-Iodo-5-isopropylphenol(42d). 3-Isopropylphenol (4.09 g, 30 mmol), sodium iodide (4.50 g, 30 mmol), and NaOH (1.20 g, 30 mmol) were dissolved in MeOH (85 mL) with stirring, cooled with an ice bath. Approximately 40 mL of 6% sodium hypochlorite solution (bleach) were added dropwise over 1 h until the sodium iodide was consumed. On completion, the reaction mixture was poured into water, and the aqueous mixture was extracted 3 times with EtOAc, and the organic phase was washed twice with water. The organic extracts were dried over MgSO4, filtered, and the solvent was removed under reduced pressure. The title product was isolated by column chromatography (5% EtOAc in hexanes) appearing as a colourless oil (2.93 g, 37%). ¹H NMR (400 MHz; CDCl3): δ 7.55 (d, J=8.3 Hz, 1H), 6.90 (d, J=2.0 Hz, 1H), 6.59 (dd, J=2.2, 8.3 Hz, 1H), 5.23 (br, 1H), 2.85 (sept, J=6.9 Hz, 1H), 1.24 (d, J=6.9 Hz, 6H). The spectra were consistent with those previously reported (Hermann, G. J.; Annis, M. C.; Edwards, P. D.; Corrales, M.; Diaz, L.; Goodnow, R. A. An Efficient Synthetic Route to Novel 3-Alkyl- and 3-Aryl-4-Iodophenols. Synthesis (Stuttg). 2008, 2008 (02), 221-224).

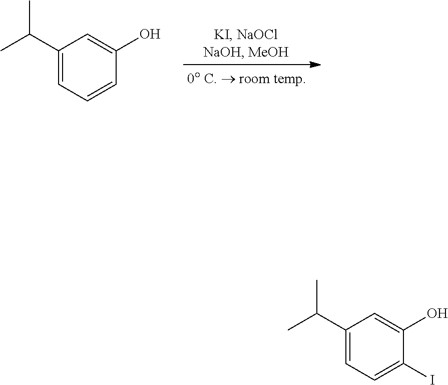

I-Iodo-4-isopropyl-2-(5-isopropyl-2-nitrophenoxy)benzene (42e). Synthesis was carried out in an analogous fashion to (8a) using 2-iodo-5-isopropylphenol (2.62 g, 10 mmol) and 2-chloro-4-isopropyl-1-nitrobenzene (2.00 g, 10 mmol). The title product was purified by column chromatography to the extent that was possible (5% EtOAc in hexanes) appearing as a light-yellow oil (2.71 g, 64%) and carried forward without further purification. ¹H NMR (400 MHz; CDCl3): δ 7.98 (d, J=8.4 Hz, 1H), 7.78 (d, J=8.0 Hz, 1H), 7.07 (dd, J=1.8, 8.5 Hz, 1H), 6.86-6.82 (m, 2H), 6.70 (d, J=1.8 Hz, 1H), 2.87 (sept, J=6.9 Hz, 2H), 1.20 (d, J=6.9 Hz, 6H), 1.19 (d, J=6.9 Hz, 6H). 13C NMR (101 MHz; CDCl3): δ 156.7, 155.0, 151.7, 150.3, 139.8, 126.2, 124.8, 121.2, 118.0, 117.5, 84.8, 34.2, 33.7, 23.7, 23.4. HRMS (EI, magnetic sector): calcd for C18H201NO3 425.04879, found 425.04885.

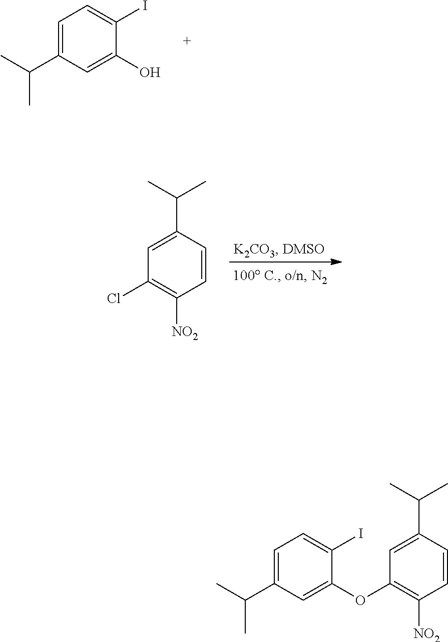 [SMILES: CC(C)c1ccc(I)c(O)c1 ; CC(C)c1ccc([N+](=O)[O-])c(Cl)c1 ; CC(C)c1ccc(I)c(Oc2cc(C(C)C)ccc2[N+](=O)[O-])c1]

N-(2-(2-iodo-5-isopropylphenoxy)-4-isopropylphenyl)acetamide (42f). To a round-bottom flask equipped with a magnetic stir bar were combined 1-iodo-4-isopropyl-2-(5-isopropyl-2-nitrophenoxy)benzene (42e) (2.67 g, 6.3 mmol), absolute ethanol (30 mL) iron powder (1.40 g, 25 mmol). With stirring the flask was heated to 60° C. and concentrated HCl (6 mL) were added by dropping funnel. The reaction was allowed to proceed until the starting material was consumed (as monitored by TLC) after which the mixture was cooled and was poured into 1M NaOH. The aqueous suspension was extracted several times with EtOAc and the organic extracts were dried over MgSO4, filtered and the solvent removed under reduced pressure. The residue was dissolved in neat acetic anhydride (3 mL) and allowed to stir overnight. The reaction was quenched with saturated Na2CO3 solution and extracted three times with EtOAc. The organic phase was washed once with water, dried over MgSO4, filtered and the solvent removed under reduced pressure. The product was isolated by silica gel chromatography (20% EtOAc in hexanes) appearing as a light-coloured oil (1.93 g, 70%). ¹H NMR (400 MHz; CDCl3): δ 8.30 (d, J=8.4 Hz, 1H), 7.77 (d, J=8.0 Hz, 1H), 7.67 (br, 1H), 7.01 (dd, J=2.0, 8.3 Hz, 1H), 6.82-6.79 (m, 2H), 6.68 (d, J=1.9 Hz, 1H), 2.87-2.77 (m, 2H), 2.18 (s, 3H), 1.18 (d, J=6.9 Hz, 6H), 1.17 (d, J=7.0 Hz, 6H). 13C NMR (151 MHz; DMSO-d6): δ 168.6, 155.7, 150.9, 147.5, 145.4, 139.2, 127.0, 124.3, 124.0, 121.0, 117.4, 115.1, 85.7, 33.0, 32.8, 23.7, 23.6, 23.5. HRMS (EI, magnetic sector): calcd for C20H24INO2 437.08517, found 437.08308.

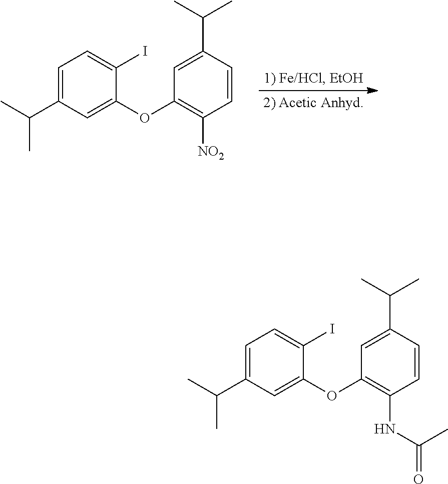 [SMILES: CC(C)c1ccc(I)c(Oc2cc(C(C)C)ccc2[N+](=O)[O-])c1 ; CC(=O)Nc1ccc(C(C)C)cc1Oc1cc(C(C)C)ccc1I]

10-Acetyl-3,7-diisopropylphenoxazine (42 g). Synthesis was carried out in an analogous fashion to (8c) using N-(2-(2-iodo-5-isopropylphenoxy)-4-isopropylphenyl)acetamide (42f). The product was purified by column chromatography (15% EtOAc in hexanes) appearing as a colourless resin (1.11 g, 88%). ¹H NMR (400 MHz; DMSO-d6): δ 7.49 (d, J=8.1 Hz, 2H), 7.06-7.03 (m, 4H), 2.89 (sept, J=6.9 Hz, 2H), 2.25 (s, 3H), 1.19 (d, J=6.9 Hz, 12H). 13C NMR (101 MHz; DMSO-d6): δ 168.7, 150.3, 147.7, 127.0, 125.0, 121.4, 114.2, 33.0, 23.7, 22.7. calcd for C20H23NO2 309.17288, found 309.17279.

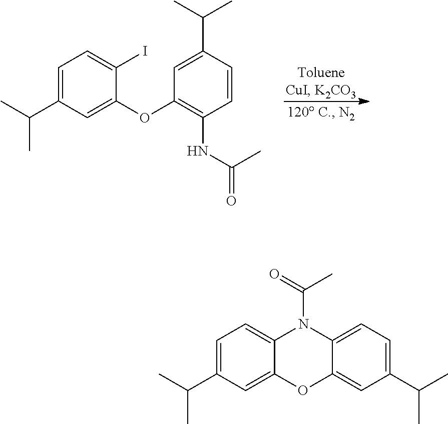 [SMILES: CC(=O)Nc1ccc(C(C)C)cc1Oc1cc(C(C)C)ccc1I ; CC(=O)N1c2ccc(C(C)C)cc2Oc2cc(C(C)C)ccc21]

3,7-diisopropylphenoxazine (42). Synthesis was carried out in an analogous fashion to (42) using 10-acetyl-3,7-diisopropylphenoxazine (42 g). The product was purified by column chromatography (10% EtOAc in hexanes) to afford the product as an off-white solid (0.67 g, 72%). ¹H NMR (400 MHz; DMSO-d6): δ 7.93 (br, 1H), 6.58 (dd, J=1.2, 7.8 Hz, 2H), 6.48 (d, J=1.2 Hz, 2H), 6.35 (d, J=7.9 Hz, 2H), 2.66 (sept, J=6.9 Hz, 2H), 1.11 (d, J=6.9 Hz, 12H). 13C NMR (101 MHz; DMSO-d6): δ 142.5, 140.6, 130.3, 121.2, 113.0, 112.9, 32.5, 23.8. calcd for C18H21NO 267.16231, found 267.16251.

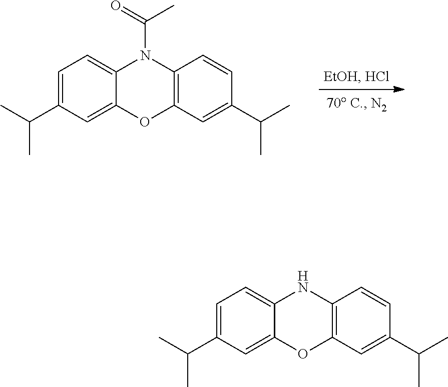

Preparation of 2-(2-Bromophenoxy)aniline Intermediates. A general 2-step procedure for the preparation of 2-(2-bromophenoxy)aniline derivatives, intermediates to the preparation of several described phenoxazine derivatives, is given. In a round bottom flask equipped with a stir bar were combined a substituted 2-bromophenol (1 eq.), K₂CO₃ (2 eq.) and DMSO (0.5M with respect to the phenol). This mixture was heated with stirring to reach the target temperature for the reaction (60-120° C., depending on the substitution partner), after which the 2-chloronitrobenzene derivative was added (1 eq.). The temperature was maintained until complete conversion was observed, after which the reaction was cooled to room temperature and poured into 6 times the volume of water. This was extracted with ethyl acetate, and the organic layer was washed with water, then 1 M HCl. The organic layer was drawn-off into a round bottom flask with a stir bar and 5 eq. of anhydrous SnCl₂ (with respect to the phenol) was added and stirred at room temperature overnight. After confirming that the reduction was successful, the reaction mixture was poured into a 1 L Erlenmeyer flask containing saturated NaHCO₃ solution. The flask was swirled (not shaken, in order to minimize emulsions of Sn(OH)Cl in the organic phase), and the organic phase was carefully decanted into a separatory funnel. More ethyl acetate was added to the Erlenmeyer flask and the process was repeated until a point of diminished returns. The combined organics were rinsed, then dried with MgSO₄, then filtered. This residue was either purified via silica gel chromatography to isolate the aniline product, recrystallized, or when applicable it was dissolved in acetic anhydride overnight and the acetamide was isolated instead.

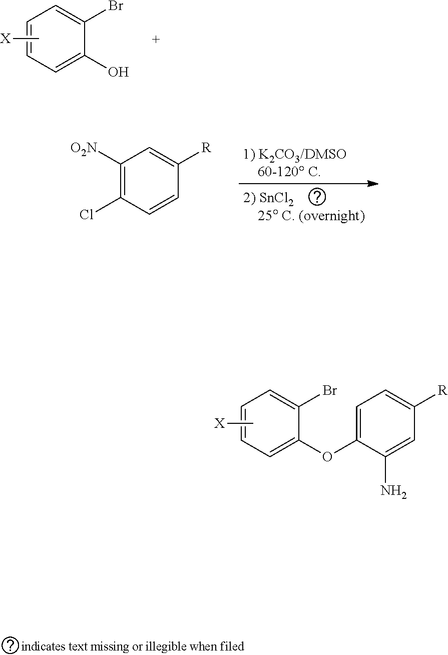

1-(3-amino-4-(2-bromo-5-(trifluoromethyl)phenoxy)phenyl)ethenone (51a). In a round bottom flask with a magnetic stir bar were combined 2-bromo-5-trifluoromethylphenol (6.02 g, 25 mmol), 4-chloro-3-nitroacetophenone (5.00 g, 25 mmol), DMSO (25 mL) and potassium carbonate (6.90 g, 50 mmol). With rapid stirring the reaction was heated to 120° C. for 20 minutes (or sooner if CO₂ evolution ceased. Even leaving it for ~1 hr under these conditions could reduce the yield by more than half as this intermediate product is unstable under these conditions.) The reaction was cooled to room temperature and poured into 150 mL 1 M HCl and extracted with ~120 mL of ethyl acetate. The organic layer was subsequently washed with 100 mL 1 M HCl, and the water saturated organic layer was transferred to a 250 mL round bottom flask containing tin chloride (23.7 g, 125 mmol). The reaction was stirred at room temperature overnight (or until the intermediate product was consumed), after which the reaction was poured into a 1 L Erlenmeyer with 300 mL of saturated NaHCO₃ solution with stirring. The organic layer was washed several times with distilled water and the initial aqueous phase was extracted several times with ethyl acetate (gently to minimize emulsion.) The organic portions were combined, dried with MgSO₄, filtered then the solvent removed under reduced pressure. The resulting residue was purified by flash chromatography (25% ethyl acetate in hexanes). The fractions containing the product were combined and reduced to ~10% of the total volume, and addition of hexanes at ~3 times that volume promoted crystallization of the title product on standing. The decanted and dried product appeared as vanilla-coloured needles (6.56 g, 70.1%); mp=125-128° C. ¹H NMR (400 MHz; DMSO-d6): 7.99 (d, J=8.3 Hz, 1H), 7.48-7.44 (m, 2H), 7.18 (dd, J=8.3, 2.2 Hz, 1H), 7.11 (d, J=2.0 Hz, 1H), 6.76 (d, J=8.3 Hz, 1H), 5.39 (br, 2H), 2.49 (s, 3H). ¹³C NMR (101 MHz; DMSO-d6): δ 197.1, 153.5, 145.0, 139.9, 135.0, 134.1, 129.7 (q, J=32.6 Hz), 123.4 (q, J=272.5 Hz), 121.6 (q, J=3.7 Hz), 118.2, 117.3, 115.2, 115.1 (q, J=3.3 Hz), 26.6. ¹⁹F NMR (376 MHz; DMSO-d6): δ −61.6. HRMS (EI, magnetic sector): calcd for C₁₅H₁₁BrF₃NO₂ 372.99253, found 372.99394.

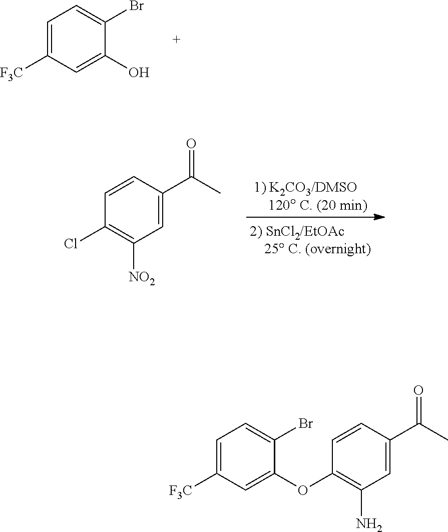 [SMILES: Oc1cc(C(F)(F)F)ccc1Br ; CC(=O)c1ccc(Cl)c([N+](=O)[O-])c1 ; CC(=O)c1ccc(Oc2cc(C(F)(F)F)ccc2Br)c(N)c1]

1-(3-amino-4-(2-bromo-4-(trifluoromethyl)phenoxy)phenyl)ethenone (68a). This synthesis was carried out at a 20 mmol scale, using 2-bromo-4-trifluoromethylphenol as the nucleophile and 4-chloro-3-nitroacetophenone as the electrophile. The substitution step was run at 110° C. for approximately 15 min. The isolated yield of title product, isolated by chromatography (25% EtOAc in hexanes), was 40% appearing as a white solid. ¹H NMR (400 MHz; DMSO-d6): δ 8.12 (d, J=1.9 Hz, 1H), 7.72 (dd, J=8.6, 1.8 Hz, 1H), 7.46 (d, J=2.2 Hz, 1H), 7.20 (d, J=8.3, 2.2 Hz, 1H), 6.92 (d, J=8.6 Hz, 1H), 6.88 (d, J=8.3 Hz, 1H), 5.38 (br, 2H), 2.51 (s, 3H). ¹³C NMR (101 MHz; DMSO-d6): δ 197.1, 156.4, 144.2, 140.3, 134.6, 130.6 (q, J=3.7 Hz), 126.6 (q, J=3.7 Hz), 124.8 (q, J=32.6 Hz), 123.3 (q, J=272.2 Hz), 119.6, 119.3, 117.9, 115.3, 113.1, 26.6. ¹⁹F NMR (283 MHz; DMSO-d6): δ −60.7. (EI, magnetic sector): calcd for C₁₅H₁₁BrF₃NO₂ 372.99253, found 372.99129.

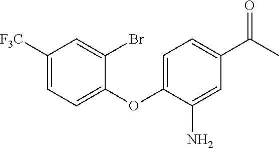 [SMILES: CC(=O)c1ccc(Oc2ccc(C(F)(F)F)cc2Br)c(N)c1]

1-(3-amino-4-(2-bromo-5-chlorophenoxy)phenyl)ethanone (71a). This synthesis was carried out at a 20 mmol scale, using 2-bromo-5-chlorophenol as the nucleophile and 4-chloro-3-nitroacetophenone as the electrophile. The substitution step was run at 110° C. for approximately 15 min. The isolated yield of title product, crystallized out of ethyl acetate, was 50% appearing as light brown crystals. ¹H NMR (400 MHz; DMSO-d6): δ 7.77 (d, J=8.5 Hz, 1H), 7.43 (d, J=2.1 Hz, 1H), 7.22-7.16 (m, 2H), 6.91 (d, J=2.4 Hz, 1H), 6.74 (d, J=8.3 Hz, 1H), 5.34 (br, 2H), 2.49 (s, 3H). ¹³C NMR (101 MHz; DMSO-d6): δ 197.0, 153.6, 145.2, 139.7, 134.8, 134.0, 133.1, 125.2, 119.0, 118.0, 117.3, 115.1, 112.0, 26.6. (EI, magnetic sector): calcd for C14H11BrClNO2 338.96617, found 338.96852.

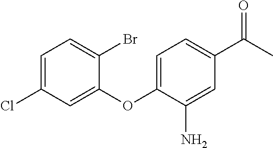 [SMILES: CC(=O)c1ccc(Oc2cc(Cl)ccc2Br)c(N)c1]

1-(3-amino-4-(2-bromo-4-chlorophenoxy)phenyl)ethanone (74a). This synthesis was carried out at a 20 mmol scale, using 2-bromo-4-chlorophenol as the nucleophile and 4-chloro-3-nitroacetophenone as the electrophile. The substitution step was run at 110° C. for approximately 15 min. The isolated yield of title product, crystallized out of ethyl acetate, was 49% appearing as light brown crystals. ¹H NMR (400 MHz; DMSO-d6): δ 7.88 (d, J=2.5 Hz, 1H), 7.46 (dd, J=8.8, 2.6 Hz, 1H) 7.41 (d, J=2.0 Hz, 1H), 7.15 (dd, J=8.3, 2.1 Hz, 1H), 6.96 (d, J=8.7 Hz, 1H), 6.65 (d, J=8.4 Hz, 1H), 5.33 (br, 2H), 2.48 (s, 3H). ¹³C NMR (101 MHz; DMSO-d6): δ 197.0, 151.8, 145.9, 139.5, 133.5, 132.8, 129.3, 128.4, 121.0, 117.3, 117.2, 114.9, 114.5, 26.5. (EI, magnetic sector): calcd for C14H11BrClNO2 338.96617, found 338.96713.

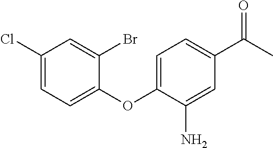 [SMILES: CC(=O)c1ccc(Oc2ccc(Cl)cc2Br)c(N)c1]

1-(3-amino-4-(2-bromo-4-fluorophenoxy)phenyl)ethanone (77a). This synthesis was carried out at a 20 mmol scale, using 2-bromo-4-fluorophenol as the nucleophile and 4-chloro-3-nitroacetophenone as the electrophile. The substitution step was run at 110° C. for approximately 15 min. The isolated yield of title product, crystallized out of ethyl acetate, was 18% appearing as light brown crystals. ¹H NMR (400 MHz; DMSO-d6): δ 7.75 (dd, J=8.1, 3.0 Hz, 1H), 7.39 (d, J=2.2 Hz, 1H), 7.30 (ddd, J=8.9, 8.2, 3.0 Hz, 1H), 7.14-7.08 (m, 2H), 6.52 (d, J=8.3 Hz, 1H), 5.32 (br, 2H), 2.47 (s, 3H). ¹³C NMR (101 MHz; DMSO-d6): δ 196.9, 158.2 (d, J=245.0 Hz), 148.9 (d, J=2.9 Hz), 146.9, 139.0, 133.0, 122.0 (d, J=8.8 Hz), 120.5 (d, J=26.0 Hz), 117.4, 116.2 (d, J=23.1 Hz) 115.7, 114.7 (d, J=10.3 Hz), 114.6, 26.5. ¹⁹F NMR (283 MHz; DMSO-d6): δ −119.5. (EI, magnetic sector): calcd for C14H11BrFNO2 322.99572, found 322.99630.

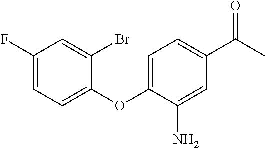 [SMILES: CC(=O)c1ccc(Oc2ccc(F)cc2Br)c(N)c1]

1-(3-amino-4-(2-bromo-5-(trifluoromethoxy)phenoxy)phenyl)ethanone (80a). This synthesis was carried out at a 20 mmol scale, using 2-bromo-5-trifluoromethoxyphenol as the nucleophile and 4-chloro-3-nitroacetophenone as the electrophile. The substitution step was run at 110° C. for approximately 15 min. The isolated yield of title product, isolated by chromatography (25% EtOAc in hexanes), was 60% appearing as off-white crystals. ¹H NMR (400 MHz; DMSO-d6): δ 7.88 (d, J=8.8 Hz, 1H), 7.43 (d, J=2.1 Hz, 1H), 7.19-7.15 (m, 2H), 6.86 (d, J=2.4 Hz, 1H), 6.74 (d, J=8.3 Hz, 1H), 5.37 (br, 2H), 2.49 (s, 3H). ¹³C NMR (101 MHz; DMSO-d6): δ 197.1, 153.8, 148.2 (q, J=1.5 Hz), 145.1, 139.8, 134.8, 134.0, 119.8 (q, J=257.5 Hz), 117.9, 117.5, 117.3, 115.9, 115.1, 112.1, 112.0, 26.5. ¹⁹F NMR (283 MHz; DMSO-d6): δ −57.4. (EI, magnetic sector): calcd for C15H11BrF3NO3 388.98744, found 388.98866.

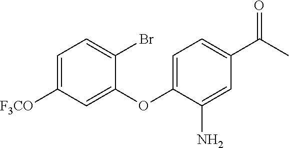 [SMILES: CC(=O)c1ccc(Oc2cc(OC(F)(F)F)ccc2Br)c(N)c1]

1-(3-amino-4-((1-bromonaphthalen-2-yl)oxy)phenyl)ethenone (83a). This synthesis was carried out at a 12 mmol scale, using 1-bromo-2-hydroxynaphthalene as the nucleophile and 4-chloro-3-nitroacetophenone as the electrophile. The substitution step was run at 110° C. for approximately 20 min. The isolated yield of title product, isolated by chromatography (25% EtOAc in hexanes), was 50% appearing as off-white crystals. ¹H NMR (400 MHz; DMSO-d6): δ 8.20 (d, J=8.5 Hz, 1H), 8.04 (s, 1H), 8.02 (s, 1H), 7.73 (ddd, J=7.0, 7.0, 1.2 Hz, 1H), 7.60 (ddd, J=7.0, 7.0, 1.2 Hz, 1H), 7.44 (d, J=2.2 Hz, 1H), 7.23 (d, J=8.9 Hz, 1H), 7.12 (dd, J=8.4, 2.3 Hz, 1H) 6.56 (d, J=8.4 Hz, 1H), 5.40 (br, 2H), 2.47 (s, 3H). ¹³C NMR (101 MHz; DMSO-d6): δ 197.0, 150.6, 146.9, 139.3, 133.0, 132.3, 131.2, 129.9, 128.6, 128.5, 126.0, 125.9, 120.1, 117.4, 116.3, 114.6, 112.0, 26.5.

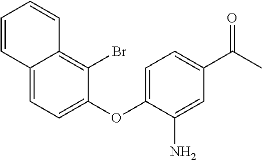 [SMILES: CC(=O)c1ccc(Oc2ccc3ccccc3c2Br)c(N)c1]

3-amino-4-(2-bromo-5-(trifluoromethyl)phenoxy)benzaldehyde (50a). This synthesis was carried out at a 20 mmol scale, using 2-bromo-5-trifluoromethylphenol as the nucleophile and 4-chloro-3-nitrobenzaldehyde as the electrophile. The substitution step was run at 110° C. for approximately 30 min. The isolated yield of title product, isolated by chromatography (25% EtOAc in hexanes), was 55% appearing as an off-white solid. ¹H NMR (400 MHz; DMSO-d6): δ 9.82 (s, 1H), 8.02 (d, J=8.4 Hz, 1H), 7.51 (dd, J=8.3, 1.5 Hz, 1H) 7.33 (d, J=2.1 Hz, 1H), 7.23 (d, J=1.9 Hz, 1H), 7.11 (dd, J=8.1, 2.0 Hz, 1H), 6.80 (d, J=8.1 Hz, 1H), 5.53 (br, 2H). ¹³C NMR (101 MHz; DMSO-d6): δ 192.2, 153.1, 146.4, 140.3, 135.1, 133.4, 129.6 (q, J=32.7 Hz), 123.3 (q, J=272.5 Hz), 122.1 (q, J=4.0 Hz), 119.5, 119.3, 118.7, 117.9, 116.1 (q, J=3.7 Hz), 114.7. ¹⁹F NMR (283 MHz; DMSO-d6): δ −61.6. (EI, magnetic sector): calcd for C14H9BrF3NO2 358.97688, found 358.97592.

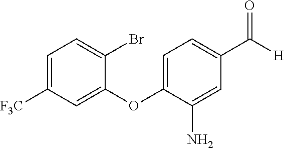 [SMILES: Nc1cc(C=O)ccc1Oc1cc(C(F)(F)F)ccc1Br]

2-(2-Bromo-5-(trifluoromethyl)phenoxy)-5-nitroaniline (54a). This synthesis was carried out at a 10 mmol scale, using 2-bromo-5-trifluoromethylphenol as the nucleophile and 1-chloro-2,4-dinitrobenzene as the electrophile. The substitution step was run at 60° C. for approximately 30 min. The isolated yield of title product was 44% appearing as a yellow crystalline solid; mp=89-91° C. ¹H NMR (400 MHz; DMSO-d6): δ 8.04 (d, J=8.3 Hz, 1H), 7.68 (d, J=2.7 Hz, 1H), 7.56 (dd, J=1.1, 8.3 Hz, 1H), 7.41 (d, J=1.6 Hz, 1H), 7.37 (dd, J=2.8, 8.8 Hz, 1H), 6.72 (d, J=8.8 Hz, 1H), 5.86 (br, 2H). ¹³C NMR (101 MHz; DMSO-d6): δ 152.6, 146.9, 144.2, 140.3, 135.2, 130.0 (q, J=32.7 Hz), 123.3 (q, J=272.5 Hz), 122.8 (q, J=3.7 Hz), 119.2, 117.3 (q, J=3.7 Hz), 116.8, 111.4, 109.2. ¹⁹F NMR (376 MHz; DMSO-d6): δ −61.6.

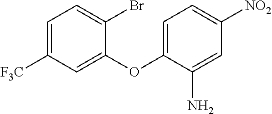 [SMILES: Nc1cc([N+](=O)[O-])ccc1Oc1cc(C(F)(F)F)ccc1Br]

3-Amino-4-(2-bromo-5-(trifluoromethyl)phenoxy)benzonitrile (56a). This synthesis was carried out at a 14 mmol scale, using 2-bromo-5-trifluoromethylphenol as the nucleophile and 4-chloro-3-nitrobenzonitrile as the electrophile. The substitution step was run at 100° C. for approximately 50 min. The isolated yield of title product was 66% appearing as a white solid; mp=145-147° C. ¹H NMR (400 MHz; DMSO-d6): δ 8.01 (d, J=8.3 Hz, 1H), 7.52 (dd, J=2.2, 8.3 Hz, 1H), 7.26 (d, J=2.0 Hz, 1H), 7.14 (d, J=2.1 Hz, 1H), 6.93 (dd, J=2.1, 8.2 Hz, 1H), 6.74 (d, J=8.2 Hz, 1H), 5.66 (br, 2H). ¹³C NMR (101 MHz; DMSO-d6): δ 152.8, 145.2, 140.6, 135.1, 129.9 (q, J=32.6 Hz), 123.3 (q, J=272.5 Hz), 122.3 (q, J=4.0 Hz), 120.2, 119.3, 118.8, 118.3, 117.9, 116.4 (q, J=4.0 Hz), 107.3. ¹⁹F NMR (376 MHz; DMSO-d6): δ −61.6. (EI, magnetic sector): calcd for C₁₄H₈BrF₃N₂O 335.97721, found 335.97707.

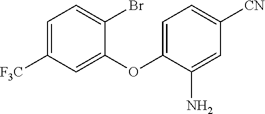 [SMILES: N#Cc1ccc(Oc2cc(C(F)(F)F)ccc2Br)c(N)c1]

N-(5-Bromo-2-(2-bromo-5-(trifluoromethyl)phenoxy)phenyl)acetamide (57a). This synthesis was carried out at a 14 mmol scale, using 2-bromo-5-trifluoromethylphenol as the nucleophile and 2-chloro-5-bromonitrobenzene as the electrophile. The substitution step was run at 100° C. for approximately 10 hr. The aniline product was not isolated, instead the crude residue was dissolved in neat acetic anhydride to make the corresponding acetamide. The title product was isolated by chromatography and was subsequently recrystallized from ethanol appearing as white crystals with a final yield of 76%; mp=123-125° C. ¹H NMR (400 MHz; DMSO-d6): δ 9.75 (s, 1H), 8.30 (d, J=2.1 Hz, 1H), 7.68 (d, J=8.2 Hz, 1H), 7.54 (dd, J=1.6, 8.3 Hz, 1H), 7.37 (d, 1.7 Hz, 1H), 7.25 (dd, 2.5, 8.7 Hz, 1H), 6.74 (d, 8.7 Hz, 1H), 2.09 (s, 3H).). ¹³C NMR (101 MHz; DMSO-d6): δ 169.2, 153.2, 145.8, 135.1, 131.0, 129.9 (q, J=32.7 Hz), 127.2, 125.6, 123.3 (q, J=272.5 Hz), 122.5 (q, J=3.7 Hz), 119.2(5), 119.1(5), 117.5 (q, J=3.3 Hz), 115.6, 23.7. ¹⁹F NMR (376 MHz; DMSO-d6): δ −61.2. (EI, magnetic sector): calcd for C₁₅H₁₀Br₂F₃NO₂ 450.90304, found 450.90456.

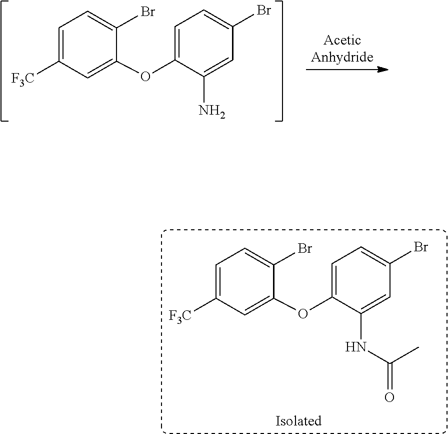

Preparation of Phenoxazines by Buchwald-Hartwig Amination. A general procedure for the preparation of phenoxazines by intramolecular coupling of 2-(2-bromophenoxy)anilines is given. In a Schlenk tube backfilled with argon were added tert-amyl alcohol or toluene (0.5M with respect to the aniline), Pd₂(dba)₃ (0.005 eq.) and BrettPhos (0.01 eq.). Stirring under a continuous flow of argon, the mixture was sparged with argon for ~10 min. To the tube were then added K₂CO₃ (2.2 eq.) and the aniline (1 eq.). The sealed Schlenk tube was heated to 105° C. and the reaction was closely monitored by TLC (usually done within 3 hr). After cooling to room temperature, the reaction mixture was passed through a plug of silica with excessive washing with hot acetone (until all phenoxazine product was washed through). In many instances, reducing the volume of acetone to <20 ml prompted crystallization of the product in a pure form which could be subsequently filtered, then rinsed with diethyl ether followed by hexanes. In cases where this did not happen, the product was simply purified by silica gel chromatography.

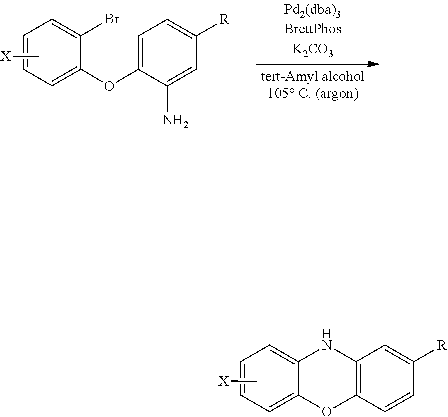

2-Acetyl-7-trifluoromethylphenoxazine (51). In a Schlenk tube backfilled with argon were added tert-amyl alcohol (28 mL), Pd₂(dba)₃ (33 mg, 36 mol) and BrettPhos (40 mg, 74 mol). Stirring under a continuous flow of argon, the mixture was sparged with argon for ~10 min. To the tube were then added K₂CO₃ (4.26 g, 30.8 mmol) and 1-(3-amino-4-(2-bromo-5-(trifluoromethyl)phenoxy)phenyl)ethenone (51a) (5.23 g, 14 mmol). The sealed Schlenk tube was heated to 105° C. for 3 hr, the product precipitating as yellow needles. After cooling, the mixture was poured over a silica plug and the product was washed through using hot acetone. The filtrate was reduced to ~15 mL under reduced pressure and ~100 mL diethyl ether was added and the flask was allowed to stand for −1 hr covered. The product was filtered and rinsed with diethyl ether then hexanes. The product, 2-acetyl-7-trifluoromethylphenoxazine (51), appeared as bright yellow needles (3.27 g, 79.7%); mp=275° C. (decomp.)¹H NMR (400 MHz; DMSO-d6): 8.94 (br, 1H), 7.30 (dd, J=8.2, 2.1 Hz, 1H), 7.10 (dd, J=8.2, 1.2 Hz, 1H), 6.98 (d, J=2.1 Hz, 1H), 6.90 (d, J=1.8 Hz, 1H), 6.71 (d, J=8.2 Hz, 1H), 6.54 (d, J=8.1 Hz, 1H), 2.44 (s, 3H). ¹³C NMR (101 MHz; DMSO-d6): δ 196.0, 146.6, 142.2, 135.6, 133.3, 131.3, 124.0 (q, J=270.7 Hz), 123.3, 122.2 (q, J=4.4 Hz), 120.5 (q, J=33.0 Hz), 115.0, 113.2, 112.4, 112.0 (q, J=3.7 Hz), 26.3. ¹⁹F NMR (376 MHz; DMSO-d6): δ −60.8. HRMS (EI, magnetic sector): calcd for C₁₅H₁₀F₃NO₂ 293.06636, found 293.06348.

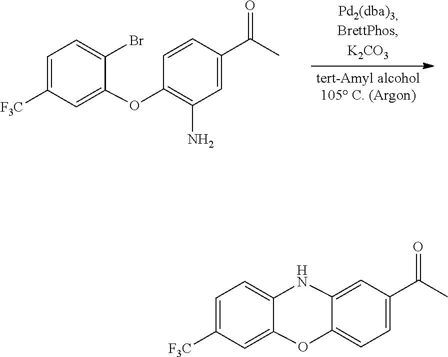 [SMILES: CC(=O)c1ccc(Oc2cc(C(F)(F)F)ccc2Br)c(N)c1 ; CC(=O)c1ccc2c(c1)Nc1ccc(C(F)(F)F)cc1O2]

2-Acetyl-8-trifluoromethylphenoxazine (68). This synthesis was carried out at a 7.6 mmol scale in tert-amyl alcohol using 1-(3-amino-4-(2-bromo-4-(trifluoromethyl)phenoxy)phenyl)101thenone (68a) for the amination. The reaction ran for 3 hrs total, but precipitated product was observed in the first 30 min. In similar fashion to (51), the product was isolated as yellow needles with a 75.6% yield. ¹H NMR (400 MHz; DMSO-d6): δ 8.73 (br, 1H), 7.27 (dd, J=8.2, 2.0 Hz, 1H), 6.94 (d, J=2.1 Hz, 1H), 6.90 (dd, J=8.2, 1.3 Hz, 1H), 6.77 (d, J=8.2 Hz, 1H), 6.72 (d, J=8.2 Hz, 1H), 6.64 (d, J=1.9 Hz, 1H), 2.43 (s, 3H). ¹³C NMR (101 MHz; DMSO-d6): δ 196.0, 146.3, 145.1, 133.5, 132.6, 131.5, 125.2 (q, J=31.9 Hz), 123.9 (q, J=271.8 Hz), 123.0, 117.7 (q, J=4.4 Hz), 115.7, 115.1, 112.2, 109.4 (q, J=3.7 Hz), 26.3. ¹⁹F NMR (283 MHz; DMSO-d6): δ −61.7. (EI, magnetic sector): calcd for C₁₅H₁₀F₃NO₂ 293.06636, found 293.06376.

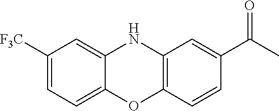 [SMILES: CC(=O)c1ccc2c(c1)Nc1cc(C(F)(F)F)ccc1O2]

2-Acetyl-7-chlorophenoxazine (71). This synthesis was carried out at a 9.7 mmol scale in tert-amyl alcohol using 1-(3-amino-4-(2-bromo-5-chlorophenoxy)phenyl)ethanone (71a) for the amination. The reaction ran for 2 hrs total, but precipitated product was observed in the first 30 min. In similar fashion to (51), though the reduced volume of acetone was filtered directly and not diluted into diethyl ether, the product was isolated as yellow needles with a 35% yield. ¹H NMR (400 MHz; DMSO-d6): δ 8.59 (br, 1H), 7.25 (dd, J=8.3, 2.1 Hz, 1H), 6.95 (d, J=2.1 Hz, 1H), 6.80 (dd, J=8.3, 2.3 Hz, 1H), 6.73 (d, J=2.3 Hz, 1H), 6.70 (d, J=8.2 Hz, 1H), 6.43 (d, J=8.3 Hz, 1H), 2.43 (s, 3H). ¹³C NMR (101 MHz; DMSO-d6): δ 196.1, 146.3, 142.8, 133.3, 132.0, 130.8, 124.2, 123.4, 122.6, 115.4, 115.0, 114.2, 112.1, 26.3. (EI, magnetic sector): calcd for C14H10ClNO2 259.04001, found 259.03971.

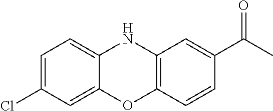 [SMILES: CC(=O)c1ccc2c(c1)Nc1ccc(Cl)cc1O2]

2-Acetyl-8-chlorophenoxazine (74). This synthesis was carried out at a 9.5 mmol scale in tert-amyl alcohol using 1-(3-amino-4-(2-bromo-4-chlorophenoxy)phenyl)ethanone (74a) for the amination. The reaction ran for 8 hrs total, but precipitated product was observed in the first 30 min. In similar fashion to (51), though the reduced volume of acetone was filtered directly and not diluted into diethyl ether, the product was isolated as yellow needles with a 30% yield. ¹H NMR (400 MHz; DMSO-d6): δ 8.64 (br, 1H), 7.27 (dd, J=8.3, 2.1 Hz, 1H), 6.95 (d, J=2.1 Hz, 1H), 6.70 (d, J=8.2 Hz, 1H), 6.64 (d, J=8.4, 1H), 6.59 (dd, J=8.4, 2.4 Hz, 1H) 6.43 (dd, J=2.4 Hz, 1H), 2.43 (s, 3H). ¹³C NMR (101 MHz; DMSO-d6): δ 196.0, 146.7, 141.1, 133.1, 131.5, 127.9, 122.9, 119.8, 116.5, 115.0, 112.7, 112.2, 26.3. (EI, magnetic sector): calcd for C14H10ClNO2 259.04001, found 259.04215.

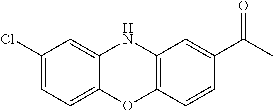 [SMILES: CC(=O)c1ccc2c(c1)Nc1cc(Cl)ccc1O2]

2-Acetyl-8-fluorophenoxazine (77). This synthesis was carried out at a 3.4 mmol scale in tert-amyl alcohol using 1-(3-amino-4-(2-bromo-4-fluorophenoxy)phenyl)ethanone (77a) for the amination. The reaction ran for 2 hrs total, but precipitated product was observed in the first 30 min. In similar fashion to (51), though the reduced volume of acetone was filtered directly and not diluted into diethyl ether, the product was isolated as yellow needles with a 68% yield. ¹H NMR (400 MHz; DMSO-d6): δ 8.65 (br, 1H), 7.27 (dd, J=8.3, 2.1 Hz, 1H), 6.96 (d, J=2.1 Hz, 1H), 6.69 (d, J=8.2 Hz, 1H), 6.64 (dd, J=8.7, 5.3 Hz, 1H), 6.37 (ddd, J=8.6, 8.6, 2.9 Hz, 1H), 6.25 (dd, J=9.6, 2.9 Hz, 1H), 2.43 (s, 3H). ¹³C NMR (101 MHz; DMSO-d6): δ 196.0, 160.1 (d, J=238.1 Hz), 146.9, 138.4 (d, J=2.6 Hz), 133.0(1) (d, J=11.7 Hz), 132.9(9), 131.3, 123.0, 116.0 (d, J=9.9 Hz), 114.9, 112.2, 105.7 (d, J=23.5 Hz), 100.5 (d, J=27.9 Hz), 26.3. ¹⁹F NMR (283 MHz; DMSO-d6): δ −121.5. (EI, magnetic sector): calcd for C14H10FNO2 243.06956, found 243.07152.

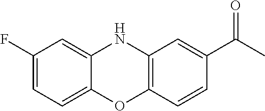 [SMILES: CC(=O)c1ccc2c(c1)Nc1cc(F)ccc1O2]

2-Acetyl-7-trifluoromethoxyphenoxazine (80). This synthesis was carried out at a 11.7 mmol scale in tert-amyl alcohol using 1-(3-amino-4-(2-bromo-5-(trifluoromethoxy)phenoxy)phenyl)ethanone (80a) for the amination. The reaction ran for 3 hrs total, but precipitated product was observed in the first 30 min. In similar fashion to (51), the product was isolated as yellow needles with an 86.5% yield. ¹H NMR (400 MHz; DMSO-d6): δ 8.64 (br, 1H), 7.26 (dd, J=8.2, 1.8 Hz, 1H), 6.95 (d, J=1.8 Hz, 1H), 6.76 (br double, J=8.5 Hz, 1H), 6.71-6.69 (m, 2H), 6.48 (d, J=8.5 Hz, 1H), 2.43 (s, 3H). ¹³C NMR (101 MHz; DMSO-d6): δ 196.1, 146.1, 142.4, 141.3 (q, J=1.8 Hz), 133.4, 131.9, 131.2, 122.7, 120.1 (q, J=255.7 Hz), 117.2, 115.0, 113.4, 112.2, 109.4, 26.3. ¹⁹F NMR (283 MHz; DMSO-d6): δ −57.8. (EI, magnetic sector): calcd for C15H10F3NO3 309.06128, found 309.06160.

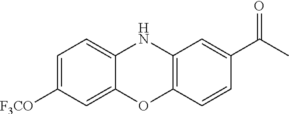 [SMILES: CC(=O)c1ccc2c(c1)Nc1ccc(OC(F)(F)F)cc1O2]

1-(12H-benzo[a]phenoxazin-10-yl)ethanone (83). This synthesis was carried out at a 4 mmol scale in tert-amyl alcohol using 1-(3-amino-4-((1-bromonaphthalen-2-yl)oxy)phenyl)ethanone (83a) for the amination. The reaction ran for 3 hrs total, but precipitated product was observed in the first 30 min. The product was isolated as a brown solid with an 80.9% yield. ¹H NMR (400 MHz; DMSO-d6): δ 8.43 (br, 1H), 7.96 (d, J=8.5 Hz, 1H), 7.75 (d, J=8.0 Hz, 1H), 7.47 (dd, J=7.3, 7.3 Hz, 1H), 7.37 (dd, J=7.7, 7.3 Hz, 1H), 7.31-7.26 (m, 3H), 6.95 (d, J=8.7 Hz, 1H), 6.72 (d, J=8.7 Hz, 1H), 2.46 (s, 3H). ¹³C NMR (101 MHz; DMSO-d6): δ 196.1, 147.7, 137.7, 133.2, 132.9, 130.8, 128.3, 125.7, 124.7(9), 124.7(6), 123.3, 121.4, 120.5, 120.4, 116.4, 114.8, 113.1, 26.3. (EI, magnetic sector): calcd for C18H13NO2 275.09463, found 275.09226.

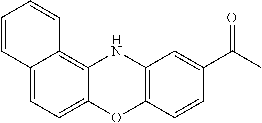 [SMILES: CC(=O)c1ccc2c(c1)Nc1c(ccc3ccccc13)O2]

2-Formyl-7-trifluoromethylphenoxazine (50). This synthesis was carried out at a 1 mmol scale in tert-amyl alcohol using 3-amino-4-(2-bromo-5-(trifluoromethyl)phenoxy)benzaldehyde (50a) for the amination. The reaction ran for 3 hrs total, but precipitated product was observed in the first 30 min. The product was isolated, by silica gel chromatography, as a yellow solid with a 40% yield. ¹H NMR (400 MHz; DMSO-d6): δ 9.71 (s, 1H), 9.03 (br, 1H), 7.23 (dd, J=8.1, 1.8 Hz, 1H), 7.10 (d, J=7.8 Hz, 1H), 6.92 (br, 1H), 6.88 (d, J=1.7 Hz, 1H), 6.80 (d, J=8.0 Hz, 1H), 6.56 (d, J=8.1 Hz, 1H). ¹³C NMR (101 MHz; DMSO-d6): δ 191.2, 147.7, 142.1, 135.3, 133.0, 132.0, 126.1, 124.0 (q, J=270.7 Hz), 122.3 (q, J=4.4 Hz), 120.7 (q, J=32.6 Hz), 115.6, 113.3, 112.1 (q, J=3.7 Hz), 111.7. ¹⁹F NMR (283 MHz; DMSO-d6): δ −60.8. (EI, magnetic sector): calcd for C14H8F3NO2 279.05071, found 279.05022.

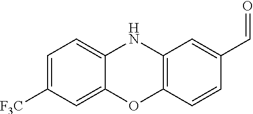 [SMILES: O=Cc1ccc2c(c1)Nc1ccc(C(F)(F)F)cc1O2]

2-Nitro-7-trifluoromethylphenoxazine (54). The reaction run for ~3 hr with 2-(2-bromo-5-(trifluoromethyl)phenoxy)-5-nitroaniline (54a) (3.55 g, 9.4 mmol) using tert-amyl alcohol as the solvent. Isolation of the title product (2.21 g, 7.5 mmol) afforded a dark red solid at a yield of 79%; mp=265° C. (decomp.). ¹H NMR (400 MHz; DMSO-d6): δ 9.16 (br, 1H) 7.47 (d, J=8.6 Hz, 1H), 7.17 (br, 1H), 7.09 (d, J=8.0 Hz, 1H), 6.91 (br, 1H), 6.75 (d, J=8.7 Hz, 1H), 6.53 (d, J=8.0 Hz, 1H). ¹³C NMR (101 MHz; DMSO-d6): δ 148.1, 143.8, 142.0, 134.6, 132.2, 123.9 (q, J=270.7 Hz), 122.6 (br), 121.2 (q, J=32.6 Hz), 117.7, 115.4, 113.5, 112.3 (br), 107.7. ¹⁹F NMR (376 MHz; DMSO-d6): δ −61.0. (EI, magnetic sector): calcd for C₁₃H₇F₃N₂O₃ 296.04088, found 296.03920.

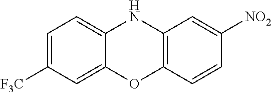 [SMILES: O=[N+]([O-])c1ccc2c(c1)Nc1ccc(C(F)(F)F)cc1O2]

2-Cyano-7-trifluoromethylphenoxazine (56). The reaction was only run for ~1 hr with 3-amino-4-(2-bromo-5-(trifluoromethyl)phenoxy)benzonitrile (56a) (1.834 g, 5.1 mmol) using toluene as the solvent. In a previous attempt it was observed that leaving the reaction for too long led to formation of a deep red by-product that would coelute when attempting chromatography. Isolation of the title product (0.64 g, 2.3 mmol) afforded white crystals. Accounting for the recovered starting material (1.54 mmol) we calculated a converted yield of 64%; mp=224-227° C. ¹H NMR (400 MHz; DMSO-d6): δ 9.05 (br, 1H), 7.11-7.07 (m, 2H), 6.90 (d, J=1.7 Hz, 1H), 6.74-6.72 (m, 2H), 6.57 (d, J=8.2 Hz, 1H). ¹³C NMR (101 MHz; DMSO-d6): δ 146.4, 142.2, 135.0, 132.4, 126.5, 123.9 (q, J=270.7 Hz), 122.3 (q, J=4.4 Hz), 121.0 (q, J=32.6 Hz), 118.5, 116.1, 115.8, 113.5, 112.1 (q, J=3.7 Hz), 106.7. ¹⁹F NMR (376 MHz; DMSO-d6): δ −61.6. (EI, magnetic sector): calcd for C₁₄H₇F₃N₂O 276.05105, found 276.05180.

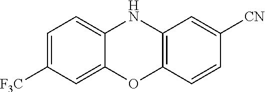 [SMILES: N#Cc1ccc2c(c1)Nc1ccc(C(F)(F)F)cc1O2]

2-Amino-7-trifluoromethylphenoxazine (55). First, a flame dried Schlenk tube with a stir bar was evacuated and backfilled with argon. Under a continuous flow of argon, the tube was charged with ethyl acetate which was sparged with argon for 10 minutes. 2-Nitro-7-trifluoromethylphenoxazine (54) (0.43 g, 1.44 mmol) and Pd/C (10 wt. %, 43 mg) were added and the tube sealed with a rubber septum. After evacuating the argon under vacuum, a H₂ filled balloon with a needle was placed through the septum and the reaction was stirred vigorously. On completion of the reduction, the Pd/C was filtered and the product was isolated/purified by column chromatography (60% ethyl acetate in hexanes). The fractions with the title product were concentrated to approximately 5-10 ml under reduced pressure, and addition of hexanes to this induced crystallization of the title product as white platelets (0.20 g, 52%); mp=204-208° C. (decomp.). ¹H NMR (400 MHz; DMSO-d6): δ 8.51 (br, 1H), 7.01 (dd, J=1.0, 8.3 Hz, 1H), 6.78 (d, J=1.4 Hz, 1H), 6.51 (d, J=8.1 Hz, 1H), 6.34 (d, J=8.1 Hz, 1H), 5.84-5.80 (m, 2H), 4.78 (br, 2H). ¹³C NMR (101 MHz; DMSO-d6): δ 145.4, 143.4, 136.3, 133.1, 130.8, 124.3 (q, J=270.3 Hz), 120.7 (q, J=4.0 Hz), 119.9 (q, J=32.3 Hz), 115.4, 112.7, 111.3 (q, J=4.0 Hz), 105.7, 99.9. (EI, magnetic sector): calcd for C₁₃H₉F₃N₂O 266.06670, found 266.06648.

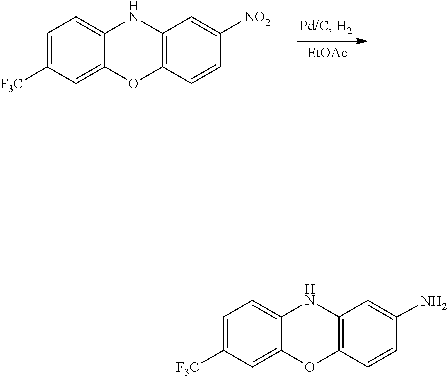 [SMILES: O=[N+]([O-])c1ccc2c(c1)Nc1ccc(C(F)(F)F)cc1O2 ; Nc1ccc2c(c1)Nc1ccc(C(F)(F)F)cc1O2]

10-Acetyl-2-bromo-7-trifluoromethylphenoxazine (57b). Into a 150 ml Teflon screw-cap sealable tube were combined copper(I) oxide (32 mg, 0.22 mmol), toluene (60 ml) and 1,2-dimethylethylenediamine (92 μl, 0.85 mmol) which were sparged with argon continuously while stirring rapidly on magnetic stir plate (approx. 10 min.). The N-(5-Bromo-2-(2-bromo-5-(trifluoromethyl)phenoxy)phenyl)acetamide (57a) starting material (3.85 g, 8.5 mmol) and 2 equivalents of K₂CO₃ (2.4 g, 17 mmol) were added after which the vessel was sealed. The vessel was heated on an oil bath to ~110° C. which was maintained for 16 hrs. After cooling, the reaction mixture was filtered and these vessel and filter paper were successively rinsed with ethyl acetate until quantitative transfer was achieved (visualized by TLC). After removing the solvent under reduced pressure, the title product was isolated by column chromatography (20% ethyl acetate in hexanes) which appeared as a white solid (2.60 g, 82.2%); mp=132-134° C. ¹H NMR (400 MHz; DMSO-d6): δ 7.87 (d, J=2.3 Hz, 1H), 7.81 (d, J=8.3 Hz, 1H), 7.61-7.57 (m 2H), 7.48 (dd, J=2.3, 8.6 Hz, 1H), 7.20 (d, J=8.7 Hz, 1H), 2.32 (s, 1H). ¹³C NMR (101 MHz; DMSO-d6): δ 168.9, 150.1, 149.1, 132.3, 130.0(0), 129.9(3), 127.8, 127.5 (q, J=32.6 Hz), 126.3, 123.5 (q, J=272.2 Hz), 120.9 (q, J=4.0 Hz), 118.5, 115.3, 113.9 (q, J=3.7 Hz), 22.8. ¹⁹F NMR (376 MHz; DMSO-d6): ¹⁹F NMR (376 MHz; DMSO-d6): δ −61.2. (EI, magnetic sector): calcd for C₁₅H₉BrF₃NO₂ 370.97688, found 370.97719.

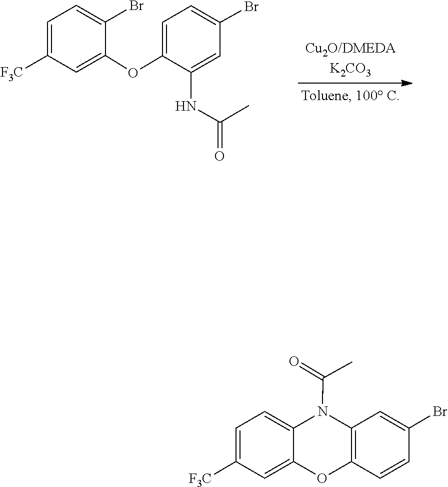 [SMILES: CC(=O)Nc1cc(Br)ccc1Oc1cc(C(F)(F)F)ccc1Br ; CC(=O)N1c2ccc(C(F)(F)F)cc2Oc2ccc(Br)cc21]

2-Bromo-7-trifluoromethylphenoxazine (57). First, a Schlenk tube was evacuated then backfilled with argon. The flask was then opened with a steady flow of argon passing through the side arm and out the top, throughout addition of reagents. The flask was charged with ethanol (13 ml), concentrated HCl (1 ml) and a magnetic stir bar, and an argon filled balloon affixed with a long needle was placed through the top to sparge the solvent while stirring (for at least 10 min.). 10-Acetyl-2-bromo-7-trifluoromethylphenoxazine (57b) was added (0.50 g, 1.34 mmol) and the suspension was stirred while heating to 70° C. (while maintaining a continuous flow of argon). Once up to temperature, flask was sealed with a septum, the gas side arm was closed, and a balloon of argon was placed in the septa and the reaction progress was closely monitored by TLC. When all of the starting material had been consumed, the reaction was cooled to room temperature after which reaction mixture was carefully quenched in a saturated NaHCO₃ solution. After extracting with ethyl acetate, the organic layer was subsequently rinsed, dried (Mg₂SO₄), filtered and the solvent was removed under reduced pressure. The title product appeared as a white solid (0.35 g, 79%); mp=132-134° C. ¹H NMR (400 MHz; DMSO-d6): δ 8.89 (br, 1H), 7.08 (dd, J=0.9, 8.1 Hz, 1H), 6.76 (dd, J=2.3, 8.4 Hz, 1H), 6.59-6.54 (m, 3H). ¹³C NMR (101 MHz; DMSO-d6): δ 142.6, 141.9, 135.3, 132.9, 124.0 (q, J=270.7 Hz), 123.5, 121.7 (q, J=4.0 Hz), 120.9 (q, J=32.7 Hz), 116.9, 115.8, 115.5, 113.3, 111.9 (q, J=4.0 Hz). ¹⁹F NMR (376 MHz; DMSO-d6): δ −60.9. (EI, magnetic sector): calcd for C₁₃H₇BrF₃NO 328.96631, found 328.96610.

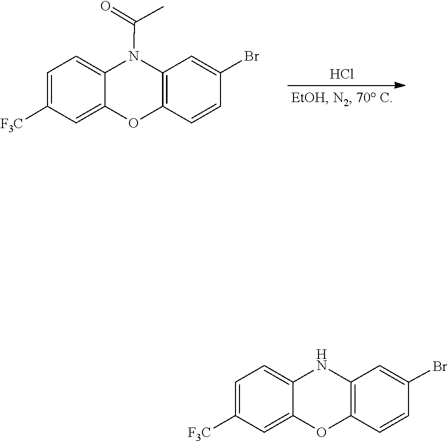 [SMILES: CC(=O)N1c2ccc(C(F)(F)F)cc2Oc2ccc(Br)cc21 ; Cl ; FC(F)(F)c1ccc2c(c1)Oc1ccc(Br)cc1N2]

2-Iodo-7-trifluoromethylphenoxazine (58). In a 10 mL sealed test tube with a stir bar were combined 10-acetyl-2-bromo-7-trifluoromethylphenoxazine (57b) (744 mg, 2 mmol), sodium iodide (600 mg, 4 mmol), copper(I) iodide (19.2 mg), 1,2-dimethylethylenediamine (32 L) and 1,4-dioxane (2 mL). After sparging with argon for 5 minutes, the vessel was sealed and the mixture was heated on a test tube block with rapid stirring. The reaction was maintained at 100° C. for ≥12 hrs, after which the reaction was cooled to room temperature (successful halogen exchange was confirmed by TLC-MS/APCI). The mixture was filtered and rinsed through a plug of silica using ethyl acetate. After the solvent was removed under reduced pressure, the intermediate product (10-acetyl-2-iodo-7-trifluoromethylphenoxazine) was subjected to amide hydrolysis (analogous to the procedure for 57). After work-up and column, the title product was obtained in a 69% yield (1.04 g) appearing as a white solid; mp=149-151° C. ¹H NMR (400 MHz; DMSO-d6): δ 8.79 (br, 1H), 7.03 (d, J=8.1 Hz, 1H), 6.89 (dd, J=2.0, 8.2 Hz, 1H), 6.83 (br, 1H), 6.70 (d, J=2.0 Hz, 1H), 6.50 (d, J=8.2 Hz, 1H), 6.39 (d, J=8.2 Hz, 1H). ¹³C NMR (101 MHz; DMSO-d6): δ 142.5(9), 142.5(5), 135.4, 133.0, 129.7, 124.0 (q, J=270.7 Hz), 121.7 (q, J=4.0 Hz), 121.5, 120.8 (q, J=32.3 Hz), 117.3, 113.3, 111.9 (q, J=3.7 Hz), 87.2. 6-60.9. (EI, magnetic sector): calcd for C₁₃H₇IF₃NO 376.95244, found 376.95245.

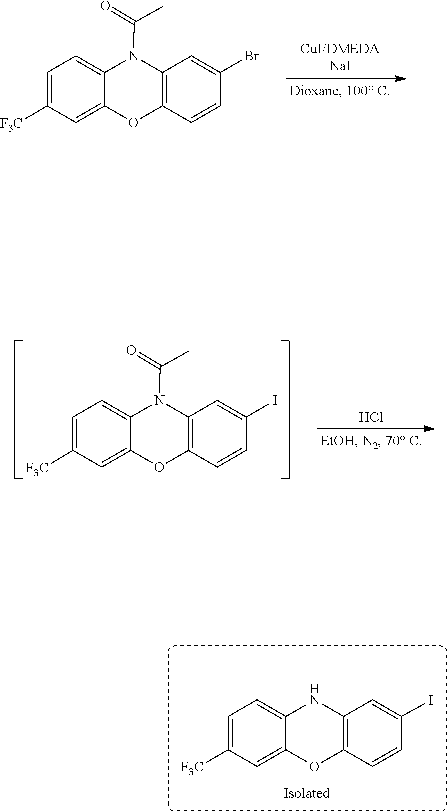

tert-Butyl-2-bromo-7-trifluoromethylphenoxazine-10-carboxylate (59a). In a flask affixed with a reflux condenser were combined 2-bromo-7-trifluoromethylphenoxazine (57) (1.84 g, 5.6 mmol), acetonitrile (11 ml, 0.5M), DMAP (68 mg, 0.56 mmol) and di-tert-butyl dicarbonate (1.48 g, 6.8 mmol). With stirring, the mixture was refluxed for 20 hrs, after which the flask was allowed to cool to room temperature. The product appeared as a colourless precipitate and the solvent was decanted. Subsequently the precipitate was recrystallized from ethanol, affording the title product as colourless needles (1.34 g, 70%). ¹H NMR (400 MHz; DMSO-d6): δ 7.74 (d, J=8.2 Hz, 1H), 7.74 (d, J=2.3 Hz, 1H), 7.53-7.50 (m, 2H), 7.40 (dd, J=8.6, 2.2 Hz, 1H), 7.10 (d, J=8.6 Hz, 1H), 1.50 (s, 9H). ¹³C NMR (101 MHz; DMSO-d6): δ 150.1, 149.3, 148.3, 131.5, 129.3, 129.1, 127.6, 126.8 (q, J=33.0 Hz), 126.1, 123.5 (q, J=272.2 Hz), 120.6 (q, J=3.7 Hz), 118.2, 115.0, 113.7 (q, J=3.7 Hz), 83.7, 27.5. ¹⁹F NMR (283 MHz; DMSO-d6): −61.3.

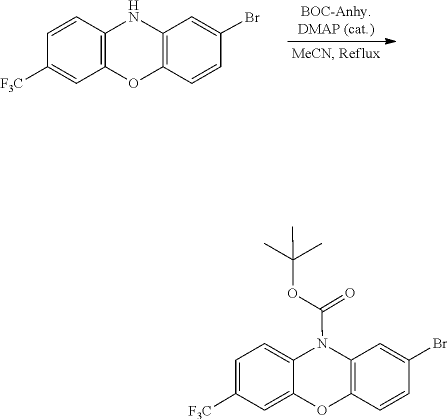 [SMILES: FC(F)(F)c1ccc2c(c1)Oc1ccc(Br)cc1N2 ; CC(C)(C)OC(=O)N1c2ccc(C(F)(F)F)cc2Oc2ccc(Br)cc21]

tert-Butyl-2-(2-oxopyrrolidin-1-yl)-7-(trifluoromethyl)-phenoxazine-10-carboxylate (59b). Into a screw-cap sealable tube were combined copper(I) iodide (14 mg, 0.075 mmol), toluene (6 ml) and 1,2-dimethylethylenediamine (16 L, 0.15 mmol) which were sparged with argon continuously while stirring rapidly on magnetic stir plate (approx. min.). 2-Pyrrolidone (153 mg, 1.8 mmol) and tert-butyl-2-bromo-7-trifluoromethylphenoxazine-10-carboxylate (59a) (0.64 g, 1.5 mmol) were then added along with K₂CO₃ (0.41 g, 3 mmol) and the vessel was sealed. The vessel was heated on an oil bath to ~110° C. which was maintained for 20 hr. After cooling, the reaction mixture was filtered and these vessel and filter paper were successively rinsed with ethyl acetate until quantitative transfer was achieved (visualized by TLC). After removing the solvent under reduced pressure, the title product was isolated by column chromatography (60% ethyl acetate in hexanes) which appeared as a white solid (0.52 g, 79%). ¹H NMR (400 MHz; DMSO-d6): δ 8.01 (d, J=2.5 Hz, 1H), 7.78 (d, J=9.0 Hz, 1H), 7.53-7.51 (m, 2H), 7.43 (dd, J=8.9, 2.6 Hz, 1H), 7.15 (d, J=8.9 Hz, 1H), 3.82 (t, J=6.9 Hz, 2H), 2.50 (t, J=8.0 Hz, 2H), 2.06 (pent, J=7.5 Hz, 2H), 1.50 (s, 9H). ¹³C NMR (101 MHz; DMSO-d6): δ 173.8, 150.5, 149.8, 145.0, 135.7, 132.0, 127.8, 126.7 (q, J=32.7 Hz), 126.1, 123.6 (q, J=272.2 Hz), 120.4 (q, J=4.0 Hz), 117.6, 116.2(9), 116.1(5), 113.6 (q, J=4.0 Hz), 83.3, 48.3, 32.2, 27.6, 17.3. ¹⁹F NMR (283 MHz; DMSO-d6): −61.2.

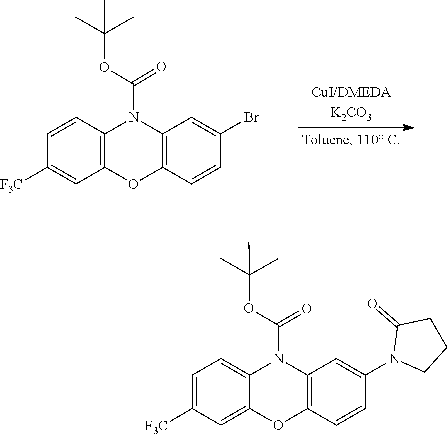 [SMILES: CC(C)(C)OC(=O)N1c2ccc(C(F)(F)F)cc2Oc2ccc(Br)cc21 ; CC(C)(C)OC(=O)N1c2ccc(C(F)(F)F)cc2Oc2ccc(N3CCCC3=O)cc21]

1-(7-(trifluoromethyl)-10H-phenoxazin-2-yl)pyrrolidin-2-one (59). In a scintillation vial, tert-Butyl-2-(2-oxopyrrolidin-1-yl)-7-(trifluoromethyl)-phenoxazine-10-carboxylate (59b) (0.39 g, 0.9 mmol) was dissolved in 9 ml of 4.0M anhydrous HCl in 1,4-dioxane. This was stirred for 4 hr, after which the reaction was quenched in a saturated NaHCO₃ solution. This was extracted with ethyl acetate, and the organic phase was washed with water 3 times and the organic was dried with MgSO₄, filtered, and the solvent removed under reduced pressure. The title product was isolated by silica gel chromatography (60% ethyl acetate in hexanes), appearing as a white solid (0.26 g, 85%). ¹H NMR (400 MHz; DMSO-d6): δ 8.86 (br, 1H), 7.19 (d, J=2.3 Hz, 1H), 7.06 (d, J=8.1, 1.1 Hz, 1H), 6.85 (d, J=1.9 Hz, 1H), 6.66 (dd, J=8.7, 2.5 Hz, 1H), 6.62 (d, J=8.6 Hz, 1H), 6.53 (d, J=7.8 Hz, 1H), 3.72 (t, J=7.1 Hz, 2H), 2.45 (t, J=8.2 Hz, 2H), 2.01 (pent, J=7.6 Hz, 2H). ¹³C NMR (101 MHz; DMSO-d6): δ 173.6, 142.8, 138.5, 136.1, 135.9, 130.8, 124.1 (q, J=270.7 Hz), 121.5 (q, J=4.0 Hz), 120.3 (q, J=32.7 Hz), 114.9, 113.0, 111.7 (q, J=3.7 Hz), 111.2, 105.2, 48.0, 32.3, 17.2. ¹⁹F NMR (283 MHz; DMSO-d6): −60.7. (EI, magnetic sector): calcd for C17H13F3N202 334.09291, found 334.08793.

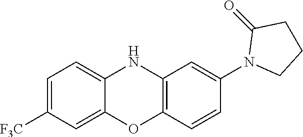 [SMILES: O=C1CCCN1c1ccc2c(c1)Nc1ccc(C(F)(F)F)cc1O2]

1-(7H-benzo[c]phenoxazin-9-yl)ethanone (86). In a round bottom flask equipped with a stir bar were combined 1-naphthol (1.44 g, 10 mmol) K₂CO₃ (2.76 g, 20 mmol) and DMSO (20 ml). This mixture was heated to 120° C., after which 4-chloro-3-nitroacetophenone was added (2.00 g, 10 mmol). The temperature was maintained for 20 min., after which the reaction vessel was cooled on an ice bath then poured into 150 ml 1M HCl. This was extracted with ethyl acetate twice, and the organic layer was dried with MgSO₃, and filtered. After removing the solvent under reduced pressure, the crude intermediate was transferred into a Schlenk tube with a stir bar, triphenylphosphine (5.2 g) and o-dichlorobenzene (10 ml), the solution was degassed and finally backfilled with argon (and the vessel sealed). This mixture was heated to 170° C. for 12 hr. after which the mixture was passed through a silica gel column (gradient of 100:0→90:10 toluene/ethyl acetate), and the title product was isolated as dark-yellow needles (335 mg, 12%). ¹H NMR (400 MHz; DMSO-d6): δ 8.48 (s, 3H), 7.77 (d, J=8.4 Hz, 1H), 7.70 (d, J=8.1 Hz, 1H), 7.44-7.40 (m, 2H), 7.31 (dd, J=8.2, 2.1 Hz, 1H), 7.27-7.23 (m, 1H), 6.99 (d, J=2.1 Hz, 1H), 6.84 (d, J=8.2 Hz, 1H), 6.81 (d, J=8.6 Hz, 1H), 2.45 (s, 3H). 13C NMR (101 MHz; DMSO-d6): δ 196.1, 147.7, 133.8, 133.3, 133.2, 129.0, 127.8, 127.4, 126.6, 124.0, 123.3, 123.1, 123.0, 118.6, 115.5, 115.1, 112.2, 26.3. (EI, magnetic sector): calcd for C18H13NO2 275.09463, found 275.09553.

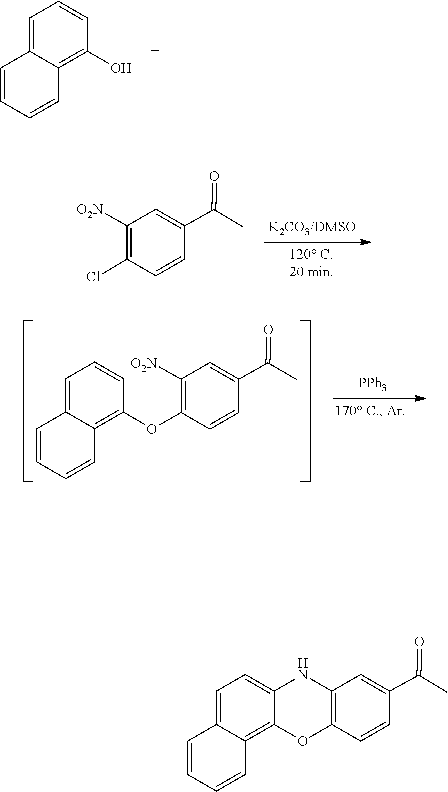

Preparation of 4-Methylbenzenesulfonohydrazide Phenoxazine Derivatives. A general method is provided. In a round bottom flask, affixed with a condenser, were added the 2-acetylphenoxazine derivative (1 eq.), 4-methylbenzenesulfonohydrazide (1.3 eq.) and either MeOH or EtOH (0.1M). The suspension was maintained at a high reflux with stirring for ~4 h (or more as necessary), and after cooling the precipitated product was filtered and subsequently rinsed with ethanol, diethyl ether and hexanes.

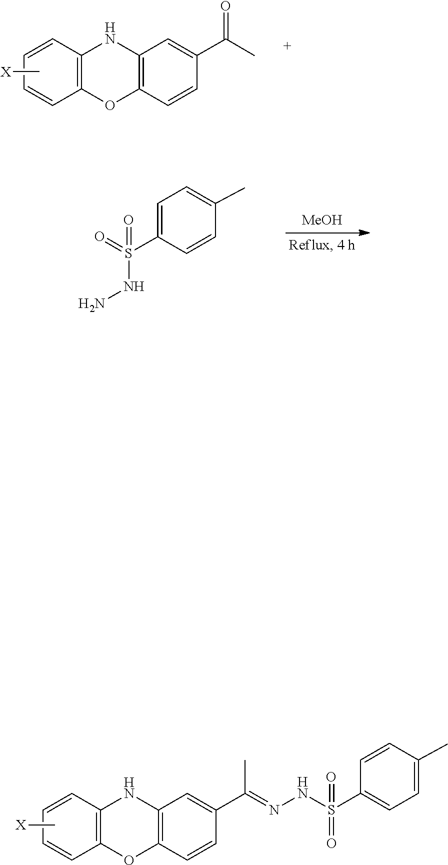

(E)-N′-(1-(10H-phenoxazin-2-yl)ethylidene)-4-methylbenzenesulfonohydrazide (52). This synthesis was carried out at a 4.4 mmol scale in MeOH using 2-acetylphenoxazine as the electrophile. The suspension was refluxed with stirring for 4 h affording the title product in an 89% yield, which appeared as a light brown solid. ¹H NMR (400 MHz; DMSO-d6): δ 10.39 (s, 1H), 8.36 (br, 1H), 7.81 (d, J=8.2 Hz, 2H), 7.41 (d, J=8.1 Hz, 2H), 6.85-6.72 (m, 3H), 6.61-6.54 (m, 3H), 6.45 (dd, J=7.6, 1.0 Hz, 1H), 2.37 (s, 3H), 2.05 (s, 3H). 13C NMR (101 MHz; DMSO-d6): δ 152.2, 144.1, 143.3, 142.4, 136.3, 133.1, 132.2, 131.9, 129.5, 127.5, 124.2, 120.4, 119.0, 115.2, 114.6, 113.3, 110.0, 21.0, 13.9. (EI, magnetic sector): calcd for C21H19N3O3S 393.11471, found 393.11389.

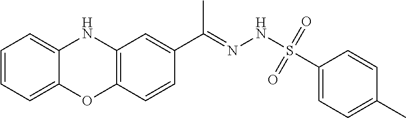 [SMILES: C/C(=N\NS(=O)(=O)c1ccc(C)cc1)c1ccc2c(c1)Nc1ccccc1O2]

(E)-4-methyl-N′-(1-(7-(trifluoromethyl)-10H-phenoxazin-2-yl)ethylidene)benzenesulfonohydrazide (53). This synthesis was carried out at a 4 mmol scale in MeOH using 2-acetyl-7-trifluoromethylphenoxazine (51) as the electrophile. The suspension was refluxed with stirring for 4 h affording the title product in an 82% yield, which appeared as an off-white solid. ¹H NMR (400 MHz; DMSO-d6): δ 10.43 (br, 1H), 8.89 (br, 1H), 7.80 (d, J=8.2 Hz, 2H), 7.41 (d, J=8.0 Hz, 2H), 7.08 (d, J=8.2 Hz, 1H), 6.89-6.87 (m, 3H), 6.62-6.60 (m, 1H), 6.55 (d, J=8.2 Hz, 1H), 2.38 (s, 3H), 2.05 (s, 3H). ¹³C NMR (101 MHz; DMSO-d6): δ 151.9, 143.7, 143.3, 142.5, 136.3, 135.8, 133.5, 130.8, 129.5, 127.5, 124.1 (q, J=270.3 Hz), 121.8 (q, J=4.4 Hz), 120.3 (q, J=32.6 Hz), 119.9, 114.8, 113.0, 111.8 (q, J=3.7 Hz), 110.4, 21.0, 13.8. ¹⁹F NMR (376 MHz; DMSO-d6): δ −60.7. HRMS (EI, magnetic sector): calcd for C₂₂H₁₈F₃N₃O₃S 461.10210, found 461.10473.

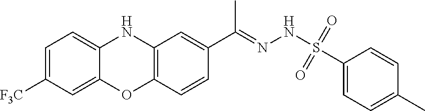 [SMILES: C/C(=N\NS(=O)(=O)c1ccc(C)cc1)c1ccc2c(c1)Nc1ccc(C(F)(F)F)cc1O2]

(E)-4-methyl-N′-(1-(8-(trifluoromethyl)-10H-phenoxazin-2-yl)ethylidene)benzenesulfonohydrazide (67). This synthesis was carried out at a 4.9 mmol scale in MeOH using 2-acetyl-8-trifluoromethylphenoxazine (68) as the electrophile. The suspension was refluxed with stirring for 4 h affording the title product in an 85% yield, which appeared as white needles. ¹H NMR (400 MHz; DMSO-d6): δ 10.45 (br, 1H), 8.69 (br, 1H), 7.80 (d, J=8.2 Hz, 2H), 7.41 (d, J=8.1 Hz, 2H), 6.90-6.84 (m, 3H), 6.75 (d, J=8.2 Hz, 1H), 6.65 (d, J=2.0 Hz, 1H), 6.62 (d, J=8.1 Hz, 1H), 2.37 (s, 3H), 2.06 (s, 3H). ¹³C NMR (101 MHz; DMSO-d6): δ 152.0, 145.4, 143.5, 143.3, 136.3, 133.7, 132.9, 131.1, 129.5, 127.5, 124.7 (q, J=31.9 Hz), 123.9 (q, J=271.4 Hz), 119.6, 117.5 (q, J=4.4 Hz), 115.6, 114.9, 110.3, 109.3 (q, J=3.7 Hz), 21.0, 13.9. ¹⁹F NMR (283 MHz; DMSO-d6): −61.6. (EI, magnetic sector): calcd for C22H18F3N3O3S 461.10210, found 461.09888.

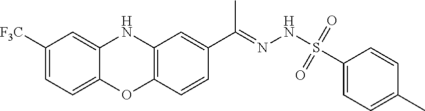 [SMILES: C/C(=N\NS(=O)(=O)c1ccc(C)cc1)c1ccc2c(c1)Nc1cc(C(F)(F)F)ccc1O2]

(E)-4-methyl-N′-(1-(7-chloro-10H-phenoxazin-2-yl)ethylidene)benzenesulfonohydrazide (70). This synthesis was carried out at a 3 mmol scale in MeOH using 2-acetyl-7-chlorophenoxazine (71) as the electrophile. The suspension was refluxed with stirring for 4 h affording the title product in an 74% yield, which appeared as an off-white solid. ¹H NMR (400 MHz; DMSO-d6): δ 10.42 (br, 1H), 8.53 (br, 1H), 7.79 (d, J=8.2 Hz, 2H), 7.41 (d, J=8.0 Hz, 2H), 6.84-6.82 (m, 2H), 6.79 (dd, J=8.3, 2.3 Hz, 1H), 6.70 (d, J=2.4 Hz, 1H), 6.59 (d, J=8.7 Hz, 1H), 6.43 (d, J=8.3 Hz, 1H), 2.37 (s, 3H), 2.05 (s, 3H). ¹³C NMR (101 MHz; DMSO-d6): δ 152.0, 143.5, 143.3, 143.1, 136.3, 133.5, 131.6, 131.1, 129.5, 127.5, 123.8, 123.1, 119.2, 115.2, 114.7, 114.1, 110.2, 21.0, 13.9. (EI, magnetic sector): calcd for C21H18ClN3O3S 427.07574, found 427.07506.

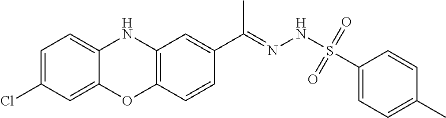 [SMILES: C/C(=N\NS(=O)(=O)c1ccc(C)cc1)c1ccc2c(c1)Nc1ccc(Cl)cc1O2]

(E)-4-methyl-N′-(1-(8-chloro-10H-phenoxazin-2-yl)ethylidene)benzenesulfonohydrazide (73). This synthesis was carried out at a 3 mmol scale in MeOH using 2-acetyl-8-chlorophenoxazine (74) as the electrophile. The suspension was refluxed with stirring for 4 h affording the title product in an 74% yield, which appeared as a white solid. ¹H NMR (400 MHz; DMSO-d6): δ 10.42 (br, 1H), 8.58 (br, 1H), 7.79 (d, J=8.2 Hz, 2H), 7.41 (d, J=8.0 Hz, 2H), 6.87-6.84 (m, 2H), 6.62-6.56 (m, 3H), 6.43 (d, J=2.4 Hz, 1H), 2.37 (s, 3H), 2.05 (s, 3H). ¹³C NMR (101 MHz; DMSO-d6): δ 152.0, 143.8, 143.3, 141.4, 136.3, 133.4, 133.3, 131.1, 129.5, 127.5(4), 127.4(5), 119.5, 116.4, 114.7, 112.6, 110.2, 21.0, 13.9. (EI, magnetic sector): calcd for C21H18ClN3O3S 427.07574, found 427.07298.

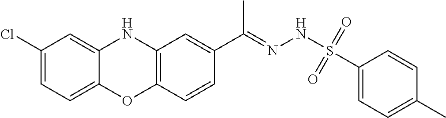 [SMILES: C/C(=N\NS(=O)(=O)c1ccc(C)cc1)c1ccc2c(c1)Nc1cc(Cl)ccc1O2]

(E)-4-methyl-N′-(1-(8-fluoro-10H-phenoxazin-2-yl)ethylidene)benzenesulfonohydrazide (76). This synthesis was carried out at a 2 mmol scale in MeOH using 2-acetyl-8-fluorophenoxazine (77) as the electrophile. The suspension was refluxed with stirring for 4 h affording the title product in an 77% yield, which appeared as white needles. ¹H NMR (400 MHz; DMSO-d6): δ 10.42 (br, 1H), 8.60 (br, 1H), 7.80 (d, J=8.0 Hz, 2H), 7.41 (d, J=8.0 Hz, 2H), 6.86-6.85 (m, 2H), 6.63-6.57 (m, 2H), 6.35 (ddd, J=8.6, 8.5, 2.7 Hz, 1H), 6.25 (dd, J=9.5, 2.6 Hz, 1H), 2.37 (s, 3H), 2.05 (s, 3H). ¹³C NMR (101 MHz; DMSO-d6): δ 158.7 (d, J=237.3 Hz), 152.1, 144.0, 143.3, 138.6 (d, J=2.2 Hz), 136.3, 133.3 (d, J=11.7 Hz), 133.1, 131.0, 129.5, 127.5, 119.6, 115.8 (d, J=10.3 Hz), 114.6, 110.2, 105.4 (d, J=23.1 Hz), 100.3 (d, J=28.2 Hz), 21.0, 13.9. ¹⁹F NMR (283 MHz; DMSO-d6): −121.9. (EI, magnetic sector): calcd for C21H18FN3O3S 411.10529, found 411.10282.

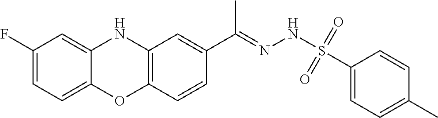 [SMILES: C/C(=N\NS(=O)(=O)c1ccc(C)cc1)c1ccc2c(c1)Nc1cc(F)ccc1O2]

(E)-4-methyl-N′-(1-(7-(trifluoromethoxy)-10H-phenoxazin-2-yl)ethylidene)benzenesulfonohydrazide (79). This synthesis was carried out at a 9.4 mmol scale in MeOH using 2-acetyl-7-trifluoromethoxyphenoxazine (80) as the electrophile. The suspension was refluxed with stirring for 4 h affording the title product in an 83% yield, which appeared as off-white needles. ¹H NMR (400 MHz; DMSO-d6): δ 10.43 (br, 1H), 8.59 (br, 1H), 7.80 (d, J=8.1 Hz, 2H), 7.41 (d, J=8.1 Hz, 2H), 6.86-6.84 (m, 2H), 6.75 (dd, J=8.5, 1.5 Hz, 1H), 6.68 (d, J=1.4 Hz, 1H), 6.60 (d, J=8.8 Hz, 1H), 6.48 (d, J=8.6 Hz, 1H), 2.37 (s, 3H), 2.05 (s, 3H). ¹³C NMR (101 MHz; DMSO-d6): δ 152.0, 143.3, 143.2, 142.7, 141.2 (q, J=1.8 Hz), 136.3, 133.6, 131.5, 129.5, 127.5, 120.2 (q, J=255.3 Hz), 119.4, 116.8, 114.7, 113.2, 110.2, 109.3, 21.0, 13.9. ¹⁹F NMR (283 MHz; DMSO-d6): −57.8. (EI, magnetic sector): calcd for C22H18F3N3O4S 477.09701, found 477.09698.

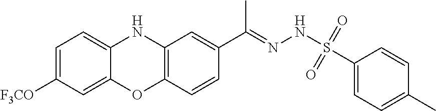 [SMILES: C/C(=N\NS(=O)(=O)c1ccc(C)cc1)c1ccc2c(c1)Nc1ccc(OC(F)(F)F)cc1O2]

(E)-N′-(1-(12H-benzo[a]phenoxazin-10-yl)ethylidene)-4-methylbenzenesulfonohydrazide (82). This synthesis was carried out at a 2.5 mmol scale in MeOH using 1-(12H-benzo[a]phenoxazin-10-yl)ethanone (83) as the electrophile. The suspension was refluxed with stirring for 8 h affording the title product in an 58% yield, which appeared as yellow needles. ¹H NMR (400 MHz; DMSO-d6): δ 10.40 (br, 1H), 8.38 (br, 1H), 8.01 (d, J=8.4 Hz, 1H), 7.85 (d, J=8.2 Hz, 2H), 7.74 (d, J=8.0 Hz, 1H), 7.47 (ddd, J=8.1, 8.1, 1.1 Hz, 1H), 7.42 (d, J=8.1 Hz, 2H), 7.36 (dd, J=7.9, 7.7 Hz, 1H), 7.25 (d, J=8.7 Hz, 1H), 7.20 (d, J=2.2 Hz, 1H), 6.93 (d, J=8.7 Hz, 1H), 6.88 (dd, J=8.3, 2.2 Hz, 1H), 6.61 (d, J=8.3 Hz, 1H), 2.37 (s, 3H), 2.08 (s, 3H). ¹³C NMR (101 MHz; DMSO-d6): δ 152.2, 144.6, 134.3, 137.9, 136.4, 133.3, 132.5, 130.7, 129.5, 128.2, 127.5, 125.5, 125.0, 124.6, 121.4, 120.7, 120.2, 119.8, 116.4, 114.5, 111.2, 21.0, 13.9. (EI, magnetic sector): calcd for C25H21N3O3S 443.13036, found 443.13229.

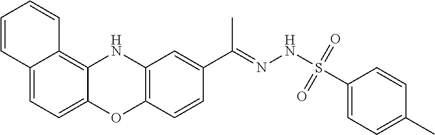 [SMILES: C/C(=N\NS(=O)(=O)c1ccc(C)cc1)c1ccc2c(c1)Nc1c(ccc3ccccc13)O2]

(E)-N′-(1-(7H-benzo[c]phenoxazin-9-yl)ethylidene)-4-methylbenzenesulfonohydrazide (83). This synthesis was carried out at a 1 mmol scale in EtOH using 1-(7H-benzo[c]phenoxazin-9-yl)ethanone (84) as the electrophile. The suspension was refluxed with stirring for 8 h affording the title product in an 72% yield, which appeared as yellow needles. ¹H NMR (400 MHz; DMSO-d6): δ 10.40 (br, 1H), 8.42 (br, 1H), 7.82 (d, J=8.2 Hz, 2H), 7.76 (d, J=8.4 Hz, 1H), 7.69 (d, J=8.2 Hz, 1H), 7.43-7.38 (m, 4H), 7.25 (dd, J=7.3 Hz, 1H), 6.90-6.88 (m, 2H), 6.82 (d, J=8.6 Hz, 1H), 6.73 (d, J=8.8 Hz, 1H), 2.38 (s, 3H), 2.07 (s, 3H). ¹³C NMR (101 MHz; DMSO-d6): δ 152.1, 144.7, 143.3, 136.4, 134.1, 133.4, 132.8, 129.5, 128.9, 127.8, 127.6, 127.5, 126.4, 123.6, 123.2, 123.1, 119.6, 118.6, 115.6, 114.9, 110.3, 21.0, 13.9. (EI, magnetic sector): calcd for C25H21N303S 443.13036, found 443.13333.

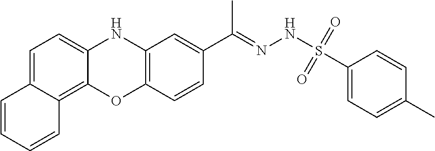 [SMILES: C/C(=N\NS(=O)(=O)c1ccc(C)cc1)c1ccc2c(c1)Nc1ccc3ccccc3c1O2]

2-(1-(piperazin-1-ylsulfonyl)ethyl)-10H-phenoxazine (45). Into a 10 mL sealable tube equipped with a stir bar were added (E)-N-(1-(10H-phenoxazin-2-yl)ethylidene)-4-methylbenzenesulfonohydrazide (52) (263 mg, 0.67 mmol), piperazine (346 mg, 4.02 mmol) and 6 mL of DMSO. While stirring, the mixture was continuously sparged with a steady stream of argon for 10 min after which DABSO (90 mg, 0.37 mmol) was added and the vessel was sealed. The tube was heated to 105° C. and was allowed to stir for 10 h. After cooling, the reaction mixture was poured into saturated NaHCO₃ solution which was subsequently extracted with ethyl acetate. The organic layer was dried with MgSO₄, filtered, then the solvent removed under reduced pressure. The product was isolated by flash chromatography (gradient of 100:0 to 98:2 DCM/MeOH) rendering the title product as a white solid (185 mg, 73%). ¹H NMR (400 MHz; DMSO-d6): δ 8.33 (br, 1H), 6.74-6.70 (m, 1H), 6.65-6.54 (m, 5H), 6.45-6.42 (m, 1H), 4.34 (q, J=7.1 Hz, 1H), 3.33 (br, 1H), 3.02-2.96 (m, 2H), 2.86-2.82 (m, 2H), 2.63-2.54 (m, 4H), 1.49 (d, J=7.1 Hz, 3H). ¹³C NMR (101 MHz; DMSO-d6): δ 142.9, 142.6, 132.3, 132.0, 130.9, 124.1, 121.3, 120.5, 115.1, 114.7, 113.5, 113.3, 60.0, 46.7, 45.5, 16.1. HRMS (EI, magnetic sector): calcd for C₁₈H₂₁N₃O₃S 359.13036, found 359.13301.

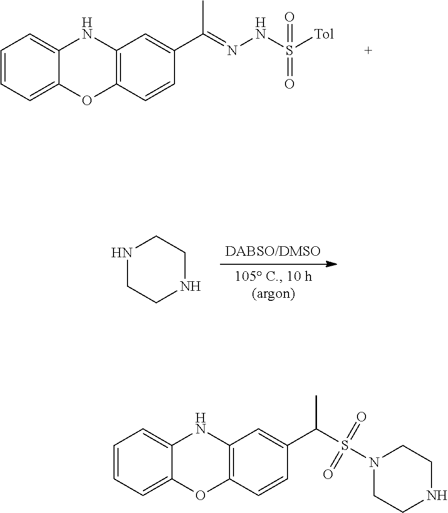

2-(1-(piperazin-1-ylsulfonyl)ethyl)-7-(trifluoromethyl)-OH-phenoxazine (46). Synthesis was carried out in the same manner as (45) using (E)-4-methyl-N-(1-(7-(trifluoromethyl)-10H-phenoxazin-2-yl)ethylidene)benzenesulfonohydrazide (53). The product was isolated by column chromatography (gradient of 100:0 to 98:2 DCM/MeOH) to afford the title product as a white solid (204 mg, 73%). ¹H NMR (400 MHz; DMSO-d6): δ 8.87 (br, 1H), 7.07 (dd, J=8.2, 0.9 Hz, 1H), 6.87 (d, J=1.6 Hz, 1H), 6.71 (dd, J=8.2, 2.0 Hz, 1H), 6.63 (d, J=8.1 Hz, 1H), 6.58 (d, J=1.9 Hz, 1H), 6.54 (d, J=8.1 Hz, 1H), 4.37 (q, J=7.1 Hz, 1H), 3.29 (br, 1H), 3.02-2.97 (m, 2H), 2.87-2.83 (m, 2H), 2.64-2.54 (m, 4H), 1.50 (d, J=7.1 Hz, 3H). ¹³C NMR (101 MHz; DMSO-d6): δ 142.6, 142.5, 136.0, 131.5, 130.9, 124.1 (q, J=270.7 Hz), 122.3, 121.7 (q, J=4.0 Hz), 120.4 (q, J=32.3 Hz), 114.9, 113.9, 113.1, 111.8 (q, J=3.7 Hz), 59.9, 46.6, 45.5, 16.0. ¹⁹F NMR (376 MHz; DMSO-d6): δ −60.7. HRMS (EI, magnetic sector): calcd for C₁₉H₂₀F₃N₃O₃S 427.11775, found 427.11607.

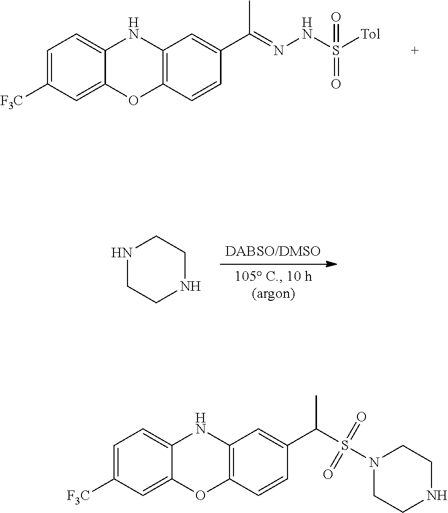

2-(1-(piperazin-1-ylsulfonyl)ethyl)-8-(trifluoromethyl)-10H-phenoxazine (66). Synthesis was carried out in the same manner as (45) using (E)-4-methyl-N′-(1-(8-(trifluoromethyl)-10H-phenoxazin-2-yl)ethylidene)benzenesulfonohydrazide (67) on a 0.8 mmol scale. The product was isolated by column chromatography (gradient of 100:0 to 95:5 DCM/MeOH), and after trituration with diethyl ether, the title product was afforded appearing as a colourless crystalline solid (253 mg, 74%). ¹H NMR (400 MHz; DMSO-d6): δ 8.66 (br, 1H), 6.90 (dd, J=8.3, 1.4 Hz, 1H), 6.75 (d, J=8.2 Hz, 1H), 6.70 (dd, J=8.2, 2.2 Hz, 1H), 6.65 (d, J=2.1 Hz, 1H), 6.64 (d, J=8.1 Hz, 1H), 6.56 (d, J=2.0 Hz, 1H), 4.37 (q, J=7.0 Hz, 1H), 3.02-2.96 (m, 2H), 2.86-2.82 (m, 2H), 2.63-2.53 (m, 4H), 1.50 (d, J=7.1 Hz, 3H). ¹³C NMR (101 MHz; DMSO-d6): δ 145.6, 142.3, 133.0, 131.7, 131.1, 124.7 (q, J=31.9 Hz), 123.9 (q, J=271.4 Hz), 122.0, 117.6 (q, J=4.4 Hz), 115.5, 115.1, 113.8, 109.3 (q, J=4.0 Hz), 59.9, 46.7, 45.6, 16.0. ¹⁹F NMR (283 MHz; DMSO-d6): δ −61.6. HRMS (EL, magnetic sector): calcd for C₁₉H₂₀F₃N₃O₃S 427.11775, found 427.11874.

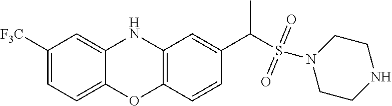 [SMILES: CC(c1ccc2c(c1)Nc1cc(C(F)(F)F)ccc1O2)S(=O)(=O)N1CCNCC1]

7-chloro-2-(1-(piperazin-1-ylsulfonyl)ethyl)-10H-phenoxazine (69). Synthesis was carried out in the same manner as (45) using (E)-N′-(1-(7-chloro-10H-phenoxazin-2-yl)ethylidene)-4-methylbenzenesulfonohydrazide (70) on a 0.8 mmol scale. The product was isolated by column chromatography (gradient of 100:0 to 95:5 DCM/MeOH), and after trituration with diethyl ether, the title product was afforded appearing as a slightly pink solid (202 mg, 64%). ¹H NMR (400 MHz; DMSO-d6): δ 8.50 (br, 1H), 6.78 (dd, J=8.4, 2.4 Hz, 1H), 6.70 (d, J=2.4 Hz, 1H), 6.67 (dd, J=8.2, 2.0 Hz, 1H), 6.61 (d, J=8.1 Hz, 1H), 6.55 (d, J=2.0 Hz, 1H), 6.42 (d, J=8.3 Hz, 1H), 4.35 (q, J=7.0 Hz, 1H), 3.01-2.96 (m, 2H), 2.86-2.81 (m, 2H), 2.63-2.53 (m, 4H), 1.49 (d, J=7.1 Hz, 3H). ¹³C NMR (101 MHz; DMSO-d6): δ 143.2, 142.3, 131.6, 131.4, 131.2, 123.7, 123.2, 121.6, 115.2, 114.9, 114.1, 113.7, 59.9, 46.7, 45.6, 16.1. HRMS (EI, magnetic sector): calcd for C18H20ClN3O3S 393.09139, found 393.08880.

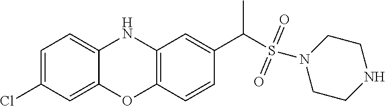 [SMILES: CC(c1ccc2c(c1)Nc1ccc(Cl)cc1O2)S(=O)(=O)N1CCNCC1]

2-chloro-8-(1-(piperazin-1-ylsulfonyl)ethyl)-10H-phenoxazine (72). Synthesis was carried out in the same manner as (45) using (E)-N′-(1-(8-chloro-10H-phenoxazin-2-yl)ethylidene)-4-methylbenzenesulfonohydrazide (73) on a 0.8 mmol scale. The product was isolated by column chromatography (gradient of 100:0 to 95:5 DCM/MeOH), and after trituration with diethyl ether, the title product was afforded appearing as an off-white solid (237 mg, 75%). ¹H NMR (400 MHz; DMSO-d6): δ 8.56 (br, 1H), 6.68 (dd, J=8.2, 2.0 Hz, 1H), 6.62-6.55 (m, 4H), 6.42 (d, J=2.2 Hz, 1H), 4.36 (q, J=7.0 Hz, 1H), 3.01-2.95 (m, 2H), 2.85-2.80 (m, 2H), 2.63-2.53 (m, 4H), 1.49 (d, J=7.1 Hz, 3H). ¹³C NMR (101 MHz; DMSO-d6): δ 142.6, 141.5, 133.6, 131.3, 131.2, 127.5, 121.9, 119.6, 116.3, 114.9, 113.7, 112.6, 59.9, 46.7, 45.6, 16.1. HRMS (EI, magnetic sector): calcd for C18H20ClN3O3S 393.09139, found 393.09211.

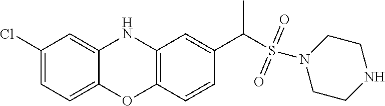 [SMILES: CC(c1ccc2c(c1)Nc1cc(Cl)ccc1O2)S(=O)(=O)N1CCNCC1]

2-fluoro-8-(1-(piperazin-1-ylsulfonyl)ethyl)-10H-phenoxazine (75). Synthesis was carried out in the same manner as (45) using (E)-N′-(1-(8-fluoro-10H-phenoxazin-2-yl)ethylidene)-4-methylbenzenesulfonohydrazide (76) on a 0.8 mmol scale. The product was isolated by column chromatography (gradient of 100:0 to 95:5 DCM/MeOH), and after trituration with diethyl ether, the title product was afforded appearing as a white solid (159 mg, 53%). ¹H NMR (400 MHz; DMSO-d6): δ 8.58 (br, 1H), 6.68 (dd, J=8.2, 2.0 Hz, 1H), 6.62-6.59 (m, 2H), 6.56 (d, J=2.0 Hz, 1H), 6.35 (ddd, J=8.6, 8.6, 3.0 Hz, 1H), 6.25 (dd, 9.7, 2.9 Hz, 1H), 4.35 (q, J=7.1 Hz, 1H), 3.01-2.95 (m, 2H), 2.85-2.80 (m, 2H), 2.62-2.52 (m, 4H), 1.49 (d, J=7.1 Hz, 3H). ¹³C NMR (101 MHz; DMSO-d6): δ 158.6 (d, J=237.3 Hz), 142.8, 138.8 (d, J=2.6 Hz), 133.4 (d, J=11.7 Hz), 131.0, 121.9, 115.7 (d, J=10.3 Hz), 114.8, 113.7, 105.5 (d, J=23.1 Hz), 100.4 (d, J=27.9 Hz), 59.9, 46.7, 45.6, 16.1. ¹⁹F NMR (283 MHz; DMSO-d6): δ −122.1. HRMS (EI, magnetic sector): calcd for C18H20FN3O3S 377.12094, found 377.12078.

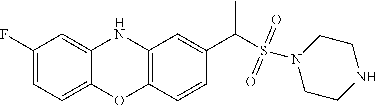 [SMILES: CC(c1ccc2c(c1)Nc1cc(F)ccc1O2)S(=O)(=O)N1CCNCC1]

2-(1-(piperazin-1-ylsulfonyl)ethyl)-7-(trifluoromethoxy)-10H-phenoxazine (78). Synthesis was carried out in the same manner as (45) using (E)-4-methyl-N′-(1-(7-(trifluoromethoxy)-10H-phenoxazin-2-yl)ethylidene)benzenesulfonohydrazide (79) on a 0.8 mmol scale. The product was isolated by column chromatography (gradient of 100:0 to 95:5 DCM/MeOH), and after trituration with diethyl ether, the title product was afforded appearing as a white solid (193 mg, 54%). ¹H NMR (400 MHz; DMSO-d6): δ 8.56 (br, 1H), 6.74 (dd, J=8.5, 1.7 Hz, 1H), 6.69-6.67 (m, 2H), 6.62 (d, J=8.1 Hz, 1H), 6.56 (d, J=2.0 Hz, 1H), 6.47 (d, 8.5 Hz, 1H), 4.35 (q, J=7.1 Hz, 1H), 3.01-2.96 (m, 2H), 2.86-2.81 (m, 2H), 2.63-2.52 (m, 4H), 1.49 (d, J=7.1 Hz, 3H). ¹³C NMR (101 MHz; DMSO-d6): δ 143.4, 142.5, 141.8 (q, J=2.2 Hz), 132.1, 132.0(4), 132.0(0), 122.23, 120.6 (q, J=255.3 Hz), 117.2, 115.4, 114.2, 113.7, 109.7, 60.4, 47.2, 46.1, 16.5. ¹⁹F NMR (283 MHz; DMSO-d6): δ −57.8. HRMS (EI, magnetic sector): calcd for C19H20F3N3O4S 443.11266, found 443.11031.

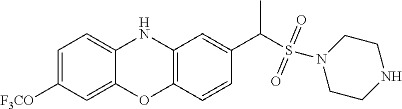 [SMILES: CC(c1ccc2c(c1)Nc1ccc(OC(F)(F)F)cc1O2)S(=O)(=O)N1CCNCC1]

10-(1-(piperazin-1-ylsulfonyl)ethyl)-12H-benzo[a]phenoxazine (81). Synthesis was carried out in the same manner as (45) using (E)-N′-(1-(12H-benzo[a]phenoxazin-10-yl)ethylidene)-4-methylbenzenesulfonohydrazide (82) on a 0.9 mmol scale. The product was isolated by column chromatography (gradient of 100:0 to 95:5 DCM/MeOH), and the title product was afforded appearing as a brownish-yellow solid (198 mg, 54%). ¹H NMR (400 MHz; DMSO-d6): δ 8.35 (br, 1H), 8.00 (d, J=8.4 Hz, 1H), 7.73 (d, J=7.9 Hz, 1H), 7.45 (ddd, J=8.1, 8. 1, 1.1 Hz, 1H), 7.35 (dd, J=7.1, 7.1 Hz), 7.25 (d, J=8.7 Hz, 1H), 6.93 (d, J=8.7 Hz, 1H), 6.87 (d, J=2.0 Hz, 1H), 6.70 (dd, J=8.1, 2.0 Hz, 1H), 6.63 (d, J=8.1 Hz, 1H), 4.36 (q, J=7.1 Hz, 1H), 3.06-3.01 (m, 2H), 2.91-2.86 (m, 2H), 2.66-2.61 (m, 4H), 1.53 (d, J=7.1 Hz, 3H). ¹³C NMR (101 MHz; DMSO-d6): δ 143.4, 138.1, 132.6, 131.1, 130.6, 128.2, 125.5, 125.1, 124.5, 122.4, 121.5, 120.7, 120.3, 116.5, 114.7, 114.4, 60.1, 46.6, 45.5, 16.1. HRMS (EI, magnetic sector): calcd for C22H23N3O3S 409.14601, found 409.14286.

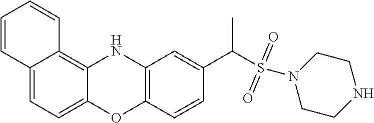 [SMILES: CC(c1ccc2c(c1)Nc1c(ccc3ccccc13)O2)S(=O)(=O)N1CCNCC1]

9-(1-(piperazin-1-ylsulfonyl)ethyl)-7H-benzo[c]phenoxazine (84). Synthesis was carried out in the same manner as (45) using (E)-N′-(1-(7H-benzo[c]phenoxazin-9-yl)ethylidene)-4-methylbenzenesulfonohydrazide (85) on a 0.55 mmol scale. The product was isolated by column chromatography (gradient of 100:0 to 95:5 DCM/MeOH), and the title product was afforded appearing as a organish-yellow solid (154 mg, 68%). ¹H NMR (400 MHz; DMSO-d6): δ 8.40 (br, 1H), 7.77 (d, J=8.4 Hz, 1H), 7.69 (d, J=8.2 Hz, 1H), 7.42-7.39 (m, 2H), 7.24 (ddd, J=8.1, 8.1, 1.1 Hz), 6.81 (d, J=8.6 Hz, 1H), 6.76-6.69 (m, 2H), 6.59 (d, J=1.9 Hz, 1H), 4.36 (q, J=7.1 Hz, 1H), 3.04-2.98 (m, 2H), 2.88-2.84 (m, 2H), 2.64-2.55 (m, 4H), 1.52 (d, J=7.1 Hz, 3H). ¹³C NMR (101 MHz; DMSO-d6): δ 143.4, 134.2, 132.9, 131.3, 128.9, 127.8, 126.4, 123.5, 123.2, 123.1, 122.0, 118.6, 115.6, 115.1, 113.8, 60.0, 46.7, 45.6, 16.1. HRMS (EI, magnetic sector): calcd for C22H23N3O3S 409.14601, found 409.14729.

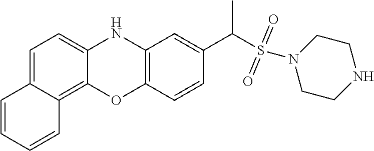 [SMILES: CC(c1ccc2c(c1)Nc1ccc3ccccc3c1O2)S(=O)(=O)N1CCNCC1]

2-(1-((4-methylpiperazin-1-yl)sulfonyl)ethyl)-10H-phenoxazine (43). Synthesis was carried out in an analogous fashion to (45) using (E)-N′-(1-(10H-phenoxazin-2-yl)ethylidene)-4-methylbenzenesulfonohydrazide (52) (1.5 mmol) and 1-methylpiperazine (7.6 mmol). The product was purified by column chromatography (gradient of 100:0 to 98:2 DCM/MeOH) to afford the product as a white solid (400 mg, 70%). ¹H NMR (400 MHz; DMSO-d6): δ 8.33 (br, 1H), 6.75-6.70 (m, 1H), 6.65-6.54 (m, 5H), 6.44 (dd, J=7.7, 1.3 Hz, 1H), 4.36 (q, J=7.1 Hz, 1H), 3.09-3.04 (m, 2H), 2.93-2.87 (m, 2H), 2.25-2.17 (m, 4H), 2.12 (s, 1H), 1.50 (d, J=7.1 Hz, 3H). ¹³C NMR (101 MHz; DMSO-d6): δ 142.9, 142.6, 132.3, 132.0, 130.8, 124.1, 121.3, 120.5, 115.1, 114.8, 113.5, 113.4, 60.2, 54.4, 45.5(3), 45.4(6), 16.0. (EI, magnetic sector): calcd for C₁₉H₂₃N₃O₃S 373.14601, found 373.15108.

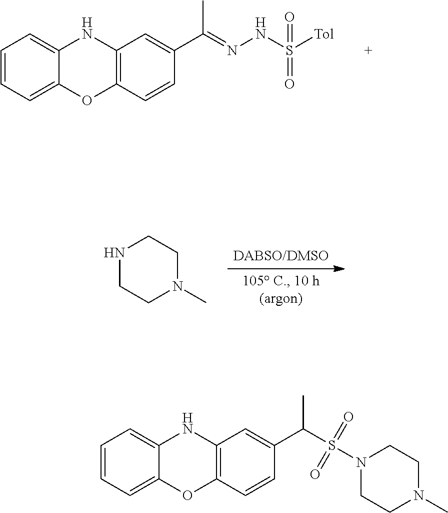

2-(1-((4-methylpiperazin-1-yl)sulfonyl)ethyl)-7-(trifluoromethyl)-10H-phenoxazine (48). Synthesis was carried out in an analogous fashion to (45) using (E)-4-methyl-N′-(1-(8-(trifluoromethyl)-10H-phenoxazin-2-yl)ethylidene)benzenesulfonohydrazide (67) (0.76 mmol) and 1-methylpiperazine (3.5 mmol). The product was purified by column chromatography (gradient of 100:0 to 98:2 DCM/MeOH) to afford the product as a white solid (210 mg, 63%). ¹H NMR (400 MHz; DMSO-d6): δ 8.86 (br, 1H), 7.08-7.06 (m, 1H), 6.87 (d, J=1.9 Hz, 1H), 6.71 (dd, J=8.2, 2.0 Hz, 1H), 6.63 (d, J=8.2 Hz, 1H), 6.58 (d, J=2.0 Hz, 1H), 6.54 (d, J=8.1 Hz, 1H), 4.40 (q, J=7.1 Hz, 1H), 3.10-3.04 (m, 2H), 2.92-2.89 (m, 2H), 2.25-2.17 (m, 4H), 2.12 (s, 3H), 1.50 (d, J=7.1 Hz, 3H). ¹³C NMR (101 MHz; DMSO-d6): δ 142.6(4), 142.5(5), 135.9, 131.4, 130.9, 124.1 (q, J=270.7), 122.3, 121.7 (q, J=4.0 Hz), 120.4 (q, J=32.6 Hz), 115.0, 114.0, 113.1, 111.8 (q, J=4.0 Hz), 60.1, 54.4, 45.5(4), 45.4(6), 15.9. ¹⁹F NMR (376 MHz; DMSO-d6): δ −60.7. (EI, magnetic sector): calcd for C₂₀H₂₂F₃N₃O₃S 441.13340, found 441.13243.

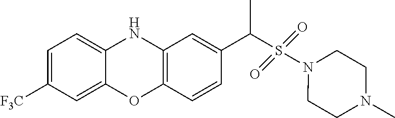 [SMILES: CC(c1ccc2c(c1)Nc1ccc(C(F)(F)F)cc1O2)S(=O)(=O)N1CCN(C)CC1]

N-(2-(dimethylamino)ethyl)-1-(10H-phenoxazin-2-yl)ethanesulfonamide (43). Synthesis was carried out in an analogous fashion to (45) using (E)-N′-(1-(10H-phenoxazin-2-yl)ethylidene)-4-methylbenzenesulfonohydrazide (52) (0.67 mmol) and N,N-dimethylethylenediamine (1.34 mmol). The product was purified by column chromatography (gradient of 100:0 to 98:2 DCM/MeOH) to afford the product as an off-white solid (120 mg, 49%). ¹H NMR (400 MHz; DMSO-d6): δ 8.29 (br, 1H), 6.91 (t, J=5.3 Hz, 1H) 6.74-6.70 (m, 1H), 6.61-6.52 (m, 5H), 6.44 (dd, J=7.7, 1.4 Hz, 1H), 4.24 (q, J=7.1 Hz, 1H), 2.91-2.82 (m, 2H), 2.26 (t, J=6.9 Hz, 2H), 2.13 (s, 6H), 1.50 (d, J=7.1 Hz, 3H). ¹³C NMR (101 MHz; DMSO-d6): δ 142.6(9), 142.6(3), 132.1(8), 132.1(7), 131.3, 124.0, 121.3, 120.4, 115.1, 114.6, 113.7, 113.3, 60.6, 59.0, 45.0, 40.8, 15.8. (EI, magnetic sector): calcd for C₁₈H₂₃N₃O₃S 361.14601, found 361.14858.

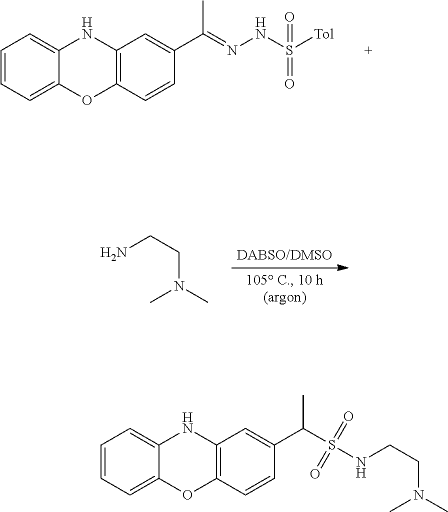

N-(2-(dimethylamino)ethyl)-1-(7-(trifluoromethyl)-10H-phenoxazin-2-yl)ethanesulfonamide (44). Synthesis was carried out in an analogous fashion to (45) using (E)-4-methyl-N-(1-(8-(trifluoromethyl)-10H-phenoxazin-2-yl)ethylidene)benzenesulfonohydrazide (67) (0.76 mmol) and N,N-dimethylethylenediamine (1.9 mmol). The product was purified by column chromatography (gradient of 100:0 to 98:2 DCM/MeOH) to afford the product as a white solid (148 mg, 45%). ¹H NMR (400 MHz; DMSO-d6): δ 8.83 (br, 1H), 7.07 (d, J=8.1 Hz, 1H), 6.94 (br, 1H), 6.87 (br, 1H), 6.67 (dd, J=8.3, 2.0 Hz, 1H), 6.61 (d, J=8.1 Hz, 1H), 6.55-6.53 (m, 2H), 4.27 (q, J=7.1 Hz, 1H), 2.88 (br, 2H), 2.25 (t, J=6.9 Hz, 2H), 2.12 (s, 6H), 1.50 (d, J=7.1 Hz, 3H). ¹³C NMR (101 MHz; DMSO-d6): δ 142.7, 142.3, 136.1, 131.9, 130.8, 124.1 (q, J=270.7 Hz), 122.4, 121.6 (q, J=3.7 Hz), 120.3 (q, J=32.7 Hz), 114.8, 114.1, 113.0, 111.8 (q, J=4.0 Hz), 60.5, 59.0, 45.1, 40.9, 15.8. ¹⁹F NMR (376 MHz; DMSO-d6): δ −60.7. (EI, magnetic sector): calcd for C₁₉H₂₂F₃N₃O₃S 429.13340, found 429.13419.

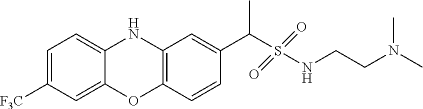 [SMILES: CC(c1ccc2c(c1)Nc1ccc(C(F)(F)F)cc1O2)S(=O)(=O)NCCN(C)C]

2-(1-(morpholinosulfonyl)ethyl)-7-(trifluoromethyl)-10H-phenoxazine (49). Synthesis was carried out in an analogous fashion to (45) using (E)-4-methyl-N-(1-(8-(trifluoromethyl)-10H-phenoxazin-2-yl)ethylidene)benzenesulfonohydrazide (67) (0.76 mmol) and morpholine (3 mmol). The product was purified by column chromatography (30% ethyl acetate in hexanes) to afford the product as a white solid (218 mg, 67%). ¹H NMR (400 MHz; DMSO-d6): δ 8.87 (br, 1H), 7.07 (dd, J=8.1, 1.2 Hz, 1H), 6.87 (d, J=1.7 Hz, 1H), 6.74 (dd, J=8.2, 1.9 Hz, 1H), 6.65 (d, J=8.2 Hz, 1H), 6.59 (d, J=2.0 Hz, 1H), 6.54 (d, J=8.1 Hz, 1H), 4.44 (q, J=7.1 Hz, 1H), 3.55-3.45 (m, 4H), 3.10-3.05 (m, 2H), 2.94-2.89 (m, 2H), 1.52 (d, J=7.1 Hz, 1H). 142.6(4), 142.6(0), 135.9, 131.2, 131.0, 124.1 (q, J=270.7 Hz), 122.4, 121.8 (q, J=3.7 Hz), 120.4 (q, J=32.3 Hz), 115.0, 113.9, 113.1, 111.8 (q, J=3.3 Hz), 66.1, 60.0, 45.9, 15.9. ¹³C NMR (101 MHz; DMSO-d6): δ 142.7, ¹⁹F NMR (283 MHz; DMSO-d6): δ −60.7. (EI, magnetic sector): calcd for C19H19F3N2O4S 428.10176, found 428.10385.

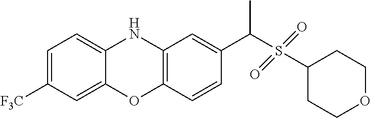 [SMILES: CC(c1ccc2c(c1)Nc1ccc(C(F)(F)F)cc1O2)S(=O)(=O)C1CCOCC1]

### Example 3. RTA Activity in Phospholipid Membranes

The intrinsic RTA activities of test compounds (PNX and PTZ derivatives) were determined in azobis(isobutyronitrile) (AIBN)-initiated co-autoxidations of PBD-BODIPY and dioxane, as described (Farmer et al., 2017). Results are shown in FIG. 2(A). PBD-BODIPY enables reaction progress to be monitored by conventional spectrophotometry, and since its rate of reaction with dioxane-derived peroxyl radicals is known (i.e., k_{PBD-BODIPY}=5310 M⁻¹s⁻¹), kᵢₙₕ of the RTA can be calculated simply from the initial rate of PBD-BODIPY oxidation, with which it competes (Eq. 1 (see FIG. 2(A)). Moreover, the length of the inhibited period (tᵢₙₕ) is related to the radical-trapping stoichiometry (n) of the RTA as in Eq. 2 (see FIG. 2(A). Using this approach, several kinetic experiments were carried out in parallel, enabling convenient systematic comparisons of several RTAs in a variety of substrates and conditions. Representative inhibited co-autoxidation data are shown for three derivatives of each of PNX (left) and PTZ (right) in FIG. 2(B). In the limited number of cases where the amine completely suppressed PBD-BODIPY oxidation, such as 3,7-tBu-PNX (34) in FIG. 2(B) (precluding determination of kᵢₙₕ from the initial rate), a strong H-bond accepting cosolvent (DMSO) was added to lower the concentration of free RTA in solution and ensure accurate determination of kᵢₙₕ (FIG. 2(D)).

To enable direct comparison of the intrinsic RTA activity for all derivatives, the rate constants were normalized for the contribution of the H-bonding pre-equilibrium of the RTA and solvent(s) (FIG. 2(C)) (Snelgrove et al., 2001). Doing so involved determination of the H-bond acidity (α₂^{H}) of the RTA by ¹H NMR (Farmer et al., 2017; Abraham et al., 2006) (values are included in Table 1) and using it along with the H-bond basicity (β₂^{H}) of the solvent system to determine kᵢₙₕ⁰ using the Abraham-Ingold equation (Eq. 4) (Snelgrove et al., 2001). The data, which are given in Table 1 and FIG. 2(G), span three orders of magnitude, from 2.5×10⁶ M⁻¹s⁻¹ for 3,7-NO₂—PTZ (29) to 1.3×10⁹ M⁻¹s⁻¹ for 3,7-MeO-PNX (40). We observed two trends: (1) the intrinsic reactivity of PNX derivatives was systematically higher than equivalently substituted PTZ derivatives, and (2) reactivity increased with increasing electron-richness of either the PNX or PTZ derivatives, consistent with previous observations (Farmer et al. 2017). These data were used to provide a baseline for comparison to the reactivity of the same set of compounds determined in liposomal egg phosphatidylcholine (PC) using the FENIX approach (Shah et al., 2019). Therein, reaction progress was monitored for Me-OAMVN-initiated autoxidations via the formation of oxidized STY-BODIPY as determined by fluorescence, and kᵢₙₕ^{lip} of the RTA was determined based upon k_{STY-BODIPY}=894 M⁻¹s⁻¹. Note that monitoring STY-BODIPY either by fluorescence or by absorbance can generally be done interchangeably, although strongly absorbing RTAs, e.g., 17 and 18, were incompatible with fluorescence assay due to fluorescence quenching.

Representative traces for the same three derivatives of each of PNX and PTZ are shown in FIG. 2(E), and the data for these and the other test compounds is tabulated alongside the kᵢₙₕ⁰ data in Table 1. While the absolute values of the rate constants were significantly suppressed in phospholipids, they spanned a similarly large range, from 4×10² M⁻¹s⁻¹ for 3,7-NO2-PTZ (29) to 2.2×10⁵ M⁻¹s⁻¹ for PNX. Electron-rich PNX derivatives were more reactive, but the complete suppression of STY-BODIPY oxidation precluded the determination of their kᵢₙₕ^{lip} values (e.g., compound 34, in FIG. 2(E)). Nevertheless, using available data for compounds 1-29 we could evaluate how well the intrinsic reactivity of the RTAs correlated to their kinetics in lipid bilayers; the plot in FIG. 2(F) (top) showed that this correlation was not strong.

Since our previous work had suggested that the phosphodiester moiety of egg phosphatidylcholine is a strong H-bond acceptor (Shah et al., 2019), the kinetic data was replotted as shown in FIG. 2(F) (bottom), normalizing for the differences in H-bond acidity (α₂^{H}) between the RTAs. The excellent correlation which resulted from this treatment clearly demonstrated that H-bonding is uniquely responsible for the attenuation in RTA activity in lipid bilayers (at least, under these experimental conditions). (Note compounds 30 and 31 were excluded from the analysis, as their low α₂^{H} resulted in substantial increases in the range of the plot with too few data points to populate the expanded range, strongly skewing the slope and intercept). Moreover, the y-intercept of the correlation provided the effective H-bond basicity (β₂^{H}) of liposomal egg phosphoatidylcholine (0.69). This value is in very good agreement with our estimate of β₂^{H}=0.79 (Shah et al., 2019) based on the results of ¹⁹F NMR studies of H-bond formation between egg PC and 4-fluorophenol in dilute solutions of CCl₄. Clearly, the intrinsic reactivity of the PNX and PTZ derivatives translated very well from organic solution to egg PC liposomes when the strong H-bonding interaction to the phosphatidylcholine headgroup was taken into account.

On the basis of the excellent correlation in FIG. 2(F), we anticipated that kᵢₙₕ^{lip} could be estimated for the 8 electron-rich PNX derivatives and the one electron-rich PTZ derivative that fully suppressed STY-BODIPY oxidation using the kᵢₙₕ⁰ values obtained from the experiments in organic solution and the effective β₂^{H} value for the egg PC liposomes. Although the correlation in FIG. 2(F) and associated β₂^{H} value was compiled from kinetic data derived from the substituted azine derivatives, the predicted kᵢₙₕ^{lip} for PMC (a phenol) was in excellent agreement with the measured value (3.5×10⁴ and 2.9×10⁴ M⁻¹s⁻¹, respectively), supporting this approach. The estimated kᵢₙₕ^{lip} values for the 9 amines (FIG. 2(G)) were up to 26-fold larger than that of PNX (i.e kᵢₙₕ^{lip}=6×10⁶ M⁻¹s⁻¹ for 3,7-MeO-PNX (40)).

### Example 4. Synergy with Ascorbate Boosts Efficacy and Enables

### Derivation of Kinetics for the Most Potent RTAs.

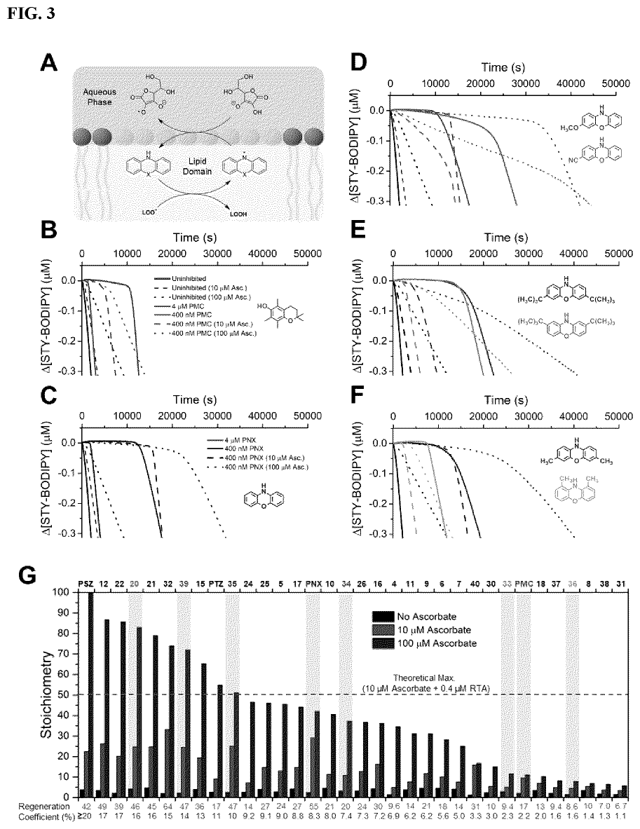

Although ascorbate is not itself an efficient inhibitor of the propagation of lipid peroxidation due to its localization in the aqueous domain, it can act as an inhibitor indirectly via regeneration of a lipophilic RTA at the lipid-water interface. The synergism between α-TOH and ascorbate has been well documented (Doba et al., 1985; Niki et al., 1985; Sato et al., 1990) but other lipid-soluble RTAs have seldom been studied in such detail. In principle, persistent aminyl radicals derived from azines such as PNX and PTZ should be substrates for reduction analogous to the phenoxyl radical derived from α-TOH (FIG. 3(A)). To systematically examine if this is indeed the case, azine-inhibited STY-BODIPY/egg PC co-autoxidations were carried out in the presence of ascorbate (at 10 and 100 μM). These experiments employed the more lipophilic initiator (di-tert-undecylhyponi-trite, DTUN) to minimize trapping of initiator-derived radicals by ascorbate (which is acutely observed when the more amphiphilic V70 initiator is used (Shah et al., 2019). Representative inhibited autoxidation traces are shown for a panel of compounds in FIGS. 3(B)-(F). Synergism is easily identified by an increase in the duration of the inhibited period, without corresponding change to the rate of the inhibited oxidation. For example, addition of ascorbate (10 M) increased the period of an autoxidation inhibited by PMC (at 400 nM) from ~1400 s to >5000s (FIG. 3(B)).

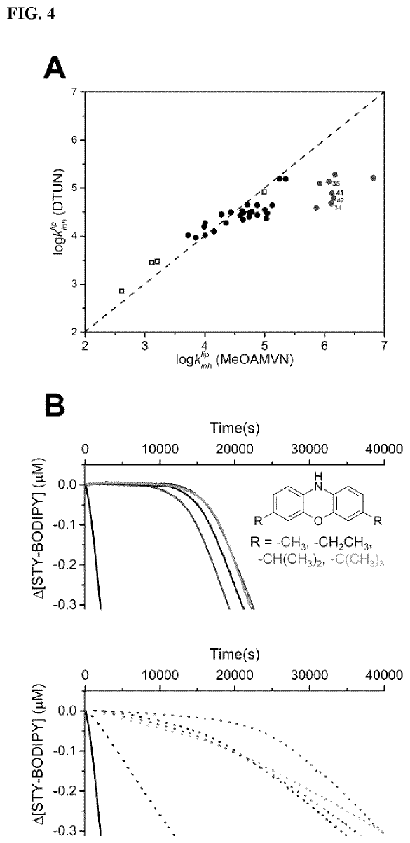

The data in FIG. 3 reveal that, in general, the PNX derivatives were readily regenerated when ascorbate was added to the system, as were PTZ derivatives. Interestingly, PNX (FIG. 3(C)) was more efficiently regenerated than PMC with inhibition times that were >3-fold longer under otherwise identical conditions. The inhibited periods for PNX derivatives with EW or ED groups were similarly extended, as shown for 3-CN-PNX (20) and 3-MeO-PNX (39) in FIG. 3(D). Interestingly, the positioning of substituents could have a pronounced effect on propensity to undergo regeneration. For instance, the tᵢₙₕ of 2,8-tBu-PNX (33) and 3,7-tBu-PNX (34), while equivalent in the absence of ascorbate, were >4-fold larger for 3,7-tBu-PNX in the presence of 100 M of ascorbate (FIG. 3(E)). Moreover, while 3,7-Me-PNX (35) exhibited the expected synergy with ascorbate, the inhibited period of 1,9-Me-PNX (36) was only marginally affected by added ascorbate (FIG. 4(F)). A similar deficit was observed for other PNX/PTZ compounds with methyl groups in the 1 and 9 positions (compounds 31 and 38). The whole library of compounds was surveyed, and efficiency of regeneration was expressed both in terms of radical-trapping stoichiometry (relative to the concentration of the RTA using Eq. 2 (see FIG. 2(A)) and in terms of a regeneration efficiency (a).

Assuming each equivalent of ascorbate can enable two reductions, the maximum stoichiometry expected from 400 nM RTA combined with 10 and 100 M ascorbate would be 50 and 500, respectively (in addition to n~2 of the RTA by itself). With 10 M of ascorbate, regeneration of PNX lead to a >10-fold increase in radical-trapping stoichiometry (n=28.9), corresponding to a regeneration efficiency of α=55%. The stoichiometry of PNX increased further with additional ascorbate (n=41.9), but with a decrease in regeneration efficiency (α=8.3%), which was presumably due to the greater opportunity for alternative fates of the PNX-derived radical as the duration of the experiment increased. With 100 M ascorbate, the largest a was observed for 3-CHO-PNX (12) at 17%, however, inhibition with PSZ exceeded the duration of the experiment (54000 s) indicating a stoichiometry that was >100 (or an a >20%). These results are summarized in FIG. 3(G).

On the basis of the foregoing, we wondered if DTUN-initiated autoxidations carried out at lower concentration of RTA (i.e., 400 nM in lieu of 4 μM) in the presence of excess ascorbate would provide inhibited periods of sufficient slope and duration to enable determination of kᵢₙₕ^{lip} values of compounds that were too reactive for quantification in the V70-initiated autoxidations (necessitating estimation using Eq. 5). Indeed, after correcting for the slight retardation in STY-BODIPY oxidation owing to the presence of ascorbate (factors of 1.6 and 4.7 for 10 and 100 M ascorbate, respectively), values of kᵢₙₕ^{lip} could be derived which were generally in good agreement with those determined at higher RTA concentrations in the absence of ascorbate. Data are shown in Table 3.

TABLE 3
Inhibition Rate Constants and Radical Trapping Stoichiometries in DTUN
Initiated Egg PC Liposomes With/Without Ascorbate.

| Compound | | kᵢₙₕ^{lip} (10³ M⁻¹s⁻¹)/n | | | |
|---|---|---|---|---|---|
| no. | RTA | No Ascorbate | 10 μM Ascorbate | 100 μM Ascorbate | LogP ^{e} |
| 1 | PNX | (183 ± 19)/2.5 ± 0.1 | (396 ± 68)/28.9 ± 0.8 | (155 ± 26)/41.9 ± | 3.84 ± |
| | | | | 0.4 | 0.44 |
| 2 | PTZ | (58 ± 6.4)/2.7 ± 0.1 | (66 ± 3.7)/9.1 ± 0.2 | (30 ± 0.4)/54.9 ± | 4.15 ± |
| | | | | 1.0 | 0.26 |
| 3 | PSZ | (37 ± 4.7)/3.8 ± 0.1 | (36 ± 0.7)/22.4 ± 0.5 | (31±0.7)/≥100 | 4.37 ± |
| | | | | | 0.70 |
| 4 | Benzo(b)- | (too fast)/1.5 ± 0.1 | (too short)/4.9 ± 0.1 | (35 ± 1.7)/34.6 ± | 5.07 ± |
| | PNX | ((10.6 ± 0.6)/ | | 0.8 | 0.44 |
| | | 0.9 ± 0.1) | | | |
| 5 | Benzo(b)-PTZ | (too fast)/2.8 ± 0.1 | (55±4.6)/12.9 ± 0.3 | (45 ±2.6)/45.5 ± | 5.38 ± |
| | | | | 1.7 | 0.26 |
| 6 | 2-CF3-PNX | (57 ± 0.8)/4.6 ± 0.1 | (131±25)/10.1 ± 0.2 | (44 ± 3.7)/28.2 ± | 5.20 ± |
| | | | | 0.2 | 0.51 |
| 7 | 2-CF₃-7-tBu- | (72 ± 13)/4.1 ± 0.3 | (82 ± 1.0)/7.6 ± 0.1 | (28 ± 0.4)/25.1 ± | 6.88 ± |
| | PNX | | | 0.6 | 0.52 |
| 8 | 2-CF₃-8-tBu- | (76 ± 7.4)/3.6 ± 0.2 | (too short)/5.5 ± 0.1 | (too short)/6.9 ± | 6.88 ± |
| | PNX | | | 0.3 | 0.52 |
| | | | | ((11.2 ± 0.4)/— b) | |
| 9 | 3-CF3-PNX | (34 ± 1.4)/4.6 ± 0.1 | (72 ± 5.0)/11.7 ± 0.5 | (31 ± 2.7)/31.1 ± | 4.98 ± |
| | | | | 0.6 | 0.50 |
| 10 | 3-CF₃-7-tBu- | (48 ± 2.5)/4.6 ± 0.3 | (68 ± 2.3)/11.3 ± 0.1 | (25 ± 0.2)/40.6 ± | 6.67 ± |
| | PNX | | | 0.6 | 0.51 |
| 11 | 3-CF₃-8-tBu- | (56 ± 11)/3.8 ± 0.1 | (71 ± 2.4)/7.7 ± 0.1 | (22 ± 0.6)/31.2 ± | 6.67 ± |
| | PNX | | | 0.3 | 0.51 |
| 12 | 3-CHO-PNX | (39 ± 5.5)/3.6 ± 0.1 | (45 ±0.6)/26.2 ± 0.6 | (33 ± 0.8)/86.7± | 3.44 ± |
| | | | | 2.3 | 0.48 |
| 13 | 3,7-CHO- | (8.8 ± 0.8)/4.5 ± 0.1 | (11.2 ± 0.1)/— ^{b} | (10.4 ± 0.2)/— ^{b} | 3.03 ± |
| | PNX | | | | 0.52 |
| 14 | 3-CHO-7-CN- | (8.6 ± 0.9)/>5 | (9.1±0.1)/— ^{b} | (9.3 ± 0.3)/— ^{b} | 3.24 ± |
| | PNX | | | | 0.53 |
| 15 | 3-NO2-PNX | (15.7 ± 1.1)/4.0 ± 0.1 | (25 ± 0.7)/19.4 ± 0.8 | (19±0.5)/65.3 ± | 3.98 ± |
| | | | | 1.2 | 0.46 |
| 16 | 3-NO2-7-Me- | (22 ± 2.7)/4.2 ± 0.4 | (37 ± 1.0)/16.2 ± 0.2 | (19 ± 0.1)/36.2 ± | 4.44 ± |
| | PNX | | | 0.9 | 0.46 |
| 17 | 3-NO₂-7- | (49 ± 11) ^{a}/4.2 ± 0.8 | (66 ± 2.0)/14.7 ± 0.2 | (32 ± 0.4)/44.1 ± | 3.90 ± |
| | MeO-PNX | | | 0.2 | 0.48 |
| 18 | 3-NO2-7- | (47 ± 9.4) ^{a}/3.6 ± 0.2 | (185 ± 10)/6.9 ± 0.1 | (157± 34)/10.3 ± | 5.31 ± |
| | NEt2-PNX | | | 0.1 | 0.48 |
| 19 | 3,7-NO₂-PNX | (2.8 ± 0.1)/— ^{b} | ((6.3± 0.1)/—) ^{b,c} | ((6.2± 0.1)/—) ^{b,c} | 4.12 ± |
| | | | | | 0.48 |
| 20 | 3-CN-PNX | (25 ± 3.0)/4.2 ± 0.1 | (41 ± 0.9)/24.7 ± 0.1 | (31 ± 1.4)/82.9 ± | 3.65 ± |
| | | | | 0.7 | 0.49 |
| 21 | 3-CN-7-Me- | (53 ± 2.5)/4.8 ± 0.2 | (69 ± 2.3)/24.8 ± 0.5 | (31 ± 0.4)/78.9 ± | 4.11 ± |
| | PNX | | | 1.0 | 0.49 |
| 22 | 3-CN-7-MeO- | (79 ± 4.8)/2.2 ± 0.1 | (107 ± 4.7)/20.2 ± 0.4 | (44 ± 2.1)/85.7 ± | 3.56 ± |
| | PNX | | | 1.0 | 0.50 |
| 23 | 3-CN-7-NO2- | (3.0 ± 0.1)/— ^{b} | ((6.6 ± 0.1)/—) ^{b,c} | ((6.2 ± 0.2)/—) ^{b,c} | 3.79 ± |
| | PNX | | | | 0.51 |
| 24 | 2-Cl-PTZ | (36 ± 2.7)/2.6 ± 0.1 | (64 ± 5.2)/7.3 ± 0.1 | (27± 0.3)/46.5 ± | 5.07 ± |
| | | | | 0.2 | 0.27 |
| 25 | 2-CF3-PTZ | (19 ± 0.7)/4.0 ± 0.2 | (42 ± 3.6)/14.7 ± 0.5 | (28 ±0.5)/46.1 ± | 5.50 ± |
| | | | | 0.4 | 0.35 |
| 26 | 3-CF3-PTZ | (11.1 ± 0.7)/3.2 ± 0.1 | (23 ± 0.6)/12.7 ± 0.1 | (15.7±0.5)/36.7 ± | 4.72 |
| | | | | 0.4 | 0.77 |
| 27 | 3-CHO-PTZ | (11.7 ± 1.1)/2.4 ± 0.1 | (16.6 ± 0.1)/— ^{b} | (12.5 ± 0.3)/— ^{b} | 3.57 ± |
| | | | | | 0.77 |
| 28 | 3-NO₂-PTZ | (5.0 ± 0.3)/1.8 ± 0.1 | (10.4 ± 0.1)/— ^{b,c} | (10.4 ± 0.1)/— ^{b,c} | 3.88 ± |
| | | | | | 0.75 |
| 29 | 3,7-NO2-PTZ | (0.26 ± 0.01)/— | (—) d | (—) d | 3.61 ± |
| | | | | | 1.03 |
| 30 | 3,7-tBu-PTZ | (46 ± 5.5)/2.5 ± 0.1 | (too short)/5.4 ± 0.1 | (23 ± 0.3)/15.0 ± | 7.53 ± |
| | | | | 0.3 | 0.29 |
| 31 | 1,9-Me-PTZ | (83 ± 8.6)/1.5 ± 0.1 | (too short)/3.5 ± 0.1 | (too short)/5.7 ± | 5.18 ± |
| | | | | 0.1 | 0.28 |
| 32 | 3,7-MeO-PTZ | (too fast)/2.0 ± 0.1 | (404± 68)/33.2 ± 0.8 | (127 ± 14)/74.0 ± | 3.98 ± |
| | | | | 0.9 | 1.03 |
| 33 | 2,8-tBu-PNX | (too fast)/2.8 ± 0.1 | (too short)/5.0 ± 0.2 | (39 ± 2.5)/11.5 ± | 7.22 ± |
| | | | | 0.1 | 0.46 |
| 34 | 3,7-tBu-PNX | (too fast)/3.1 ± 0.1 | (98 ± 4.9)/10.8 ± 0.1 | (48 ± 4.3)/37.1 ± | 7.22 ± |
| | | | | 0.8 | 0.46 |
| 35 | 3,7-Me-PNX | (too fast)/2.5 ± 0.1 | (435 ± 27)/25.0 ± 0.3 | (135 ± 8.6)/51.1 ± | 4.76 ± |
| | | | | 1.4 | 0.44 |
| 36 | 1,9-Me-PNX | (too fast)/1.4 ± 0.1 | (too short)/4.4 ± 0.1 | (too short)/7.8 ± | 4.76 ± |
| | | | | 0.1 | 0.44 |
| 37 | 2,4,6,8-Me- | (too fast)/2.1 ± 0.1 | (too short)/4.9 ± 0.1 | (too short)/8.2 ± | 5.68 ± |
| | PNX | | | 0.2 | 0.45 |
| 38 | 1,3,7,9-Me- | (too fast)/1.8 ± 0.1 | (too short)/3.7 ± 0.7 | (too short)/6.5 ± | 5.68 ± |
| | PNX | | | 0.3 | 0.45 |
| 39 | 3-MeO-PNX | (too fast)/2.1 ± 0.1 | (480 ± 179)/24.4 ± 0.5 | (189 ± 37)/71.8 ± | 3.76 ± |
| | | | | 4.0 | 0.45 |
| 40 | 3,7-MeO- | (too fast)/1.2 ± 0.1 | (491± 69)/15.9 ± 0.2 | (162 ± 6.7)/16.7 ± | 3.67 ± |
| | PNX | | | 0.1 | 0.47 |
| 41 | 3,7-Et-PNX | (too fast)/2.9 ± 0.1 | (149 ± 6.4)/15.9 ± 0.3 | (77 ± 5.0)/35.5 ± | 5.83 ± |
| | | | | 0.7 | 0.44 |
| 42 | 3,7-iPr-PNX | (too fast)/3.0 ± 0.1 | (108 ± 3.7)/13.2 ± 0.1 | (61 ± 2.8)/30.8 ± | 6.52 ± |
| | | | | 0.1 | 0.45 |

Typical conditions: Egg-PC liposomes (1 mM); [STY-BOIDPY] = 1 μM (λex/λem = 488/518 nm); 0.2 mM DTUN; [RTA] = 4 μM or [RTA] = 0.4 μM + 10/100 μM ascorbate. Where inhibition was completely suppressed (at [RTA] = 4 μM, kᵢₙₕ > 2 × 10⁵ M⁻¹ s⁻¹), a label of “too fast” was applied.
^{a} Measured by monitoring loss of absorbance of STY-BOIDPY (λmax = 565 nm) at [RTA] = 2 μM.
^{b} Definitive end/inflection to inhibition/retardation not observed for calculation of tinh/n.
^{c} At low rates of inhibition, the assumption that kinh[RTA] >> 2 kt[ROO.] may breakdown leading to an overestimated kinh.
d No change in—[BODIPY]dt/was observed compared to an uninhibited experiment.
^{e} LogP values were calculated in ACD/ChemSketch 2020 (v. 14.50), ACD/Labs.

The kᵢₙₕ^{lip} values determined for the more reactive PNX and PTZ derivatives indicated that the increased inherent reactivity of electron-rich compounds did not translate from organic solution to liposomes. For example, the most inherently reactive derivative, 3,7-MeO-PNX (40), was characterized by kᵢₙₕ^{lip} of only 1.6×10⁵ M⁻¹s⁻¹, i.e., 30-fold lower than the prediction made based on its reactivity in solution and indistinguishable from that determined for unsubstituted PNX (kᵢₙₕ^{lip}=1.6×10⁵ M⁻¹s⁻¹). Likewise, 3,7-MeO-PTZ (32), 3,7-Me-PNX (35) and 3-MeO-PNX (39) were found to also have essentially equivalent rate constants (1.3-1.9×10⁵ M⁻¹s⁻¹), again in contrast to their predicted increase in kᵢₙₕ^{lip} relative to PNX (of 3.8-fold, 5.1-fold and 6.7-fold, respectively). It is important to note that the rate of STY-BODIPY consumption (-d[BODIPY]dt/) in the presence of these compounds did not drop below a slow (but consistently measurable) ~1×10−12 M⁻¹s⁻¹, seemingly agnostic to their differences in intrinsic reactivity. Thus, while the reactivity of electron-poor PNX and PTZ derivatives in phospholipid bilayers could be predicted from their solution phase kinetics, the reactivity of electron-rich PNX and PTZ derivatives could not. Instead, these compounds never appeared to exceed the reactivity exhibited by ‘electron-neutral’ PNX. This was most evident when kᵢₙₕ^{lip} values determined from the DTUN-initiated autoxidations were plotted against the values determined (or predicted) from the MeOAMVN-initiated autoxidations (FIG. 4(A)). The saturation-like behavior suggests some inherent limit to kᵢₙₕ^{lip}.

Surprisingly, certain electron-rich compounds such as 3,7-tBu-PNX (34) and 2,8-tBu-PNX (33) appeared to be substantially slower inhibitors than expected based upon their complete suppression of the MeOAMVN-initiated autoxidations. Their kᵢₙₕ^{lip} values (4.8×10⁴ and 3.9×10⁴ M⁻¹s⁻¹, respectively) did not correspond to the plateau of ~1-2×10⁵ M⁻¹s⁻¹ observed for unsubstituted PNX and the other activated PNX derivatives, including the electronically analogous 3,7-Me-PNX (35) (log P=4.76). We wondered if the increased lipid-solubility and smaller diffusion-coefficient of 34 (log P=7.22), and ostensibly 33, could render its regeneration by ascorbate at least partially rate-limiting. To examine this further, 3,7-Et-PNX (41) and 3,7-i-Pr-PNX (42) (log P=5.83 and 6.52, respectively) were prepared and tested to see if kᵢₙₕ^{lip} systematically changed along the simple alkyl series. Indeed, while in the absence of ascorbate regeneration all four compounds (at 4 μM) fully suppressed the autoxidation of STY-BODIPY (FIG. 4(B), top), but under synergistic conditions (FIG. 4(B), bottom) the kᵢₙₕ^{lip} of 3,7-Et-PNX (41) (7.7×10⁴ M⁻¹s⁻¹) and 3,7-iPr-PNX (42) (6.1×10⁴ M⁻¹s⁻¹) were found to be incrementally, and reproducibly, larger than that of 3,7-tBu-PNX (34) (4.8×10⁴ M⁻¹s⁻¹), and smaller than that of 3,7-Me-PNX (35) (1.4×10⁵ M⁻¹s⁻¹).

In each of the liposome experiments using the lipophilic initiator DTUN, addition of these compounds had a strong inhibitory effect (with the exception of 29 due to its intrinsically poor RTA kinetics), indicating the nearly universal lipophilic-nature of the PNX/PTZ cores, as water-soluble RTAs cannot affect the level of inhibition observed with 1-28 and 30-42 due to inaccessibility to LOO^{·} in the lipid-domain. This is an important property, with respect to the design of ferroptosis inhibitors, as water-soluble compounds which cannot inhibit LOO^{·} also show negligible activity against ferroptosis in cell-based assays (Shah et al., 2019), nearly without exception. Additional data for the inhibition of DTUN-initiated liposome autoxidation (see Table 4) aptly shows that: (a) intrinsic inhibitory activity and (b) lipophilicity are retained for compounds 43-95, which cover a broad range of additional substituent combinations and positions on the PNX scaffold. Of particular note within this subset are 43-48, 66, 69, 72, 75, 78, 81 and 84 which contain basic aminic moeities (pKa ≥10) and under the present conditions (pH 7.4), which endeavour to simulate physiological conditions, these would be in an ionized state (cationic). Nevertheless, these all demonstrate excellent inhibition of LOO^{·} further underscoring the intrinsically lipophilic-nature of these PNX-based inhibitors.

TABLE 4
Inhibition Rate Constants and Radical Trapping
Stoichiometries in DTUN Initiated Egg PC
Liposomes for Compounds 43-95.

| Compound | kᵢₙₕ^{lip} | |
|---|---|---|
| no. | (10³ M⁻¹s⁻¹) | n |
| 43 | (65 ± 6.6) | (5.0 ± 0.1) |
| 44 | (25 ± 0.3) | (7.3 ± 0.1) |
| 45 | (93 ± 7.3) | (2.5 ± 0.2) |
| 46 | (24 ± 3) | (4.2 ± 0.3) |
| 47 | (78 ± 6.6) | (2.4 ± 0.2) |
| 48 | (25 ± 3.9) | (4.2 ± 0.4) |
| 49 | (26) | (4.1) |
| 50 | (25 ± 0.6) | (5.2 ± 0.1) |
| 51 | (31 ± 2.4) | (4.6 ± 0.2) |
| 52 | (≥150) | (3.3) |
| 53 | (30 ± 0.8) | (3.4 ± 0.1) |
| 54 | (14 ± 0.6) | (3.4 ± 0.1) |
| 55 | (≥150) | (3.8 ± 0.1) |
| 56 | (24 ± 1.4) | (5.5 ± 0.1) |
| 57 | (49 ± 2.3) | (4.0 ± 0.1) |
| 58 | (54 ± 0.9) | (3.5 ± 0.1) |
| 59 | (55 ± 3.1) | (2.5 ± 0.1) |
| 60 | (63) | (2.9) |
| 61 | (61) | (2.7) |
| 62 | (56) | (3.3) |
| 63 | (62) | (3.5) |
| 64 | (17) | (2.4) |
| 65 | (60) | (2.8) |
| 66 | (38) | (3.7) |
| 67 | (49) | (2.6) |
| 68 | (34) | (4.1) |
| 69 | (44) | (2.8) |
| 70 | (88) | (3.1) |
| 71 | (84) | (2.5) |
| 72 | (54) | (3.1) |
| 73 | (83) | (3.4) |
| 74 | (65) | (3.3) |
| 75 | (42) | (2.4) |
| 76 | (95) | (2.9) |
| 77 | (44) | (3.4) |
| 78 | (42 ± 2) | (3.3 ± 0.1) |
| 79 | (71) | (2.1) |
| 80 | (64) | (2.6) |
| 81 | (≥150) | (1.6 ± 0.1) |
| 82 | (15 ± 0.7) | (2.2 ± 0.2) |
| 83 | (38 ± 3.9) | (1.8 ± 0.3) |
| 84 | (62 ± 3.5) | (2.0 ± 0.1) |
| 85 | (26) | (1.7) |
| 86 | (73) | (1.7) |
| 87 | (64 ± 1.5) | (2.1 ± 0.1) |
| 88 | (≥150) | (2.1 ± 0.1) |
| 89 | (67 ± 3.7) | (2.0 ± 0.1) |
| 90 | (142 ± 21) | (2.3 ± 0.1) |
| 91 | (48) | (3.8) |
| 92 | (72 ± 12) | (1.8 ± 0.1) |
| 93 | (13) | (2.2) |
| 94 | (≥150) | (2.4 ± 0.1) |
| 95 | (75 ± 4.8) | (1.8 ± 0.1) |

Typical conditions: Egg-PC liposomes (1 mM); [STY-BOIDPY] = 1 μM (λex/λem = 488/518 nm); 0.2 mM DTUN; [RTA] = 4 μM. No ascorbate was added. kᵢₙₕ and n are calculated from the average of at least two replicate experiments. Standard deviations calculated from a minimum of three replicate experiments. In cases where oxidation was completely suppressed by the inhibitor, the kᵢₙₕ is described as ≥150 × 10³ M⁻¹ s⁻¹, which is believed to be the upper limit of detection.

### Example 5. Ferroptosis Inhibition in Cells Induced Toward Ferroptotic Cell Death

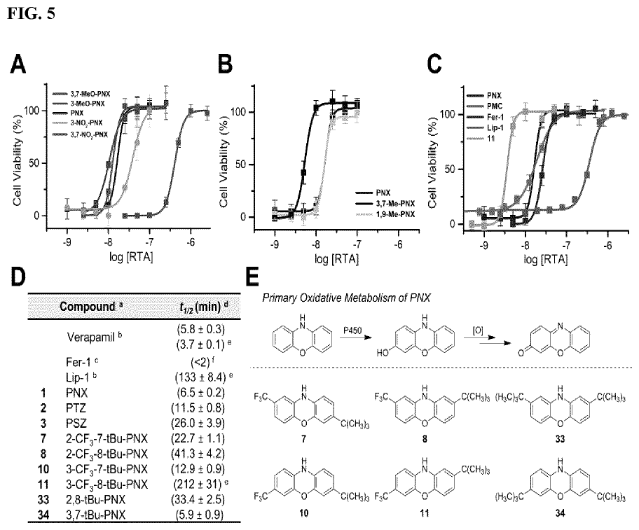

The potencies of the PNX and PTZ derivatives as ferroptosis inhibitors were evaluated in Pfa-1 mouse embryonic fibroblasts (MEFs) (Friedmann et al., 2014). Protection from RSL3-induced cell death (150 nM for 5 hours) was assessed by Aqua-Bluer assay and EC₅₀ values were derived from the corresponding survival curves. Representative examples are shown in FIGS. 5(A)-5(C) and results are also shown in Table 5. EC₅₀ values ranged over three orders of magnitude, from 4 nM to upwards of 1 M, but importantly, all of the PNX and PTZ derivatives—regardless of inherent RTA activity—inhibited cell death. Furthermore, many of the compounds were more potent than the ‘benchmark’ ferroptosis inhibitors Fer-1 and Lip-1, which had EC₅₀ values of 27 and 18 nM, respectively, under the assay conditions. Furthermore, in nearly every direct comparison between a PNX and a PTZ with the same substituents, the PNX compounds were systematically more potent. For example, the PNX compounds 1, 4, 6, 9, 15, 19, 34 and 36 were significantly more potent than their PTZ counterparts, 2, 5, 25, 26, 28, 29, 30 and 31. The only anomalies in this trend were 40 vs. 32, which were very similar to each other, and 12 vs. 27 where 12 was found to be less potent.

Introduction of EW groups on PNX (FIG. 5(A)) led to a clear decrease in potency (e.g., EC₅₀s of 16, 43 and 405 nM were determined for PNX, 3-NO2-PNX 15 and 3,7-NO2-PNX 19, respectively). Consistent with the decrease in inherent RTA activity (see above), the introduction of ED groups did not lead to a corresponding increase in potency (e.g., EC₅₀s of 16, 12 and 11 nM were determined for PNX, 3-MeO-PNX 39 and 3,7-MeO-PNX 40, respectively). A similar trend was observed with the PTZ derivatives, e.g., EC₅₀s for 3,7-NO2-PTZ 28, PTZ and 3,7-MeO-PTZ 32 were >1000 nM, 24 and 9, respectively. Further, when comparing compounds with similar electronics, derivatives of greater lipophilicity possessed greater potency. This was most evident from compounds featuring CF₃ substitution. Despite being an EW group, CF₃ introduction lead to more potent derivatives, e.g., 2-CF3-PNX 6 and 3-CF-PNX 9 had lower EC₅₀s (9 and 12 nM, respectively) than PNX (16 nM). Finally, steric hindrance around the reactive N—H appeared to have only a marginal impact on potency, as 3,7-di-Me-PNX 35 and 1,9-di-Me-PNX 36 (FIG. 5(B)) had EC₅₀s of 6 and 14 nM, respectively, suggesting that the modest electronic effect of methyl substitution on PNX potency was effectively negated by the steric effect.

Overall, consistently the most potent examples were ones that contained a CF₃ substituent (e.g., 7, 8, 10, 11, 44, and 46), all achieving single-digit nanomolar potency in the RSL3-induced cell death assay. Further to this point, systematically testing PNX compounds with (e.g., 44, 46 and 48) and without (e.g., 43, 45 and 47) the CF₃ group, it was found that those with this feature were significantly more potent in a systematic fashion.

TABLE 5
Anti-Ferroptosis Activity in RSL3 Induced
Mouse-Embryonic Fibroblasts.

| | | Pfa-1 MEF |
|---|---|---|
| | | Ferroptosis |
| Compound | | EC₅₀ |
| no. | RTA | (nM) |
| | Fer-1 | 27 ± 2 |
| | Lip-1 | 18 ± 1 |
| | PMC | 360 ± 30 |
| 1 | PNX | 16 ± 1 |
| 2 | PTZ | 24 ± 1 |
| 3 | PSZ | 23 ± 2 |
| 4 | Benzo(b)-PNX | 5.2 ± 0.9 |
| 5 | Benzo(b)-PTZ | 7.8 ± 0.2 |
| 6 | 2-CF3-PNX | 8.8 ± 0.5 |
| 7 | 2-CF3-7-tBu-PNX | 4.1 ± 0.1 |
| 8 | 2-CF3-8-tBu-PNX | 5.6 ± 0.3 |
| 9 | 3-CF3-PNX | 12 ± 1 |
| 10 | 3-CF3-7-tBu-PNX | 5.2 ± 0.2 |
| 11 | 3-CF3-8-tBu-PNX | 3.6 ± 0.2 |
| 12 | 3-CHO-PNX | 240 ± 23 |
| 13 | 3,7-CHO-PNX | 340 ± 49 |
| 14 | 3-CHO-7-CN-PNX | 240 ± 16 |
| 15 | 3-NO2-PNX | 43 ± 5 |
| 16 | 3-NO2-7-Me-PNX | 15 ± 1 |
| 17 | 3-NO2-7-MeO-PNX | 31 ± 1 |
| 18 | 3-NO2-7-NEt2-PNX | 14 ± 2 |
| 19 | 3,7-NO2-PNX | 405 ± 16 |
| 20 | 3-CN-PNX | 58 ± 1 |
| 21 | 3-CN-7-Me-PNX | 22 ± 5 |
| 22 | 3-CN-7-MeO-PNX | 42 ± 6 |
| 23 | 3-CN-7-NO2-PNX | 455 ± 19 |
| 24 | 2-C1-PTZ | 56 ± 10 |
| 25 | 2-CF3-PTZ | 16 ± 1 |
| 26 | 3-CF3-PTZ | 41 ± 5 |
| 27 | 3-CHO-PTZ | 140 ± 10 |
| 28 | 3-NO2-PTZ | 140 ± 18 |
| 29 | 3,7-NO2-PTZ ^{a} | ≥1000 |
| 30 | 3,7-tBu-PTZ | 8.9 ± 0.2 |
| 31 | 1,9-Me-PTZ | 35 ± 2 |
| 32 | 3,7-MeO-PTZ | 9.0 ± 0.5 |
| 33 | 2,8-tBu-PNX | 7.2 ± 0.4 |
| 34 | 3,7-tBu-PNX | 7.4 ± 0.1 |
| 35 | 3,7-Me-PNX | 5.4 ± 0.2 |
| 36 | 1,9-Me-PNX | 14 ± 2 |
| 37 | 2,4,6,8-Me-PNX | 9.6 ± 0.2 |
| 38 | 1,3,7,9-Me-PNX | 7.6 ± 0.2 |
| 39 | 3-MeO-PNX | 12 ± 1 |
| 40 | 3,7-MeO-PNX | 11 ± 1 |
| 43 | — | 15 ± 1 |
| 44 | — | 2.2 ± 0.1 |
| 45 | — | 13 ± 1 |
| 46 | — | 3.0 ± 0.1 |
| 47 | — | 45 ± 6 |
| 48 | — | 16 ± 2 |
| 60 | — | 3.0 ± 0.2 |
| 61 | — | 15 ± 1 |
| 65 | — | 31 ± 1 |

^{a} Compound 29 showed~50% viability at 1000 μM but a decrease in apparent cell viability above 1000 μM.

On the basis of our observations, the potency of ferroptosis inhibition was largely predicated on the following chemical properties: (a) RTA kinetics in the context of lipid-peroxidation (kᵢₙₕ^{lip}) and (b) relative lipophilicity. We assessed the contribution of these factors to the potency of the PNX and PTZ derivatives to inhibit ferroptosis in Pfa1 MEFs in terms of log kᵢₙₕ^{lip} and log P, respectively. Using only the compounds which had directly measurable kᵢₙₕ^{lip} values (for autoxidations initiated with MeOAMVN, Table 1), we found positive, but modest, correlations between potency (|log EC50|) and log kᵢₙₕ^{lip} (R²=0.506) or calculated log P (Table 3) (R²=0.587). However, performing a multiple linear regression incorporating both variables rendered an excellent correlation, with an adjusted R² of 0.796 (FIG. (6)), demonstrating that these chemical parameters (log kᵢₙₕ^{lip} and log P) can be useful for the prediction of ferroptosis inhibitor potency in further examples.

### Example 6. Metabolic Stability and the Relationship to Structure

One of the challenges in the development of ferroptosis inhibitors has been to design inhibitors that are resilient toward P450 enzymes, which facilitate first pass metabolic activity and are predominantly expressed in liver tissue, while retaining their inhibition activity. For example, Fer-1 is frequently used as a positive control in ferroptosis rescue assays because it demonstrates excellent potency, but it is not useful as an in vivo inhibitor of ferroptosis due to its complete lack of resilience toward P450 enzymes in the liver (Devisscher et al., 2018).

PNX is known to be metabolically labile, undergoing P450 mediated hydroxylation at the 3- and/or 7-positions to yield phenoxazone and resorufin derivatives (FIG. 5(E)) (Sutherland et al., 2001; Burke and Mayer, 1983). Results from liver microsomal stability experiments (BALB/c mouse) are also shown in Table 6.

Since all but the very electron-poor PNX derivatives were potent ferroptosis inhibitors, we expected that appropriately substituted derivatives may exhibit good metabolic stability and remain sufficiently potent to display activity in vivo. Thus, an initial subset of PNX derivatives with non-oxidizable substituents (i.e., tBu and CF₃) were subjected to liver microsome (BALB/c mouse) stability assays (FIG. 5(D)). To maintain a common basis for comparison, verapamil hydrochloride was used as a high clearance standard. Fer-1 and Lip-1 were also included for comparison.

TABLE 6
Summary of Liver Microsome Stability Data

| | |
|---|---|
| | (1) |
| 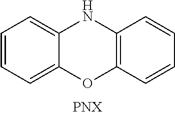 [SMILES: c1ccc2c(c1)Nc1ccccc1O2] | |
| | (34) |
| 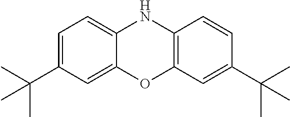 [SMILES: CC(C)(C)c1ccc2c(c1)Oc1cc(C(C)(C)C)ccc1N2] | |
| | (33) |
| 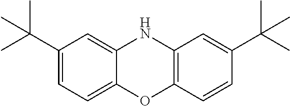 [SMILES: CC(C)(C)c1ccc2c(c1)Nc1cc(C(C)(C)C)ccc1O2] | |
| | (9) |
| 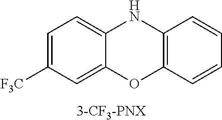 [SMILES: FC(F)(F)c1ccc2c(c1)Oc1ccccc1N2] | |
| | (7) |
| 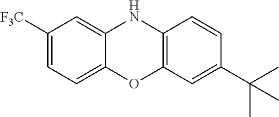 [SMILES: CC(C)(C)c1ccc2c(c1)Oc1ccc(C(F)(F)F)cc1N2] | |
| | (8) |
| 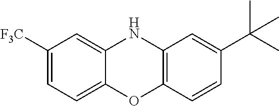 [SMILES: CC(C)(C)c1ccc2c(c1)Nc1cc(C(F)(F)F)ccc1O2] | |
| | (10) |
| 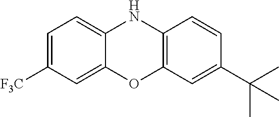 [SMILES: CC(C)(C)c1ccc2c(c1)Oc1cc(C(F)(F)F)ccc1N2] | |
| | (11) |
| 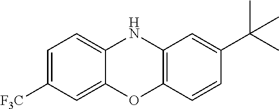 [SMILES: CC(C)(C)c1ccc2c(c1)Nc1ccc(C(F)(F)F)cc1O2] | |
| 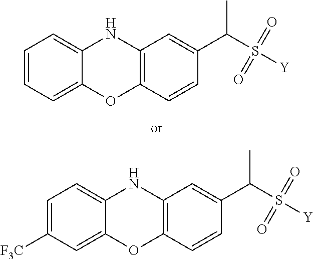 | |
| 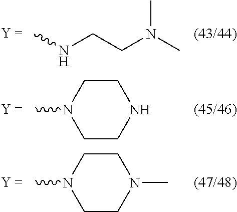 | |
| | (60) |
| 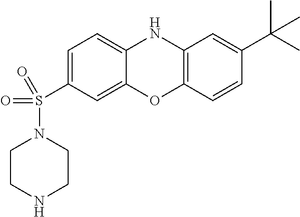 [SMILES: CC(C)(C)c1ccc2c(c1)Nc1ccc(S(=O)(=O)N3CCNCC3)cc1O2] | |

| Compound ^{a} | t_{1/2} (min) ^{c} | Relative t_{1/2} ^{f} | EC₅₀ (nM) ^{g} |
|---|---|---|---|
| Verapamil ^{b} | (5.8 ± 0.3) | 1.1 | N/A |
| | (3.7 ± 0.1) ^{d} | 1.0 | |
| PNX (1) | (6.5 ± 0.2) | (1) | 16 ± 1 |
| | (3.7) ^{d} | (1) | |
| 3-CF₃-PNX (9) | (9.1) | 1.4 | 12 ± 1 |
| 34 | (5.9 ± 0.9) | 0.9 | 7.4 ± 0.1 |
| 33 | (33.4 ± 2.5) | 5.1 | 7.2 ± 0.4 |
| 7 | (22.7 ± 1.1) | 3.5 | 4.1 ± 0.1 |
| 8 | (41.3 ± 4.2) | 6.4 | 5.6 ± 0.3 |
| 10 | (12.9 ± 0.9) | 2.0 | 5.2 ± 0.2 |
| 11 | (212 ± 31) ^{d} | 57 | 3.6 ± 0.2 |
| 60 | (97 ± 3) ^{d} | 26 | 3.0 ± 0.2 |
| 43 | (15.1) ^{d} | 4.1 | 15 ± 1 |
| 44 | (209) ^{d} | 56 | 2.2 ± 0.1 |
| 45 | (18.8) ^{d} | 5.1 | 13 ± 1 |
| 46 | (276) ^{d} | 75 | 3.0 ± 0.1 |
| 47 | (3.8) ^{d} | 1.0 | 45 ± 6 |
| 48 | (56.8) ^{d} | 15 | 16 ± 2 |
| Fer-1 | (>2) ^{e} | — | 27 ± 2 |
| Lip-1 | (133 ± 8.4) ^{d} | 36 | 18 ± 1 |
| PTZ (2) | (11.5 ± 0.8) | 1.8 | 24 ± 1 |
| 96 | (10.7 ± 0.4) | 1.6 | 6.5 ± 0.5 |

^{a} Results from liver microsomal stability experiments (BALB/c mouse). 10 μM of
compound (separated by RP-UPLC and monitored by PDA.)
^{b} Verapamil high-clearance standard at 1 μM (monitored by MS-ESI+)
^{c} Standard conditions: 0.5 mg/mL liver microsomes; 1 mM NADPH; 100 mM
phosphate buffer (pH 7.4); 37° C.
^{d} Liver microsomes increased (1.0 mg/mL).
^{e} At 0.5 mg/mL, Fer-1 was undetectable at 5 min., decreased to 0.1 mg/mL microsomes
where t_{1/2} = 5.3 ± 0.1 min was observed.
^{f} Half-life relative to PNX under equivalent experimental conditions.
^{g} EC50 derived from survival curve of RSL3 induced ferroptosis of pfa-1 MEFS
inhibited by ferroptosis inhibitors.

As expected, PNX was rapidly metabolized, with a half-life similar to that of verapamil. Fer-1 was even more rapidly metabolized and had to be evaluated at lower microsome concentration (0.1 mg/mL) to determine a reliable half-life. The lack of first pass metabolic stability of Fer-1, particularly in mouse liver microsomes, has been well documented (Devisscher et al., 2018). While PNX derivatives featuring substitution at the 3- and 7-positions (10 and 34) were no more stable than PNX itself, other isomers that would protect from hydroxylation at both ortho and para positions relative to the key N—H (i.e., 7, 8, 11 and 33), demonstrated greater stability. The most stable azine examined in this panel was 11 (t_{1/2}=212±31 min), so much so that the concentration of liver microsomes was increased (1 mg/mL) to more reliably measure its low t_{1/2}.

Further systematic testing, emulating the structural motif of 11, revealed that this was a uniquely favourable arrangement which enhanced metabolic stability >15-fold greater than the parent PNX (1), as demonstrated with compounds 44, 46, and 48. Removal of —CF₃ at the 7-position (e.g., 43, 45 and 47) led to significantly lower t_{1/2} (>10-fold decrease) compared to their aforementioned counterparts. On the other hand, in the absence of a benzylic carbon at the 2-position and only having the —CF₃ in the 7-position, as is the case for compound 9, was only marginally more stable than the parent compound 1 (~1.4-fold).

Inverting the positions of the substituents on 11, such as seen with 7 where CF₃ is at the 2-position and the benzylic alkyl is at the 7-position, did not lead to the same/similar effect with respect to stability. Indeed, the relative stability of 7 was >10-fold lower than that of 11.

Replacing the CF₃ on 11 with a different electron-withdrawing (EW) substituent such as a sulfonamide, as seen with 60, lead to a similar result where 60 was 26-fold more stable than the parent 1. This result suggests that the motif of 11 could be more generalized to other EW substituents outside of CF₃.

For each of compounds 11, 44, 46, 48, and 60, the potency for rescuing cells from RSL3 induced ferroptosis of pfa-1 MEFS was either retained or greatly enhanced compared to the parent 1. These compounds were also improved compared to other optimized ferroptosis inhibitors on other scaffolds. While Lip-1 has comparable stability, it was significantly less potent in the cell-based assay compared to compounds 11, 44, 46, and 60.

To further demonstrate the uniquely favourable stability/activity profile of the aforementioned motif, we elected to compare these to the lead PTZ-based ferroptosis inhibitor identified by Yang et al.: 2-(1-(4-(4-methylpiperazin-1-yl)phenyl)ethyl)-10H-phenothiazine (96) (Yang et al., 2021). While indeed 96 was more potent than its parent scaffold 2 in the RSL3-induced cell assay (EC₅₀ 6.5 nM vs. 24 nM) (though less potent compared to 11, 44, 46, and 60), 96 suffered from the same lack of stability as 2, both being only nominally more stable than the high clearance standard Verapamil and 1 (1.6 and 1.8-fold, respectively).

In summary, we found a particular motif, as demonstrated with compounds 11, 44, 46, 48 and 60, which not only enhances stability and lowers the intrinsic clearance of the ferroptosis inhibitors in an in vivo context (an essential aspect for therapeutic use), but also retains excellent potency with respect to inhibiting ferroptosis.

### Materials and Methods/Procedures for the Examples

Inhibited Co-autoxidation of PBD-BODIPY and 1,4-Dioxane. Into a 3 mL quartz cuvette were added 620 μL 1,4-dioxane (ACS grade) to either 1.80 mL of PhCl or 1.18 mL of PhCl with 620 μL of DMSO (ACS grade). The cuvette was placed in the sample holder of a UV-visible spectrophotometer and equilibrated to 37° C. PBD-BODIPY (12.5 L of a 2.0 mM solution in 1,2,4-trichlorobenzene), 50 μL AIBN solution (0.3 M in PhCl) followed by 10 μL of RTA solution (0.5 mM or desired concentration.) The contents were thoroughly mixed and the loss of PBD-BODIPY absorbance was monitored at 587 nm (without DMSO ε=123 000 M−1 cm−1, with DMSO ε=118 200 M−1 cm−1.) In parallel with the compounds of interest, an uninhibited experiment (no RTA) and one with a known concentration of PMC (2-6 μM) were recorded (the latter being used to calculate the rate of initiation assuming the n=2 for PMC.) The rate constants of inhibition (kinhdioxane or kinhDMSO), radical trapping stoichiometries (n) and associated standard deviations were ascertained from a minimum of three experimental replicates.

Inhibited Co-autoxidation of STY-BODIPY in Egg-PC Liposomes (Absorbance). Into a 3 mL quartz cuvette were combined 2.34 mL of 10 mM PBS (pH 7.4) and 125 μL of a suspension of egg-PC unilamellar liposomes (100 nm; 20 mM in the same PBS.) The cuvette was placed in the sample holder of a UV-visible spectrophotometer and equilibrated to 37° C. STY-BODIPY (12.5 μL of a 2.0 mM solution in DMSO), 10 μL MeOAMVN solution (0.05 M in MeCN) followed by 10 μL of RTA solution (0.5 mM or desired concentration.) The contents were thoroughly mixed and the loss of STY-BODIPY absorbance was monitored at 565 nm (ε=123 676 M−1 cm−1.) In parallel with the compounds of interest, an uninhibited experiment (no RTA) and one with a known concentration of PMC (2-6 μM) were recorded (the latter being used to calculate the rate of initiation assuming the n=2 for PMC.) The rate constants of inhibition (kᵢₙₕ^{lip}), radical trapping stoichiometries (n) and associated standard deviations were ascertained from a minimum of three experimental replicates.

Inhibited Co-autoxidation of STY-BODIPY in Egg-PC Liposomes (Fluorescence). Into a conical centrifuge tube of appropriate volume (15-50 mL) were charged with 10 mM PBS (pH 7.4), 1.04 mM egg-PC unilamellar liposomes (100 nm; 20 mM initial concentration) and 0.2 mM initiator (30 mM MeOAMVN in MeCN or 30 mM DTUN in EtOH) after which the tube was vortexed. When the RTA stock solutions were prepared and ready for addition by multi-channel pipette (240 M or desired concentration in MeCN), the 1.04 M STY-BODIPY (2 mM in DMSO initial concentration) was added to the liposome/initiator mixture and was subsequently vortexed (from this point the experiment should be set up as quickly as possible as the STY-BODIPY will progressively decompose). The liposome/initiator/dye mixture was then transferred to a reservoir and using a 300 μL multi-channel pipette a 96-well plate (black, Nunc) was charged with the mixture (295 μL per well). Using a 10 μL, the RTA solutions were added (5 L) bringing the final volume with 300 μL. The contents were mixed manually by taking up/dispensing the well contents 3-4 times with the 300 μL multi-channel pipette. The plate was mixed in the microplate reader for 1 min. at 37° C., after which monitoring loss of STY-BODIPY by fluorescence (λex/λem=488/518 nm) commenced for 15 h. In parallel with the compounds of interest, uninhibited experiments (no RTA) at multiple concentrations of STY-BODIPY (0.25, 0.5, 0.75, and 1 M) and experiments with a known concentration of PMC (4 M) were recorded. The former was used to determine the response factor of the oxidized STY-BODIPY at their maximum, while the latter was used to calculate the rate of initiation (Ri) assuming the n=2 for PMC. The rate constants of inhibition (kinhlip), radical trapping stoichiometries (n) and associated standard deviations were ascertained from a minimum of three experimental replicates.

Ascorbate Variant/Modification. Immediately before adding the STY-BODIPY, 10.4 or 104 M of ascorbate solution (10 or 100 mM initial concentration in PBS) is added to the conical tube. The RTA stocks are diluted further (from 240 M to 24 M) before addition to 96-well plate. The response factor and Ri are determined by the DTUN initiated autoxidation in the absence of ascorbate using the aforementioned uninhibited/PMC experiments.

Cell Culture. Cells were cultured at 37° C. under an atmosphere of 5% CO2. Pfa-1 mouse embryonic fibroblasts (MEFs) were cultured in high glucose DMEM (no phenol red) with 10% FBS, 10 mM glutamine, 100 μg/mL streptomycin and 100 IU/mL penicillin. The cells were passaged every other day by dissociation with 0.05% trypsin and 0.2% EDTA.

Inhibition of Ferroptosis in Cells Induced by (1S,3R)-RSL3. Into 96-well culture plates were seeded Pfa-1 MEFs (3000 in 100 μL of media) which were subsequently incubated overnight. The following day aliquots (1 μL) of test compound (i.e. RTA) serial diluted from a 10-50 mM DMSO stock were added and the plates were subsequently incubated for 1 h. This incubation was followed by the addition of (1S,3R)-RSL3 (10 μL at 1.65 μM in DPBS). The plate was incubated for 5 h after which cell viability was determined using the AquaBluer assay (MultiTarget Pharmaceuticals, LLC). Each experiment was carried out with a minimum of three technical replicates repeated three times.

Liver Microsome Stability Assay. A suspension of mouse (Balb-c) liver microsomes (Gibco™; BCMCPL; 20 mg/mL) are initially brought out of −78° C. storage and allowed to thaw in a container of crushed ice. Meanwhile, stock solutions of the test compounds are prepared and diluted to 0.1-1 mM in acetonitrile. A solution of 3:1 (v/v) MeCN/MeOH containing an HPLC standard (compound 25) at 15 μM was prepared and subsequently aliquoted (100 μL) into labelled microcentrifuge tubes. The tubes were placed in a freezer for later use. At this point, to 1155 μL of phosphate buffer (100 mM) was added 11.3 μL of MgCl2 solution (0.36 M) and 33.6 μL (or 67.2 μL) of the thawed liver microsomes. A 10 mM solution of beta-nicotinamide adenine dinucleotide phosphate reduced tetrasodium salt (NADPH) in phosphate buffer was prepared. The microsome solution was aliquoted into 4 wells in a 96-well culture plate (267 μL each) and the NADPH solution into a fifth well and the plate was heated in a microplate reader for ~5 min (with a lid.) The NADPH solution (30 μL), three test compounds, and Verapamil (positive control) were loaded (3 μL) into the wells containing the microsomes for a final concentration of 10 μM (or 1 μM) of test compound (or Verapamil), 1 mM NADPH, 3 mM MgCl2 and 0.5 mg/mL (or 1.0) microsomes. The suspensions were well-mixed, incubated for ~1 min. in the reader (37° C. with double-orbital shaking) and the first aliquots (50 μL) were quenched into the previously prepared cold MeCN/MeOH microcentrifuge tubes. The process was repeated four times at the desired time intervals after which the quenched samples were centrifuged (14 000 G, 10 min.) and the supernatant was transferred to HPLC vials with low volume inserts.

UPLC Analysis (Microsome Assay). The percent remaining concentration present in the samples was determined by the change of test compounds' integral (normalized to the HPLC standard) relative to the initial ‘0 min.’sample. Apart from Verapamil and Lip-1, which were monitored by ESI+ mass scan, the compounds were monitored by PDA (190-390 nm). The chromatography conditions were as follows: Column: Hypersil GOLD C-18 reverse phase column (250 mm×4.6 mm, 5 mm). UPLC method: injection volume of 10 mL; mobile phase A (pH 5.5 10 mM ammonium acetate buffer) and mobile phase B (methanol); flow rate: 0.5 mL/min; run time: 30 min; the gradient elution method: 80% to 100% B from 0 to 10 min, hold at 100% B from 10 to 25 min, 100% to 80% B from 25 to 30 min.

Stoichiometry and Rate Constant Determination. Rate of STY/PBD-BODIPY consumption during inhibition by the RTA:

$$\frac{- d [ BODIPY ]}{dt} = \frac{{k}_{BODIPY} [ BODIPY ] {R}_{\text{?}}}{{nk}_{inh} [ RTA ]} \qquad ( 1 )$$

$$\text{?} \text{indicates text missing or illegible when filed}$$

Rearranged to isolate the rate constant of inhibition (k_{ing}):

$${k}_{inh} = \frac{{k}_{BODIPY} [ BODIPY ] {R}_{i}}{n \frac{- d [ BODIPY \text{?}}{dt} [ RTA ]} \qquad ( 2 )$$

$$\text{?} \text{indicates text missing or illegible when filed}$$

where: ‘kBODIPY’ is the rate constant of radical addition to STY or PBD-BODIPY (M⁻¹ s⁻¹); ‘[BODIPY]’ is the initial concentration (M) of STY or PBD-BODIPY; ‘R₁’ is the rate of initiation (determined from the tᵢₙₕ of 2,2,5,7,8-Pentamethyl-6-chromanol (PMC) using Eq. 4 assuming n=2); ‘n’ is the stoichiometry of the radical trapping antioxidant (RTA) calculated using Eq. 3; ‘[RTA]’ is the initial concentration of the radical trapping antioxidant (M). Note stoichiometry for the determination of kᵢₙₕ was assumed to be 2 when the observed n≥2; when n<2, the observed n was used in the calculation of kᵢₙₕ.

### RTA Stoichiometry (n):

$$n = \frac{{\text{?}}_{inh} \cdot {R}_{\text{?}}}{[ RTA ]} \qquad ( 3 )$$

$$\text{?} \text{indicates text missing or illegible when filed}$$

Rearranged to isolate the rate of initiation (Ri):

$$\text{?} \\ {R}_{\text{?}} = \frac{n [ RTA ]}{{\text{?}}_{inh}} \qquad ( 4 )$$

$$\text{?} \text{indicates text missing or illegible when filed}$$

where: ‘tᵢₙₕ’ is the time of inhibition (which is the intercept between the tangent of the slope inhibition and slope of post-inhibition).

### REFERENCES

Abraham, M. H.; Abraham, R. J.; Byrne, J.; Griffiths, L. NMR Method for the Determination of Solute Hydrogen Bond Acidity. J. Org. Chem. 2006, 71, 3389-3394.
Barclay, L. R. C.; Baskin, K. A.; Dakin, K. A.; Locke, S. J.; Vinqvist, M. R. The Antioxidant Activities of Phenolic Antioxidants in Free Radical Peroxidation of Phospholipid Membranes. Can. J. Chem. 1990, 68 (12), 2258-2269.
Burton, G. W.; Doba, T.; Gabe, E. J.; Hughes, L.; Lee, F. L.; Prasad, L.; Ingoldo, K. U. Autoxidation of Biological Molecules. 4. Maximizing the Antioxidant Activity of Phenols. J. Am. Chem. Soc. 1985, 107 (24), 7053-7065.
Conrad, M.; Pratt, D. A. The Chemical Basis of Ferroptosis. Nat. Chem. Biol. 2019, 15 (12), 1137-1147.
Danny Burke, M.; Mayer, R. T. Differential Effects of Phenobarbitone and 3-Methylcholanthrene Induction on the Hepatic Microsomal Metabolism and Cytochrome P-450-Binding of Phenoxazone and a Homologous Series of Its n-Alkyl Ethers (Alkoxyresorufins). Chem. Biol. Interact. 1983, 45 (2), 243-258.
Devisscher, L.; Van Coillie, S.; Hofmans, S.; Van Rompaey, D.; Goossens, K.; Meul, E.; Maes, L.; De Winter, H.; Van Der Veken, P.; Vandenabeele, P.; Berghe, T. Vanden; Augustyns, K. Discovery of Novel, Drug-Like Ferroptosis Inhibitors with in Vivo Efficacy. J. Med. Chem. 2018, 61 (22), 10126-10140.
Dixon, S. J.; Lemberg, K. M.; Lamprecht, M. R.; Skouta, R.; Zaitsev, E. M.; Gleason, C. E.; Patel, D. N.; Bauer, A. J.; Cantley, A. M.; Yang, W. S.; Morrison, B.; Stockwell, B. R. Ferroptosis: An Iron-Dependent Form of Nonapoptotic Cell Death. Cell 2012, 149 (5), 1060-1072.
Doba, T.; Burton, G. W.; Ingold, K. U. Antioxidant and Co-Antioxidant Activity of Vitamin C. The Effect of Vitamin C, Either Alone or in the Presence of Vitamin E or a Water-Soluble Vitamin E Analogue, upon the Peroxidation of Aqueous Multilamellar Phospholipid Liposomes. Biochim. Biophys. Acta—Lipids Lipid Metab. 1985, 835 (2), 298-303.
Doll, S.; Freitas, F. P.; Shah, R.; Aldrovandi, M.; da Silva, M. C.; Ingold, I.; Goya Grocin, A.; Xavier da Silva, T. N.; Panzilius, E.; Scheel, C. H.; Mourão, A.; Buday, K.; Sato, M.; Wanninger, J.; Vignane, T.; Mohana, V.; Rehberg, M.; Flatley, A.; Schepers, A.; Kurz, A.; White, D.; Sauer, M.; Sattler, M.; Tate, E. W.; Schmitz, W.; Schulze, A.; O'Donnell, V.; Proneth, B.; Popowicz, G. M.; Pratt, D. A.; Angeli, J. P. F.; Conrad, M. FSP1 Is a Glutathione-Independent Ferroptosis Suppressor. Nature 2019, 575 (7784), 693-698.
Farmer, L. A.; Haidasz, E. A.; Griesser, M.; Pratt, D. A. Phenoxazine: A Privileged Scaffold for Radical-Trapping Antioxidants. J. Org. Chem. 2017, 82 (19), 10523-10536. Friedmann Angeli, J. P.; Schneider, M.; Proneth, B.; Tyurina, Y. Y.; Tyurin, V. A.; Hammond, V. J.; Herbach, N.; Aichler, M.; Walch, A.; Eggenhofer, E.; Basavarajappa, D.; Radmark, O.; Kobayashi, S.; Seibt, T.; Beck, H.; Neff, F.; Esposito, I.; Wanke, R.; Forster, H.; Yefremova, O.; Heinrichmeyer, M.; Bornkamm, G. W.; Geissler, E. K.; Thomas, S. B.; Stockwell, B. R.; O'Donnell, V. B.; Kagan, V. E.; Schick, J. A.; Conrad, M. Inactivation of the Ferroptosis Regulator Gpx4 Triggers Acute Renal Failure in Mice. Nat. Cell Biol. 2014, 16 (12), 1180-1191.
Haeggström, J. Z.; Funk, C. D. Lipoxygenase and Leukotriene Pathways: Biochemistry, Biology, and Roles in Disease. Chem. Rev. 2011, 111 (10), 5866-5898.
Hajieva, P., Mocko, J. B., Moosmann, B., and Behl, C. Novel imine antioxidants at low nanomolar concentrations protect dopaminergic cells from oxidative neurotoxicity. Journal of Neurochemistry 2009, 110: 118-132.
Helberg, J.; Pratt, D. A. Autoxidation vs. Antioxidants—the Fight for Forever. Chem. Soc. Rev. 2021, 50 (13), 7343-7358.
Jiang, X.; Stockwell, B. R.; Conrad, M. Ferroptosis: Mechanisms, Biology and Role in Disease. Nat. Rev. Mol. Cell Biol. 2021, 22 (4), 266-282.
Lewerenz, J., Ates, G., Methner, A., Conrad, M. and Maher, P. Oxytosis/Ferroptosis-(Re-) Emerging Roles for Oxidative Stress-Dependent Non-apoptotic Cell Death in Diseases of the Central Nervous System. Front. Neurosci., 2018, 12: 1-23.
Lucarini, M.; Pedrielli, P.; Pedulli, G. F.; Valgimigli, L.; Gigmes, D.; Tordo, P. Bond Dissociation Energies of the N—H Bond and Rate Constants for the Reaction with Alkyl, Alkoxyl, and Peroxyl Radicals of Phenothiazines and Related Compounds. J. Am. Chem. Soc. 1999, 121, 11546-11553.
Masaldan, S.; Bush, A. I.; Devos, D.; Rolland, A. S.; Moreau, C. Striking While the Iron Is Hot: Iron Metabolism and Ferroptosis in Neurodegeneration. Free Radic. Biol. Med. 2019, 133, 221-233.
Mocko, J. B., Kern, A., Moosmann, B., Behl, C., and Hajieva, O. Phenothiazines interfere with dopaminergic neurodegeneration in Caenorhabditis elegans models of Parkinson's disease. Neurobiology of Diseaes 2010, 40 (1), 120-129.
Niki, E.; Kawakami, A.; Yamamoto, Y.; Kamiya, Y. Oxidation of Lipids. VIII. Synergistic Inhibition of Oxidation of Phosphatidylcholine Liposome in Aqueous Dispersion by Vitamin E and Vitamin C. Bull. Chem. Soc. Jpn. 1985, 58 (7), 1971-1975.
Riegman, M.; Sagie, L.; Galed, C.; Levin, T.; Steinberg, N.; Dixon, S. J.; Wiesner, U.; Bradbury, M. S.; Niethammer, P.; Zaritsky, A.; Overholtzer, M. Ferroptosis Occurs through an Osmotic Mechanism and Propagates Independently of Cell Rupture. Nat. Cell Biol. 2020, 22 (9), 1042-1048.
Sato, K.; Niki, E.; Shimasaki, H. Free Radical-Mediated Chain Oxidation of Low Density Lipoprotein and Its Synergistic Inhibition by Vitamin E and Vitamin C. Arch. Biochem. Biophys. 1990, 279 (2), 402-405.
Shah, R.; Margison, K.; Pratt, D. A. The Potency of Diarylamine Radical-Trapping Antioxidants as Inhibitors of Ferroptosis Underscores the Role of Autoxidation in the Mechanism of Cell Death. ACS Chem. Biol. 2017, 12 (10), 2538-2545.
Shah, R.; Farmer, L. A.; Zilka, O.; Van Kessel, A. T. M.; Pratt, D. A. Beyond DPPH: Use of Fluorescence-Enabled Inhibited Autoxidation to Predict Oxidative Cell Death Rescue. Cell Chemical Biology. 2019, pp 1594-1607.e7.
Snelgrove, D. W.; Lusztyk, J.; Banks, J. T.; Mulder, P.; Ingold, K. U. Kinetic Solvent Effects on Hydrogen-Atom Abstractions: Reliable, Quantitative Predictions via a Single Empirical Equation. J. Am. Chem. Soc. 2001, 123 (3), 469-477.
Soula, M.; Weber, R. A.; Zilka, O.; Alwaseem, H.; La, K.; Yen, F.; Molina, H.; Garcia-Bermudez, J.; Pratt, D. A.; Birsoy, K. Metabolic Determinants of Cancer Cell Sensitivity to Canonical Ferroptosis Inducers. Nat. Chem. Biol. 2020, 16 (12), 1351-1360.
Sutherland, J. B.; Freeman, J. P.; Heinze, T. M.; Moody, J. D.; Parshikov, I. A.; Williams, A. J.; Zhang, D. Oxidation of Phenothiazine and Phenoxazine by Cunninghamella Elegans. Xenobiotica 2001, 31 (11), 799-809.
Stockwell, B. R.; Jiang, X. The Chemistry and Biology of Ferroptosis. Cell Chem. Biol. 2020, 27 (4), 365-375.
Stockwell, B. R.; Friedmann Angeli, J. P.; Bayir, H.; Bush, A. I.; Conrad, M.; Dixon, S. J.; Fulda, S.; Gascón, S.; Hatzios, S. K.; Kagan, V. E.; Noel, K.; Jiang, X.; Linkermann, A.; Murphy, M. E.; Overholtzer, M.; Oyagi, A.; Pagnussat, G. C.; Park, J.; Ran, Q.; Rosenfeld, C. S.; Salnikow, K.; Tang, D.; Torti, F. M.; Torti, S. V; Toyokuni, S.; Woerpel, K. A.; Zhang, D. D. Ferroptosis: A Regulated Cell Death Nexus Linking Metabolism, Redox Biology, and Disease. Cell 2017, 171 (2), 273-285.
Yang, W.; Liu, X.; Song, C.; Ji, S.; Yang, J.; Liu, Y.; You, J.; Zhang, J.; Huang, S.; Cheng, W.; Shao, Z.; Li, L.; Yang, S. Structure-Activity Relationship Studies of Phenothiazine Derivatives as a New Class of Ferroptosis Inhibitors Together with the Therapeutic Effect in an Ischemic Stroke Model. Eur. J. Med. Chem. 2021, 209, 112842. https://doi.org/https://doi.org/10.1016/j.ejmech.2020.112842.
Yin, H.; Xu, L.; Porter, N. A. Free Radical Lipid Peroxidation: Mechanisms and Analysis. Chem. Rev. 2011, 111 (10), 5944-5972.
Yu, M. J., McCowan, J. R., Bertsch, B., and Ho, P. P. K. Enzymic and nonenzymic lipid peroxidation: inhibition by substituted phenoxazines. Bioorganic & Medicinal Chemistry Letters. 1991. 1(2): 107-110.
Zielinski, Z. A. M.; Pratt, D. A. Lipid Peroxidation: Kinetics, Mechanisms, and Products. J. Org. Chem. 2017, 82 (6), 2817-2825.
Zilka, O.; Shah, R.; Li, B.; Friedmann Angeli, J. P.; Griesser, M.; Conrad, M.; Pratt, D. A. On the Mechanism of Cytoprotection by Ferrostatin-1 and Liproxstatin-1 and the Role of Lipid Peroxidation in Ferroptotic Cell Death. ACS Cent. Sci. 2017, 3 (3), 232-243.

Although this invention is described in detail with reference to embodiments thereof, these embodiments are offered to illustrate but not to limit the invention. It is possible to make other embodiments that employ the principles of the invention and that fall within its spirit and scope as defined by the claims appended hereto.

The contents of all documents and references cited herein are hereby incorporated by reference in their entirety.

What is claimed is:

1. A compound of Formula A, or a pharmaceutically acceptable salt thereof: 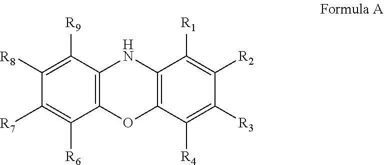
wherein:
R₁, R₂, R₃, and R₄ are independently hydrogen (H), substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid, an ester, an amide, nitro (NO₂), fluoro (F), chloro (Cl), bromo (Br), iodo (I), formyl (CH(O)), acetyl (C(O)CH₃), a phosphonyl group, or a sulfonyl group; or
R₁ and R₂, taken together with the atoms to which they are attached, form a substituted or unsubstituted carbocyclic ring; or
R₂ and R₃, taken together with the atoms to which they are attached, form a substituted or unsubstituted carbocyclic ring; or
R₃ and R₄, taken together with the atoms to which they are attached, form a substituted or unsubstituted carbocyclic ring; or
R₆ and R₇, taken together with the atoms to which they are attached, form a substituted or unsubstituted carbocyclic ring; or
R₇ and R₈, taken together with the atoms to which they are attached, form a substituted or unsubstituted carbocyclic ring; or
R₈ and R₉, taken together with the atoms to which they are attached, form a substituted or unsubstituted carbocyclic ring;
provided that:
R₁, R₂, R₃, R₄, R₆, R₇, R₈, and R₉ are not all H at the same time;
R₁ and R₂ are not COOH, COO—, or (CH₂)₂COO—;
when R₁, R₂ and R₄ are hydrogen, R₃ is not N(CH₂CH₃)₂; and
when at least one of R₇ and R₈ is NO₂, R₂ and R₃ are not OCH₃.

2. The compound or pharmaceutically acceptable salt of claim 1, wherein, when at least one of R₇ and R₈ is CF₃, R₃ is not also CF₃ and R₂ is not NO₂.

3. The compound or pharmaceutically acceptable salt of claim 1 or 2, wherein, when at least one of R₇ and R₈ is CN, R₂ and R₃ are not also CN, and R₃ is not F.

4. A compound of Formula I or Formula II, or a pharmaceutically acceptable salt thereof: 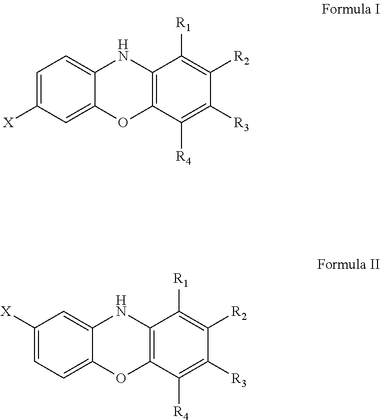
wherein:
X is trifluoromethyl (CF₃), a perfluoroalkyl (CₙF₂ₙ₊₁ where n is an integer from 1 to 6), nitro (NO₂), cyano (CN), sulfonamide (SO₂N(R)₂), trifluoromethyl ether (OCF₃), a perfluoroalkyl ether (—OCₘF₂ₘ₊₁ where m is an integer from 1 to 6), fluoro (F), chloro (Cl), bromo (Br), iodo (I), formyl (CH(O)), acetyl (C(O)CH₃), an ester, an amide, or a ketone; and
R₁, R₂, R₃, and R₄ are independently hydrogen (H), substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid, an ester, an amide, nitro (NO₂), fluoro (F), chloro (Cl), bromo (Br), iodo (I), formyl (CH(O)), acetyl (C(O)CH₃), a phosphonyl group, or a sulfonyl group; or
R₁ and R₂, taken together with the atoms to which they are attached, form a substituted or unsubstituted carbocyclic ring; or
R₂ and R₃, taken together with the atoms to which they are attached, form a substituted or unsubstituted carbocyclic ring; or
R₃ and R₄, taken together with the atoms to which they are attached, form a substituted or unsubstituted carbocyclic ring;
provided that:
R₁ is not COOH;
R₁, R₂, R₃, and R₄ are not all H at the same time;
when R₁, R₂ and R₄ are hydrogen, R₃ is not N(CH₂CH₃)₂; and
when X is NO₂, R₂ and R₃ are not OCH₃.

5. The compound or pharmaceutically acceptable salt of claim 4, wherein, when X is CF₃, R₃ is not also CF₃ and R₂ is not NO₂.

6. The compound or pharmaceutically acceptable salt of claim 4 or 5, wherein, when X is CN, R₂ and R₃ are not also CN, and R₃ is not F.

7. The compound or pharmaceutically acceptable salt of any one of claims 1 to 6, wherein R₁ and R₄ are hydrogen.

8. The compound or pharmaceutically acceptable salt of any one of claims 1 to 7, wherein R₁, R₃ and R₄ are hydrogen.

9. The compound or pharmaceutically acceptable salt of any one of claims 1 to 7, wherein R₁, R₂ and R₄ are hydrogen.

10. The compound or pharmaceutically acceptable salt of any one of claims 4 to 9, wherein X is trifluoromethyl (CF₃).

11. The compound or pharmaceutically acceptable salt of any one of the preceding claims, wherein R₆ and R₉ are hydrogen.

12. The compound or pharmaceutically acceptable salt of any one of the preceding claims, wherein R₆, R₇ and R₉ are hydrogen.

13. The compound or pharmaceutically acceptable salt of any one of the preceding claims, wherein R₆, R₈ and R₉ are hydrogen.

14. The compound or pharmaceutically acceptable salt of any one of the preceding claims, wherein at least one of R₂, R₃, R₇ and R₈ is trifluoromethyl (CF₃).

15. The compound or pharmaceutically acceptable salt of any one of claims 4 to 14, wherein:
when X is an ester, R₂ is not an ester; or,
when X is an amide, R₂ and/or R₃ is not an amide; or,
when X is an amide, R₂ and/or R₃ is not an amide, and R₃ is not an acid; or, when X is an amide, R₂ and R₃ are not an amide, and R₃ is not an acid.

16. A compound of Formula III or Formula IV, or a pharmaceutically acceptable salt thereof: 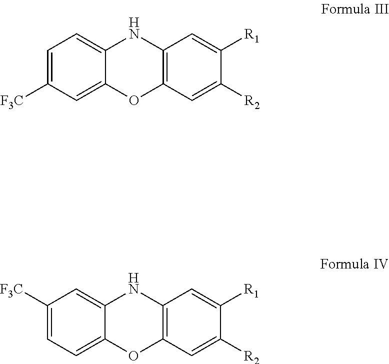
wherein:
R₁ and R₂ are independently selected from hydrogen (H), substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid, nitro (NO₂), fluoro (F), chloro (Cl), bromo (Br), iodo (I), formyl (CH(O)), acetyl (C(O)CH₃), a phosphonyl group, and a sulfonyl group; and
at least one of R₁ and R₂ is not H.

17. The compound or pharmaceutically acceptable salt of claim 16, wherein R₁ is hydrogen (H).

18. The compound or pharmaceutically acceptable salt of claim 16, wherein R₂ is hydrogen (H).

19. The compound or pharmaceutically acceptable salt of claim 16, wherein R₁ and R₂ are both not hydrogen (H).

20. The compound or pharmaceutically acceptable salt thereof of any one of the claims 1 to 19, wherein the compound is a compound shown in Table 1; Table 2; or a pharmaceutically acceptable salt thereof.

21. A compound shown in Table 1 or Table 2, or a pharmaceutically acceptable salt thereof.

22. The compound or pharmaceutically acceptable salt of any one of claims 1 to 21, wherein the compound is any one of compounds 4, 6-23, 33-95, or a pharmaceutically acceptable salt thereof.

23. The compound or pharmaceutically acceptable salt of claim 22, wherein the compound is compound 11, or a pharmaceutically acceptable salt thereof.

24. The compound or pharmaceutically acceptable salt of claim 22, wherein the compound is compound 44, or a pharmaceutically acceptable salt thereof.

25. The compound or pharmaceutically acceptable salt of claim 22, wherein the compound is compound 46, or a pharmaceutically acceptable salt thereof.

26. The compound or pharmaceutically acceptable salt of claim 22, wherein the compound is compound 48, or a pharmaceutically acceptable salt thereof.

27. The compound or pharmaceutically acceptable salt of claim 22, wherein the compound is compound 60, or a pharmaceutically acceptable salt thereof.

28. The compound or pharmaceutically acceptable salt of claim 22, wherein the compound is compound 66, or a pharmaceutically acceptable salt thereof.

29. The compound or pharmaceutically acceptable salt of any one of claims 1 to 28, wherein the compound is a ferroptosis inhibitor.

30. The compound or pharmaceutically acceptable salt of any one of claims 1 to 29, wherein the compound can inhibit lipid autoxidation, is a radical-trapping antioxidant, and/or can reduce oxidative stress.

31. A pharmaceutical composition comprising the compound of any one of claims 1 to 30 or a pharmaceutically acceptable salt thereof, and a pharmaceutically acceptable carrier.

32. The pharmaceutical composition of claim 31, wherein the composition is suitable for administration orally, intravenously, intraperitoneally, intramuscularly, subcutaneously, sublingually, topically, mucosally, nasally, or via inhalation.

33. A method of inhibiting ferroptosis and/or oxytosis in a subject, comprising administering to the subject the compound or pharmaceutically acceptable salt of any one of claims 1 to 30, or the pharmaceutical composition of claim 31 or 32, such that ferroptosis and/or oxytosis is inhibited in the subject.

34. The method of claim 33, wherein lipid autoxidation is inhibited and/or oxidative stress is reduced in the subject.

35. A method of reducing oxidative stress in a subject, comprising administering to the subject the compound or pharmaceutically acceptable salt of any one of claims 1 to 30, or the pharmaceutical composition of claim 31 or 32, such that oxidative stress is reduced in the subject.

36. The method of any one of claims 33 to 35, wherein the subject suffers from, or is at risk of developing, a ferroptosis-associated disease or disorder.

37. A method of preventing or treating a ferroptosis-associated disease or disorder in a subject in need thereof, comprising administering to the subject an effective amount of the compound or pharmaceutically acceptable salt of any one of claims 1 to 30, or the pharmaceutical composition of claim 31 or 32.

38. The method of claim 36 or 37, wherein the ferroptosis-associated disease or disorder is stroke, myocardial infarction, diabetes, sepsis, transplant rejection, a neurodegenerative disease, a central nervous system (CNS) injury, an ischemic event, or a cancer.

39. The method of claim 38, wherein the neurodegenerative disease is Alzheimer's disease, Huntington's disease, Parkinson's disease, amyotrophic lateral sclerosis (ALS), HIV-associated dementia, cerebral ischemia, multiple sclerosis, Lewy body disease, Menke's disease, Wilson's disease, Creutzfeldt-Jakob disease, Fahr disease, Friedreich's ataxia, and frontotemporal dementia, amyloidosis, Tay-Sachs disease or periventricular leukomalacia.

40. The method of claim 38, wherein the CNS injury is traumatic brain injury, spinal cord injury, or stroke.

41. The method of claim 38, wherein the ischemic event is cerebral ischemia, ischemic stroke, organ ischemia, myocardial infarction, mesenteric, retinal, hepatic or brain ischemic injury, or ischemic-reperfusion injury.

42. A method for preventing or treating a neurodegenerative disease in a subject in need thereof, comprising administering to the subject an effective amount of the compound or pharmaceutically acceptable salt of any one of claims 1 to 30, or the pharmaceutical composition of claim 31 or 32.

43. The method of claim 42, wherein the neurodegenerative disease is Alzheimer's disease, Huntington's disease, Parkinson's disease, amyotrophic lateral sclerosis (ALS), HIV-associated dementia, cerebral ischemia, multiple sclerosis, Lewy body disease, Menke's disease, Wilson's disease, Creutzfeldt-Jakob disease, Fahr disease, Friedreich's ataxia, and frontotemporal dementia, amyloidosis, Tay-Sachs disease or periventricular leukomalacia.

44. A method for preventing or treating ischemic-reperfusion injury in a subject in need thereof, comprising administering to the subject an effective amount of the compound or pharmaceutically acceptable salt of any one of claims 1 to 30, or the pharmaceutical composition of claim 31 or 32.

45. The method of any one of claims 35 to 44, wherein one or more of the following is inhibited or reduced in the subject: ferroptosis and/or oxytosis; oxidative stress; and lipid autoxidation.

46. The method of any one of claims 33 to 45, wherein said compound or pharmaceutically acceptable salt thereof is administered orally.

47. The method of any one of claims 33 to 45, wherein said compound or pharmaceutically acceptable salt thereof is administered topically and/or locally at the site of an injury.

48. The method of any one of claims 33 to 45, wherein said compound or pharmaceutically acceptable salt thereof is administered parenterally or by injection.

49. The method of claim 48, wherein said compound or pharmaceutically acceptable salt thereof is administered intravenously, intraperitoneally, intramuscularly, or subcutaneously.

50. The method of any one of claims 33 to 45, wherein said compound or pharmaceutically acceptable salt thereof is administered, intranasally, mucosally, or by inhalation.

51. The method of any one of claims 33 to 50, wherein said subject is a human.

52. A method of inhibiting ferroptosis and/or oxytosis in a subject, comprising administering to the subject a compound of Formula A or a pharmaceutically acceptable salt thereof: 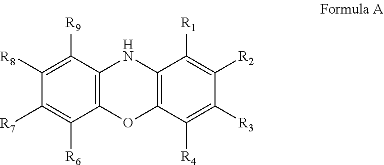
wherein:
R₁, R₂, R₃, and R₄ are independently hydrogen (H), substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid, an ester, an amide, nitro (NO₂), fluoro (F), chloro (Cl), bromo (Br), iodo (I), formyl (CH(O)), acetyl (C(O)CH₃), a phosphonyl group, or a sulfonyl group; or
R₁ and R₂, taken together with the atoms to which they are attached, form a substituted or unsubstituted carbocyclic ring; or
R₂ and R₃, taken together with the atoms to which they are attached, form a substituted or unsubstituted carbocyclic ring; or
R₃ and R₄, taken together with the atoms to which they are attached, form a substituted or unsubstituted carbocyclic ring; or
R₆ and R₇, taken together with the atoms to which they are attached, form a substituted or unsubstituted carbocyclic ring; or
R₇ and R₈, taken together with the atoms to which they are attached, form a substituted or unsubstituted carbocyclic ring; or
R₈ and R₉, taken together with the atoms to which they are attached, form a substituted or unsubstituted carbocyclic ring;
provided that R₁, R₂, R₃, R₄, R₆, R₇, R₈, and R₉ are not all H at the same time.

53. The method of claim 52, wherein, when R₆ or R₇ is CF₃, R₃ is not also CF₃ and R₂ is not NO₂.

54. The method of claim 52 or 53, wherein, when R₆ or R₇ is CN, R₂ and R₃ are not also CN, and R₃ is not F.

55. The method of any one of claims 52 to 54, wherein, when R₆ or R₇ is NO₂, R₂ and R₃ are not OCH₃.

56. The method of any one of claims 52 to 55, wherein at least one of R₆ and R₇ is trifluoromethyl (CF₃).

57. The method of any one of claims 52 to 56, wherein at least one of R₂ and R₃ is not H.

58. A method of inhibiting ferroptosis and/or oxytosis in a subject, comprising administering to the subject a compound of Formula I or Formula II, or a pharmaceutically acceptable salt thereof: 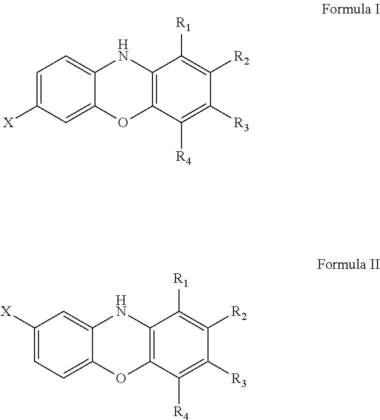
wherein:
X is trifluoromethyl (CF₃), a perfluoroalkyl (CₙF₂ₙ₊₁ where n is an integer from 1 to 6), nitro (NO₂), cyano (CN), sulfonamide (SO₂N(R)₂), trifluoromethyl ether (OCF₃), a perfluoroalkyl ether (—OCₘF₂ₘ₊₁ where m is an integer from 1 to 6), fluoro (F), chloro (Cl), bromo (Br), iodo (I), formyl (CH(O)), acetyl (C(O)CH₃), an ester, an amide, or a ketone; and
R₁, R₂, R₃, and R₄ are independently hydrogen (H), substituted or unsubstituted alkyl, substituted or unsubstituted aryl, substituted or unsubstituted arylalkyl, substituted or unsubstituted alkylaryl, substituted or unsubstituted alkoxycarbonyl, substituted or unsubstituted aryloxycarbonyl, substituted or unsubstituted arylalkoxycarbonyl, a cyclic or heterocyclic moiety, an acyl group derived from a carboxylic acid, an ester, an amide, nitro (NO₂), fluoro (F), chloro (Cl), bromo (Br), iodo (I), formyl (CH(O)), acetyl (C(O)CH₃), a phosphonyl group, or a sulfonyl group; or
R₁ and R₂, taken together with the atoms to which they are attached, form a substituted or unsubstituted carbocyclic ring; or
R₂ and R₃, taken together with the atoms to which they are attached, form a substituted or unsubstituted carbocyclic ring; or
R₃ and R₄, taken together with the atoms to which they are attached, form a substituted or unsubstituted carbocyclic ring;
provided that R₁ is not COOH, and R₁, R₂, R₃, and R₄ are not all H at the same time.

59. The method of claim 58, wherein, when R₁, R₂ and R₄ are hydrogen, R₃ is not N(CH₂CH₃)₂.

60. The method of claim 59, wherein, when X is NO₂, R₂ and R₃ are not OCH₃.

61. The method of any one of claims 52 to 60, wherein the compound is compound 18, or a pharmaceutically acceptable salt thereof.

62. The method of any one of claims 52 to 61, wherein lipid autoxidation is inhibited and/or oxidative stress is reduced in the subject.

63. A method of reducing oxidative stress in a subject, comprising administering to the subject the compound or pharmaceutically acceptable salt as defined in any one of claims 52 to 61.

64. The method of any one of claims 52 to 63, wherein the subject suffers from, or is at risk of developing, a ferroptosis-associated disease or disorder.

65. A method of preventing or treating a ferroptosis-associated disease or disorder in a subject in need thereof, comprising administering to the subject an effective amount of the compound or pharmaceutically acceptable salt as defined in any one of claims 52 to 61.

66. The method of claim 64 or 65, wherein the ferroptosis-associated disease or disorder is stroke, myocardial infarction, diabetes, sepsis, transplant rejection, a neurodegenerative disease, a central nervous system (CNS) injury, an ischemic event, or a cancer.

67. The method of claim 66, wherein the neurodegenerative disease is Alzheimer's disease, Huntington's disease, Parkinson's disease, amyotrophic lateral sclerosis (ALS), HIV-associated dementia, cerebral ischemia, multiple sclerosis, Lewy body disease, Menke's disease, Wilson's disease, Creutzfeldt-Jakob disease, Fahr disease, Friedreich's ataxia, and frontotemporal dementia, amyloidosis, Tay-Sachs disease or periventricular leukomalacia.

68. The method of claim 67, wherein the CNS injury is traumatic brain injury, spinal cord injury, or stroke.

69. The method of claim 66, wherein the ischemic event is cerebral ischemia, ischemic stroke, organ ischemia, myocardial infarction, mesenteric, retinal, hepatic or brain ischemic injury, or ischemic-reperfusion injury.

70. A method for preventing or treating a neurodegenerative disease in a subject in need thereof, comprising administering to the subject an effective amount of the compound or pharmaceutically acceptable salt as defined in any one of claims 52 to 61.

71. The method of claim 70, wherein the neurodegenerative disease is Alzheimer's disease, Huntington's disease, Parkinson's disease, amyotrophic lateral sclerosis (ALS), HIV-associated dementia, cerebral ischemia, multiple sclerosis, Lewy body disease, Menke's disease, Wilson's disease, Creutzfeldt-Jakob disease, Fahr disease, Friedreich's ataxia, and frontotemporal dementia, amyloidosis, Tay-Sachs disease or periventricular leukomalacia.

72. A method for preventing or treating ischemic-reperfusion injury in a subject in need thereof, comprising administering to the subject an effective amount of the compound or pharmaceutically acceptable salt as defined in any one of claims 52 to 61.

73. The method of any one of claims 52 to 72, wherein one or more of the following is inhibited or reduced in the subject: ferroptosis and/or oxytosis; oxidative stress; and lipid autoxidation.

74. The method of any one of claims 52 to 73, wherein said compound or pharmaceutically acceptable salt thereof is administered orally.

75. The method of any one of claims 52 to 73, wherein said compound or pharmaceutically acceptable salt thereof is administered topically and/or locally at the site of an injury.

76. The method of any one of claims 52 to 73, wherein said compound or pharmaceutically acceptable salt thereof is administered parenterally or by injection.

77. The method of claim 76, wherein said compound or pharmaceutically acceptable salt thereof is administered intravenously, intraperitoneally, intramuscularly, or subcutaneously.

78. The method of any one of claims 52 to 73, wherein said compound or pharmaceutically acceptable salt thereof is administered, intranasally, mucosally, or by inhalation.

79. The method of any one of claims 52 to 78, wherein said subject is a human.
